SEQUENCE LISTING (130 sequences)

SEQ ID NO: 1 - RNA, 47 nt, synthetic construct
  source 1..47: mol_type = other RNA
  modified_base 1: mod_base = OTHER; note = Azide modified nucleotide
aaaaacgattcgagaacgtgactgccatgccagctcgtactatcgaa

SEQ ID NO: 2 - RNA, 44 nt, synthetic construct
  source 1..44: mol_type = other RNA
  modified_base 1: mod_base = OTHER; note = Biotin modified nucleotide
cgatagtacgagctggcatggcagtcacgttctcgaatcgtttt

SEQ ID NO: 3 - RNA, 21 nt, Arabidopsis thaliana
  source 1..21: mol_type = genomic RNA
tttggattgaagggagctcta

SEQ ID NO: 4 - RNA, 22 nt, Homo sapiens
  source 1..22: mol_type = genomic RNA
tagcttatcagactgatgttga

SEQ ID NO: 5 - RNA, 22 nt, Homo sapiens
  source 1..22: mol_type = genomic RNA
taatactgccgggtaatgatgg

SEQ ID NO: 6 - RNA, 22 nt, Homo sapiens
  source 1..22: mol_type = genomic RNA
tcgtaccgtgagtaataatgcg

SEQ ID NO: 7 - RNA, 22 nt, Homo sapiens
  source 1..22: mol_type = genomic RNA
cctctgaaattcagttcttcag

SEQ ID NO: 8 - RNA, 21 nt, Homo sapiens
  source 1..21: mol_type = genomic RNA
cattattacttttggtacgcg

SEQ ID NO: 9 - RNA, 22 nt, Homo sapiens
  source 1..22: mol_type = genomic RNA
tccttcattccaccggagtctg

SEQ ID NO: 10 - RNA, 24 nt, Homo sapiens
  source 1..24: mol_type = genomic RNA
tccctgagaccctttaacctgtga

SEQ ID NO: 11 - RNA, 23 nt, Homo sapiens
  source 1..23: mol_type = genomic RNA
taccctgtagaaccgaatttgtg

SEQ ID NO: 12 - RNA, 22 nt, Homo sapiens
  source 1..22: mol_type = genomic RNA
aaaccgttaccattactgagtt

SEQ ID NO: 13 - RNA, 21 nt, Homo sapiens
  source 1..21: mol_type = genomic RNA
cattattacttttggtacgcg

SEQ ID NO: 14 - RNA, 22 nt, Homo sapiens
  source 1..22: mol_type = genomic RNA
tgtaaacatccccgactggaag

SEQ ID NO: 15 - RNA, 22 nt, Homo sapiens
  source 1..22: mol_type = genomic RNA
tgaggtagtagtttgtacagtt

SEQ ID NO: 16 - RNA, 22 nt, Homo sapiens
  source 1..22: mol_type = genomic RNA
tgtaaacatccttgactggaag

SEQ ID NO: 17 - RNA, 22 nt, Homo sapiens
  source 1..22: mol_type = genomic RNA
acctggcatacaatgtagattt

SEQ ID NO: 18 - RNA, 22 nt, Homo sapiens
  source 1..22: mol_type = genomic RNA
tgaggtagtagattgtatagtt

SEQ ID NO: 19 - RNA, 22 nt, Homo sapiens
  source 1..22: mol_type = genomic RNA
tgcctactgagctgaaacacag

SEQ ID NO: 20 - RNA, 22 nt, Homo sapiens
  source 1..22: mol_type = genomic RNA
ttcaagtaatccaggataggct

SEQ ID NO: 21 - RNA, 23 nt, Homo sapiens
  source 1..23: mol_type = genomic RNA
tgaggggcagagagcgagacttt

SEQ ID NO: 22 - RNA, 22 nt, Homo sapiens
  source 1..22: mol_type = genomic RNA
agttcttcagtggcaagcttta

SEQ ID NO: 23 - RNA, 22 nt, Homo sapiens
  source 1..22: mol_type = genomic RNA
gccttctcttcccggttcttcc

SEQ ID NO: 24 - RNA, 22 nt, Homo sapiens
  source 1..22: mol_type = genomic RNA
tgaggtagtaggttgtgtggtt

SEQ ID NO: 25 - RNA, 21 nt, Homo sapiens
  source 1..21: mol_type = genomic RNA
cattattacttttggtacgcg

SEQ ID NO: 26 - RNA, 22 nt, Homo sapiens
  source 1..22: mol_type = genomic RNA
tgaggtagtaggttgtatagtt

SEQ ID NO: 27 - RNA, 25 nt, Homo sapiens
  source 1..25: mol_type = genomic RNA
agtggttcttaacagttcaacagtt

SEQ ID NO: 28 - RNA, 22 nt, Homo sapiens
  source 1..22: mol_type = genomic RNA
tcctgtactgagctgccccgag

SEQ ID NO: 29 - RNA, 22 nt, Homo sapiens
  source 1..22: mol_type = genomic RNA
gggtggggatttgttgcattac

SEQ ID NO: 30 - RNA, 22 nt, Homo sapiens
  source 1..22: mol_type = genomic RNA
actgatttcttttggtgttcag

SEQ ID NO: 31 - RNA, 22 nt, Homo sapiens
  source 1..22: mol_type = genomic RNA
agggcttagctgcttgtgagca

SEQ ID NO: 32 - RNA, 22 nt, Homo sapiens
  source 1..22: mol_type = genomic RNA
cagtggttttaccctatggtag

SEQ ID NO: 33 - RNA, 23 nt, Homo sapiens
  source 1..23: mol_type = genomic RNA
taccctgtagatccgaatttgtg

SEQ ID NO: 34 - RNA, 22 nt, Homo sapiens
  source 1..22: mol_type = genomic RNA
ggggttcctggggatgggattt

SEQ ID NO: 35 - RNA, 22 nt, Homo sapiens
  source 1..22: mol_type = genomic RNA
aaagttctgagacactccgact

SEQ ID NO: 36 - RNA, 23 nt, Homo sapiens
  source 1..23: mol_type = genomic RNA
agcttctttacagtgctgccttg

SEQ ID NO: 37 - RNA, 23 nt, Homo sapiens
  source 1..23: mol_type = genomic RNA
caaagtgctgttcgtgcaggtag

SEQ ID NO: 38 - RNA, 22 nt, Homo sapiens
  source 1..22: mol_type = genomic RNA
tagcagcacgtaaatattggcg

SEQ ID NO: 39 - RNA, 22 nt, Homo sapiens
  source 1..22: mol_type = genomic RNA
tccctgagaccctaacttgtga

SEQ ID NO: 40 - RNA, 23 nt, Homo sapiens
  source 1..23: mol_type = genomic RNA
tgaggggcagagagcgagacttt

SEQ ID NO: 41 - RNA, 23 nt, Homo sapiens
  source 1..23: mol_type = genomic RNA
caacggaatcccaaaagcagctg

SEQ ID NO: 42 - RNA, 22 nt, Homo sapiens
  source 1..22: mol_type = genomic RNA
tgtaaacatcctcgactggaag

SEQ ID NO: 43 - RNA, 22 nt, Homo sapiens
  source 1..22: mol_type = genomic RNA
tgaggtagtagtttgtgctgtt

SEQ ID NO: 44 - RNA, 22 nt, Homo sapiens
  source 1..22: mol_type = genomic RNA
tcggttatcatggtaccgatgc

SEQ ID NO: 45 - RNA, 21 nt, Homo sapiens
  source 1..21: mol_type = genomic RNA
aggcggagacttgggcaattg

SEQ ID NO: 46 - RNA, 22 nt, Homo sapiens
  source 1..22: mol_type = genomic RNA
tcaggctcagtcccctcccgat

SEQ ID NO: 47 - RNA, 23 nt, Homo sapiens
  source 1..23: mol_type = genomic RNA
aacattcaacgctgtcggtgagt

SEQ ID NO: 48 - RNA, 22 nt, Homo sapiens
  source 1..22: mol_type = genomic RNA
ctcctgactccaggtcctgtgt

SEQ ID NO: 49 - RNA, 23 nt, Homo sapiens
  source 1..23: mol_type = genomic RNA
tgtaaacatcctacactctcagc

SEQ ID NO: 50 - RNA, 22 nt, Homo sapiens
  source 1..22: mol_type = genomic RNA
agagcttagctgattggtgaac

SEQ ID NO: 51 - RNA, 22 nt, Homo sapiens
  source 1..22: mol_type = genomic RNA
tggagtgtgacaatggtgtttg

SEQ ID NO: 52 - RNA, 22 nt, Homo sapiens
  source 1..22: mol_type = genomic RNA
agaggtagtaggttgcatagtt

SEQ ID NO: 53 - RNA, 22 nt, Homo sapiens
  source 1..22: mol_type = genomic RNA
agttttgcaggtttgcatttca

SEQ ID NO: 54 - RNA, 21 nt, Homo sapiens
  source 1..21: mol_type = genomic RNA
ttcaagtaattcaggataggt

SEQ ID NO: 55 - RNA, 22 nt, Homo sapiens
  source 1..22: mol_type = genomic RNA
gggagccaggaagtattgatgt

SEQ ID NO: 56 - RNA, 24 nt, Homo sapiens
  source 1..24: mol_type = genomic RNA
tggaagactagtgattttgttgtt

SEQ ID NO: 57 - RNA, 23 nt, Homo sapiens
  source 1..23: mol_type = genomic RNA
agctcggtctgaggcccctcagt

SEQ ID NO: 58 - RNA, 22 nt, Homo sapiens
  source 1..22: mol_type = genomic RNA
caaagaattctccttttgggct

SEQ ID NO: 59 - RNA, 22 nt, Homo sapiens
  source 1..22: mol_type = genomic RNA
tgaggtagtaggttgtgtggtt

SEQ ID NO: 60 - RNA, 22 nt, Homo sapiens
  source 1..22: mol_type = genomic RNA
aacccgtagatccgatcttgtg

SEQ ID NO: 61 - RNA, 22 nt, Homo sapiens
  source 1..22: mol_type = genomic RNA
cacccgtagaaccgaccttgcg

SEQ ID NO: 62 - RNA, 22 nt, Homo sapiens
  source 1..22: mol_type = genomic RNA
tggagtgtgacaatggtgtttg

SEQ ID NO: 63 - RNA, 22 nt, Homo sapiens
  source 1..22: mol_type = genomic RNA
ctcctgactccaggtcctgtgt

SEQ ID NO: 64 - RNA, 22 nt, Homo sapiens
  source 1..22: mol_type = genomic RNA
ctcctgactccaggtcctgtgt

SEQ ID NO: 65 - RNA, 22 nt, Homo sapiens
  source 1..22: mol_type = genomic RNA
tggagagaaaggcagttcctga

SEQ ID NO: 66 - RNA, 21 nt, Homo sapiens
  source 1..21: mol_type = genomic RNA
tatggaggtctctgtctgact

SEQ ID NO: 67 - RNA, 22 nt, Homo sapiens
  source 1..22: mol_type = genomic RNA
tatggcactggtagaattcact

SEQ ID NO: 68 - RNA, 22 nt, Homo sapiens
  source 1..22: mol_type = genomic RNA
tggcagtgtcttagctggttgt

SEQ ID NO: 69 - RNA, 22 nt, Homo sapiens
  source 1..22: mol_type = genomic RNA
aaccgtggctttcgattgttac

SEQ ID NO: 70 - RNA, 23 nt, Homo sapiens
  source 1..23: mol_type = genomic RNA
tggaagactagtgattttgttgt

SEQ ID NO: 71 - RNA, 22 nt, Homo sapiens
  source 1..22: mol_type = genomic RNA
tgaggtagtaggttgtatggtt

SEQ ID NO: 72 - RNA, 23 nt, Homo sapiens
  source 1..23: mol_type = genomic RNA
taaggtgcatctagtgcagatag

SEQ ID NO: 73 - RNA, 21 nt, Homo sapiens
  source 1..21: mol_type = genomic RNA
actctttccctgttgcactac

SEQ ID NO: 74 - RNA, 22 nt, Homo sapiens
  source 1..22: mol_type = genomic RNA
agccactgcccaccgcacactg

SEQ ID NO: 75 - RNA, 23 nt, Homo sapiens
  source 1..23: mol_type = genomic RNA
tgagaactgaattccataggctg

SEQ ID NO: 76 - RNA, 24 nt, Homo sapiens
  source 1..24: mol_type = genomic RNA
gctggtttcatatggtggtttaga

SEQ ID NO: 77 - RNA, 24 nt, Homo sapiens
  source 1..24: mol_type = genomic RNA
gctggtttcatatggtggtttaga

SEQ ID NO: 78 - RNA, 23 nt, Homo sapiens
  source 1..23: mol_type = genomic RNA
taaagtgcttatagtgcaggtag

SEQ ID NO: 79 - RNA, 22 nt, Homo sapiens
  source 1..22: mol_type = genomic RNA
ctttcagtcggatgtttacagc

SEQ ID NO: 80 - RNA, 22 nt, Homo sapiens
  source 1..22: mol_type = genomic RNA
atataatacaacctgctaagtg

SEQ ID NO: 81 - RNA, 25 nt, Homo sapiens
  source 1..25: mol_type = genomic RNA
agtggttcttaacagttcaacagtt

SEQ ID NO: 82 - RNA, 22 nt, Homo sapiens
  source 1..22: mol_type = genomic RNA
ggatatcatcatatactgtaag

SEQ ID NO: 83 - RNA, 22 nt, Homo sapiens
  source 1..22: mol_type = genomic RNA
ttataatacaacctgataagtg

SEQ ID NO: 84 - RNA, 22 nt, Homo sapiens
  source 1..22: mol_type = genomic RNA
ttatggtttgcctgggactgag

SEQ ID NO: 85 - RNA, 22 nt, Homo sapiens
  source 1..22: mol_type = genomic RNA
ggtgcagtgctgcatctctggt

SEQ ID NO: 86 - RNA, 22 nt, Homo sapiens
  source 1..22: mol_type = genomic RNA
ggatatcatcatatactgtaag

SEQ ID NO: 87 - RNA, 22 nt, Homo sapiens
  source 1..22: mol_type = genomic RNA
ctatacgacctgctgcctttct

SEQ ID NO: 88 - RNA, 22 nt, Homo sapiens
  source 1..22: mol_type = genomic RNA
tgtaaacatcctacactcagct

SEQ ID NO: 89 - RNA, 22 nt, Homo sapiens
  source 1..22: mol_type = genomic RNA
aacccgtagatccgaacttgtg

SEQ ID NO: 90 - RNA, 23 nt, Homo sapiens
  source 1..23: mol_type = genomic RNA
cccagtgttcagactacctgttc

SEQ ID NO: 91 - RNA, 21 nt, Homo sapiens
  source 1..21: mol_type = genomic RNA
ctgacctatgaattgacagcc

SEQ ID NO: 92 - RNA, 22 nt, Homo sapiens
  source 1..22: mol_type = genomic RNA
aaagttctgagacactccgact

SEQ ID NO: 93 - RNA, 21 nt, Homo sapiens
  source 1..21: mol_type = genomic RNA
aggggtgctatctgtgattga

SEQ ID NO: 94 - RNA, 23 nt, Homo sapiens
  source 1..23: mol_type = genomic RNA
gcgacgagcccctcgcacaaacc

SEQ ID NO: 95 - RNA, 22 nt, Homo sapiens
  source 1..22: mol_type = genomic RNA
gaatgttgctcggtgaacccct

SEQ ID NO: 96 - RNA, 22 nt, Homo sapiens
  source 1..22: mol_type = genomic RNA
accctgtagaaccgaatttgtg

SEQ ID NO: 97 - RNA, 22 nt, Homo sapiens
  source 1..22: mol_type = genomic RNA
gctcttttcacattgtgctact

SEQ ID NO: 98 - RNA, 21 nt, Homo sapiens
  source 1..21: mol_type = genomic RNA
cattattacttttggtacgcg

SEQ ID NO: 99 - RNA, 22 nt, Homo sapiens
  source 1..22: mol_type = genomic RNA
tctcccaacccttgtaccagtg

SEQ ID NO: 100 - RNA, 24 nt, Homo sapiens
  source 1..24: mol_type = genomic RNA
ttaatgctaatcgtgataggggtt

SEQ ID NO: 101 - RNA, 22 nt, Homo sapiens
  source 1..22: mol_type = genomic RNA
tattgcacattactaagttgca

SEQ ID NO: 102 - RNA, 23 nt, Homo sapiens
  source 1..23: mol_type = genomic RNA
aggcagtgtagttagctgattgc

SEQ ID NO: 103 - RNA, 22 nt, Homo sapiens
  source 1..22: mol_type = genomic RNA
aactggcctacaaagtcccagt

SEQ ID NO: 104 - RNA, 23 nt, Homo sapiens
  source 1..23: mol_type = genomic RNA
tgtgcttgctcgtcccgcccgca

SEQ ID NO: 105 - RNA, 22 nt, Homo sapiens
  source 1..22: mol_type = genomic RNA
acatacttctttatatgcccat

SEQ ID NO: 106 - RNA, 21 nt, Homo sapiens
  source 1..21: mol_type = genomic RNA
gctggtaaaatggaaccaaat

SEQ ID NO: 107 - RNA, 22 nt, Homo sapiens
  source 1..22: mol_type = genomic RNA
tccttcattccaccggagtctg

SEQ ID NO: 108 - skipped

SEQ ID NO: 109 - RNA, 22 nt, Homo sapiens
  source 1..22: mol_type = genomic RNA
catcttccagtgcagtgttgga

SEQ ID NO: 110 - RNA, 21 nt, Homo sapiens
  source 1..21: mol_type = genomic RNA
catcttaccggacagtgctgg

SEQ ID NO: 111 - RNA, 22 nt, Homo sapiens
  source 1..22: mol_type = genomic RNA
catcttactgggcagcattgga

SEQ ID NO: 112 - RNA, 22 nt, Homo sapiens
  source 1..22: mol_type = genomic RNA
ggattcctggaaatactgttct

SEQ ID NO: 113 - RNA, 22 nt, Homo sapiens
  source 1..22: mol_type = genomic RNA
tcctgtactgagctgccccgag

SEQ ID NO: 114 - RNA, 22 nt, Homo sapiens
  source 1..22: mol_type = genomic RNA
tcgtaccgtgagtaataatgcg

SEQ ID NO: 115 - RNA, 23 nt, Homo sapiens
  source 1..23: mol_type = genomic RNA
cgtgtatttgacaagctgagttg

SEQ ID NO: 116 - RNA, 22 nt, Homo sapiens
  source 1..22: mol_type = genomic RNA
aaaccgttaccattactgagtt

SEQ ID NO: 117 - RNA, 22 nt, Homo sapiens
  source 1..22: mol_type = genomic RNA
tcctgtactgagctgccccgag

SEQ ID NO: 118 - RNA, 22 nt, Homo sapiens
  source 1..22: mol_type = genomic RNA
cctctgaaattcagttcttcag

SEQ ID NO: 119 - RNA, 22 nt, Homo sapiens
  source 1..22: mol_type = genomic RNA
ctcagtagccagtgtagatcct

SEQ ID NO: 120 - DNA, 86 bp, synthetic construct
  source 1..86: mol_type = other DNA
  modified_base 1: mod_base = OTHER; note = Azide modified nucleotide
aaaaacgattcgagaacgtgaatctcgttaaccgctcaactggatccccagctcgtactccgcgattcgtgctccgtactccaatc

SEQ ID NO: 121 - DNA, 85 bp, synthetic construct
  source 1..85: mol_type = other DNA
  modified_base 1: mod_base = OTHER; note = Biotin modified nucleotide
cgattggagtacggagcacgaatcgccgagtacgagctggggatccagttgagcggttaacgagattcacgttctcgaatcgttt

SEQ ID NO: 122 - DNA, 86 bp, synthetic construct
  source 1..86: mol_type = other DNA
  modified_base 1: mod_base = OTHER; note = Azide modified nucleotide
aaaaacgattcgagaacgtgaatctcgttaaccgctcaactaagcttccagctcgtactccgcgattcgtgctccgtactccaatc

SEQ ID NO: 123 - DNA, 85 bp, synthetic construct
  source 1..85: mol_type = other DNA
  modified_base 1: mod_base = OTHER; note = Biotin modified nucleotide
cgattggagtacggagcacgaatcgccgagtacgagctggaagcttagttgagcggttaacgagattcacgttctcgaatcgttt

SEQ ID NO: 124 - DNA, 86 bp, synthetic construct
  source 1..86: mol_type = other DNA
  modified_base 1: mod_base = OTHER; note = Azide modified nucleotide
aaaaacgattcgagaacgtgaatctcgttaaccgctcaactactagtccagctcgtactccgcgattcgtgctccgtactccaatc

SEQ ID NO: 125 - DNA, 85 bp, synthetic construct
  source 1..85: mol_type = other DNA
  modified_base 1: mod_base = OTHER; note = Biotin modified nucleotide
cgattggagtacggagcacgaatcgccgagtacgagctggactagtagttgagcggttaacgagattcacgttctcgaatcgttt

SEQ ID NO: 126 - DNA, 22 bp, synthetic construct
  source 1..22: mol_type = other DNA
acgattcgagaacgtgaatctc

SEQ ID NO: 127 - DNA, 21 bp, synthetic construct
  source 1..21: mol_type = other DNA
ttaaccgctcaactggatccc

SEQ ID NO: 128 - DNA, 18 bp, synthetic construct
  source 1..18: mol_type = other DNA
tggagtacggagcacgaa

SEQ ID NO: 129 - DNA, 24 bp, synthetic construct
  source 1..24: mol_type = other DNA
aaccgctcaactaagcttccagct

SEQ ID NO: 130 - DNA, 24 bp, synthetic construct
  source 1..24: mol_type = other DNA
tactagtccagctcgtactccgcg

(19) United States
(12) Patent Application Publication
(10) Pub. No.: US 2024/0410022 A1
(43) Pub. Date: Dec. 12, 2024

(54) COLLECTION, ASSESSMENT AND EARLY DETECTION OF HUMAN LUNG CANCER BIOMARKERS IN EXHALED BREATH CONDENSATES OF MOUSE ANIMAL MODELS

(71) Applicant: HACKENSACK MERIDIAN HEALTH, INC., Edison, NJ (US)

(72) Inventors: Olivier Loudig, Yorktown Heights, NY (US); Megan Irette Mitchell, Clifton, NJ (US)

(73) Assignee: HACKENSACK MERIDIAN HEALTH, INC., Edison, NJ (US)

(21) Appl. No.: 18/777,426

(22) Filed: Jul. 18, 2024

Related U.S. Application Data
(63) Continuation-in-part of application No. 17/560,909, filed on Dec. 23, 2021.
(60) Provisional application No. 63/552,768, filed on Feb. 13, 2024.
(60) Provisional application No. 63/514,300, filed on Jul. 18, 2023.
(60) Provisional application No. 63/130,545, filed on Dec. 24, 2020.

(51) Int. Cl. C12Q 1/6886; A61B 5/097; C12Q 1/6804; C12Q 1/6874

(52) U.S. Cl. CPC C12Q 1/6886; A61B 5/097; C12Q 1/6804; C12Q 1/6874; A61B 2503/40; C12Q 2600/178

(57) ABSTRACT

The present disclosure provides a single mouse exhaled breath collection device and proof-of-concept analyses utilizing an ultra-sensitive and customizable EV purification assay, EV-CATCHER® (Extracellular Vesicle Capture by AnTibody of CHoice and Enzymatic Release) assay. Preliminary studies show that this assay work well for the isolation of exhaled extracellular vesicles (exh-EVs) from exhaled breath condensates (EBC) to capture and characterize miRNAs contained in exh-EVs. Because assembling a large enough cohort of asymptomatic subjects requires time, a complementary approach of using humanized animal models has been chosen to comprehensively assess the robustness and sensitivity of transcriptomic (miRNA and mRNA) and proteomic biomarkers contained in tumor exh-EVs and to enable sensitive detection of human primary and secondary lung cancers during their early development. The results of preliminary analyses demonstrate that the purification of exh-EVs with EV-CATCHER® offers specificity and sensitivity, which will be critical for the targeted purification of tumor exh-EVs from the EBC of transgenic, orthotopic, and Patient-Derived Xenograft (PDX) animal models of human lung cancers.

### CROSS-REFERENCE TO RELATED APPLICATIONS

This application claims the benefit of priority to U.S. provisional application 63/552,768 (filed Feb. 13, 2024, entitled ‘NON-INVASIVE DETECTION OF ORTHOTOPIC HUMAN LUNG TUMORS BY MIRNA EXPRESSION PROFILING OF MOUSE EXHALED BREATH CONDENSATES AND EXHALED EXTRACELLULAR VESICLES, and to U.S. provisional application 63/514,300 (filed Jul. 18, 2023), entitled COLLECTION, ASSESSMENT AND EARLY DETECTION OF HUMAN LUNG CANCER BIOMARKERS IN EXHALED BREATH CONDENSATES OF MOUSE ANIMAL MODELS. It is also a continuation in part of U.S. application Ser. No. 17/560,909 (filed 23 Dec. 2021), which claims priority to provisional application 63/130,545 (filed 24 Dec. 2020), each of which is incorporated by reference herein in its entirety.

### SEQUENCE LISTING

The instant application contains a Sequence Listing which has been submitted electronically in XML format and is hereby incorporated by reference in its entirety. Said XML copy, created on Jul. 18, 2023, is named 129642-01101_SL.xml and is 119,110 bytes in size.

### FIELD OF THE INVENTION

The present disclosure relates to collection, assessment, and early detection of human lung cancer biomarkers in exhaled breath condensates of mouse animal models.

### BACKGROUND OF THE INVENTION

Lung cancer, in men and women, is globally the second leading cause of cancer incidence (2.1 million cases per year) and mortality (1.8 million deaths per year) [1,2]. By 2040, the estimated number of people diagnosed with lung cancer and those who die from it will nearly double (World Health Organization, 2021).

The two main types of lung cancer, non-small-cell lung cancer (NSCLC) (representing 85% of cases) and small-cell-lung cancer (SCLC) (representing 15% to 20%) are identified based on histological, clinical and neuroendocrine characteristics. NSCLC can be further histologically subdivided into adenocarcinoma, squamous carcinoma, large-cell carcinoma (including large-cell neuroendocrine lung cancers), bronchioloalveolar lung cancer, and mixed histologic types (e.g., adenosquamous carcinoma).

Although about 85% of lung cancers are caused by carcinogens present in tobacco smoke, 15% to 25% of lung cancer cases occur in lifetime “never smokers” (defined as fewer than 100 cigarettes in a lifetime). Never-smoking lung cancers represent a disease that is epidemiologically, clinically, and molecularly distinct from smoking lung cancers; it occurs more frequently in women and East Asians, has a peak incidence at a younger age, targets the distal airways, is usually adenocarcinoma, and frequently is epidermal growth factor receptor (EGFR) mutant, and therefore responsive to EGFR-targeted therapies.

Furthermore, more than 50% of newly diagnosed lung cancers in the United States occur in former smokers in whom damage caused by past smoking still led to the development of lung cancer. [Larsen, J E et al. Molecular Basis of Lung Cancer, Chapter 32 in The Molecular Basis of Cancer. Mendelsohn, J., Gray, J W, Howley, P M, Israel, MA and Thompson, C B Eds., (2015) Elsevier Saunders, pp. 475-89].

### Staging of NSCLC

Tumor staging is important for treatment and prognosis, because patients with early-stage lung cancer have a better prognosis than do those with a more advanced stage. [Li, M-Y et al. Molecular Cancer (2021) 20:22]. The staging system most often used for non-small cell lung cancer (NSCLC) is the American Joint Committee on Cancer (AJCC) TNM system, which is based on the size and extent of the main tumor (T); the spread to nearby lymph nodes (N); and the spread (metastasis) to distant sites (M).

Table 1 describes stages 0 to IV of NSCLC (www.cancer.org/cancer/types/lung-cancer/detection-diagnosis-staging/staging-nsclc.html, visited 6.21.2023). The following additional categories are not listed: TO: there is no evidence of a primary tumor; NX: nearby lymph nodes cannot be assessed due to lack of information.

TABLE 1
AJCC Staging for NSCLC

| | Stage Grouping | |
|---|---|---|
| AJCC Stage | (Tumor, Nodes, Metastasis) | Stage Description |
| Occult (hidden) carcinoma | TX, N0, M0 | The main tumor can't be |
| | | assessed for some reason, or |
| | | cancer cells are seen in a |
| | | sample of sputum or other |
| | | lung fluids, but the cancer |
| | | isn't found with other tests, so |
| | | its location can't be |
| | | determined (TX). The cancer |
| | | is not thought to have spread |
| | | to nearby lymph nodes (N0) |
| | | or to distant parts of the body |
| | | (M0). |
| 0 | Tis, N0, M0 | The tumor is found only in |
| | | the top layers of cells lining |
| | | the air passages, but it has not |
| | | invaded deeper into other |
| | | lung tissues (Tis). The cancer |
| | | has not spread to nearby |
| | | lymph nodes (N0) or to |
| | | distant parts of the body |
| | | (M0). |
| IA1 | T1mi, N0, M0 | The cancer is a minimally |
| | | invasive adenocarcinoma. |
| | | The tumor is no larger than 3 |
| | | centimeters (cm) across, and |
| | | the part that has invaded into |
| | | deeper lung tissues is no more |
| | | than ½ cm across. The cancer |
| | | has not spread to nearby |
| | | lymph nodes (N0) or to |
| | | distant parts of the body |
| | | (M0). |
| | T1a, N0, M0 | The tumor is no larger than 1 |
| | | cm across, it has not reached |
| | | the membranes that surround |
| | | the lungs, and it does not |
| | | affect the main branches of |
| | | the bronchi (T1a). The cancer |
| | | has not spread to nearby |
| | | lymph nodes (N0) or to |
| | | distant parts of the body |
| | | (M0). |

OR

| | | |
|---|---|---|
| 1A2 | T1b, N0, M0 | The tumor is larger than 1 cm |
| | | but no larger than 2 cm |
| | | across. It has not reached the |
| | | membranes that surround the |
| | | lungs, and it does not affect |
| | | the main branches of the |
| | | bronchi (T1b). The cancer has |
| | | not spread to nearby lymph |
| | | nodes (N0) or to distant parts |
| | | of the body (M0). |
| 1A3 | T1c, N0, M0 | The tumor is larger than 2 cm |
| | | but no larger than 3 cm |
| | | across. It has not reached the |
| | | membranes that surround the |
| | | lungs, and it does not affect |
| | | the main branches of the |
| | | bronchi (T1c). The cancer has |
| | | not spread to nearby lymph |
| | | nodes (N0) or to distant parts |
| | | of the body (M0). |
| IB | T2a, N0, M0 | The tumor has one or more |
| | | of the following features |
| | | (T2a): |
| | | It is larger than 3 cm but |
| | | not larger than 4 cm |
| | | across. |
| | | It has grown into a main |
| | | bronchus, but is not within |
| | | 2 cm of the carina (the |
| | | point where the windpipe |
| | | splits into the left and |
| | | right main bronchi) and it |
| | | is not larger than 4 cm |
| | | across. |
| | | It has grown into the |
| | | visceral pleura (the |
| | | membranes surrounding |
| | | the lungs) and is not larger |
| | | than 4 cm across. |
| | | It is partially clogging the |
| | | airways (and is not larger |
| | | than 4 cm across). |
| | | The cancer has not spread to |
| | | nearby lymph nodes (N0) or |
| | | to distant parts of the body |
| | | (M0). |
| IIA | T2b, N0, M0 | The tumor has one or more |
| | | of the following features |
| | | (T2b): |
| | | It is larger than 4 cm but |
| | | not larger than 5 cm across. |
| | | It has grown into a main |
| | | bronchus, but is not within |
| | | 2 cm of the carina (the |
| | | point where the windpipe |
| | | splits into the left and right |
| | | main bronchi) and it is |
| | | larger than 4 cm but not |
| | | larger than 5 cm across. |
| | | The tumor has grown into |
| | | the visceral pleura (the |
| | | membranes surrounding |
| | | the lungs) and is larger |
| | | than 4 cm but not larger |
| | | than 5 cm across. |
| | | The tumor is partially |
| | | clogging the airways (and |
| | | is larger than 4 cm but not |
| | | larger than 5 cm across). |
| | | The cancer has not spread to |
| | | nearby lymph nodes (N0) or |
| | | to distant parts of the body |
| | | (M0). |
| | T1a/T1b/T1c | The tumor is no larger than 3 |
| | | cm across, has not grown into |
| | | the membranes that surround |
| | | the lungs, and does not affect |
| | | the main branches of the |
| | | bronchi (T1). It has spread to |
| | | lymph nodes within the lung |
| | | and/or around the area where |
| | | the bronchus enters the lung |
| | | (hilar lymph nodes). These |
| | | lymph nodes are on the same |
| | | side as the cancer (N1). The |
| | | cancer has not spread to |
| | | distant parts of the body |
| | | (M0). |

OR

| | | |
|---|---|---|
| 11B | T2a/T2b, N1, M0 | The tumor has one or more |
| | | of the following features |
| | | (T2): |
| | | It is larger than 3 cm but |
| | | not larger than 5 cm |
| | | across. |
| | | It has grown into a main |
| | | bronchus, but is not within |
| | | 2 cm of the carina (the |
| | | point where the windpipe |
| | | splits into the left and |
| | | right main bronchi) and it |
| | | is not larger than 5 cm |
| | | across. |
| | | It has grown into the |
| | | visceral pleura (the |
| | | membranes surrounding |
| | | the lungs) and is not larger |
| | | than 5 cm. |
| | | It is partially clogging the |
| | | airways (and is not larger |
| | | than 5 cm). |
| | | The cancer has also spread to |
| | | lymph nodes within the lung |
| | | and/or around the area where |
| | | the bronchus enters the lung |
| | | (hilar lymph nodes). These |
| | | lymph nodes are on the same |
| | | side as the cancer (N1). The |
| | | cancer has not spread to |
| | | distant parts of the body |
| | | (M0). |

OR

| | | |
|---|---|---|
| IIB | T3, N0, M0 | The tumor has one or more |
| | | of the following features |
| | | (T3): |
| | | It is larger than 5 cm but |
| | | not larger than 7 cm across. |
| | | It has grown into the chest |
| | | wall, the inner lining of the |
| | | chest wall (parietal pleura), |
| | | the phrenic nerve, or |
| | | membranes of the sac |
| | | surrounding the heart |
| | | (parietal pericardium). |
| | | There are 2 or more |
| | | separate tumor nodules in |
| | | the same lobe of a lung. |
| | | The cancer has not spread to |
| | | nearby lymph nodes (N0) or |
| | | distant parts of the body |
| | | (M0). |
| IIB | T1a/T1b/t1c, N2, M0 | The cancer is no larger than 3 |
| | | cm across, has not grown into |
| | | the membranes that surround |
| | | the lungs, and does not affect |
| | | the main branches of the |
| | | bronchi (T1). The cancer has |
| | | spread to lymph nodes around |
| | | the carina (the point where |
| | | the windpipe splits into the |
| | | left and right bronchi) or in |
| | | the space between the lungs |
| | | (mediastinum). These lymph |
| | | nodes are on the same side as |
| | | the main lung tumor (N2). |
| | | The cancer has not spread to |
| | | distant parts of the body |
| | | (M0). |

OR

| | |
|---|---|
| T2a/T2b, N2, M0 | The tumor has one or more |
| | of the following features |
| | (T2): |
| | It is larger than 3 cm but |
| | not larger than 5 cm across. |
| | It has grown into a main |
| | bronchus, but is not within |
| | 2 cm of the carina (the |
| | point where the windpipe |
| | splits into the left and right |
| | main bronchi) and it is not |
| | larger than 5 cm across. |
| | It has grown into the |
| | visceral pleura (the |
| | membranes surrounding |
| | the lungs) and is not larger |
| | than 5 cm. |
| | It is partially clogging the |
| | airways (and is not larger |
| | than 5 cm). |
| | The cancer has spread to |
| | lymph nodes around the |
| | carina (the point where the |
| | windpipe splits into the left |
| | and right bronchi) or in the |
| | space between the lungs |
| | (mediastinum). These lymph |
| | nodes are on the same side as |
| | the main lung tumor (N2). |
| | The cancer has not spread to |
| | distant parts of the body |
| | (M0). |

OR

| | | |
|---|---|---|
| IIIA | T3, N1, M0 | The tumor has one or more |
| | | of the following features |
| | | (T3): |
| | | It is larger than 5 cm but |
| | | not larger than 7 cm |
| | | across. |
| | | It has grown into the chest |
| | | wall, the inner lining of |
| | | the chest wall (parietal |
| | | pleura), the phrenic nerve, |
| | | or membranes of the sac |
| | | surrounding the heart |
| | | (parietal pericardium). |
| | | There are 2 or more |
| | | separate tumor nodules in |
| | | the same lobe of a lung. |
| | | The cancer has also spread to |
| | | lymph nodes within the lung |
| | | and/or around the area where |
| | | the bronchus enters the lung |
| | | (hilar lymph nodes). These |
| | | lymph nodes are on the same |
| | | side as the cancer (N1). The |
| | | cancer has not spread to |
| | | distant parts of the body |
| | | (M0). |

OR

| | |
|---|---|
| T4, N0 or N1, M0 | The tumor has one or more |
| | of the following features |
| | (T4): |
| | It is larger than 7 cm |
| | across. |
| | It has grown into the space |
| | between the lungs |
| | (mediastinum), the heart, |
| | the large blood vessels |
| | near the heart (such as the |
| | aorta), the windpipe |
| | (trachea), the tube |
| | connecting the throat to the |
| | stomach (esophagus), the |
| | thin muscle separating the |
| | chest from the abdomen |
| | (diaphragm), the backbone, |
| | or the carina. |
| | There are 2 or more |
| | separate tumor nodules in |
| | different lobes of the same |
| | lung. |
| | The cancer may or may not |
| | have spread to lymph nodes |
| | within the lung and/or around |
| | the area where the bronchus |
| | enters the lung (hilar lymph |
| | nodes). Any affected lymph |
| | nodes are on the same side as |
| | the cancer (N0 or N1). The |
| | cancer has not spread to |
| | distant parts of the body |
| | (M0). |
| T1a/T1b/T1c, N3, Mo | The cancer is no larger than |
| | 3 cm across, has not grown |
| | into the membranes that |
| | surround the lungs, and |
| | does not affect the main |
| | branches of the bronchi |
| | (T1). The cancer has spread |
| | to lymph nodes near the |
| | collarbone on either side of |
| | the body, and/or has spread |
| | to hilar or mediastinal |
| | lymph nodes on the other |
| | side of the body from the |
| | main tumor (N3). The |
| | cancer has not spread to |
| | distant parts of the body |
| | (M0). |

OR

| | |
|---|---|
| T2a/T2b, N3, M0 | The tumor has one or more of |
| | the following features (T2): |
| | It is larger than 3 cm but |
| | not larger than 5 cm |
| | across. |
| | It has grown into a main |
| | bronchus, but is not within |
| | 2 cm of the carina (the |
| | point where the windpipe |
| | splits into the left and |
| | right main bronchi) and it |
| | is not larger than 5 cm |
| | across. |
| | It has grown into the |
| | visceral pleura (the |
| | membranes surrounding |
| | the lungs) and is not larger |
| | than 5 cm. |
| | It is partially clogging the |
| | airways (and is not larger |
| | than 5 cm). |
| | The cancer has spread to |
| | lymph nodes near the |
| | collarbone on either side of |
| | the body, and/or has spread |
| | to hilar or mediastinal |
| | lymph nodes on the other |
| | side of the body from the |
| | main tumor (N3). The |
| | cancer has not spread to |
| | distant parts of the body |
| | (M0). |

OR

| | | |
|---|---|---|
| IIIB | T3, N2, M0 | [symbol] he tumor has one or more of |
| | | [symbol] he following features (T3): |
| | | It is larger than 5 cm but |
| | | not larger than 7 cm |
| | | across. |
| | | It has grown into the chest |
| | | wall, the inner lining of |
| | | the chest wall (parietal |
| | | pleura), the phrenic nerve, |
| | | or membranes of the sac |
| | | surrounding the heart |
| | | (parietal pericardium). |
| | | There are 2 or more |
| | | separate tumor nodules in |
| | | the same lobe of a lung. |
| | | The cancer has spread to |
| | | lymph nodes around the |
| | | carina (the point where the |
| | | windpipe splits into the left |
| | | and right bronchi) or in the |
| | | space between the lungs |
| | | (mediastinum). These |
| | | lymph nodes are on the |
| | | same side as the main lung |
| | | tumor (N2). The cancer has |
| | | not spread to distant parts of |
| | | the body (M0). |

OR

| | |
|---|---|
| T4, N2, M0 | [symbol] he tumor has one or more of |
| | [symbol] he following features (T4): |
| | It is larger than 7 cm |
| | across. |
| | It has grown into the space |
| | between the lungs |
| | (mediastinum), the heart, |
| | the large blood vessels near |
| | the heart (such as the |
| | aorta), the windpipe |
| | (trachea), the tube |
| | connecting the throat to the |
| | stomach (esophagus), the |
| | thin muscle separating the |
| | chest from the abdomen |
| | (diaphragm), the backbone, |
| | or the carina (the point |
| | where the windpipe splits |
| | into the left and right main |
| | bronchi). |
| | There are 2 or more |
| | separate tumor nodules in |
| | different lobes of the same |
| | lung. |
| | The cancer has spread to |
| | lymph nodes around the |
| | carina (the point where the |
| | windpipe splits into the left |
| | and right bronchi) or in the |
| | space between the lungs |
| | (mediastinum). These |
| | lymph nodes are on the |
| | same side as the main lung |
| | tumor (N2). The cancer has |
| | not spread to distant parts of |
| | the body (M0). |
| T3, N3, M0 | The tumor has one or more of |
| | he following features (T3): |
| | It is larger than 5 cm but |
| | not larger than 7 cm |
| | across. |
| | It has grown into the |
| | chest wall, the inner |
| | lining of the chest wall |
| | (parietal pleura), the |
| | phrenic nerve, or |
| | membranes of the sac |
| | surrounding the heart |
| | (parietal pericardium). |
| | There are 2 or more |
| | separate tumor nodules |
| | in the same lobe of a |
| | lung. |
| | The cancer has spread to |
| | lymph nodes near the |
| | collarbone on either side of |
| | the body, and/or has spread |
| | to hilar or mediastinal |
| | lymph nodes on the other |
| | side of the body from the |
| | main tumor (N3). The |
| | cancer has not spread to |
| | distant parts of the body |
| | (M0) |

OR

| | | |
|---|---|---|
| IIIC | T4, N3, M0 | [symbol] he tumor has one or more of |
| | | [symbol] he following features (T4): |
| | | It is larger than 7 cm |
| | | across. |
| | | It has grown into the space |
| | | between the lungs |
| | | (mediastinum), the heart, |
| | | the large blood vessels |
| | | near the heart (such as the |
| | | aorta), the windpipe |
| | | (trachea), the tube |
| | | connecting the throat to |
| | | the stomach (esophagus), |
| | | the thin muscle separating |
| | | the chest from the |
| | | abdomen (diaphragm), the |
| | | backbone (spine), or the |
| | | carina (the point where the |
| | | windpipe splits into the |
| | | left and right main |
| | | bronchi). |
| | | There are 2 or more |
| | | separate tumor nodules in |
| | | different lobes of the same |
| | | lung. |
| | | The cancer has spread to |
| | | lymph nodes near the |
| | | collarbone on either side of |
| | | the body, and/or has spread |
| | | to hilar or mediastinal |
| | | lymph nodes on the other |
| | | side of the body from the |
| | | main tumor (N3). The |
| | | cancer has not spread to |
| | | distant parts of the body |
| | | (M0). |
| IVA | Any T, Any N, M1a | [symbol] he cancer can be any size and |
| | | [symbol] ay or may not have grown |
| | | [symbol] nto nearby structures (any T). |
| | | [symbol] t may or may not have reached |
| | | [symbol] earby lymph nodes (any N). In |
| | | [symbol] ddition, any of the following is |
| | | [symbol] rue (M1a): |
| | | The cancer has spread to |
| | | the other lung. |
| | | Cancer cells are found in |
| | | the fluid around the lung |
| | | (called a malignant pleural |
| | | effusion). |
| | | Cancer cells are found in |
| | | the fluid around the heart |
| | | (called a malignant |
| | | pericardial effusion). |

OR

| | | |
|---|---|---|
| | Any T, Any N M1b | The cancer can be any size |
| | | and may or may not have |
| | | grown into nearby |
| | | structures (any T). It may or |
| | | may not have reached |
| | | nearby lymph nodes (any |
| | | N). It has spread as a single |
| | | tumor outside of the chest, |
| | | such as to a distant lymph |
| | | node or an organ such as the |
| | | liver, bones, or brain (M1b). |
| IVB | Any T, Any N, M1c | The cancer can be any size |
| | | and may or may not have |
| | | grown into nearby |
| | | structures (any T). It may or |
| | | may not have reached |
| | | nearby lymph nodes (any |
| | | N). It has spread as more |
| | | than one tumor outside the |
| | | chest, such as to distant |
| | | lymph nodes and/or to other |
| | | organs such as the liver, |
| | | bones, or brain (M1c). |

[symbol]

indicates data missing or illegible when filed

Unfortunately, the lung is also a major target for secondary cancers metastasizing from breast, colorectal, urologic (bladder, kidney, and prostate), melanoma, and other primary sites [14], and the 5-year survival for patients with lung metastases is very low (21% for patients with a primary breast cancer and ≤10% for patients with a primary colorectal cancer [15-17]). A major challenge with early detection of lung-colonizing cells (micrometastases) is that early invading cells are spread out in the tissue and cannot be individually detected by current imaging modalities (X-ray or Computed Tomography (CT)) [18]. Compared to primary lung cancer, only early interventions (metastasectomy, radiation therapy, and chemotherapy) have been successful at improving secondary lung cancer patient outcome, thus underscoring a critical need for development of more sensitive detection assays [19,20].

To complement lung cancer imaging technologies and address detection gaps, molecular assays are being designed for early identification of genomic and epigenomic lung tissue alterations by detection of circulating tumor cells (CTCs), circulating tumor DNA (CtDNA), circulating transcripts (circular RNAs, mRNAs, microRNAs (miRNAs), long non-coding RNAs), and more recently biomarkers (DNA, RNAs, proteins) packaged in circulating extracellular vesicles [21-24].

Lung cancer is a heterogeneous disease clinically, biologically, histologically and medically.

Genome instability is an underlying enabling characteristic of lung cancer cells where alterations such as chromosomal rearrangements can generate rare genetic events that eventually give rise to cancer.

The National Cancer Institute's The Cancer Genome Atlas (TCGA) project has revealed regions of significant copy number alterations in lung squamous cell carcinomas (SCCs). These include regions of copy number alteration containing SOX2, PDGFRA/KT, EGFR, FGFR1/WHSCIL1, CCND1, and CDKN2A genes and amplifications containing NFE2L2, MYC, CDK6, MDM2, BCL2L1, and EYS, and deletions of FOXP1, PTEN, and NF1. [Hammerman, P S et al. Nature (2012) 489 (7417): 519-25].

Whole genome genomic approaches have yielded further insight into the complexities of the lung cancer genome with the identification of driver mutations (EGFR, KRAS, and EML4-ALK] resulting in oncogene addiction, in which the cell becomes dependent on the aberrant oncogene signaling for survival, and key signaling pathways including RAS/RAF/MAPK, P13K/AKT/mTOR, p53 and p16/RB). Commonly inactivated tumor suppressor genes (TSGs) in lung cancer include TP53, RB1, STK11, CDKN2A, FHIT, RASSFIA and PTEN. [Larsen, J E et al. Molecular Basis of Lung Cancer, Chapter 32 in Molecular Basis of Cancer, 4^{th} Ed. Mendelsohn, Gray, Howley, Israel and Thompson, Eds. Elsevier Saunders (2015), pp. 475-89].

Table 2 shows significantly mutated genes in lung cancer subtypes identified by exome sequencing. [Taken from Table 32-3 in Larsen, J E et al. Molecular Basis of Lung Cancer, Chapter 32 in Molecular Basis of Cancer, 4^{th} Ed. Mendelsohn, Gray, Howley, Israel and Thompson, Eds. Elsevier Saunders (2015), pp. 475-89]. Genes in bold are present in more than one histological subtype. The data was generated through analysis of 183 lung adenocarcinomas [Imielinski, M. et al. Cell (2012) 150:1107-20], TCGA project of 178 previously untreated, stage I-IV primary lung squamous cell carcinoma [Hammerman, P S et al. Nature (2012) 489 (7417) 519-25], and 34 primary SCLC tumors and 17 SCLC cell lines [Rudin, C M et al. Nat. Genet. (2012) 44:1111-165].

TABLE 2
Significantly mutated genes in lung cancer
subtypes identified by exome sequencing.

Non-Small Cell Lung Carcinoma

| | Squamous-Cell | |
|---|---|---|
| Adenocarcinoma (AD | Carcinoma (SCC) | SCLC |
| ARID1A | ANP32C | ADCY1 |
| ATM | APC | BCLAF1 |
| BRAF | BCL11A | C17orf108 |
| BRD3 | BCL2L1 | CDYL |
| CBL | BRAF | CNTNAP2 |
| CTNNB1 | CDK6 | COL22A1 |
| EGFR | CDKN2A | COL4A2 |
| FBXW7 | CREBBP | DIP2C |
| FGFR3 | CSMD1 | ELAVL2 |
| GOPC | DDR2 | GRIK3 |
| KEAP1 | EGFR | GRM8 |
| KIAA0427 | EYS | KHSRP |
| KRAS | FAM123B | KIF21A |
| NF1 | FBXW7 | PLSCR4 |
| PIK3CA | FGFR1 | RASSF8 |
| PPP2R1A | FOXP1 | RB1 |
| PTEN | HLA-A | RIMS2 |
| RB1 | HRAS | RUNX1T1 |
| RBM10 | KEAP1 | SATB2 |
| SETD2 | MLL2 | TMEM132D |
| SMAD4 | MUC16 | TP53 |
| SMARCA4 | NF1 | ZDBF2 |
| STL11 | NFE2L2 | |
| TP53 | NOTCH1 | |
| U2AF1 | PIK3CA | |
| | PTEN | |
| | RB1 | |
| | REL | |
| | SMAD4 | |
| | SMARCA4 | |
| | TNFAIP3 | |
| | TP53 | |
| | TSC1 | |
| | VGLL4 | |
| | WHSC1L1 | |
| | WWOX | |

microRNA-Mediated Regulation

miRNAs are a class of non-protein-encoding small RNAs capable of regulating gene expression by cither directly cleaving a targeted mRNA or inhibiting translation by interacting with the 3′ untranslated region (UTR) of a target mRNA. A single miRNA often targets multiple genes and multiple miRNAs may target the same mRNA, which results in a complex network of molecular pathways where a single miRNA can potentially affect multiple processes. Aberrant expression of miRNAs has been found to play an important role in the pathogenesis of lung cancer as either oncogenes or tumor suppressor genes.

Table 3 summarizes some experimentally validated miRNAs important in lung cancer. [Taken from Table 32-5 in Larsen, J E et al. Molecular Basis of Lung Cancer, Chapter 32 in Molecular Basis of Cancer, 4^{th} Ed. Mendelsohn, Gray, Howley, Israel and Thompson, Eds. Elsevier Saunders (2015), pp. 475-89].

TABLE 3
Some experimentally validated miRNAs important in lung cancer

| | | Correlation | |
|---|---|---|---|
| | | with poor | |
| | Expression | prognosis | |
| | in lung | and/or | |
| miRNA | cancer | predictive role | Validated Targets |
| Tumor-promoting | | | |
| miRNAs | | | |
| miR-17-92 cluster | Up | | PTEN, E2F1-3, |
| | | | BIM |
| miR-21 | Up | Positive^{b} | PTEN, SPRY1, |
| | | | PDCD4, |
| | | | RALGDS |
| miR-155 | Up^{a} | Positive | CK1α, TP53/ |
| | | | NP1, MMR |
| miR0221/222 | Up | Positive^{c} | PTEN, TIMP3, |
| | | | CKIT, P27Kip1 |
| Tumor-Suppressing | | | |
| miRNAs | | | |
| Let-7/miR-98 | Down | Negative | RAS, MYC, |
| | | | HMGA2, |
| | | | CDC25A, CDK6, |
| | | | CCND1 |
| miR-15/16 | Down | | BCL1, MCL1, |
| | | | CCND1, WNT3A |
| miR-29 | Down | | DNMT3A-3B, |
| | | | MCL1 |
| miR-34a-v | Down | Negative | SIRT1, BCL2, |
| | | | CD44, CDK4/6, |
| | | | CCNE2, MYC, |
| | | | E2F3 |
| miR-128b | Down | Negative | EGFR |
| | | (gefitinib | |
| | | treated) | |
| miR-200 family | Down | | ZEB1/ZEB2 |

^{a}up in KRAS and EFFR wt tumors
^{b}resistance to EGFR targeted therapy
^{c}resistance to TRAIL treatment

Blood-based biomarkers generally have insufficient sensitivity to enable early detection of primary and secondary lung cancers [25]. Thus, to improve early detection of lung tumors, direct airway sample collections (i.e., nasal epithelial brushing, sputum, bronchial brushing, and bronchioalveolar lavage (BAL)) have been evaluated by high throughput analyses of miRNAs, mRNAs, proteins, DNA mutations and methylation patterns [26-33]. Although bronchoscopy with BAL has become the most reliable method for collection and evaluation of lining fluid from the lower respiratory tract, the invasive nature of this procedure has limited its implementation as a screening approach for high-risk individuals. Thus, it has led to an intense search for less intrusive airway sampling to gain direct access to more detectable lung cancer biomarkers.

Exhaled breath, in particular, carries vapors that are released by the lungs into the external environment during normal tidal respiration and that can be condensed into a biofluid. Studies have shown that Exhaled Breath Condensate (EBC) contains metabolites (e.g., nitrite, urea, amino acid) and macromolecules (i.e., proteins, RNA, DNA) that become aerosolized within droplets evaporating from the lining fluid of the lower respiratory tract [34,35]. The condensation of exhaled aqueous droplets into EBC can be done non-invasively by use of commercially available FDA-approved disposable or reusable collection devices [36,37]. The growing field of breathomics has been focused on quantifying exhaled compounds and correlating their contents with the physiology and pathology of the lung [38-41]. That EBC contains exhaled extracellular vesicles (exh-EVs), whose microRNA (miRNA) contents confirms their deep lung tissue origin and suggests that they contain lung cancer biomarkers has recently been determined (see Example 1 below “Preliminary studies”, a1-a5).

Multiple studies have demonstrated that the deregulation of miRNA expression can be directly associated with the initiation and development of lung cancers [48-50]. It has been shown that miRNA expression changes in lung tumors can also provide important prognostic information [51-53]. Recent studies have also evaluated the potential of circulating free miRNAs for non-invasive detection and prognostication of lung cancers [54-56]. However, inconsistencies between the miRNA signatures of these studies have highlighted major technical issues with the global acquisition and processing of free circulating miRNAs [57]. Thus, in a refined approach, studies have examined miRNAs contained within circulating extracellular vesicles (EVs), because they are protected by a membrane and maintain the expression ratios acquired from the cell-of-origin [58,59]. A subtype of EVs, often referred to as exosomes, are robust phospholipid bi-layered nanosized particles (ranging from about 50 nm to about 150 nm in diameter), which are released by virtually all human cells and can mediate long-distance intercellular communication via the delivery of a variety of pre-packaged functional biomolecules [60,61]. Importantly, EVs can diffuse into tissues and circulate in any biofluid, and the analysis of their multi-omic cargoes can provide a snapshot of potentially unique biomarkers from their cell-of-origin [62,63]. Many studies have shown that tumor-derived EVs play key roles in cancer progression and metastasis [64]. For example, functional miRNA studies have demonstrated that EVs produced by primary lung tumor cells transport and deliver miRNA cargoes that can modulate angiogenesis [65], cellular proliferation [66], immune response of recipient cells, and educate distal pre-metastatic lung niche cells to promote the uptake of CTCs [68]. Cancer EVs contain mRNA fragments as well as intact mRNAs that can be translated into functional proteins to regulate target cells [69,70].

To date, no studies have evaluated the biomarker potential of mRNAs contained in tumor exh-EVs.

In addition to containing functional nucleic acids, studies have also shown that EVs circulating in the blood of lung cancer patients contain unique and functional proteins with tremendous potential for the diagnosis of lung cancer [71-73]. Using on optimized procedure for mass-spectrometric analysis of EVs, the discriminatory potential of EV proteomics for identifying protein biomarkers with metastatic regulatory properties has been demonstrated [74,75].

No studies have thus far evaluated exh-EV proteomics to improve the targeted antibody-purification of human lung tumor exh-EVs from exhaled breath condensate (EBC) and to enable sensitive early detection of lung cancer.

Despite broad agreement on the importance of biomarker testing for patients with lung cancer, there is variable uptake in clinical practice. KRAS mutations are the most common oncogenic mutations in lung adenocarcinoma; testing for EGFR mutation in the United States, which has been part of standard clinical practice since 2011, is not always assessed, although testing rates have improved over time. Outside the United States, testing is even more variable, with data limited to only a few countries and no central data available, making extrapolations based on limited reports difficult [Pennell, N A et al. Am. Socy Clinical Oncology Educational Book (2019): doi.org/10.1200/EDBK_237863]. The lack of universal testing has been attributed, among other things, to tests failure for technical reasons, tissue samples from biopsies often being sufficient for diagnosis but inadequate for biomarker testing [Id., citing Gutierrez, M E et al. Clin. Lung Cancer (2017) 18:651-9; Ferry-Galow, K V et al. J. Oncol. Pract. (2018) 14: e722-8]; the need to optimize tissue handling after biopsy to maximize available material for molecular studies, and the variability of information provided by assays and analytical sensitivity (limit of detection), specificity, comprehensiveness/diagnostic sensitivity, tissue requirements and turnaround time.

The present disclosure provides proof-of-concept analyses utlizing our ultra-sensitive and customizable EV purification assay, EV-CATCHER® (Extracellular Vesicle Capture by AnTibody of CHoice and Enzymatic Release) assay, which has already been demonstrated to work well for the isolation of exh-EVs from exhaled breath condensates (EBC) to capture and characterize miRNAs contained in exh-EVs. A recent miRNA study of human EBC demonstrates their potential for detection of lung cancer [42-47].

Although a small number of studies have suggested that exhaled miRNAs contain biomarkers for detection of lung cancer [76-79], including our recent study [47], none have evaluated the targeted capture of lung tumor exh-EVs from whole EBC for a direct evaluation of their multi-omic biomarkers. Because assembling a large enough cohort of asymptomatic subjects is requiring time, a complementary approach of using humanized animal models has been chosen to comprehensively assess the robustness and sensitivity of transcriptomic (miRNA and mRNA) and proteomic biomarkers contained in tumor exh-EVs and to enable sensitive detection of human primary and secondary lung cancers during their early development. Preliminary analyses described in Example 1, subsections b1 and b2 below, demonstrate that the purification of exh-EVs with EV-CATCHER®, which will be critical for the targeted purification of tumor exh-EVs from the EBC of our transgenic, orthotopic, and Patient-Derived Xenograft (PDX) animal models of human lung cancers, offers both specificity and sensitivity.

### SUMMARY OF THE INVENTION

According to one aspect, the present disclosure provides a non-invasive method for targeted capture of a purified population of extracellular vesicles (EVs) derived from lung cancer cells exhaled breath obtained from a subject at risk for lung cancer for evaluating a cargo of the purified population of EVs comprising: a) obtaining an expressed breath sample from the subject; b) condensing the exhaled breath sample in a cooling chamber and collecting the exhaled breath condensate (EBC); c) preparing a purified population of EVs by contacting the exhaled breath condensate comprising EVs from the subject with a binding agent directed to one or more EV surface antigen, wherein the binding agent is linked to a nucleic acid, and wherein the nucleic acid is immobilized on a solid support; d) isolating the EV bound by the binding agent from the exhaled breath condensate; c) releasing the EV bound to the binding agent; f) eluting the bound EV from the binding agent to form a population of free purified EVs; and g) evaluating cargo and surface molecules comprising protein, nucleic acids or lipids of the purified population of EVs.

According to some embodiments of the non-invasive method, the subject is a primary lung tumor bearing animal model wherein the lung tumor is a human tumor comprising mutations including EGFR, KRAS, p53, Ret, Her2, ROS1, Met, BRAF, NRAS, ALK, MAP2K1 or PI3KCA mutations and control animal colonies are established with primary human small airway epithelial cells (HSAECs); or the animal model is a transgenic mouse model, and human tumor cells comprising mutations including EGFR, KRAS, p53, Ret, Her2, ROS1, Met, BRAF, NRAS, ALK, MAP2K1 or PI3KCA mutations and a bioluminescent construct are instilled through the trachea of the animals; the animal model is a TA-CCSP transgenic model, wherein the CCSP promoter is active in Clara cells, in alveolar type II cells or both; or the animal model is an orthotopic human tumor NOD/SCID mouse model bearing a human lung tumor comprising mutations including EGFR, KRAS, p53, Ret, Her2, ROS1, Met, BRAF, NRAS, ALK, MAP2K1 or PI3KCA mutations, and wherein human NSCLC lung cancer cell lines are transduced for ex vivo bioluminescence, expanded in vitro and administered into the lungs of the NOD/SCID nude mice by tracheal instillation for lung uptake, or the animal model is a PDX human tumor NOD/SCID mouse model established using NSCLC patient-derived tumor cells comprising mutations including EGFR, KRAS, p53, Ret, Her2, ROS1, Met, BRAF, NRAS, ALK, MAP2K1 or PI3KCA mutations, and wherein the transgenic model, the orthotopic model and the PDX model are complementary.

According to some embodiments of the non-invasive method, the transgenic mouse is transduced to express EGFRL858R protein in bronchiolar Clara cells; or the transgenic mouse is transfected to express KRASG12D protein in Clara cells and alveolar type II cells; or the transgenic mouse is transfected to express CC10 protein for the study of multifocal bronchioloalveolar hyperplasias which develop into mixed solid and papillary adenocarcinomas, adenocarcinomas with focal NE differentiation, epithelial cell hyperplasia and adenomatous hyperplasia and bronchogenic adenocarcinomas; or the transgenic mouse is transfected to express SP-C protein for the study of bronchioloalveolar adenomas and adenocarcinomas; the transgenic mouse is transfected to express Trp5 for the study of adenocarcinomas, NE hyperplasia and small-cell carcinoma with metastases; or the transgenic mouse is transfected to express Rb for the study of NE hyperplasia and small-cell carcinoma with metastases.

According to some embodiments of the non-invasive method, step (g) evaluating cargo and surface molecules further comprises one or more of: (i) identifying proteins specific to a surface of the biological particles; or (ii) identifying protein cargos; or (iii) identifying DNA molecules; or (iv) extracting RNA from the purified population of EVs, and identifying and quantifying expression of small non-coding RNAs comprising microRNAs (miRNAs) encapsulated by the purified population of EVs. According to some embodiments, the proteins are identified by mass spectrometry; the DNA is identified by sequencing or quantitative PCR; and the RNA is identified by digital drop PCR.

According to some embodiments, the noninvasive method comprises an initial ultrafiltration or ultracentrifugation step to provide a starting pooled heterogeneous population of EVs. According to some embodiments, the binding agent that binds to one or more EV surface antigen is an antibody, an antibody binding fragment, or an aptamer. According to some embodiments, (a) the aptamer comprises two complementary primers including 5′-Azide (5′Az-AAAAACGAUUCGAGAACGUGACUGCCAUGCCAGCUCGUACUAU CGAA (SEQ ID NO: 1)) and 3′-Biotin (5′Bio-CGAUAGUACGAGCUGGCAUGGCAGUCACGUUCUCGAA UCGUUUU (SEQ ID NO: 2)); or
(b) the aptomer comprises two complementary primers containing specific restriction enzyme recognition sites used for EV-CATCHER including: BamHI: 5′-Azide (5′Az-AAAAACGATTCGAGAACGTGAATCTCGTTAACCGCTCAACTGGATCCCCAGCTCGTA CTCCGCGATTCGTGCTCCGTACTCCAATC (SEQ ID NO: 120)) and

BamHI: 3'-Biotin (5'Bio-
CGATTGGAGTACGGAGCACGAATCGCCGAGTACGAGCTGGGGATCCAGT
TGAGCGGTTAACGAGATTCACGTTCTCGAATCGTTT (SEQ ID NO:
121)); and
HindIII: 5'-Azide
(5'Az- AAAAACGATTCGAGAACGTGAATCTCGTTAACCGCTCAACTA
AGCTTCCAGCTCGTACTCCGCGATTCGTGCTCCGTACTCCAATC (SEQ
ID NO: 122));
HindIII: 3'-Biotin (5'Bio-
CGATTGGAGTACGGAGCACGAATCGCCGAGTACGAGCTGGAAGCTTAGT
TGAGCGGTTAACGAGATTCACGTTCTCGAATCGTTT (SEQ ID NO:
123)); or
Spel: 5'-Azide
(5'Az- AAAAACGATTCGAGAACGTGAATCTCGTTAACCGCTCAACTA
CTAGTCCAGCTCGTACTCCGCGATTCGTGCTCCGTACTCCAATC (SEQ
ID NO: 124)) and
Spel: 3'-Biotin (5'Bio-
CGATTGGAGTACGGAGCACGAATCGCCGAGTACGAGCTGGACTAGTAGT
TGAGCGGTTAACGAGATTCACGTTCTCGAATCGTTT
(SEQ ID NO: 125)).

According to some embodiments, the EVsurface antigen comprises CD9, CD63, CD81, CD37, CD82, Alix, Tim4, PLAP, Adiponectin, FABP4, Caveolin-1, Cytokeratins, EPCAM, E-Cadherin, P63, a heterologous cell surface polypeptide, a cell surface marker inherited by the EVs, club cell secretory protein (CCSP), an SFTPC-encoded surface protein or a variant thereof. According to some embodiments the EV surface antigen is specific to Clara cells or AT2 respiratory cells. According to some embodiments, the EV surface antigen is a club cell secretory protein (CCSP) variant or surfactant protein C (SP-C) variant encoded by the SFTPC gene.

According to some embodiments, the nucleic acid comprises DNA, RNA, or a combination thereof. According to some embodiments, the nucleic acid comprises non-natural nucleotides. According to some embodiments, the nucleic acid comprises DNA. According to some embodiments, the DNA comprises a restriction enzyme recognition site. According to some embodiments, the DNA comprises one or more ribonucleic acid nucleotide. According to some embodiments, the one or more ribonucleic acid nucleotide is uracil. According to some embodiments, the nucleic acid further comprises a binding moiety on a first end of the nucleic acid and a binding moiety on a second end of the nucleic acid, and wherein the binding moiety on the first end of the nucleic acid and the binding moiety on the second end of the nucleic acid are different. According to some embodiments, the binding moiety on the first end of the nucleic acid is an avidin, streptavidin or carboxyl binding moiety. According to some embodiments, the binding moiety is biotin. According to some embodiments, the binding moiety on the second end of the nucleic acid is an amine moiety. According to some embodiments, the amine moiety is azide.

According to some embodiments, the binding agent to one or more EV surface antigens comprises a dibenzocyclooctyne (DBCO) molecule, 2-IT (2-iminothiolane), MBS (3-maleimidobenzoic acid N-hydroxysuccinimide ester), SPDP (N-succinimidyl 3-(2-pyridyldithio) propionate), SATA (N-succinimidyl S-acetylthioacetate), SMCC (succinimidyl 4-(N-maleimidomethyl) cyclohexane-1-carboxylate), Sulfo-SMCC, or derivatives thereof.

According to some embodiment,s the solid support is a well plate, polymer, or a surface.

According to some embodiments, releasing the isolated EV comprises: (i) enzymatically cleaving the nucleic acid; or (ii) displacing a first strand of the nucleic acids connected to the antibody from the second strand of the nucleic acids connected to the support by strand displacement with a complementary nucleic acid to the first or second strand of the nucleic acid and an enzyme having strand displacement activity to release the antibody from the support; or (iii) separating the annealed DNA strands to allow release of the antibody from the platform without damaging the DNA strand attached to the antibody by a polymerase chain reaction using an oligonucleotide complementary to the region of the DNA attached to the antibody.

According to some embodiments, the enzymatic cleaving is with uracil glycosylase; or the enzymatic cleaving is with a restriction enzyme; or the enzyme having strand displacement activity is DNA polymerase, topoisomerase, or helicase.

According to some embodiments the non-invasive method comprises detecting, identifying and measuring a level of mRNA or the one or more small non-coding RNAs comprising miRNAs encapsulated in the EVs by next generation sequencing. According to some embodiments, the miRNA encapsulated in the EVs is one or more miRNA listed in Table 5 or in Tables 8-11.

According to another aspect, the present disclosure provides a non-invasive method for optimizing therapeutic benefit for a subject at risk of lung cancer, comprising a) obtaining an exhaled breath sample from the subject and from a healthy control; b) condensing the exhaled breath in a cooling chamber and collecting the exhaled breath condensate (EBC); c) purifying EVs derived from the exhaled breath sample contained in the EBC obtained from the subject and the healthy control; d) measuring a level of expression of each of a plurality of mRNAs, miRNAs or protein cargo in the EVs contained in the EBC sample from the subject and in the EVs contained in the EBC sample from the healthy control; e) determining that expression of the one or more of the mRNAs, miRNAs or protein cargo in the EVs contained in the EBC sample from the subject is dysregulated compared to the healthy control; f) identifying the patient as one that can benefit therapeutically from being treated for lung cancer, when the presence of one or more dysregulated mRNAs, miRNAs or protein cargo in the EVs contained in the EBC sample obtained from the subject is detected; wherein the detection may correlate with tumor burden; and g) tailoring an effective medical treatment for the lung cancer based on genetic, environmental and lifestyle factors of the subject and based on the detection in (f).

According to some embodiments, the noninvasive method further comprises: (h) monitoring the lung cancer response or resistance to the treatment in (g) by obtaining exhaled breath samples comprising EVs from the subject over time; and (i) adjusting the medical treatment as needed to improve clinical outcome.

According to some embodiments, the miRNA encapsulated by the EVs is one or more miRNA listed in Table 5 or in tables 8-11. According to some embodiments, the one or more miRNAs is downregulated compared to the healthy control. According to some embodiments the one or more miRNAs is upregulated compared to the healthy control.

According to some embodiments, a) the subject is a mammalian subject; or b) the subject is a human subject.

According to some embodiments, the detecting in (e) is earlier than detecting of lung cancer by imaging thresholds.

According to some embodiments, the noninvasive method further comprises detecting a level of expression of a protein in the EVs from the EBC sample and determining that expression of the protein is dysregulated compared to the healthy control, wherein the protein includes α-enolase, vimentin, brain abundant membrane attached signal protein 1 (BASP1), aldolase (ALDOA), calreticulin (CALR), proteasome activator subunit 1 (PSME1), proteasome activator subunit (2), major histocompatibility complex class 1C (HLA-C) or glucose 6-phosphate dehydrogenase (G6PD), SH3 domain-containing protein 21, Arf-GAP with SH3 domain, ANK repeat and PH domain-containing protein 2, Histone H4, Vimentin, AHNAK nucleoprotein (desmoyokin), Heat shock protein HSP 90-beta, Annexin A5, Protein S100-A4, Heat shock protein HSP 90-alpha, Alpha-cnolase, High mobility group protein HMG-I/HMG-Y, 14-3-3 protein zeta/delta, Hepatoma-derived growth factor, Gelsolin, Integrin alpha-3, 14-3-3 protein epsilon, Annexin A3, 14-3-3 protein theta, Proliferation-associated protein 2G4, 60 kDa heat shock protein, mitochondrial, Protein S100-A14, Vinculin, Ras-related protein Rab-7a, Integrin beta-1, Integrin alpha-6, Cytochrome c, somatic, Interleukin enhancer-binding factor 3, Cell division cycle 34B Protein phosphatase 1 regulatory inhibitor subunit 16B (Fragment); Protein phosphatase 1 regulatory inhibitor subunit 16B and Serine/threonine-protein kinase D.

According to some embodiments, the subject at risk is a smoker, a former smoker, or a non-smoker that is chemonaive; the subject at risk has been treated for lung cancer and is in remission; the subject at risk is at risk for a recurrence of lung cancer; or the subject at risk is at risk for progression of lung cancer.

According to another aspect, the present disclosure provides a method for detecting lung colonizing cells derived from a primary tumor during early development of a secondary lung cancer comprising: (a) identifying a unique cancer surface protein derived from the primary tumor in a subject 1 by: (i) obtaining an exhaled breath sample from the subject with a primary tumor, wherein the primary tumor has not metastasized and from a healthy control; (ii) condensing the exhaled breath sample in a cooling chamber and collecting the exhaled breath condensate (EBC); (iii) purifying EVs derived from the primary tumor and contained in the EBC obtained from the subject 1 and the healthy control; (iv) evaluating a level of expression of miRNAs, mRNAs, surface proteins or a ratio of any two thereof included in the EVs contained in the EBC sample from the subject 1 and in the EVs contained in the EBC sample from the healthy control; (v) identifying the unique cancer protein derived from the primary tumor in subject 1; (b) using the unique cancer protein derived from the primary tumor in (a), obtaining an exhaled breath sample from a subject 2, wherein the subject 2 is at risk for a secondary lung tumor derived from the primary tumor in (a); (c) condensing the exhaled breath from the subject 2 in a cooling chamber and collecting the exhaled breath condensate; (d) purifying a population of EVs contained in the EBC from the subject 2, (e) identifying a therapeutic biosignature for the secondary lung cancer in the EVs contained in the EBCs comprising expression of one or more of miRNAs, mRNAs and surface proteins derived from the secondary lung cancer; and (f) identifying the patient as one that can benefit therapeutically from being treated for the secondary lung cancer at an early stage.

According to some embodiments the first subject is an orthotopic animal model and the second subject is an orthotopic model; or the first subject is a orthotopic animal model and the second subject is a PDX animal model; or the first subject is a PDX animal model and the second subject is an orthotopic animal model; or the first subject is a PDX animal model and the second subject is a PDX animal model; and control animal colonies are established with primary human small airway epithelial cells (HSAECs).

According to some embodiments; the orthotopic human tumor NOD/SCID mouse model bearing a human lung tumor comprising mutations including EGFR and KRAS mutations, and wherein human NSCLC lung cancer cell lines are transduced for ex vivo bioluminescence, expanded in vitro and administered into the lungs of the NOD/SCID nude mice by tracheal instillation for lung uptake, or the PDX model is a human tumor NOD/SCID mouse model established using NSCLC patient-derived tumor cells comprising mutations including EGFR and KRAS mutations,

According to some embodiments the primary tumor is a colorectal cancer, a breast cancer or a bladder cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic illustration showing the EV-CATCHER™ assay for targeted capture of exhaled EVs from mouse exhaled breath condensate (EBC).

FIG. 2 shows single animal exhaled breath condensate (EBC) collectors. Using two air pumps to push and pull air into an air chamber in contact with an animal nose, animal exhaled breath is directed into a condenser sitting on ice to generate EBC.

FIG. 3A, FIG. 3B, FIG. 3C and FIG. 3D-1, FIG. 3D-2, and FIG. 3D-3 show data from evaluations of EV-CATCHER®. FIG. 3A shows qPCR detection of the plant ath-miR-159a miRNA (100 pg) spiked in 100 μl human serum when using EV-CATCHER® and 11 laboratory-based EV purification assays; FIG. 3B shows transmission electron microscopy (TEM) of human plasma EVs purified with EV-CATCHER®; FIG. 3C shows Western blot validation of anti-mouse (top panel) and anti-human (lower panel) anti-CD63 antibodies against EVs purified from human (MDA-MB-231, HEK293) and mouse (Bone marrow Endothelial cells (BMECs) cell lines; FIG. 3D-1, FIG. 3D-2, and FIG. 3D-3 show next generation sequencing (NGS) profiles of differentially expressed small-RNAs, between human plasma (1a, 1b), mouse RAW264.7 EVs (2a, 2b), and EVs (RAW264.7) spiked and purified with a mouse anti-CD63 EV-CATCHER® assay from human plasma. [42].

FIG. 4A, FIG. 4B, FIG. 4C, FIG. 4D, FIG. 4E and FIG. 4F show mRNA next generation sequencing (NGS) and miRNA quantitative polymerase chain reaction (qPCR) and droplet digital PCT (ddPCR) from exhaled EVs (exh-Evs). FIG. 4A shows EV miRNA and cDNA profiles after SMARTer kit amplification (Takara Bio); FIG. 4B shows mRNA NGS reproducibility for RNA extracted from MDA_MB-231 LMC2 cell EVs purified by EV-CATCHER® (1 and 2) or by ultracentrifugation (UC1 and UC2); FIG. 4C shows a comparison of mRNA NGS data of RNA from MDA-MB-231 LMC2 well EVs between EV-CATCHER® (EV-CAT) and ultracentrifugation (UC). FIG. 4D shows qPCR detection of mir-21, miR-142-3p and miR 141 using RNA extracted from exh-EVs purified with EV-CATCHER® from human EBC (n=9). FIG. 4E shows ddPCR Positive droplet (blue) profiles of miR-21 detected upon serial dilutions of exh-EV RNA (one human donor). FIG. 4F shows miR-21 detected by ddPCR.

FIG. 5A and FIG. 5B-1 and FIG. 5B-2, show Proteomic analyses. FIG. 5A shows Mass spectrometry (MS) of EVs from breast cancer cells. Clustering analysis of EV protcomes of MCF-10A (n=3) and MDA-MB-231 cells (n=3) [74]. FIG. 5B-1 and FIG. 5B-2 show MS of secondary lung cancer exh-EVs. Protcomes of exh-EVs from healthy control (n=6) and lung tumor-bearing animals (n=6) (i.e., tail-vein injection of MDA-MD-231 LM2 cells).

FIG. 6A, FIG. 6B, FIG. 6C, FIG. 6D, FIG. 6E, and FIG. 6F show analysis of human exh-EVs. FIG. 6A shows TEM (top) and Super Resolution Microscopy (Oni Bio) (bottom) analysis of human exh-EVs with validation of CD9, CD63 and CCSP proteins on their surface. FIG. 6B shows Western blot identity validation of human exh-EVs (ultracentrifuged (UC) or EV-CATCHER). FIG. 6C shows miRNA NGS of 5 airway samples (mouth rinse, buccal brush, human-PAN EV-CATCHER exh-EVs, Bronchial brush, and Bronchioalveolar lavage from 18 subjects. FIG. 6D shows miRNA reads from whole EBC, human-PAN exh-EVs, and anti-CCSP exh-EVs. FIG. 6E shows Z-score of human-PAN and anti-CCSP exh-EVs for top 14 miRNAs between healthy subjects (ctl, n=12) and patient with stage IV lung cancer (LC, n=6), by NGS. FIG. 6F shows top 4 miRNAs (p-val <0.05) in human-PAN purified exh-EVs between ctl and LC.

FIG. 7A, FIG. 7B, FIG. 7C, FIG. 7D, FIG. 7E, FIG. 7F-1, FIG. 7F-2, FIG. 7F-3, FIG. 7F-4, FIG. 7F-5, FIG. 7F-6, and FIG. 7F-7 and FIG. 7G. show collection and analysis of human tumor exh-EVs in mouse EBC. FIG. 7A shows TdT-Luc bioluminescence in tumor-bearing animals at weeks 1, 6, and 12. FIG. 7B shows H & E sections of healthy lungs (control (Ctl)) and lung metastatic tumor foci (lung cancer (LC)) at 16 weeks. FIG. 7C shows mouse exh-EVs purified from EBC by UC and analyzed by TEM. FIG. 7D shows miRNA Z-score of top 30 differentially expressed miRNAs (p-val <0.05) between control (Ctl) and tumor-bearing animals (LC). miRNA NGS analyses were performed using RNA extracted from whole EBC, from human tumor exh-EVs purified with the human-PAN EV-CATCHER assay, and mouse exh-EVs purified with the mourse-PAN EV-CATCHER assay. FIG. 7E shows miRNA pathway enrichment analysis (miRNet) of the top 28 upregulated and 37 downregulated miRNAs in human tumor exh-EVs. FIG. 7F-1, FIG. 7F-2, FIG. 7F-3, FIG. 7F-4, FIG. 7F-5, FIG. 7F-6, and FIG. 7F-7 show miRNA expression heatmap of exh-EVs between control (blue) and lung tumor bearing mice (red). FIG. 7G shows qPCT validations of miR-210 and miR-222 using RNA from exh-EVs purified from EBC collected at weeks 1, 8, and 15 from control and lung tumor-bearing mice (males vs. females).

FIG. 8 shows the EBC collection scheme for all animal models.

FIG. 9A, FIG. 9B, FIG. 9C-1 and FIG. 9C-2, FIG. 9D-1 and FIG. 9D-2, and FIG. 9E show miRNA expression profiling of exh-EVs purified from EBC of healthy controls and patients with advanced lung cancer. FIG. 9A shows a table detailing sample ID, gender, and age of the subjects involved in this comparative analysis, which includes 12 controls and 6 treatment naïve patients diagnosed with stage IV lung cancer. The first patient was diagnosed with Squamous Cell lung Carcinoma, the second with Large Cell Lung Carcinoma, and the third, fourth, fifth, and sixth patients were diagnosed with adenocarcinoma (Non-small Cell Lung Carcinoma). FIG. 9B shows a PCA plot displaying the similarities in miRNA expression between the 12 controls (Healthy) and the 6 cases (Lung cancer) for exh-EVs that were purified from EBC with the PAN (CD63/CD9/CD81) EV-CATCHER assay or with the CCSP/SFTPC EV-CATCHER assay from the same samples. FIG. 9C-1 and FIG. 9C-2 show Heatmap expression analysis of the top 19 most differentially expressed miRNAs between the 12 controls (Healthy) and the 6 cases (Lung cancer) for exh-EVs that were purified from EBC using with the PAN EV-CATCHER assay or the CCSP/SFTPC EV-CATCHER assays. FIG. 9D-1 and FIG. 9D-2 show Box plot analyses of the top 15 most differentially expressed miRNAs between the 12 controls (Healthy) and the 6 cases (Lung cancer), displaying significant log 2 fold expression differences, as detected by small-RNA sequencing analyses. FIG. 9E shows Box plot analyses of the Z-score obtained from the top 14 most differentially expressed miRNAs between the 12 controls (left; healthy) and the 6 cases (right; lung cancer), for miRNA profiles after small-RNA sequencing of exh-EVs purified with the PAN EV-CATCHER assay (PAN; left) or the CCSP/SFTPC EV-CATCHER assay (CCSP/SFTPC; right). The p-value for differences between controls (n=12) and the cases (lung cancer; n=6) is displayed in the panels.

FIG. 10 is a perspective view of a first embodiment of an EBC collection device.

FIG. 11 is a detailed view of the EBC collection device of FIG. 10.

FIG. 12 is a detailed view of the EBC collection device of FIG. 10.

FIG. 13 is a detailed view of the EBC collection device of FIG. 10.

FIG. 14 is a mass flow meter system for use with the EBC collection device of FIG. 10.

FIG. 15 is a detailed view of the EBC collection device of FIG. 10.

FIG. 16 is a perspective and detailed view of a second embodiment of an EBC collection device.

FIG. 17A, FIG. 17B, FIG. 17C, FIG. 17D, FIG. 17E, FIG. 17F and FIG. 17G show establishment of the mouse model of human secondary lung cancer. FIG. 17A. Lentiviral construct that was stably transduced in the MDA-MB-231 subline 3475 cell line for the expression of TdTomato-luciferase and CD63-GFP. FIG. 17B. Fluorescence-activated cell sorting (FACS) for single-cell cloning of high intensity double positive TdTomato-Luc and CD63-GFP MDA-MB-231 subline 3475 cells. FIG. 17C. Confocal imaging of the highly metastatic MDA-MB-231 subline 3475 cell clone sorted by FACS, with DAPI staining to locate cellular nuclei (Blue color, left panel) and stably expressing the Td-tomato Luciferase protein (Red colored cells, second panel from the left) and the CD63-GFP protein on extracellular vesicles (Green dots, third panel from the left), with a Merged image all staining and fluorescent imaging on the right panel. FIG. 17D. Schematic description of the tail-vein injection process utilized to inoculate individual mice with 1×10⁶ TdTomato-Luc+/CD63-GFP+ MDA-MB-231 subline 3475 cells for the development of lung tumors in athymic BALB/c mice. FIG. 17E. Bioluminescent in vivo imaging of tumor-bearing mice at weeks 0, 6, and 12 after inoculation of TdTomato-Luc+/CD63-GFP+ MDA-MB-231 subline 3475 cells. Bioluminescence intensity is indicated by means of radiant efficiency (photons/sec/cm²/sr) scale bars with red being the most intense (See scale bar). FIG. 17F. Representative formalin-fixed whole lung tissue images collected from two healthy mice (control; left) and two TdTomato-Luc+/CD63-GFP+ MDA-MB-231 subline 3475 inoculated lung tumor-bearing mice (case; right). FIG. 17G. Representative images of Hematoxylin and Eosin (H&E) stained 5 μm tissue sections of lungs harvested from one control (left panels; 0× and 20× magnifications) and one lung tumor-bearing animal (right panels; 0× and 20× magnifications). The image right panel from one lung tumor-bearing animal shows extensive infiltration of metastatic carcinoma legions with a few rare immature lymphocytes seen interspersed.

FIG. 18 shows a whole mouse exhaled breath condensate (EBC) collection system version 1.0. For this system, EBC is collected from two unrestrained mice roaming in a sealed glass chamber, which contains a removable metal grate that allows animals to move freely with normal postural movement. Airflow throughout the system is maintained and directed toward a condenser. The components of this mouse EBC collection system include: part a. an air pump that controls airflow (2 ml/minute) of compressed breathing-grade air that is transported through ¼ inch plastic tubes; part b. a one-way Balston 0.01 mic 93% airflow filter that maintains air sterility, part c. a glass mouse chamber (containing two mice); that is part d. securely sealed on both ends by caps with gaskets; connected to part e. a glass condenser sealed on both ends by caps, which is placed on ice to allowfor the collection of EBC. It is estimated that ˜62.5 μL of EBC can be captured from two mice within one-hour of collection.

FIG. 19A, FIG. 19B, FIG. 19C-1, FIG. 19C-2, FIG. 19C-3, FIG. 19C-4, FIG. 19C-5, FIG. 19C-6, FIG. 19C-7, FIG. 19C-8, FIG. 19C-9, FIG. 19C-10, FIG. 19C-11, FIG. 19C-12, and FIG. 19C-13, and FIG. 19D show MicroRNA analysis of EBC collected from unrestrained animals. FIG. 19A. Timeline of the weekly EBC collections from animal pairs, separated by sex (circles for females, triangles for males), between healthy control mice (blue) and lung tumor-bearing mice (red), for a period of 16 weeks. Discovery analyses were performed using total small-RNA extracted from EBC collected at weeks 0, 5, 9, and 13 using Next-Generation Sequencing (NGS). Validation analyses were conducted using total small-RNA extracted from EBC at weeks 1, 8, and 15 using quantitative reverse transcription PCR (RT-qPCR). Proteomic analyses were conducted on EBC collected and pooled for weeks 12, 14 and 16 from control and tumor-bearing mice groups. FIG. 19B. PCA plots for miRNA expression of healthy control mice measured at weeks 0, 5, 9 and 13 for both females and males (top), and lung tumor-bearing mice at the same timepoints for both females and males (bottom) FIG. 19C-1, FIG. 19C-2, FIG. 19C-3, FIG. 19C-4, FIG. 19C-5, FIG. 19C-6, FIG. 19C-7, FIG. 19C-8, FIG. 19C-9, FIG. 19C-10, FIG. 19C-11, FIG. 19C-12, and FIG. 19C-13. Heatmap classification of the top 233 miRNAs detected by NGS using small-RNA extracted from EBC of healthy control (blue) and lung tumor-bearing (red) mice at weeks 0 (yellow), 5 (green), 9 (pink), and 13 (purple) for both females (grey) and males (black). FIG. 19D. Taqman© qPCR analyses of hsa-miR-222 and has-miR-210 using total small-RNA purified from EBC collected at weeks 1 (blue), 8 (light purple), and 15 (dark purple), separately for females and for males, with data calculated using the 244Ct formula between healthy mice (i.e., using week 1 Ct values as the reference), and lung tumor-bearing mice, both normalized to exogenous ath-miR-159a (100 pg) as an internal “housekeeping” control that was spiked in EBC before RNA extractions and qPCR analyses.

FIG. 20A-1 and FIG. 20A-2 and FIG. 20B show proteomic analysis of EBC from healthy controls and lung tumor-bearing mice. FIG. 20A-1 and FIG. 20A-2. Heatmap analysis of the top 55 most differentially expressed proteins detected using coupling suspension trapping based sample preparation with label-free data-independent acquisition mass spectrometry (S-Trap coupled DIA-MS) [100]. Seven of the top 55 selected proteins have been associated with metastasis in our previous study and are individually enhanced [100]. FIG. 20B. Pie chart distribution of all 286 identified proteins in pooled EBC samples of control (n=6) and lung tumor-bearing (n=6) mice, stratified based on the preferential organ/tissue origin of each individual protein as informed by ProteinAtlas, and distributed as either from lung, skin, urine, breast, testis, colon, or undetermined tissue origins (i.e., Other).

FIG. 21 show nose and mouth EBC collection system v2.0 for restrained individual animals. The system designed and described here includes additional devices that enable air flow and direct collection of EBC from the nose and mouth of restrained mice. The system is composed of: part a. an air pump controlling airflow (2 ml/minute) from an air tank transported by ¼ inch tubes; part b. an airflow valve to maintain air directionality and sterility; part c. an exhaled chamber that is tightly connected with gaskets; to the part d. mouse immobilization chamber where the animal is restrained and through ¼ inch tubes; toward part e. a glass condenser sealed on both ends by caps and sitting on ice for the accumulation of EBC droplets; which is connected to part e. a second air pump set up in vacuum mode to enhance air circulation through the entire system (i.e., set at 1 ml/min). This system allows for individual collection of ˜29 μL EBC from restrained mice within two hours. part e shows the mouse chamber. part f shows the vacuum pump.

FIG. 22A, FIG. 22B and FIG. 22C show purification and analysis of exhaled extracellular vesicles from mouse EBC. EBC collected directly from the nose and mouth of individual animals was evaluated for the presence of exhaled EVS. FIG. 22A. EBC from healthy control (n=3) and lung tumor-bearing (n=3) mice collected at weeks 2, 6 and 11, was evaluated using the Spectradyne nCS1 nanoparticle analyzer, using C400 cartridges for the detection of nanoparticles between 65 nm and 400 nm. FIG. 22B. EBC samples from 6 control (left; 3 ml) and 3 lung tumor-bearing (right; 1 ml) mice, collected over 4 weeks were subjected to ultracentrifugation and the pellets analyzed imaging using transmission electron microscopy (TEM). FIG. 22C EV pellets from healthy control (top panels) and from lung tumor-bearing mice were analyzed using Super-Resolution Microscopy (ONi instrument) using anti-human anti-CD63, anti-CD9, and anti-CD81 anti-tetraspanin antibodies to evaluate the size and identity of exhaled EVs contained in the ultracentrifuged EBC pellets of control (top panels) and lung tumor-bearing mice (bottom panels).

FIG. 23A, FIG. 23B, FIG. 23C-1, FIG. 23C-2, FIG. 23C-3 and FIG. 23C-4, FIG. 23D, and FIG. 23E show miRNA analyses of EBC and exhaled EVs collected from control and lung tumor-bearing mice. FIG. 23A. Western blot evaluation of anti-human and anti-mouse CD63 antibodies using EVs purified from tissue culture media of human breast cancer MDA-MB-231 cells, human kidney cancer HEK293 cells, and normal mouse bone marrow endothelial cells (BMECs). FIG. 23B. Pathology of lung tissues from female control (left panel) and lung tumor-bearing (right panel) mice after 24 weeks, showing extensive infiltration of metastatic carcinoma legions in the H&E sections obtained from one representative tumor-bearing animal (image right panel). FIG. 23C-1, FIG. 23C-2, FIG. 23C-3 and FIG. 23C-4. Heatmap analysis of the top 142 most detectable miRNAs between small-RNA extracted from whole EBC (green), and sequentially purified human exhaled EVs using the anti-human anti-CD63 EV-CATCHER assay from whole EBC (orange), and mouse exhaled EVs using the anti-mouse anti-CD63 EV-CATCHER assay from the same whole EBC samples (purple), collected at weeks 21 (light grey), 22 (brown), and 23 (dark grey) from female control (blue) and lung tumor-bearing (red) mice detectable at study end (week 24). FIG. 23D. MiRNA Z-score analysis of whole EBC, human exhaled EVs, and mouse exhaled EVs. The top three panels display the cumulative expression distribution of a human tumor EV miRNA Z-score when these miRNAs are detected from whole EBC, from EVs purified with the anti-human anti-CD63 EV-CATCHER assay, and from EVs purified with the anti-mouse anti-CD63 EV-CATCHER assay. The bottom three panels display the cumulative expression distribution of a mouse lung EV miRNA Z-score when these miRNAs are detected from whole EBC, from EVs purified with the anti-human anti-CD63 EV-CATCHER assay, and from EVs purified with the anti-mouse anti-CD63 EV-CATCHER assay. FIG. 23E shows qPCR using EBC samples collected at earlier stages of disease (i.e., weeks 2, 6 and 11) for animals that developed significant disease.

FIG. 24A-1, FIG. 24A-2, FIG. 24A-3, FIG. 24A-4, FIG. 24A-5, FIG. 24A-6, FIG. 24A-7, FIG. 24A-8, FIG. 24A-9, FIG. 24A-10, and FIG. 24A-11 and FIG. 24B show proteomic analysis of EBC pooled from controls and lung tumor-bearing mice. FIG. 24A-1, FIG. 24A-2, FIG. 24A-3, FIG. 24A-4, FIG. 24A-5, FIG. 24A-6, FIG. 24A-7, FIG. 24A-8, FIG. 24A-9, FIG. 24A-10, and FIG. 24A-11. Heatmap analysis of all 231 proteins detectable in EBC separately pooled from 3 control and 3 lung tumor-bearing mice, collected at week 10, using coupling suspension trapping based sample preparation with label-free data-independent acquisition mass spectrometry (S-Trap coupled DIA-MS; [100]). Based on all identified proteins, 4 were identified to have known associations with the metastatic process and are individually enhanced, as identified in FIG. 20A-1, FIG. 20A-2 and FIG. 20B and in our previous study

FIG. 24B. Pie chart distribution of the 231 identified proteins stratified based on the preferential organ/tissue expression of each individual protein based on ProteinAtlas, and classified as either from lung, skin, urine, breast, testis, upper digestive tract, colon and other undetermined tissue origins (i.e., Other).

FIG. 25. MiRNA enrichment analysis of the top 33 upregulated miRNAs identified in human tumor exhaled EVs. In silico analysis using miRNet network analysis freeware displaying the predicted miRNA-gene interactions for the top 33 upregulated miRNAs detected in human exhaled tumor EVs obtained from restrained female lung tumor-bearing mice when compared to control animals. Each analysis utilized miTarBase v8.0 as the selected gene target database and filtering was performed using a degree filter of 2.0 for all network nodes and the minimum network selection. The left most cluster identifies possible genes (i.e., purple and yellow dots) that interact with several of our 33 upregulated miRNAs (i.e., blue squares). The genes highlighted in yellow reflect genes that have published data showing their involvement in lung cancer. The right most cluster highlights the predicted miRNA-gene interactions and their involvement in human diseases (i.e., green squares), particularly prominent are those related to lung neoplasms, lung adenocarcinomas and non-small cell lung carcinoma (NSCLC).

### DETAILED DESCRIPTION OF THE INVENTION

As used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

As used herein, the term “about” means plus or minus 10% of the numerical value of the number with which it is being used. Therefore, about 50% means in the range of 40%-60%.

The term “airway” as used herein refers to the respiratory tract through which air passes during breathing. The extrapulmonary (upper) airway includes the nasopharynx and trachea, which are cartilaginous, do not change shape significantly with ventilation, and do not participate in gas exchange. The intrapulmonary airways are divided into three major groups: bronchi, bronchioles (including the terminal bronchioles), and respiratory bronchioles. By definition, bronchi have cartilage in their walls, whereas bronchioles do not. Respiratory bronchioles serve a dual function as airways and as part of the alveolar volume (gas exchange). The respiratory bronchioles and alveolar ducts participate in gas exchange. The cellular complexity of the airways is indicated by the nearly 50 distinct cell types found there, of which at least 12 are epithelial cells on the airways surface. Nearly half of the epithelial cells in the normal human airway are ciliated down to bronchioles. [Murray & Nadel's Textbook of Respirtory Medicine, 6^{th} Ed., Broaddus, Mason, Ernst, King, Jr., Laxarus, Murray, Nadel, Slutsky and Gotway, Eds. (2016) Elsevier Saunders, Vol. 1, Chapter 1, pp. 1-21].

The term “airway resistance” as used herein refers to the change in transpulmonary pressure needed to produce a unit flow of gas through the airways of the lung Generally speaking, it is the pressure difference between the mouth and alveoli of the lung, divided by airflow. In the anatomical lung, airflow is primarily laminar (meaning smooth and not turbulent), which allows for the application of Poiseuille's Law: Q=πPr⁴/8η1 (where Q=flow rate, P=pressure, r=radius, n=viscosity, l=length) where flow is proportional to pressure difference and inversely proportional to resistance. Multiple factors can influence airway resistance, including airflow velocity, the diameter of the airway, and lung volume. In disease, airways may narrow, which increases resistance.

Anatomical Terms. When referring to animals, that typically have one end with a head and mouth, with the opposite end often having the anus and tail, the head end is referred to as the cranial end, while the tail end is referred to as the caudal end. Within the head itself, rostral refers to the direction toward the end of the nose, and caudal is used to refer to the tail direction. The surface or side of an animal's body that is normally oriented upwards, away from the pull of gravity, is the dorsal side; the opposite side, typically the one closest to the ground when walking on all legs, swimming or flying, is the ventral side. On the limbs or other appendages, a point closer to the main body is “proximal”; a point farther away is “distal”. Three basic reference planes are used in zoological anatomy. A “sagittal” plane divides the body into left and right portions. The “midsagittal” plane is in the midline, i.e. it would pass through midline structures such as the spine, and all other sagittal planes are parallel to it. A “coronal” plane divides the body into dorsal and ventral portions. A “transverse” plane divides the body into cranial and caudal portions.

When referring to humans, the body and its parts are always described using the assumption that the body is standing upright. Portions of the body which are closer to the head end are “superior” (corresponding to cranial in animals), while those farther away are “inferior” (corresponding to caudal in animals). Objects near the front of the body are referred to as “anterior” (corresponding to ventral in animals); those near the rear of the body are referred to as “posterior” (corresponding to dorsal in animals). A transverse, axial, or horizontal plane is an X-Y plane, parallel to the ground, which separates the superior/head from the inferior/feet. A coronal or frontal plane is an Y-Z plane, perpendicular to the ground, which separates the anterior from the posterior. A sagittal plane is an X-Z plane, perpendicular to the ground and to the coronal plane, which separates left from right. The midsagittal plane is the specific sagittal plane that is exactly in the middle of the body.

Structures near the midline are called medial and those near the sides of animals are called lateral. Therefore, medial structures are closer to the midsagittal plane, lateral structures are further from the midsagittal plane. Structures in the midline of the body are median. For example, the tip of a human subject's nose is in the median line.

Ipsilateral means on the same side, contralateral means on the other side and bilateral means on both sides. Structures that are close to the center of the body are proximal or central, while ones more distant are distal or peripheral. For example, the hands are at the distal end of the arms, while the shoulders are at the proximal ends.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer in one embodiment to A without B (optionally including elements other than B); in another embodiment, to B without A (optionally including elements other than A); and, in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

As used herein, when used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps.

The term “adenocarcinoma” as used herein refers to a cancer that forms in the glandular tissue that lines certain internal organs and makes and releases substances in the body, such as mucus, digestive juices, and other fluids. Most cancers of the breast, lung, esophagus, stomach, colon, rectum, pancreas, prostate, and uterus are adenocarcinomas.

As used herein the term “administering”, when used in conjunction with a therapeutic, means to give or apply a therapeutic directly into or onto a target organ, tissue or cell, or to administer a therapeutic to a subject, whereby the therapeutic positively impacts the organ, tissue, cell, or subject to which it is targeted. Thus, as used herein, the term “administering”, when used in conjunction with compositions comprising, for example, an exosome and/or extracellular vesicle, can include, but is not limited to, providing the composition into or onto the target organ, tissue or cell; or providing a composition to a patient so that the therapeutic reaches the target organ, tissue or cell. “Administering” may be accomplished parenterally, e.g., by intravenous administration or infusion techniques, inhalation or insufflation, or by such methods in combination with other known techniques.

The term “Alix” as used herein refers to an accessory protein of endosomal sorting complex required for transport (ESCRT). Evidence indicates that Alix is involved in the packaging of miRNAs during extracellular vesicle (EV) biogenesis [Iavello, A. et al. Int. J. Mol. Med. (2016) 37 (4): 958-66].

The term “alveolar type II cells (AT2 cells) as used herein refers to the progenitors for alveolar type I cells. Alveolar type I cells cover 95 percent of the alveolar surface of the lung; they comprise the major gas exchange surface of the alveolus and are integral to the maintenance of the permeability barrier function of the alveolar membrane. AT2 cells are the only pulmonary cells that synthesize, store, and secrete all components of pulmonary surfactant important to regulate surface tension, preventing the collapse of part or all of a lung (atelectasis) and maintaining alveolar fluid balance within the alveolus.

The term “alveoli” (singular, alveolus) as used herein refer to microscopic balloon-shaped air sacs located at the end of the respiratory tree. They expand during inhalation, taking in oxygen, and shrink during exhalation, expelling carbon dioxide. The alveoli are the site where gas exchange between inspired air and the blood takes place.

The terms “animal,” “patient,” and “subject” as used herein include, but are not limited to, humans and non-human vertebrates such as wild, domestic, and farm animals. The terms “animal,” “patient,” and “subject” may refer to mammals, including, but not limited to, humans.

The term “antibody” as used herein refers to a polypeptide or group of polypeptides comprised of at least one binding domain that is formed from the folding of polypeptide chains having three-dimensional binding spaces with internal surface shapes and charge distributions complementary to the features of an antigenic determinant of an antigen.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface. Human antibodies show two kinds of light chains, K and 2; individual molecules of immunoglobulin generally are only one or the other.

An antibody may be an oligoclonal antibody, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody, and an antibody that can be labeled in soluble or bound form, as well as fragments, variants or derivatives thereof, either alone or in combination with other amino acid sequences provided by known techniques. Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies. Diverse libraries of immunoglobulin heavy (VH) and light (VK and Vλ) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage. The technique of guided selection is based on human immunoglobulin V gene shuffling with rodent immunoglobulin V genes. The method entails (i) shuffling a repertoire of human λ light chains with the heavy chain variable region (VH) domain of a mouse monoclonal antibody reactive with an antigen of interest; (ii) selecting half-human Fabs on that antigen (iii) using the selected λ light chain genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1, λ antibody molecule in the mouse myeloma. An antibody may be from any species. The term antibody also includes binding fragments of the antibodies of the invention; exemplary fragments include Fv, Fab, Fab′, single stranded antibody (svFC), dimeric variable region (Diabody) and di-sulphide stabilized variable region (dsFv). Structural and functional domains can be identified by comparison of the nucleotide and/or amino acid sequence data to public or proprietary sequence databases. For example, computerized comparison methods can be used to identify sequence motifs or predicted protein conformation domains that occur in other proteins of known structure and/or function. Methods to identify protein sequences that fold into a known three-dimensional structure are known. See, for example, Bowie et al. Science 253:164 (1991), which is incorporated by reference in its entirety.

As used herein, the terms “antigen” refers to any substance that elicits an immune response.

The term “antigen-binding site” as used herein refers to the site at the tip of each arm of an antibody that makes physical contact with an antigen and binds it noncovalently. The antigen specificity of the antigen-binding site is determined by its shape and the amino acids present.

The term “antigenic determinant” or “epitope” as used herein refers to that portion of an antigenic molecule that is contacted by the antigen-binding site of a given antibody or antigen receptor.

The term “aptamer” as used herein refers to single stranded synthetic oligonucleotides that fold into three-dimensional shapes capable of binding non-covalently with high affinity and specificity to a target molecule. Rather than primary sequence, binding of an aptamer is determined by its tertiary structure. Target recognition and binding involve three dimensional, shape-dependent interactions as well as hydrophobic interactions, base-stacking, and intercalation. According to some embodiments, the aptomer comprises two complementary primers used for EV-CATCHER. According to some embodiments, the aptomer comprises two complementary primers containing specific restriction enzyme recognition sites used for EV-CATCHER. According to some embodiments, the efficiency of each restriction enzyme digestion can be evaluated using quantitative PCR using site specific primer/probe sequences.

The term “binding” and its other grammatical forms as used herein means a lasting attraction between chemical substances. Binding specificity involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

As used herein, the term “binding agent” refer to a substance that can bind to a chemical or other substance, e.g., an antigen.

The term “biomarker” (or “biosignature”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (e.g., choices of drug treatment or administration regimes). In evaluating potential therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

A “predictive biomarker” is a biomolecule that indicates therapeutic efficacy, i.e., an interaction exists between the biomolecule and therapy that impacts patient outcome.

A “prognostic biomarker”, which is an indicator of innate tumor aggressiveness, is a biomolecule that indicates patient survival independent of the treatment received.

The term “breathing” as used herein refers to the process that moves air in (inhalation) and out exhalation) of the lungs.

The term “bronchial brushing” as sued herein refers to a biopsy procedure used to find cancer and changes in cells that may lead to cancer. A bronchoscope (a thin, tube-like instrument with a light and a lens for viewing) is inserted through the nose or mouth into the lungs. A small brush is then used to remove cells from the airways that lead to the lungs. These cells are then examined under a microscope.

The term “bronchoalveolar lavage” (BAL) is used herein to refer to a medical procedure in which a bronchoscope is passed through the mouth or nose into the lungs and fluid is squirted into a small part of the lung and then collected for examination. “Bronchoalveolar lavage fluid” (BALF) is used herein to refer to the fluid collected from a BAL procedure.

The term “cancer” as used herein refers to diseases in which abnormal cells divide without control and can invade nearby tissues. Cancer cells can also spread to other parts of the body through the blood and lymph systems.

The term “carcinoma” as used herein refers to a cancer that begins in the skin or in tissues that line or cover internal organs.

The term “cargo” as used herein refers to a load or that which is conveyed. With respect to exosomes and/or extracellular vesicles, the term cargo refers to a substance encapsulated in the exosome and/or extracellular vesicle. The compound or substance can be, e.g., a nucleic acid (e.g., nucleotides, DNA, RNA), a polypeptide, a lipid, a protein, or a metabolite, or any other substance that can be encapsulated in an exosome and/or extracellular vesicle. With respect to exosomes and/or extracellular vesicles, the term “cargo profile” as used herein refers to the measurement of the abundance of cargo components (e.g., a nucleic acid (e.g., nucleotides, DNA, RNA), a polypeptide, a lipid, a protein, or a metabolite) that characterize the population of exosomes and/or extracellular vesicles.

The term “CD9” as used herein refers to a member of the tetraspanin protein family whose crystal structure shows a reversed cone-like molecular shape, which generates membrane curvature in the crystalline lipid layers. (Umeda, R. et al. Nature Communic. (2020) 11: article 1606).

The term “CD37” as used herein refers to a member of the tetraspanin protein family exclusively expressed on immune cells. (Zuidscherwoude, M. et al. Scientific Reports (2015) 5:12201).

The term “CD63” as used herein refers to a member of the tetraspanin protein family, the C-terminal domain of which interacts with several subunits of adaptor protein (AP) complexes, linking the traffic of this tetraspanin to clathrin-dependent pathways (Andreu, Z. & Yanez-Mo, M., citing Rous, B A et al. Mol. Biol. Cell (2002) 13 (3): 1071-82). Among intracellular interacting proteins, CD63 was shown to directly bind to syntenin-1, a double PDZ domain-containing protein (Id., citing Latysheva, N. et al. Mol. Cell Biol. (2006) 26 (20): 7707-18). A major role in exosome biogenesis has been reported for Syntenin-1 (Id., citing Baietti, M F et al. Nat. Cell Biol. (2012) 14 (7): 677-85).

The term “CD81” as used herein refers to a member of the tetraspanin protein family whose crystal structure shows a reversed teepee-like arrangement of the four transmembrane (TM) helices, which create a central pocket in the intramembranous region that appears to bind cholesterol in the central cavity. (Zimmerman, B. et al. Cell (2016) 167:1041-51). During development, CD81 regulates the trafficking of CD19, an essential co-stimulatory molecule of lymphoid B cells and a well-characterized CD81 partner, along the secretory pathway. (Shoham, T. et al. J. Imunol. (2003) 171:4062-72). CD9 and CD81 have been shown to regulate several cell-cell fusion processes. (Charrin, S. et al. J. Cell Science (2014) 127:3641-48).

The term “CD82” as used herein refers to a member of the tetraspanin protein family that has been implicated in the regulation of protein sorting into EVs and in antigen presentation by antigen presenting cells. (Andreu, Z. and Yanez-Mo, M. Front. Immunol. (2014) doi.org/10.3389/fimmu.2014.00442).

As used herein, the term “cell proliferation” is meant to refer to the process that results in an increase of the number of cells, and is defined by the balance between cell divisions and cell loss through cell death or differentiation.

The term “chemonaïve” as used herein means having or showing no experience with chemotherapy.

The term “chemotherapy” as used herein refers to a treatment that uses drugs to stop the growth of cancer cells.

The term “circular RNAs” and “CirRNAs” as used herein refer to a special class of non-coding RNAs characterized with a covalently closed structure, without 5′ caps and 3′ poly(A) tails, produced by non-canonical splicing event, which cannot be cleaved by must ribonucleases. CircRNAs are primarily generated from primary transcripts through back-splicing and lariat-driven circularization, which compete with mRNA splicing. Back-splicing circularization depends on cis-acting elements and trans-acting factors. Cis-acting elements in the upstream introns are base-paired with the downstream introns for circularization. The term “trans-acting factors” refers to the RNA binding proteins (RBPs), which bind to specific motifs within the flanking introns to promote circularization. Lariat-driven circularization occurs in exon skipping or intron removal from pre-mRNAs. [Wang, C. et al. Cancer Letters (2020) 492:106-15].

CircRNAs are classified into exonic cirRNAs (EcRNAs), Extron-intron circRNAs (ElciRNAs), and circular intronic RNAs (ciRNAs). ElciRNAs and ciRNAs are circRNAs containing introns located in the nucleus, while the majority of EcRNAs are transported to the cytoplasm after biogenesis. The main functions of circRNAs include modulating the parental gene expression, regulating gene transcription, acting as miRNA sponges, binding with protein, and encoding small functional peptides. [Id., citing Chen, LL. Nat. Rev. Mol. Cell Biol. (2016) 17:205-11]. Emerging evidence suggests that dysregulated expression of cirRNAs is universal in tumorigenesis and progression of lung cancer, but the mechanisms are unkown. Chromosomal translocations have been found to produce a special type of cirRNA called fusioncirRNA [Id., citing Alderton, GK. Nat. Rev. Canc. (2016) 16:273; Guarnerio, J. et al. Cell (2016) 165:289-302]. It has been reported that quite a few cirRNAs, such as cirRNAs F-circEA and F-circEA-2a, are generated from oncogenic fusion genes with dysregulated expression in lung cancer [Id., citing Tan, S. et al. Mol. Canc. (20108) 17:138; Tan, S. et al. Cell Res. (2018) 78:2839-51], indicating that aberrant chromosomal translocations may be the universal mechanism ofr dysregulated expression of cirRNAs in lung cancer. Somatic copy number variations have been confirmed to produce cirRNAs; for example, amplification of the 3q26.2 locus causes overexpression of cirPRKCI in lung cancer [Id., citing Qiu, M. et al. Cance Res. (2018) 78:2839-51].

The terms “circulating nucleic acids”, “cell-free nucleic acids”, and “cfNAs” are used interchangeably herein to refer to DNA and RNA released into the circulation by tumor cells either passively (e.g., via apoptosis and necrosis) or through active secretion via extracellular vesicles (EVs) from viable cells.

The terms “circulating tumor cells” and “CTCs” are used interchangeably to refer to tumor cells that have sloughed off the primary tumor or metastases and extravasate into and circulate in the blood. CTCs are very rare in the bloodstream. To assess their characteristics it is necessary to separate them from the predominating mononuclear cells. CTC specific markers include epithelial cell adhesion molecule (Ep-CAM), human epidermal growth factor reccotr 2 (HER2); mucin 1 (MUC1), cytokeratins, etc. [Maly, V. et al. In vivo. (2019) 33:1027-37, citing Joosse, S A et al EMBO Mol. Med. (2015) 7 (1): 1-11; Wang, Y. et al. Int. J. Nanomedicine (2012) 7:2315-24]. CellSearch® (Menarini Silicon Biosystems), which is based on the anti-EpCAM immunomagnetic enrichment of CTCs is the only FDA approved system used clinically for CTC detection in patients with metastatic breast, colorectal and prostate cancer. [Lin, D. et al. Signal Transduction and Targeted Therapy (2021) 6: article 404].

The terms “circulating tumor DNA”, “ctDNA”, “cell-free DNA”, and “cfDNA” are used interchangeably to refer to tumor-derived fragmented DNA in the bloodstream that comes from primary or metastatic cancer sites.

The terms “circulating tumor RNA” and “ctRNA” and “cell-free RNA” and “cfRNA” are used interchangeably to refer messenger RNA (mRNA) and non-coding RNA (ncRNA) released into the circulation by tumor cells. ncRNAs can be secreted into extracellular space as cell-free RNAs either encapsulated inside macrovesicles (such as extracellular vesicles) or in association with proteins (as RNA-protein complexes) or high-density lipoproteins (HDLs), and can subsequently be detected in blood samples as circulating ncRNAs. Altered levels of circulating ncRNAs are found in body fluids, including serum, plasma, urine, and saliva, from patients with cancer compared with individuals without cancer. [Anfossi, S. et al. Nature Reviews Clinical Oncol. (2018) 15:541-63].

The term “Clara cell” or “Club cells” as used herein refers to non-ciliated, non-mucous, secretory cells in respiratory epithelium that secrete several distinctive proteins, including Clara cell 10-kDa secretory protein (CCSP). While the Clara cell is the principal secretory cell type throughout the tracheobronchial airways of the laboratory mouse, they are most predominant in the terminal and respiratory bronchioles of humans and monkeys. The primary functions of Clara cells are: (1) to provide secretory surfactants (surfactant proteins A, B and D) and other specific proteins (e.g., CCSP) that contribute to the airway epithelial lining fluid; (2) to serve as progenitor cells for ciliated and secretory epithelial cells; and (3) to metabolize xenobiotic compounds through P450 cytochrome-dependent mixed-function oxygenases associated with the smooth endoplasmic reticulum (SER).

The term “Clara cell secretory protein” (also known as “CCSP” or “CC16” or CC10″ or “Uteroglobin”) as used herein refers to an abundant protein of the airway lining fluid, which is secreted by club cells, as well as by serous and goblet cells of the proximal airways. Structurally, it is a small homodimeric protein consisting of two 8-kDa subunits aligned by disulfide links to form a hydrophobic pocket, which can bind phosphatidylcholine and phosphatidylinositol. CCSP has been noted to modulate inflammatory responses, suggesting a potential immunoregulatory role within the airways and alveoli. In vitro, CCSP has been shown to inhibit PMN chemotaxis, as well as macrophage phagocytosis. Its hydrophobic pocket allows CCSP to scavenge phospholipase A2 within the milieu, further limiting PMN activation. CCSP has also been shown to inhibit the production and bioactivity of IFN-γ. [Good, M. et al. Chapter 130 in Fetal and Neonatal Physiology (5^{th} Ed.) (2017) 2: pp. 1262-93.c12. doi.org/10.1016/B978-0-323-35214-7.00130-x].

The term “click chemistry” as used herein refers to chemical synthetic methods for making compounds using reagents that can be joined together using efficient reagent conditions and that can be performed in benign solvents or solvents that can be removed or extracted using facile methods, such as evaporation, extraction, or distillation. Several types of reactions that fulfill these criteria have been identified, including nucleophilic ring opening reactions of epoxides and aziridines, non-aldol type carbonyl reactions, such as formation of hydrazones and heterocycles, additions to carbon-carbon multiple bonds, such as oxidative formation of epoxides and Michael additions, and cycloaddition reactions. A representative example of click chemistry is a reaction depicted in Formula I below that couples an azide and an alkyne to form a triazole. The copper-catalyzed azide-alkyne cycloaddition (CuAAC) features an enormous rate acceleration of 107 to 108 compared to the uncatalyzed 1,3-dipolar cycloaddition. It succeeds over a broad temperature range, is insensitive to aqueous conditions and pH range over 4 to 12, and tolerates a broad range of functional groups. Pure products can be isolated by simple filtration or extraction without the need for chromatography or recrystallization.

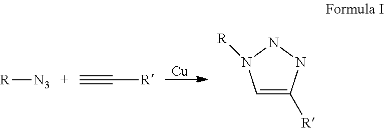

A representative example of copper-free click chemistry is a reaction that couples a dibenzocyclo-octyl (DBCO)-tagged DNA molecule to an azide-functionalized surface by cycloaddition without copper as shown in Formula II:

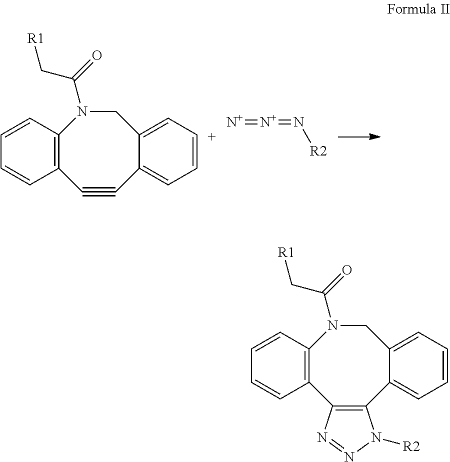

[Eeftens, J M, et al. BMC Biophys. (2015) 8:9].

According to some embodiments, the click chemistry is accomplished by SiteClick™ (Invitrogen, Catalog #S10467) a kit which allows direct activation of a single NH₂ group on each Fc domain of the antibody, which ensures unaffected antigen binding after IgG labelling. SiteClick Chemistry relies on site specific removal of a terminal Gal residue with β-galactosidase present on the two N-linked glycans located in IgG antibody heavy chain Fc domain. The unmasking of these two terminal GlcNAc labeling sites for the subsequent enzymatic β-galactosyl transferase (GalT) reaction allows for the azide-activated antibody to be labeled with a dibenzocyclooctyne (DIBO)-functionalized probe using copper-free click chemistry.

The term “clickable functional group” as used herein refers to a functional group that can be used in click chemistry to form a product. According to some embodiments, the clickable functional group is an azide or an alkyne.

The term “clinical outcome” or “outcome” is used to refer to a specific result or effect that can be measured. Examples of outcomes include progression-free survival, overall survival, complete response; and stable disease.

The term “complementary” with regard to base pairing as used herein refers to DNA base pairing wherein adenine pair specifically with thymine (A-T), guanine with cytosine (G-C) and uracil with adenine (U-A); the two bases in a classic base pair are said to be complementary to each other.

The terms “complete response” or “complete remission” or “CR” as used herein refer to the disappearance of all signs of cancer in response to treatment. This does not always mean the cancer has been cured.

The term “conjugate” as used herein refers to a compound formed by the joining or linking together of two or more chemical compounds.

The term “contact” and its various grammatical forms as used herein refers to a state or condition of touching or of immediate or local proximity.

As used herein, the term “derived from” refers to any method for receiving, obtaining, or modifying something from a source of origin.

The terms “disease progression” or “progressive disease” or “PD” as used herein refer to a cancer that continues to grow or spread.

The term “DNA library” as used herein refers to a collection of DNA fragments that have been cloned into vectors so that researchers can identify and isolate the DNA fragments that interest them for further study.

The term “domain” as used herein refers to a region of a protein with a characteristic tertiary structure and function and to any of the three-dimensional subunits of a protein that together make up its tertiary structure formed by folding its linear peptide chain.

The term “drug resistance” as used herein refers to a situation where cancer cells no longer respond to an agent that was able to kill or weaken them. At the tumor level, mechanisms of drug resistance include, without limitation, activation of prosurvival pathways; ineffective induction of cell death; positive selection of a drug-resistant tumor subpopulation, and mutations, amplifications or deletions in drug targets. [Holohan, C. et al. Nat. Rev. Cancer (2013) 13:714-26].

The term “encapsulated” as used herein refers to being enclosed in a capsule (meaning a membranous envelope enclosing a part). The term “encapsulate” and its various grammatical forms as used herein refers to enclosure of one or more active agents within another material regardless of shape or design. In a nonlimiting example, the basic schematic structure of core-shell encapsulation involves a material in the center or core, and an encapsulating agent, such as a membrane, surrounds the core.

The term “Endosomal Sorting Complexes required for transport” (ESCRTs) refers to components involved in multivesicular body (MVB) and intraluminal vesicle (ILV) biogenesis. ESCRTs consist of approximately twenty proteins that assemble into four complexes (ESCRT-0, -I, -II and -III) with associated proteins (VPS4, VTA1, ALIX), which are conserved from yeast to mammals (Colombo, M. et al. J. Cell Science (2013) 126:5553-65, citing Henne, W. M., et al. (2011). Dev. Cell 21, 77-91; Henne et al., 2011; Roxrud, I. et al. (2010). ESCRT & Co. Biol. Cell 102, 293-318). The ESCRT-0 complex recognizes and sequesters ubiquitylated proteins in the endosomal membrane, whereas the ESCRT-I and -II complexes appear to be responsible for membrane deformation into buds with sequestered cargo, and ESCRT-III components subsequently drive vesicle scission (Id., citing Hurley, J. H. and Hanson, P. I. (2010). Nat. Rev. Mol. Cell Biol. 11, 556-566; Wollert, T. et al. Nature (2009) 458:172-77). ESCRT-0 comprises hepatocyte growth factor-regulated tyrosine kinase dubstrate (HRS) protein that recognizes the mono-ubiquitylated cargo proteins and associates in a complex with signal-transducing adaptor molecule (STAM), Eps15 and clathrin. HRS recruits TSG101 of the ESCRT-I complex, and ESCRT-I is then involved in the recruitment of ESCRT-III, through ESCRT-II or ALIX, an ESCRT-accessory protein. Finally, the dissociation and recycling of the ESCRT machinery requires interaction with the ATPase associated with various cellular activities (AAA-ATPase) Vps4; Vps4 releases ESCRT-III from the MVB membrane for additional sorting events. It is unclear whether ESCRT-II has a direct role in ILV biogenesis or whether its function is limited to particular cargo (Id., citing Bowers, K. et al. (2006) J. Biol. Chem. 281, 5094-5105; Malerød, L. et al. Traffic 8, 1617-1629).

Concomitant depletion of ESCRT subunits belonging to the four ESCRT complexes does not totally impair the formation of MVBs, indicating that other mechanisms may operate in the formation of ILVs and thereby of exosomes and/or extracellular vesicles (Id., citing Stuffers, S. et al., (2009) Traffic 10, 925-937). One of these pathways requires a type II sphingomyelinase that hydrolyses sphingomyelin to ceramide (Id., citing Trajkovic, K. et al. (2008) Science 319, 1244-1247). Although the depletion of different ESCRT components does not lead to a clear reduction in the formation of MVBs and in the secretion of proteolipid protein (PLP) associated to exosomes and/or extracellular vesicles, silencing of neutral sphingomyelinase expression with siRNA or its activity with the drug GW4869 decreases exosome and/or extracellular vesicle formation and release. However, whether such dependence on ceramides is generalizable to other cell types producing exosomes and/or extracellular vesicles and additional cargos has yet to be determined. The depletion of type II sphingomyelinase in melanoma cells does not impair MVB biogenesis (Id., citing van Niel, G. et al., (2011) Dev. Cell 21, 708-721) or exosome and/or extracellular vesicle secretion, but in these cells the tetraspanin CD63 is required for an ESCRT-independent sorting of the luminal domain of the melanosomal protein PMEL (van Niel et al., (2011) Dev. Cell 21, 708-721). Moreover, tetraspanin-enriched domains have been proposed to function as sorting machineries allowing exosome and/or extracellular vesicle formation (Percz-Hernandez, D. et al. J. Biol. Chem. (2013) 288, 11649-11661).

Despite evidence for ESCRT-independent mechanisms of exosome and/or extracellular vesicle formation, proteomic analyses of purified exosomes and/or extracellular vesicles from various cell types have identified ESCRT components (TSG101, ALIX) and ubiquitylated proteins (Id., citing Buschow, S. et al., (2005) Blood Cells Mol. Dis. 35, 398-403; Théry, C. et al., (2006). Curr. Protoc. Cell Biol. Chapter 3, Unit 3.22). It has also been reported that the ESCRT-0 component HRS could be required for exosome and/or extracellular vesicle formation and/or secretion by dendritic cells (DCs), and thereby impact on their antigen-presenting capacity (Id., citing Tamai, K. et al., (2010) Biochem. Biophys. Res. Commun. 399, 384-390). The transferrin receptor (TfR) in reticulocytes that is generally fated for exosome and/or extracellular vesicle secretion, although not ubiquitylated, interacts with ALIX for MVB sorting (Id., citing Géminard, C. et al., (2004). Traffic 5, 181-193). It was also shown that ALIX is involved in exosome and/or extracellular vesicle biogenesis and exosomal sorting of syndecans (a major family of cell surface heparan sulfate proteoglycans (HSPGs) composed of sulfated glycosaminoglycans (GAGs), heparan sulfate (HS) or both HS and chondroitin sulfate (CS), attached covalently to core proteins-see Park, P W. Methods Cell Biol. (2018) 143:317-33) through its interaction with syntenin (Colombo, M. et al. J. Cell Science (2013) 126:5553-65, citing Baictti, M F et al., (2012). Nat. Cell Biol. 14, 677-685). Silencing of genes for two components of ESCRT-0 (HRS, STAM1) and one of ESCRT-I (TSG101), as well as a late acting component (VPS4B) induced consistent alterations in exosome and/or extracellular vesicle secretion. [Colombo, M. et al. J. Cell Sci. (2013) 126:5553-65].

The term “epidermal growth factor receptor” or EGFR″ as used herein refers to a protein found on certain types of cells that bind to a substance called epidermal growth factor. The epidermal growth factor family of receptor tyrosine kinases (ErbBs) plays essential roles in regulating cell regulating cell proliferation, survival, differentiation and migration. The epidermal growth factor family of receptor tyrosine kinases (ErbBs) consists of four members: EGFR (ErbB1, HER1), ErbB2 (HER2, neu in rodents), ErbB3 (HER3) and ErbB4 (HER4). These structurally related receptors are single chain transmembrane glycoproteins consisting of an extracellular ligand-binding ectodomain, a transmembrane domain, a short juxtamembrane section, a tyrosine kinase domain and a tyrosine-containing C-terminal tail. Binding of soluble ligand to the ectodomain of the receptor promotes homo- and heterodimer formation between receptors. Receptor dimerization is essential for activation of the intracellular tyrosine kinase domain and phosphorylation of the C-terminal tail [Wieduwilt, M J and Moasser, MM, Cell Mol. Life Ci. (2008) 65 (10): 1566-84, citing Linggi, B. and Carpenter, G. Trends Cell Biol. (2006) 16:649-56]. Phosphotyrosine residues then activate, either directly or through adaptor proteins, downstream components of signaling pathways including Ras/RAF/MAPK, PLCy1/PKC, PI (3) Kinase/Akt, mTOR, and STAT pathways [Id., citing Scaltriti, M. and Baselga, J. Clin. Cancer Res. (2006) 12:5268-72]. EGFR is overexpressed or aberrantly activated in 50% to 90% of NSCLCs.

The term “epigenetic changes” as used herein refers to heritable changes in gene expression that are not due to any alteration in the DNA sequence.

The term “exhalation” as used herein refers to expulsion of air from the lungs in breathing. Exhalation generally occurs passively.

The term “exosomes and/or extracellular vesicles” as used herein refers to extracellular bilayered membrane-bound vesicles of endosomal origin in a size range of ˜40 to 160 nm in diameter (˜100 nm on average) generated by all cells that are actively secreted.

Biogenesis. Exosomes and/or extracellular vesicles are generated in a process that involves double invagination of the plasma membrane and the formation of intracellular multivesicular bodies (MVBs) containing intraluminal vesicles (ILVs). ILVs are ultimately secreted as exosomes and/or extracellular vesicles with a size range of ˜40 to 160 nm in diameter through MVB fusion to the plasma membrane and exocytosis. The first invagination of the plasma membrane forms a cup-shaped structure that includes cell-surface proteins and soluble proteins associated with the extracellular milieu. This leads to the de novo formation of an early-sorting endosome (ESE) and in some cases may directly merge with a preexisting ESE. The trans-Golgi network and endoplasmic reticulum can also contribute to the formation and the content of the ESE (Kalluri, R. and LeBleu, VS. Science (2020) 367 (6478): eaau6977, citing Kalluri, R. J. Clin. Invest. (2016) 126:1208-15; van Neil, G., et al. Nat. Rev. Mol. Cell Biol. (2018) 19:213-28; McAndrews, K M and Kalluri, R. Mol. Cancer (2019) 18:52; Mathieu, M., et al. Nat. Cell Biol. (2019) 21:9-17; Willms, E., et al. Front. Immunol. (2018) 9:738; Hessvik, N P and Llorente, A. Cell Mol. Life Sci. (2018) 75:193-208). ESEs can mature into late-sorting endosomes (LSEs) and eventually generate MVBs, which are also called multivesicular endosomes. MVBs form by inward invagination of the endosomal limiting membrane (that is, double invagination of the plasma membrane). This process results in MVBs containing several ILVs (future exosomes and/or extracellular vesicles). The MVB can either fuse with lysosomes or autophagosomes to be degraded or fuse with the plasma membrane to release the contained ILVs as exosomes and/or extracellular vesicles [Id., citing Kahler, C., Kalluri, R. J. Mol. Med. (2013) 91:431037].

Heterogeneity: The heterogeneity of extracellular vesicles is thought to be reflective of their size, content, functional impact on recipient cells, and cellular origin. During their secretion they acquire surface proteins from their cell of origin. They naturally transport mRNA, miRNA, and proteins between cells.

Biomarkers. There is general agreement that their membranes are specifically enriched in tetraspanins CD9, CD37, CD63, CD81, and CD82.

Role. Extracellular vesicles are mediators of near and long-distance intercellular communication in health and disease and affect various aspects of cell biology.

The term “expand” or “amplify” as used herein with respect to cells refers to increasing in cell number.

As used herein, the term “expression” and its other grammatical forms refers to production of an observable phenotype by a gene, usually by directing the synthesis of a protein. It includes the biosynthesis of mRNA, polypeptide biosynthesis, polypeptide activation, e.g., by post-translational modification, or an activation of expression by changing the subcellular location or by recruitment to chromatin.

The term “extracellular vesicles (EVs)” as used herein refers to nanosized, membrane-bound vesicles released from cells that can transport cargo-including DNA, RNA, and proteins-between cells as a form of intercellular communication. Different EV types, including microvesicles (MVs), exosomes, oncosomes, and apoptotic bodies, have been characterized on the basis of their biogenesis or release pathways. Microvesicles bud directly from the plasma membrane, are 100 nanometers (nm) to 1 micrometer (μm) in size, and contain cytoplasmic cargo (Zaborowski, M P et al. BioScience (2015) 65 (8): 783-97, citing Heijnen, H F et al. Blood (1999) 94:3791-99). Another EV subtype, exosomes, is formed by the fusion between multivesicular bodies and the plasma membrane, by which multivesicular bodies release smaller vesicles (exosomes) whose diameters range from 40 to 160 nm (Id., citing El Andaloussi, S., et al. Nature Reviews Drug Discovery (2013) 12:347-57; Cocucci, E. and Meldolesi J. Trends in Cell Biology (2015) 25:364-72). Dying cells release vesicular apoptotic bodies (50 nm-2 μm) that can be more abundant than exosomes or MVs under specific conditions and can vary in content between biofluids (Id., citing Thery, C., et al. J. Immunology (2001) 1666:7309-18; El Andaloussi, S., et al. Nature Reviews Drug Discovery (2013) 12:347-57). Membrane protrusions can also give rise to large EVs, termed oncosomes (1-10 μm), which are produced primarily by malignant cells in contrast to their nontransformed counterparts (Id., citing Di Vizio, D., et al. Am. J. Pathol. (2012) 181:1573-84; Morello, M. et al. Cell Cycle (2013) 12:3526-36).

Collectively, EVs contain an abundance of cellular cargos [Shao, H., et al. Chem Rev. (2018) 118 (4): 1917-50., citing Kalra, H. et al. PLOS Biol. (2012) 10: e100450; Keerthikumar, S., et al. J. Mol. Biol. (2016) 428:688-92; Choi, D S., et al. Mass Spectrom. Rev. (2015) 34:474-90]. Consistent with their biogenesis, the membrane composition of microvesicles reflects most closely the plasma membrane of the parent cells [Id., citing Yanez-Mo, M. et al. J. Extracell. Vesicles (2015) 4:27066]. Consistent with their endosomal origin, the lipid membrane of exosomes is rich in cholesterol, sphingomyelin, and ceramide that are typical of lipid rafts. [Tan, S S H et al. Tissue Engineering: Part B (2020) doi: 10.1089/ten.teb.2019.0326].

In contrast, a specific subset of endosomal proteins has been identified in exosomes, suggesting a sorting mechanism during exosomal development. The endosomal sorting complex required for transport (ESCRT) has been extensively characterized for regulating and channeling specific molecules into the intraluminal vesicles of the MVBs [Shao, H., et al. Chem Rev. (2018) 118 (4): 1917-50, citing Hurley, J H and Hanson, PI. Nat. Rev. Mol. Cell Biol. (2010) 11:556-66; Henne, W M, et al. Dev. Cell (2011) 21:77-91]. The ESCRT, with its four main complexes (ESCRT 0, I, II, and III) is responsible for delivering ubiquitinated proteins for lysosomal degradation and protein recycling [Id., citing Wollert, T. and Hurley, JH. Nature (2010) 464:864-9]. Studies have shown that the depletion of specific ESCRT-family proteins can alter the protein content of exosomes and the rate of exosome release from cells [Id., citing Colombo, M., et al., J. Cell Sci. (2013) 126:5553-65]. Components of the ESCRT system, such as TSG101 and Alix [Id., citing Kowal, J., et al. Curr. Opin. Cell Biol. (2014) 29:116-25] are found enriched in exosomes and thus are used as markers for exosome identification [Id., citing Lotvall, J., et al. J. Extracell. Vesicles (2014) 3:26913].

Other ESCRT-independent processes also seem to participate, possibly in an intertwined manner, in exosome formation and release. As such, exosomes are also enriched with molecules involved in ESCRT-independent mechanisms. For example, the tetraspanin proteins such as CD9, CD63 and CD81 have been shown to participate in endosomal vesicle trafficking [Id., citing van Neil, G., et al. Dev. Cell. (2011) 21:708-21; Verweij, F J et al., EMBO J. (2011) 30:2115-29] The involvement of the Rab family of small GTPases in vesicle trafficking and fusion with the plasma membrane also suggests a role of these proteins in exosome release [Id., citing Vanlandingham, PA and Ceresa, BP. J. Biol. Chem. (2009) 284:12110-24; Ostrowski, M. et al. Nat. Cell Biol. (2010) 12:19-30; Zeigerer, A., et al. Nature (2012) 485:465-70]. In addition, sphingomyelinase has been demonstrated to be involved in vesicle release, as supported by elevated levels of ceramide in exosomes and a reduction in exosome release upon inhibition of sphigomyelinase [Id., citing Trajkovic, K., et al. Science (2008) 319:1244-7].

Proteins Enriched in EVs. EV proteins derive mainly from cellular plasma membrane, cytosol, but not from other intracellular organelles (e.g., Golgi apparatus, endoplasmic reticulum, and nucleus) [Id., citing Simpson, R J., et al. Expert Rev. Protcomics (2009) 6:267-83; Raimondo, F., et al. Proteomics (2011) 11:709-20; Choi, D S., et al. Mass Spectrum Rev. (2015) 34:474-90]. This protein constitution of EV is indicative of vesicle biogenesis and cargo sorting [Id., citing Kowal, J., et al. Proc. Natl Acad. Sci. USA (2016) 113: E968-77]

Membrane Proteins. In mammalian EVs, both transmembrane and lipid-bound extracellular proteins (e.g., lactadherin) are found associated with microvesicles and exosomes [Id., citing Lotvall, J., et al. J. Extracell Vesicles. (2014) 3:26913] Within the group of transmembrane proteins, exosomes are enriched in tetraspanins (e.g., CD9, CD63, CD81), a superfamily of proteins with four transmembrane domains [Id., citing van Niel, G., et al. Dev. Cell (2011) 21:708-21; Velrweij, F J., et al. EMBOJ. (2011) 30:2115-29]. Tetraspanins are involved in membrane trafficking and biosynthetic maturation [Id., citing Perez-Hernandez, D., et al. J. Biol. Chem. (2013) 288:11649-61; Andreu, Z. and Yanez-Mo, M. Front. Immunol (2014) 5:442] and are highly expressed in exosomes. Tetraspanins, however, are not uniquely expressed in exosomes alone. [Id., citing Lotvall, J., et al. J. Extracell. Vesicles (2014) 3:26913]. Reflecting their derivation from the plasma membrane of cells, EVs are enriched with specific transmembrane protein receptors (e.g., epidermal growth factor receptors/EGFRs6 [Id., citing A1-Nedawi, K., et al. Proc. Natl Acad. Sci. USA (2009) 106:3794-9] and adhesion proteins (e.g., epithelial cell adhesion molecule/EpCAM [Id., citing Im, H., et al. Nat. Biotechnol. (2014) 32:490-5; Tauro, B J., et al. Mol. Cell Proteomics (2013) 12:587-98].

Intravesicular Proteins. EV-associated intravesicular proteins have diverse functions. They include cytosolic proteins that have membrane- or receptor binding capacity, such as TSG101, ALIX, annexins and Rabs, which are involved in vesicle trafficking. EVs are also enriched with cytoskeletal proteins (e.g., actins, myosins, tubulins), molecular chaperones (e.g., heat-shock proteins/HSPs), metabolic enzymes (e.g., enolases, glyceraldehyde 3-phosphate dehydrogenase/GAPDH) and ribosomal proteins [Id, citing Lotvall, J., et al. J. Extracell. Vesicles (2014) 3:26913; Choi, D S., et al. Mass Spectrom. Rev. (2015) 34:474-90]. It has been reported that EV protein cargoes can be effectively transported to and received by recipient cells to elicit potent cellular responses in vitro and in vivo [Lai, C P., et al. Nat. Communic. (2015) 6:7029; Mittelbrunn, M. and Sanchez-Madrid, F. Nat. Rev. Mol. Cell Biol. (2012) 13:328-35].

Nucleic Acids. Both exosomes and microvesicles also contain nucleic acids include miRNAs, mRNAs [Id., citing Valadi, H., et al. Nat. Cell Biol. (2007) 9:654-9; Skog, J., et al. Nat. Cell Biol. (2008) 10:1470-6], DNA [Id., citing Balaj, L., et al. Nat. Commun. (2011) 2:180; Thakur, B K., et al. Cell Res. (2014) 24:766-9] and other non-coding RNAs [Id., citing Wei, Z., et al. Nat. Commun. (2017) 8:1145] RNA types are summarized in Table 4.

TABLE 4
Types of RNA found in EVs [Taken from Shao,
H. et al. Chem. Rev. (2018) 118 (4): 1917-50]

| RNA | Functions | Coding | Typical Size | |
|---|---|---|---|---|
| mRNA | Protein translation | Yes | 400-12,000 nt, | |
| | | | average ≈ 2100 nt | |
| microRNA | Post-transcriptional | No | 17-24 | nt |
| (miRNA) | gene silencing | | | |
| Y RNA | Component of Ro60 | No | ≈100 | nt |
| | ribonucleoprotein | | | |
| | particle; initiation | | | |
| | factor for DNA | | | |
| | replication | | | |
| Signal Recognition | Component of SRP | No | ≈280 | nt |
| particle RNA (SRP | ribonucleoprotein | | | |
| RNA) | complex that directs | | | |
| | protein trafficking | | | |
| Transfer RNA | Adapter for matching | No | 76-90 | nt |
| (tRNA) | amino acid to mRNA | | | |
| Ribosomal RNA | RNA component of | No | 185 | (1.9 kb) |
| (rRNA) | ribosomes | | 28S | (5.0 kb) |
| Small nuclear RNA | RNA processing such | No | ≈150 | nt |
| (snRNA) | as mRNA splicing | | | |
| Small nucleolar | Guiding chemical | No | 20-24 | nt |
| RNA (snoRNA) | modifications of | | | |
| | other RNAs | | | |
| Long noncoding | Many, including in- | No | >100 | nt |
| RNA (lncRNA) | transcription and | | | |
| | post-transcription | | | |
| | regulation | | | |

mRNA. mRNAs are a large family of coding RNA molecules that specify protein sequence information. Studies have reported that EVs contain a substantial proportion of their parent cells' mRNA pool, many of which are cell type-specific mRNA. [Shao, H., et al. Chem Rev. (2018) 118 (4): 1917-50, citing Wei, Z., et al. Nat. Commun. (2017) 8:1145; Batagov, A O and Kurochkin, IF. Biol. Direct (2013) 8:12]. These mRNA molecules, often in fragmented form, reside within EVs and are protected from RNase degradation. Furthermore, the fraction of polyadenylated mRNA molecules in EVs suggest that some of them (<2 kb) are capable of encoding polypeptides in support of protein synthesis (i.e., functionality in protein translation). This has been confirmed in multiple studies through different translation assays in recipient cells [Id., citing Valadi, H., et al. Nat. Cell Biol. (2007) 9:654-9; Skog, J., et al. Nat. Cell Biol. (2008) 10:1470-6; Lai, C P., et al. Nat. Commun. (2015) 6:7029]

miRNA. miRNAs are a class of small, noncoding RNAs (typically 17-24 nucleotides) which mediate post-transcriptional gene silencing usually by targeting the 3′ untranslated region of mRNAs. By suppressing protein translation, EV miRNAs are powerful regulators for a wide range of biological processes [Id., citing Mittelbrunn, M., et al. Nat. Commun. (2011) 2:282; Redzic, J S., et al. Semin. Cancer Biol. (2014) 28:14-23]. miRNAs can also exist in multiple stable forms when circulating in bodily fluids. For example, in addition to being packaged into EVs, circulating miRNAs can also be loaded onto high-density lipoprotein [Id., citing Vickers, K C., et al. Nat. Cell Biol. (2011) 13:423-33; Wagner, J., et al. Arterioscler. Thromb. Vasc. Biol. (2013) 33:1392-400] or bound to AGO2 protein outside the vesicles [Id., citing Arroyo, et al. Proc. Natl Acad. Sci. USA (2011) 108:5003-8; Turchinovich, A., et al. Methods Mol. Biol. (2013) 1024:97-107]. The distribution of miRNAs within EVs remains unclear [Id., citing Min, PK & Chan, SY. Eur. J. Clin. Invest. (2015) 45:860-74; Turchinovich, A., et al. Methods Mol. Biol. (2013) 1024:97-107; Chevillet, J R., et al. Proc. Natl. Acad. Sci. USA (2014) 111:14888-93]. As in the case of mRNA, miRNA profiles in EVs reflect their cell of origin but differs somewhat from their parental cells. Some miRNAs have been found preferentially sorted into EVs and remaining functional in recipient cells to regulate protein translation. [Id., citing Villarroya-Beltri, C., et al. Nat. Commun. (2013) 4:2980; Koppers-Lalic, D., et al. Cell Rep. (2014) 8:1649-58; Santangelo, L., et al. Cell Rep. (2016) 17:799-808; Teng, Y, et al. Nat. Commu. (2017) 8:14448]

Other RNA Types. In addition to mRNA and miRNA, many noncoding RNA types have been identified in EVs through next generation sequencing [Id., citing Huang, X., et al. BMC Genomics (2013) 14:319; Conley, A., et al. RNA Biol. (2017) 14:305-16]. These RNAs include transfer RNA (tRNA), ribosomal RNA (rRNA), small nuclear RNA (snRNA), small nucleolar RNA (snoRNA), as well as long noncoding RNA (lncRNA) [Id., citing Wei, Z., et al. Nat. Commun. (2017) 8:1145; Huang, X., et al. BMC Genomics (2013) 14:319; Crescitelli, R., et al. J. Extracell. Vesicles (2013) 2:20677].

The term “Fab fragment” as used herein refers to an antibody fragment composed of a single antigen-binding arm of an antibody without the Fc region, produced by cleavage of IgG by the enzyme papain. It contains the complete light chain plus the amino-terminal variable region and first constant region of the heavy chain, held together by an interchain disulfide bond.

The term “F(ab′)₂ fragment as used herein refers to an antibody fragment composed of two linked antigen-binding arms (Fab fragments) without the Fc regions, produced by cleavage of IgG with pepsin.

The term “forced vital capacity” or “FVC” is an objective measurement of respiratory muscle function. It refers to the maximal volume of gas that can be exhaled from full inhalation by exhaling as forcefully and rapidly as possible.

The term “fragment” or “peptide fragment” as used herein refers to a small part derived, cut off, or broken from a larger peptide, polypeptide or protein, which retains the desired biological activity of the larger peptide, polypeptide or protein. Antibody binding fragments (e.g., Fab, Fab′, F(ab′)₂, Fv, and single-chain (sc) antibodies) can be produced by recombinant DNA techniques, or by enzymatic or chemical cleavage of intact antibodies.

The term “healthy subject” as used herein refers to a subject having no signs or symptoms of a cancer.

The term “heterogeneous” as used herein refers to being composed of unrelated or unlike elements or parts; varied; miscellaneous; of different kinds; differing or opposite in structure, quality etc.; dissimilar.

The term “high throughput screening” or “HTS” as used herein refers to the use of automated equipment to rapidly test thousands to millions of samples for biological activity at the model organism, cellular, pathway, or molecular level.

The term “homogeneous” as used herein refers to being of the same character, structure, quality; etc.; essentially like; of the same nature; composed of similar or identical elements or parts; uniform.

As used herein, the term “immune checkpoints” refers to the array of inhibitory pathways necessary for maintaining self-tolerance and that modulate the duration and extent of immune responses to minimize damage to normal tissue. In T cells, the ultimate amplitude and quality of the immune response, which is initiated through antigen recognition by the TCR, is regulated by a balance between co-stimulatory and inhibitory signals (immune checkpoints). [Pardoll, DM. Nat. Rev. Cancer (2012) 12 (4): 252-64]. Immune checkpoint molecules such as PD-1, PD-L1, CTLA-4 are cell surface signaling receptors that play a role in modulating the T-cell response in the tumor microenvironment. Tumor cells have been shown to utilize these checkpoints to their benefit by up-regulating their expression and activity. With the tumor cell's ability to commandeer some immune checkpoint pathways as a mechanism of immune resistance, it has been hypothesized that checkpoint inhibitors that bind to molecules of immune cells to activate or inactivate them may relieve their inhibition of an immune response. Immune checkpoint inhibitors have been reported to block discrete checkpoints in an active host immune response allowing an endogenous anti-cancer immune response to be sustained. Recent discoveries have identified immune checkpoints or targets, like PD-1, PD-L1, PD-L2, CTLA-4, TIGIT, TIM-3, LAG-3, CCR4, OX40, OX40L, IDO, and A2AR, as proteins responsible for immune evasion.

The terms “immune escape” or “immune evasion” as used herein refers to a strategy to evade a host's immune response. It is characterized by the inability of the immune system to eliminate transformed cells prior to and after tumor development. The host's contribution is manifested by its inability to recognize antigens expressed by tumor cells, a phenomenon known as “host ignorance.” It happens because of defects in both the innate and adaptive arms of the immune system. The tumor's contribution is manifested by the adaptation of tumor cells to evade the immune system or by developing a microenvironment that suppresses the immune system. [Qian J., et al. (2011) Immune Escape. In: Schwab M. (eds) Encyclopedia of Cancer. Springer, Berlin, Heidelberg. doi.org/10.1007/978-3-642-16483-5_2975].

The term “immune homeostasis” refers to the delicate and finely regulated balance of appropriate immune activation and suppression in tissues and organs, driven by a myriad of cellular players and chemical factors. [da Gama Duarte, J., et al. Immunology and Cell Biology (2018) 96:497-506]

The terms “immune response” and “immune-mediated” are used interchangeably herein to refer to any functional expression of a subject's immune system, against either foreign or self-antigens, whether the consequences of these reactions are beneficial or harmful to the subject.

The term “immune system” as used herein refers to the body's system of defenses against disease, which comprises the innate immune system and the adaptive immune system. The innate immune system provides a non-specific first line of defense against pathogens. It comprises physical barriers (e.g. the skin) and both cellular (granulocytes, natural killer cells) and humoral (complement system) defense mechanisms. The reaction of the innate immune system is immediate, but unlike the adaptive immune system, it does not provide permanent immunity against pathogens. The adaptive immune response is the response of the vertebrate immune system to a specific antigen that typically generates immunological memory.

The term “immunomodulatory” as used herein refers to a substance or agent that is capable of augmenting or diminishing immune responses directly or indirectly, e.g., by expressing chemokines, cytokines and other mediators of immune responses.

The term “induce” and its various grammatical forms as used herein with respect to immunity refers to a process or action of bringing about or giving rise to an immune response.

The term “inhalation” as used herein refers to the process by which gases or air enter the lungs.

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to more than about 95%, 96%, 97%, 98%, 99% or 100% free. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state.

The term “join” as used herein means to link, couple, or connect one thing with another. Each of these terms is used interchangeably with the others.

The term “labeling” as used herein refers to a process of distinguishing a compound, structure, protein, peptide, antibody, cell or cell component by introducing a traceable constituent. Common traceable constituents include, but are not limited to, a fluorescent antibody, a fluorophore, a dye or a fluorescent dye, a stain or a fluorescent stain, a marker, a fluorescent marker, a chemical stain, a differential stain, a differential label, and a radioisotope.

The term “long noncoding RNA” (“lncRNAs”) as used herein refers to a class of transcribed RNA molecules that are longer than 200 nucleotides and yet do not encode proteins. LncRNAs can fold into complex structures and interact with proteins, DNA and other RNAs, modulating the activity, DNA targets or partners of multiprotein complexes. Crosstalk of lncRNAs with miRNAs creates an intricate network that exerts post-transcriptional regulation of gene expression. For example, lncRNAs can harbor miRNA binding sites and act as molecular decoys or sponges that sequester miRNAs away from other transcripts. Competition between lncRNAs and miRNAs for binding to target mRNAs has been reported and leads to de-repression of gene expression (Zampetaki, A. et al. Front. Physiol. (2018) doi.org/10.3389/fphys.2018.01201, citing Yoon, J H., et al. Semin. Cell Dev. Bio. (2014) 34:9-14; Ballantyne, MD., et al. Clin. Pharmacol. Ther. (2016) 99:494-501). Finally, lncRNAs may contain embedded miRNA sequences and serve as a source of miRNAs (Id., citing Piccoli, MT., et al. Cir. Res. (2017) 121:575-83).

The term “lung” as used herein refers to one of a pair of organs occupying the pulmonary cavities of the thorax, and are the organs of respiration in which aeration of the blood takes place. Each lung is irregularly conical in shape, presenting a blunt upper extremity (the apex), a concave base following the curve of the diaphragm, an outer convex surface (costal surface), an inner or mediastinal surface, a thin and sharp anterior border, and a thick and rounded posterior border.

The term “lung function” is used herein to refer to a measure of how well the lung is working. There are several types of lung function tests, including spirometry, pulse oximetry, exercise stress test or arterial blood gas test. Additionally, hydroxyproline levels, lung density and total cell count in bronchoalveolar lavage fluid (BAL) may be used to assess lung function. It is to be understood that any one of these tests may be used in combination with another.

For example, lung function may be assessed by determining the amounts of polymorphonuclear leukocytes, neutrophil products, eosinophils, eosinophil products, activated alveolar macrophages, alveolar macrophage products, cytokines, chemokines, growth factors for fibroblasts, and immune complexes in BAL fluid in an untreated control, or relative to a patient at a time point prior to treatment, where a decrease in the amounts of polymorphonuclear leukocytes, neutrophil products, eosinophils, eosinophil products, activated alveolar macrophages, alveolar macrophage products, cytokines, chemokines, growth factors for fibroblasts, and/or immune complexes is indicative of an increase in lung function.

Hydroxyproline is a major component of collagen, where it serves to stabilize the helical structure. Because hydroxyproline is largely restricted to collagen, the measurement of hydroxyproline levels can be used as an indicator of collagen content. A decrease in hydroxyproline levels relative to an untreated control subject are indicative of an increase in lung function.

The terms “Major Histocompatibility Complex (MHC), MHC-like molecule” and “HLA” are used interchangeably herein to refer to cell-surface molecules that display a molecular fraction known as an epitope or an antigen and mediate interactions of leukocytes with other leukocyte or body cells. MHCs are encoded by a large gene group and can be organized into three subgroups-class I, class II, and class III. In humans, the MHC gene complex is called HLA (“Human leukocyte antigen”); in mice, it is called H-2 (for “histocompatibility”). Both species have three main MHC class I genes, which are called HLA-A, HLA-B, and HLA-C in humans, and H2-K, H2-D and H2-L in the mouse. These encode the a chain of the respective MHC class I proteins. The other subunit of an MHC class I molecule is B2-microglobulin. The class II region includes the genes for the α and β chains (designated A and B) of the MHC class II molecules HLA-DR, HLA-DP, and HLA-DQ in humans. Also in the MHC class II region are the genes for the TAP1: TAP2 peptide transporter, the PSMB (or LMP) genes that encode proteasome subunits, the genes encoding the DMα and BMβ chains (DMA and DMB), the genes encoding the α and β chains of the DO molecule (DOA and DOB, respectively), and the gene encoding tapasin (TAPBP). The class II genes encode various other proteins with functions in immunity. The DMA and DMB genes encoding the subunits of the HLA-DM molecule that catalyzes peptide binding to MHC class II molecules are related to the MHC class II genes, as are the DOA and DOB genes that encode the subunits of the regulatory HLA-DO molecule. [Janeway's Immunobiology. 9^{th} ed., GS, Garland Science, Taylor & Francis Group, 2017. pps. 232-233]. In humans, there are three MHC class II isotypes: HLA-DR, HLA-DP, and HLA-DQ, encoded by a and β chain genes within the Human Leukocyte Antigen (HLA) locus on chromosome 6 [Wosen, J E., et al. Front. Immunol. (2018) doi.10.3389/fimmu.2018.02144].

As used herein, the terms “marker” or “cell surface marker” are used interchangeably to refer to an antigenic determinant or epitope found on the surface of a specific type of cell. Cell surface markers can facilitate the characterization of a cell type, its identification, and eventually its isolation. Cell sorting techniques are based on cellular biomarkers where a cell surface marker(s) may be used for either positive selection or negative selection, i.e., for inclusion or exclusion, from a cell population.

The term “messenger RNA” (“mRNA”) as used herein refers to a coding RNA, which functions in protein translation.

The term “metastasis” as used herein refers to spread of cancer cells from the place where they first formed to another part of the body. In metastasis, cancer cells break away from the original (primary) tumor, travel through the blood or lymph system, and form a new tumor in other organs or tissues of the body. The new, metastatic tumor is the same type of cancer as the primary tumor. For example, if breast cancer spreads to the lung, the cancer cells in the lung are breast cancer cells, not lung cancer cells.

The term “microRNA” (or “miRNA”) as used herein refers to a class of small, 18- to 28-nucleotide-long, noncoding RNA molecules. Their major role is in the posttranscriptional regulation of protein expression

The term “minimum residual disease” or “measurable residual disease” are used interchangeably to refer to the small number of cancer cells that remain in a patient that share phenotypic similarity (e.g., histologic appearance, lineage markers) and genetic heritage (e.g., mutations and rearrangements) with the original tumor cells. This definition excludes residual cells that harbor somatic alterations and/or phenotypic alterations but are not fully malignant. [Luskin, M R., et al. Nat. Rev. Cancer (2018) 18 (4): 255-63].

As used herein, the term “mutation” refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Exemplary mechanisms of mutation include substitution (exchange of one base pair for another), addition (the insertion of one or more bases into a sequence), and deletion (loss of one or more base pairs).

The terms “next generation sequencing”, “NGS”, “massively parallel sequencing”, or “deep sequencing” as used herein describe a high-throughput method used to determine the nucleotide sequence of an individual's whole genome at once in an automated process. First a DNA library is prepared from a patient's sample by fragmentation, purification and amplification of the DNA sample. Individual fragments are then physically isolated by attachment to solid surfaces or small beads. The sequence of each of these fragments is resolved simultaneously by such techniques as sequencing by synthesis. The resulting sequence data are computationally aligned against a ‘normal reference’ genome.

The term “non-coding RNA” (“ncRNA”) as used herein refers to a functional RNA molecule that is transcribed from DNA but not translated into proteins. They are classified into housekeeping and regulatory noncoding RNAs. Housekeeping ncRNAs include ribosomal RNA (rRNA, the RNA component of ribosomes), transfer RNA (IRNA, which functions as an adapter for matching amino acids to mRNA), small nuclear RNA (snRNA, which functions in RNA processing such as mRNA splicing), and small nucleolar RNAs (snoRNAs, which functions in guiding chemical modification of other RNAs). Regulatory noncoding RNAs are divided into short ncRNAs (<200 nt) and long ncRNAs (>200 nts). Short noncoding RNAs <200 nt include microRNA (miRNA), small interfering RNAs (siRNAs) and piwi-associated RNAs (piRNAs), and long noncoding RNAs (>200 nt). [Losko, M., et al. Mediators of Inflammation (2016) 1-12. 10.1155/2016/5365209].

The term “non-invasive” as used herein refers to a medical procedure that does not require insertion of an instrument or device through the skin or a body orifice for diagnosis or treatment.

The term “nucleic acid” as used herein refers to a deoxyribonucleotide or ribonucleotide polymer in either single- or double-stranded form, and, unless otherwise limited, encompasses known analogues having the essential nature of natural nucleotides in that they hybridize to single-stranded nucleic acids in a manner similar to naturally occurring nucleotides (e.g., peptide nucleic acids).

The term “nucleotide” as used herein refers to a molecule consisting of a nitrogen-containing base (adenine, guanine, thymine, or cytosine in DNA; adenine, guanine, uracil, or cytosine in RNA), a phosphate group, and a sugar (deoxyribose in DNA; ribose in RNA).

The term “objective response rate” or “ORR” as used herein refers to the percentage of people in a study or treatment group who have a partial response or complete response to the treatment within a certain period of time.

The term “orthotopic model” as used herein refers to the seeding of tumor cell lines or patient-derived cell xenografts into animal models.

The term “overall survival” as used herein refers to the length of time from either the date of diagnosis or the start of treatment for a disease, such as cancer, that patients diagnosed with the disease are still alive.

The term “particle” as used herein refers to an extremely small constituent, e.g., nanoparticles.

The term “patient-derived xenograft” or “PDX” as used herein is a model initially created by implanting a fragment of a human tumor into a mouse. The resulting population of these PDX mice largely retain the genetics of the human tumors from which they were initially created.

The term “PDZ domain” as used herein refers to abundant protein interaction modules that often recognize short amino acid motifs at the C-termini of target proteins. They have been implicated in regulating multiple biological processes, such as transport, ion channel signaling and other signal transduction systems. (see Lee, H-J & Zheng, JJ. Cell Communication & Signaling (2010) 8: article 8).

The term “placental alkaline phosphatase” or “PLAP” refers to an enzyme normally produced by primordial germ cells and syncytiotrophoblasts; the detection of its expression has been useful in the diagnosis of germ cell tumors. PLAP immunoreactivity in normal human adult and fetal muscle tissue has been observed. This immunoreactivity seems to relate to the degree of myogenic differentiation in soft tissue tumors and is more frequently expressed in cells with skeletal muscle differentiation and least in those with myofibroblastic features. (Goldsmith, J D, et al., Am J. Surgical Pathol (2002) 26 (12): 1627-33).

The term “plasma” as used herein refers to the fluid (noncellular) portion of circulating blood, and the fluid portion of lymph.

The term “pleural effusion” as used herein refers to a buildup of fluid between the layers of tissue that line the lungs and chest cavity that can pevent the lungs frum fully inflating, making it hard to breathe.

The term “pleural fluid” refers to a liquid located between the layers of the pleura, a two-layer membrane that covers the lungs and lines the chest cavity. Pleural fluid keeps the pleura moist and reduces frictaon between the membranes when breathing. The area that contains pleural fluid is known as the pleural space. In healthy subjects, there is small amount of pleural fluid in the pleural space.

The term “polymer” refers to a large molecule, or macromolecule, composed of many repeated subunits. The term “monomer” refers to a molecule that may bind chemically to other molecules to form a polymer. The term “copolymer” as used herein refers to a polymer derived from more than one species of monomer.

The terms “polypeptide” and “protein” are used herein in their broadest sense to refer to a sequence of subunit amino acids, amino acid analogs, or peptidomimetics. The subunits are linked by peptide bonds, except where noted. These terms also apply to amino acid polymers in which one or more amino acid residue is an artificial chemical analogue of a corresponding naturally occurring amino acid, as well as to naturally occurring amino acid polymers. The terms also are inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides may not be entirely linear. For instance, polypeptides may be branched as a result of ubiquitination, or they may be circular, with or without branching, generally as a result of posttranslational events, whether by natural processing or by events brought about by human manipulation, which do not occur naturally. Circular, branched and branched circular polypeptides may be synthesized by entirely synthetic methods.

The term “proliferate” and its various grammatical forms as used herein is meant to refer to the process that results in an increase of the number of cells, and is defined by the balance between cell division and cell loss through cell death or differentiation.

The term “purification” and its various grammatical forms as used herein refers to a process of isolating or freeing from foreign, extraneous, or objectionable elements. The composition is nonetheless substantially pure in that it has been substantially separated from the substances with which it may be associated in living systems or during synthesis. As used herein, the term “substantially pure” refers purity of at least 75%, at least 80%, at least 85%, at least 90%, at least 95% or at least 99% pure as determined by an analytical protocol. Such protocols may include, for example, without limitation, flow cytometry, electrophoresis, small-RNA sequencing, quantitative PCR, nanoparticle tracking, electron microscopy, mass spectrometry (MS), Western blotting, ELISA, and various metabolic assays.

The term “quantitative PCR” (or “qPCR”), also called “real time-PCR” or “quantitative real-time PCR” refers to a polymerase chain reaction-based technique that couples amplification of a target DNA sequence with quantification of the concentration of that DNA species in the reaction.

The term “restriction enzyme” as used herein refers to an enzyme produced by bacteria that recognizes a short sequence in a nucleic acid molecule and cleaves the nucleic acid molecule at or near that sequence, resulting in a double-stranded or single-stranded break. The term “restriction enzyme recognition site” or “restriction site” as used herein is a short specific sequence of base pairs of DNA recognized by each restriction enzyme.

The term “serum” as used herein refers to the fluid portion of the blood obtained after removal of the fibrin clot and blood cells.

The term “solid tumor” as used herein refers to an abnormal mass of tissue that usually does not contain cysts or liquid areas. Solid tumors may be benign (a growth that does not invade nearby tissue or spread to other parts of the body) or malignant (meaning to grow in an uncontrolled way); malignant tumors can invade nearby tissues and spread to other parts of the body through the blood and lymph system). Different types of solid tumors are named for the type of cells that form them.

The term “splice variant”, also known as a “splice-site mutation” as used herein refers to a genetic alteration in the DNA sequence that occurs at the boundary of an exon and an intron (splice site). This change can disrupt RNA splicing resulting in the loss of exons or the inclusion of introns and an altered protein-coding sequence.

The term “strand displacement” as used herein refers to a reaction in DNA homologous recombination and DNA mismatch repair. It is a reaction where one of the strands in a double-stranded DNA is replaced with another nearly identical strand. DNA strand displacement involves three single strands named the “invader”, the “incumbent” and the “substrate strands. Basically, it is a swapping reaction between the invader and the incumbent strands on the substrate strand. A double-stranded nucleic acid molecule is separated, partially or completely into two separate single-stranded nucleic acid strands by an enzyme having strand displacement activity (e.g., unwinding activity), such as a topoisomerase or helicase, and an invader strand complementary to the incumbent strand in the double-stranded nucleic acid. The enzyme unwinds the double-stranded nucleic acid allowing for the invader complementary strand to hybridize to one of the separated strands from the double-stranded nucleic acid, thereby blocking the incumbent double-stranded nucleic acid strand from reannealing with its matching substrate strand in the original double stranded DNA molecule. The incumbent single stranded DNA that was displaced then undergoes branch-migration until the incumbent strand is fully displaced by the invader strand. (Broadwater, Jr. and DWB, Kim, HD, Biophys. J. (2016) 110 (7): 1476-84). The described and claimed assay comprises DNA displacement, whereby a small oligonucleotide hybridizes to and overhang at the end of the double stranded DNA duplex, onto the DNA strand attached to the antibody and a DNA polymerase reaction allows the initial DNA strands (one connected to the antibody and one connected to the platform via the biotin) to separate so a unique antibody with a unique DNA strand may be released specifically from a group of different antibodies targeting different surface markers of exosomes.

The term “surfactant protein C” (or “SP-C”) as used herein refers to an extremely hydrophobic surfactant protein essential for lung function and homeostasis after birth. It is a surface-active lipoprotein complex composed of 90% lipids and 10% proteins which include plasma proteins and apolipoproteins SPA, SPB, SPC and SPD. The surfactant is secreted by the alveolar cells of the lung and maintains the stability of pulmonary tissue by reducing the surface tension of fluids that coat the lung.

The terms “tailored therapy,” “personalized medicine” or “precision medicine” are used interchangeably to refer to an approach for disease treatment and prevention that takes into account individual variability in genes, environment and lifestyle. A precision medicine approach allows for a more accurate prediction of which treatment and prevention strategies for a particular disease will work in which groups of patients. This is in contrast to a one-size-fits-all approach, in which disease treatment and prevention strategies are developed for the average person with less consideration for differences between individuals.

The terms “target cell” or “cell of origin” as used herein refers to the first cell that undergoes a tumorigenic mutation during the initiation stage of carcinogenesis.

The term “targeted” with regard to capture of EVs as used herein refers to the specific and selective isolation and purification of EVs by the EV-CATCHER technology.

The term “terminal respiratory unit” as used herein refers to all of the alveolar ducts, together with their accompanying alveoli that stem from the most proximal (first) respiratory bronchiole and contains approximately 100 alveolar ducts and 2000 alveoli. Because gse phase diffusin is so rapid, the partial pressures of oxygen and carbon dioxide are uniform throughout the unit.

The term “tetraspanin” as used herein refers to membrane-spanning proteins with a conserved structure that function primarily as membrane protein organizers. Members of the tetraspanin family of proteins have four transmembrane domains, which contribute to the creation of a small (EC1) and large (EC2) extracellular loop [Termini, CM, Gillette, J M, Front. Cell Dev. Biol. (2017) 5:34, citing Abe, M. et al. Cancer Lett. (2008) 266:163-70). The large extracellular loop contains a conserved Cys-Cys-Gly amino acid motif (CCG-motif), as well as two other conserved cysteine residues. Many members of the tetraspanin family also contain post-translational modifications.

The term “therapeutic agent” as used herein refers to a drug, molecule, nucleic acid, protein, metabolite, cell, composition or other substance that provides a therapeutic effect. The term “active” as used herein refers to the ingredient, component or constituent of the compositions of the described invention responsible for the intended therapeutic effect. The terms “therapeutic agent” and “active agent” are used interchangeably herein. For any therapeutic agent described herein, the therapeutic amount initially may be determined from preliminary in vitro studies and/or animal models. An effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50 which describes the dose in a particular dosage that is therapeutically effective for a particular disease manifestation in 50% of a population.

The terms “therapeutic amount”, an “amount effective”, or “pharmaceutically effective amount” of an active agent are used interchangeably to refer to an amount that is sufficient to provide the intended benefit of treatment. However, dosage levels are based on a variety of factors, including the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

The term “therapeutic effect” as used herein refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

In most clinical situations, drugs are administered in a series of repetitive doses or as a continuous infusion to maintain a steady-state concentration of drug associated with the therapeutic window. To maintain the chosen steady-state or target concentration (“maintenance dose”), the rate of drug administration is adjusted such that the rate of input equals the rate of loss. If the clinician chooses the desired concentration of drug in plasma and knows the clearance and bioavailability for that drug in a particular patient, the appropriate dose and dosing interval can be calculated. However, living cellular therapies break this concept, since they divide and may even take up permanent residence in the body in the case of autologous cellular therapy. Hence what is initially administered can bear little correlation to what is present in the recipient over time.

The term “tidal volume” as used herein refers to the amount of air that moves in or out of the lungs during a normal breath. It measures about 500 mL in an average healthy adult human male, and approximately 400 mL in an average healthy adult human female.

The term “transduction” and its various grammatical forms as used herein refers to a process whereby foreign DNA is introduced into another cell via a viral vector.

The term “transfection” and its various grammatical forms as used herein refers to the process of introducing a foreign DNA molecule into a eukaryotic cell by nonviral methods.

The term “transgenic mouse model” as used herein refers to mice that have had DNA from another source inserted into their DNA. The foreign DNA, which is inserted into the nucleus of a fertilized mouse egg, becomes part of every cell and tissue of the mouse.

The terms “tumor burden” and “tumor load” are used interchangeably to refer to the number of cancer cells, the size of a tumor, or the amount of cancer in the body.

The terms “tumorigenesis” “oncogenesis” and “carcinogenesis” are used interchangeably to refer to the transformation of normal cells into cells-of-origin (COOs) and the development of cells-of-origin into tumors.

The term “variant” with respect to a gene or nucleic acid sequence is a sequence having at least 65% identity with a reference gene or nucleic acid sequence, and can include one or more base deletions, additions, or substitutions with respect to the referenced sequence. The term “variant” with respect to a peptide or protein sequence is a sequence that varies at one or more amino acid positions with respect to the reference peptide or protein and can include single or multiple amino acid substitutions, deletions, additions or replacements.

### Embodiments

According to one aspect, the present disclosure provides a non-invasive method for targeted capture of a purified population of extracellular vesicles (EVs) derived from lung cancer cells and contained in exhaled breath obtained from a subject for evaluating a cargo of the purified population of EVs comprising: a) obtaining an expressed breath sample from the subject; b) condensing the exhaled breath sample in a cooling chamber and collecting the exhaled breath condensate (EBC); c) preparing a purified population of EVs by: contacting the EBC comprising EVs from the subject with a binding agent directed to one or more EV surface antigens; wherein the binding agent is linked to a nucleic acid, and wherein the nucleic acid is immobilized on a solid support; d) isolating the EVs bound by the binding agent from the exhaled breath condensate; e) releasing the EVs bound to the binding agent; f) eluting the bound EV from the binding agent to form a population of free purified EVs; and g) evaluating cargo and surface molecules comprising protein, nucleic acids or lipids, of the purified population of EVs.

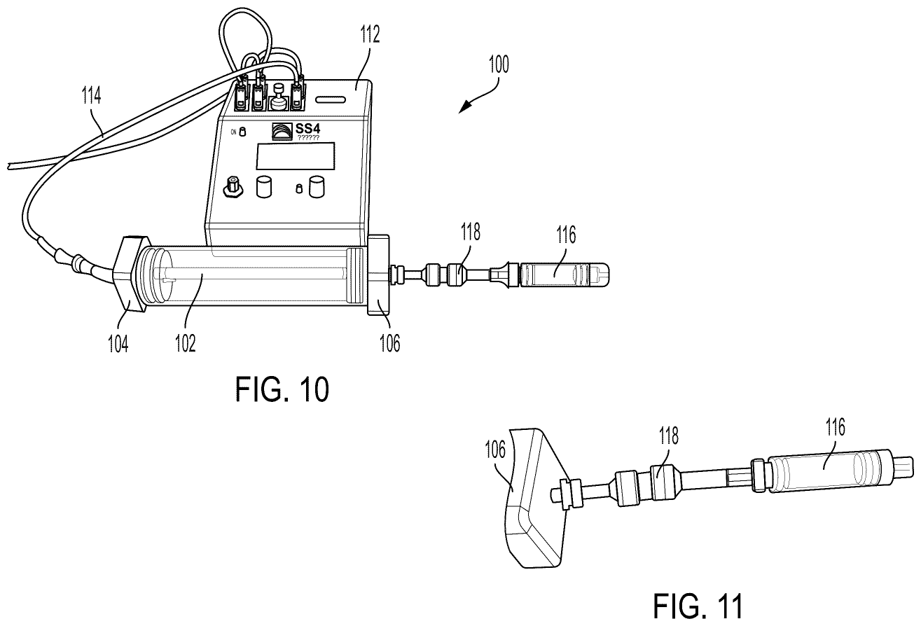

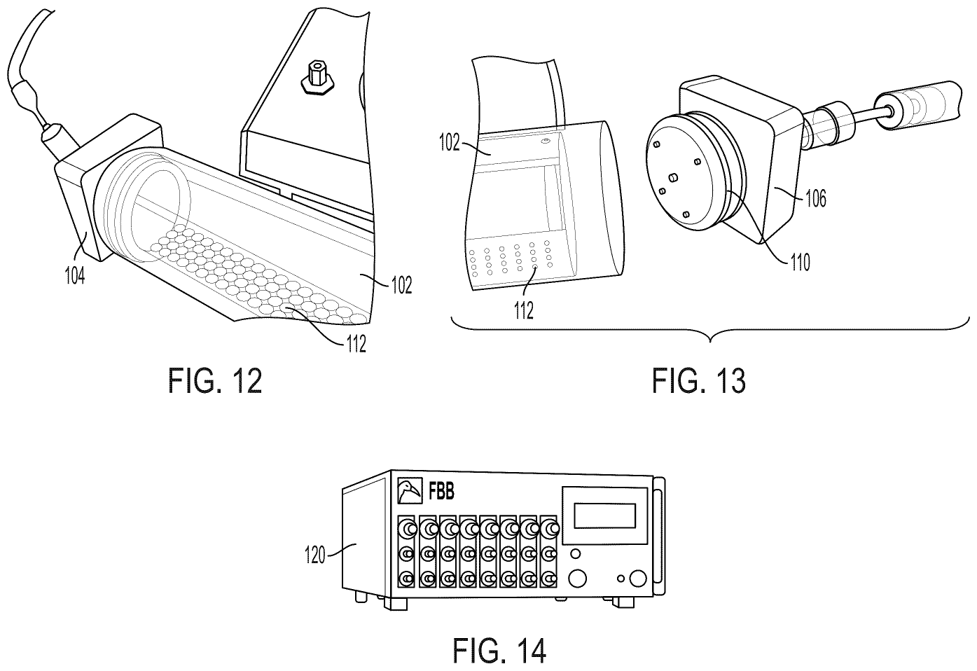

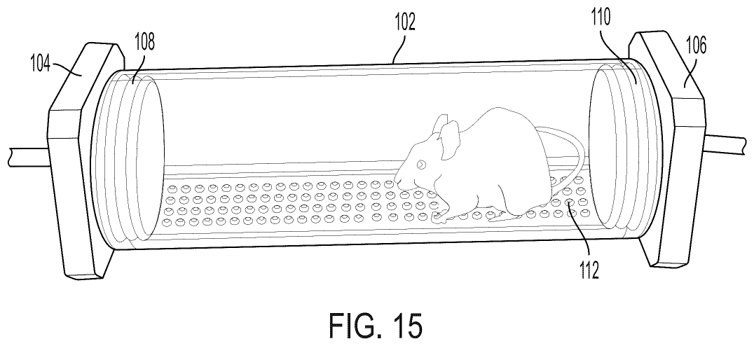

FIGS. 10-15 are perspective and detailed views of an exemplary Exhaled Breath Condensate (EBC) collection device 100. The device 100 includes a sterile glass RC3 respirometer chamber 102 (e.g., manufactured by Sable Systems International). The chamber 102 includes two endcaps 104, 106 on opposing ends with seals 108, 110 to seal the interior volume of the chamber 102. The chamber 102 includes a platform 112 along the bottom surface to support mice positioned therein. The chamber 102 is configured to receive two mice simultaneously and allows the mice to move within the chamber 102 during collection of the condensate.

The chamber 102 is attached to a SS4 flow pump/meter 114 via tubing 116, which is set to a rate of about 20 ml/min. A mass flow meter system 120 (FIG. 14) can be used in combination with the flow pump/meter 114. The flow pump/meter 114 sets the rate of inlet air into the chamber 102, with the about 20 ml/min setting being the recommended flow rate needed to allow for enough air flow into the chamber 102 to not have animals suffocate during the collection period. The respiratory chamber 102 does not lead to significant restraint of the animal, as the animals still have freedom to move around with normal postural movement (i.e., walk and turn around freely) within the chamber 102. In some embodiments, the chamber 102 can be about 10 inches in length and 3 inches in diameter. Mice are kept in the chamber 102 for about 4 hours to allow for adequate volumes of EBC to be collected (in this time frame an estimated volume of about 100-200 μl of EBC can be collected). The EBC is collected in collection chamber 116, which is connected to the opposing end of the chamber 102 via joints and/or tubing 118.

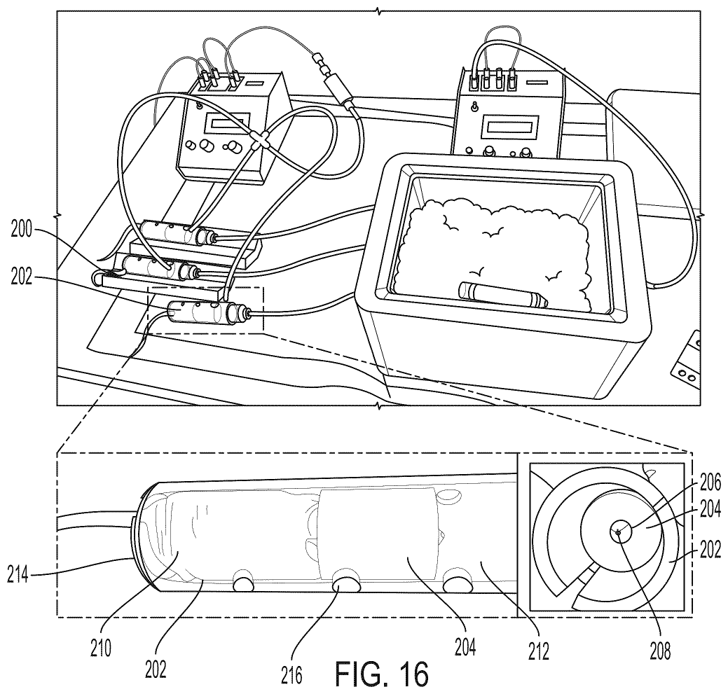

FIG. 16 is a perspective and detailed view of an exemplary Exhaled Breath Condensate (EBC) collection device 200. Because the device 100 (discussed above) allows mice to move freely within the chamber 102, the device 100 does not inhibit their ability to scratch themselves and/or urinate and defecate within the chamber 102. Collected data has revealed the presence of significant contamination from both skin and urinary proteins in EBC specimens collected using the device 100. However, for animals that have undergone a lung injury (for evaluation of response to toxic chemicals), the device allows for the collection of EBC without restraint or manipulation of the animals with biological material in the EBC that contains exhaled EVs. Thus, in order to combat and prevent such contamination in EBC specimens which, in turn, allows for enhanced sensitivity to the detection of cancer biomarkers, the device 200 was developed.

The device 200 provides a design and setup for receiving a single mouse in a restrained manner to prevent contamination issues observed when using the device 100. The device 200 and its operations have been described in, e.g., Liu et al., “A device to collect exhaled breath to study biomarkers in small animal models,” (2019) (doi: doi.org/10.1101/511931), which is incorporated herein by reference in its entirety. The device 200 includes a sterile glass chamber 202 configured and dimensioned to receive only a single mouse. In some embodiments, the chamber 202 can be about, e.g., 1 inch at the inside diameter and 3.5 inches in length for small mice weighing up to 25 g, 1.25 at the inside diameter and 4.25 inches in length for medium sized mice weighing between 25-50 g, and 1.5 inches at the inside diameter and 5.25 in length for larger mice weighing between 50-75 g, The interior of the chamber 202 includes a restrainer cylinder or ring 204 along one section of the inner wall to assist in restricting movement of the mouse. The ring 204 can be fabricated from a rubber material to create friction for restricting movement of the mouse. The ring 204 includes a central opening 206 configured and dimensioned to at least partially receive the nose 208 of the mouse therethrough. The ring 204 can include different inner dimensions. For example, one section of the ring 204 can have an inner diameter opening configured to at least partially receive the body and/or head of the mouse therein, and another section of the ring 204 can have a smaller diameter to form the opening 206. The ring 204 thereby separates the interior of the chamber 202 into a first section 210 configured to contain the rear section of the mouse, and a second section 212 configured to collection EBC. The opposing ends of the chamber 202 can include caps 214 to assist with sealing the interior of the chamber 202 (only one cap 214 is shown for clarity). The chamber 202 can include multiple holes 216 formed along its length. The holes 216 allow for better restraining of the mice as the cap 214 keeping the mice in the chamber 202 can be secured into the appropriate hole based on mouse length, e.g., younger or sick mice tend to be smaller and therefore tend to require the cap 214 to be secured at a hole 216 closer to the nose 208 cone in order to prevent them from being able to wriggle and pull their nose 208 out of the nose 208 cone opening 206.

The device 200 allows for placement of a single mouse in the restrainer chamber 202 which only exposes the nose 208, allowing for uncontaminated collection of EBC. The device 200 utilizes the same flow pump/meter described for the device 100, and a rate of inlet air into the chamber 202 is also set to about 20 ml/min. In order to ensure one-way air flow, a second flow meter can be attached to the other end of the chamber 202 and is set to a flow rate of about 2 ml/min. Based on experiments, the device 200 allows for the collection of nose-only EBC volumes around about 20 μl over the course of about 2 hours, and improves the overall quality of the EBC sample. In some embodiments, the device 200 can be sterilized between each use. In some embodiments, the device 200 can be a single use device to prevent potential contamination.

According to some embodiments, the subject is a primary lung tumor bearing animal model wherein the lung tumor is a human tumor comprising mutations including EGFR and KRAS mutations, as well as p53, Ret, Her2, ROS1, Met, BRAF, NRAS, ALK, MAP2K1 or PI3KCA mutations and other mutations associated with NSCLC and SCLC. According to some embodiments, for each model, control animal colonies is established with primary human small airway epithelial cells (HSAECs).

According to some embodiments, the animal model is a transgenic mouse model. According to some embodiments of the transgenic mouse model, human tumor cells comprising mutations including EGFR and KRAS mutations and a bioluminescent construct are instilled through the trachea of the animals. According to some embodiments, the transgenic mouse is transduced or transfected to express EGFRL858R protein in bronchiolar Clara cells. According to some embodiments, the transgenic mouse is transduced or transfected to express KRASG12D protein in Clara cells and alveolar type II cells. The KRAS-G12D mutation has been reported to drive immune suppression and the primary resistance of anti-PD-1/PD-L1 immunotherapy in non-small cell lung cancer. It is associated with a low/never-smoking status [Liu, C. et al. Cancer Commun. (2022) 42 (9): 828-47].

According to some embodiments, the transgenic mouse is transfected to express CC10 protein for the study of multifocal bronchioloalveolar hyperplasias which develop into mixed solid and papillary adenocarcinomas, adenocarcinomas with focal NE differentiation, epithelial cell hyperplasia and adenomatous hyperplasia and bronchogenic adenocarcinomas. According to some embodiments, the transgenic mouse is transfected to express SP-C protein for the study of bronchioloalveolar adenomas and adenocarcinomas.e. the transgenic mouse is transfected to express Trp5 for the study of adenocarcinomas, NE hyperplasia and small-cell carcinoma with metastases. According to some embodiments, the transgenic mouse is transfected to express Rb for the study of NE hyperplasia and small-cell carcinoma with metastases.

According to some embodiments, the transgenic model is a TA-CCSP model that expresses the reverse tetracycline-controlled transactivator (rtTA) protein under the control of the rat CCSP (or SCGblal, secretoglobin, family 1A, member 1 (uteroglobin)) gene promoter, and provide a “Tet-On tool that allows the inducible expression of genes in the developing and respiratory epithelium (The Jackson Laboratory). Both SPC-ItTA and CCSP-rtTA transgenes [Inoue, K., et al. Genetically engineered mouse models for human lung cancer (2013) IntechOpen Chapter 2, dooi.org/10/5772/53721, citing Perl, AK., et al., Transgenic Res. (2002) 11:21-9] have been used for directing dox-responsive rtTA to either alveolar type II or Clara cells. Although both of these promoters have been used to create lung cancer models of mice, CCSP-rtTA has more widely been used than SPC-rtTA since the CCSP promoter is active in both Clara cells and alveolar type II cells while the SPC promoter is active only in alveolar type II cells [Id., citing Floyd, H S., et al. Carcinogenesis (2005) 26:2196-206]. According to some embodiments, the transgenic TA-CCSP model allows for production of the oncogene only in Clara cells. According to some embodiments, exhaled EVs can be evaluated at the stages of hyperplasia and non-invasive disease.

According to some embodiments, the animal model is an orthotopic human tumor NOD/SCID mouse model bearing a human lung tumor. According to some embodiments, human NSCLC lung cancer cell lines are transduced or transfected for ex vivo bioluminescence, expanded in vitro and administered directly into the lungs of NOD/SCID nude mice by tracheal instillation for lung uptake. According to some embodiments, human cell lines from other non-lung tissue origins (i.e., breast, colon, melanoma and/or liver) are transduced or transfected for ex vivo bioluminescence, expanded in vitro and administered via tail vein injection into NOD/SCID nude mice for the assessment of secondary lung cancer.

According to some embodiments, the animal model is a PDX human tumor NOD/SCID mouse model established using NSCLC patient-derived tumor cells comprising mutations including EGFR and KRAS mutations.

According to some embodiments, the transgenic model, the orthotopic model and the PDX model are complementary, meaning when considered together they enhance or emphasize the qualities of each other to form a complete unit.

According to some embodiments, step (g) evaluating cargo and surface molecules further comprises: (i) identifying proteins specific to a surface of the biological particles, for example, by mass spectrometry; and/or (ii) identifying protein cargos, for example, by mass spectrometry; and/or (iii) identifying DNA molecules by sequencing, or quantitative PCR; and/or (iv) extracting RNA from the purified population of EVs, and identifying and quantifying expression of small non-coding RNAs comprising microRNAs (miRNAs) encapsulated by the purified population of EVs, for example, by digital drop PCT (ddPCR).

According to some embodiments, the method comprises an initial ultrafiltration or ultracentrifugation step to provide a starting pooled heterogeneous population of EVs.

According to some embodiments, the binding agent that binds to one or more biological particle surface antigens is an antibody, an antibody binding fragment, or an aptamer. According to some embodiments, the aptamer is a nucleic acid or a polypeptide.

According to some embodiments, the aptamer comprises two complementary primers used for EV-Catcher, for example:

5'-Azide (5'Az-
AAAAACGAUUCGAGAACGUGACUGCCAUGCCAGCUCGUACUAU CGAA
(SEQ ID NO: 1)) and
3'-Biotin (5'Bio-CGAUAGUACGAGCUGGCAUGGCAGUCACGUUC
UCGAAUCGUUUU (SEQ ID NO: 2)).

According to some embodiments, the aptomer comprises two complementary primers containing specific restriction enzyme recognition sites used for EV-CATCHER, including, without limitation:

BamHI: 5'-Azide (5'Az- AAAAACGATTCGAGAACGTGAATCTC
GTTAACCGCTCAACTGGATCCCCAGCTCGTACTCCGCGATTCGTGCTCC
GTACTCCAATC (SEQ ID NO: 120)) and
BamHI: 3'-Biotin (5'Bio-CGATTGGAGTACGGAGCACGAATCG
CCGAGTACGAGCTGGGGATCCAGTTGAGCGGTTAACGAGATTCACGTTC
TCGAATCGTTT (SEQ ID NO: 121));
HindIII: 5'-Azide
(5'Az- AAAAACGATTCGAGAACGTGAATCTCGTTAACCGCTCAACTA
AGCTTCCAGCTCGTACTCCGCGATTCGTGCTCCGTACTCCAATC (SEQ
ID NO: 122)) and
HindIII: 3'-Biotin (5'Bio-
CGATTGGAGTACGGAGCACGAATCGCCGAGTACGAGCTGGAAGCTTAGT
TGAGCGGTTAACGAGATTCACGTTCTCGAATCGTTT (SEQ ID NO:
123)); or
SpeI: 5'-Azide
(5'Az- AAAAACGATTCGAGAACGTGAATCTCGTTAACCGCTCAACTA
CTAGTCCAGCTCGTACTCCGCGATTCGTGCTCCGTACTCCAATC (SEQ
ID NO: 124)) and
SpeI: 3'-Biotin (5'Bio-
CGATTGGAGTACGGAGCACGAATCGCCGAGTACGAGCTGGACTAGTAGT
TGAGCGGTTAACGAGATTCACGTTCTCGAATCGTTT (SEQ ID NO:
125)).

According to some embodiments, the efficiency of each restriction enzyme digestion can be evaluated using quantitative PCR using site specific primer/probe sequences, including, without limitation:

BamHI: Forward Primer ACGATTCGAGAACGTGAATCTC (SEQ
ID NO: 126),
Probe TTAACCGCTCAACTGGATCCC (SEQ ID NO: 127),
Reverse Primer TGGAGTACGGAGCACGAA (SEQ ID NO:
128);
HindIII: Forward Primer ACGATTCGAGAACGTGAATCTC
(SEQ ID NO: 126), Probe AACCGCTCAACTAAGCTTCCAGCT
(SEQ ID NO: 129), Reverse Primer TGGAGTACGGAGCACG
AA (SEQ ID NO: 128);
Spel: Forward Primer ACGATTCGAGAACGTGAATCTC (SEQ
ID NO: 126), Probe TACTAGTCCAGCTCGTACTCCGCG (SEQ
ID NO: 130), Reverse Primer TGGAGTACGGAGCACGAA
(SEQ ID NO: 128).

According to some embodiments, the EV surface antigen comprises one or more cell marker inherited by the exh-EV. According to some embodiments the cell marker inherited by the exh-EV includes CD9, CD63, CD81, CD37, CD82, Alix, Tim4, PLAP, Adiponectin, FABP4, Caveolin-1, Cytokeratins, EPCAM, E-Cadherin, P63, CCSP, or an SFTPC encoded protein specific to Clara cells or to alveolar type II respiratory cells, or a variant thereof.

According to some embodiments, the EV surface antigen is a surface protein specific to Clara cells, aleveolar type 2 (AT2) respiratory cells or both. According to some embodiments, the EV surface antigen is a club cell secretory protein (CCSP) variant or surfactant protein C (SP-C) variant encoded by the SFTPC gene.

Proteomic biomarkers are those discovered using technology capable of analyzing many proteins simultaneously, such as protein microarray and mass spectrometry (MS). According to some embodiments, the exhaled EVs are captured on a surface (e.g., EV-CATCHER) and assayed by ELISA to detect the presence of one or a combination of proteins. According to some embodiments, sample preparation comprises suspension-trapping-based sample preparation and label-free data-independent acquisition (DIA) mass spectrometry. [Wu, C. et al. Anal. Bioanal. Chem. (2022) 4141 (8): 2585-95]. According to some embodiments, the combination of proteomic biomarkers is specific to a cancer.

According to some embodiments, the nucleic acid comprises DNA, RNA, or a combination thereof. According to some embodiments, the nucleic acid comprises non-natural nucleotides. According to some embodiments, the nucleic acid comprises DNA. According to some embodiments, the DNA comprises a restriction enzyme recognition site. According to some embodiments, the DNA comprises one or more ribonucleic acid nucleotide. According to some embodiments, the one or more ribonucleic acid nucleotide is uracil. According to some embodiments, the nucleic acid further comprises a binding moiety on a first end of the nucleic acid and a binding moiety on a second end of the nucleic acid, wherein the binding moiety on the first end of the nucleic acid and the binding moiety on the second end of the nucleic acid are different. According to some embodiments, the binding moiety on the first end of the nucleic acid is an avidin, streptavidin or carboxyl binding moiety. According to some embodiments, the binding moiety is biotin. According to some embodiments, the binding moiety on the second end of the nucleic acid is an amine moiety. According to some embodiments, the amine moiety is azide. According to some embodiments, the binding agent to one or more biological particle surface antigens comprises a dibenzocyclooctyne (DBCO) molecule, 2-IT (2-iminothiolane), MBS (3-maleimidobenzoic acid N-hydroxysuccinimide ester), SPDP (N-succinimidyl 3-(2-pyridyldithio) propionate), SATA (N-succinimidyl S-acetylthioacetate), SMCC (succinimidyl 4-(N-maleimidomethyl) cyclohexane-1-carboxylate), Sulfo-SMCC, or derivatives thereof.

According to some embodiments, the solid support is a well plate, polymer, or a surface.

According to some embodiments, releasing the isolated biological particle comprises: (i) enzymatically cleaving the nucleic acid; or (ii) displacing a first strand of the nucleic acids connected to the antibody from the second strand of the nucleic acids connected to the support by strand displacement with a complementary nucleic acid to the first or second strand of the nucleic acid and an enzyme having strand displacement activity to release the antibody from the support; or (iii) separating the annealed DNA strands to allow release of the antibody from the platform without damaging the DNA strand attached to the antibody by a polymerase chain reaction using an oligonucleotide complementary to the region of the DNA attached to the antibody. According to some embodiments, the enzymatic cleaving is with uracil glycosylase. According to some embodiments, the enzymatic cleaving is with a restriction enzyme. According to some embodiments, the enzyme having strand displacement activity is DNA polymerase, topoisomerase, or helicase.

According to some embodiments, the method comprises detecting, identifying and measuring a level of the one or more small non-coding RNAs comprising miRNAs encapsulated in the EVs by next generation sequencing. According to some embodiments, the next generation sequencing (NGS) includes deep sequencing.

According to some embodiments, the miRNA encapsulated in the EVs is one or more miRNA listed in Table 5.

According to another aspect, the present disclosure provides a non-invasive method for optimizing therapeutic benefit for a subject at risk of lung cancer, comprising
a) obtaining an exhaled breath sample from the subject and from a healthy control;
b) condensing the exhaled breath in a cooling chamber and collecting the exhaled breath condensate (EBC);
c) purifying EVs contained in the EBC obtained from the subject and healthy control;
d) measuring a level of expression of each of a plurality of mRNAs, miRNAs, or protein cargo in the EVs contained in the EBC sample from the subject and in the EVs contained in the EBC sample from the healthy control;
e) determining that expression of the one or more of the mRNAs, miRNAs or protein cargo in the EVs contained in the EBC sample from the subject is dysregulated compared to the healthy control;
f) identifying the patient as one that can benefit therapeutically from being treated for lung cancer, when the presence of one or more dysregulated mRNAs, miRNAs or protein cargo in the EVs contained in the EB sample obtained from the subject is detected, wherein the detection may correlate with tumor burden.

According to some embodiments, the non-invasive method further comprises:
g) tailoring an effective medical treatment for the lung cancer based on genetic, environmental and lifestyle factors and based on the detection in (f);
(h) monitoring the lung cancer's response or resistance to the treatment in (g) by obtaining exhaled breath (EB) samples from the subject over time; and
(i) adjusting the medical treatment as needed to improve clinical outcome.

According to some embodiments, the miRNA encapsulated in the EVs is one or more miRNA listed in Table 5 in the Examples. Lists of dysregulated miRNAs found from human and mouse studies are provided in Tables 8-11 in the Examples.

According to some embodiments, the one or more miRNAs is downregulated compared to the healthy control.

According to some embodiments, the one or more miRNAs is upregulated compared to the healthy control.

According to some embodiments, the subject is a mammalian subject. According to some embodiments, the subject is a human subject.

According to some embodiments, the detecting in (f) is earlier than detecting of lung cancer by imaging thresholds.

According to some embodiments, the method further comprises detecting a level of expression of a protein in the EVs derived from the EBC sample and determining that expression of the protein is dysregulated compared to the healthy control. Examples may include, without limitation,-α-enolase, vimentin, brain abundant membrane attached signal protein 1 (BASP1), aldolase A (ALDOA), caleticulin (CALR), proteasome activator subunit 1 (PSME1), proteasome activator subunit 2 (PSME2), major histocompatibility complex, class I C (HLA-C), and glucose 6 phosphate dehydrogenase (G6PD (found in EVs isolated from MDA-MB-231 and MCF-10a breast cancer cells) [Wu, C. et al. Anal. Bioanal. Chem. (2022) 414 (8): 2585-95], SH3 domain-containing protein 21, Arf-GAP with SH3 domain, ANK repeat and PH domain-containing protein 2, Histone H4, Vimentin, AHNAK nucleoprotein (desmoyokin), Heat shock protein HSP 90-beta, Annexin A5, Protein S100-A4, Heat shock protein HSP 90-alpha, Alpha-enolase, High mobility group protein HMG-I/HMG-Y, 14-3-3 protein zeta/delta, Hepatoma-derived growth factor, Gelsolin, Integrin alpha-3, 14-3-3 protein epsilon, Annexin A3, 14-3-3 protein theta, Proliferation-associated protein 2G4, 60 kDa heat shock protein, mitochondrial, Protein S100-A14, Vinculin, Ras-related protein Rab-7a, Integrin beta-1, Integrin alpha-6, Cytochrome c, somatic, Interleukin enhancer-binding factor 3, Cell division cycle 34B Protein phosphatase 1 regulatory inhibitor subunit 16B (Fragment); Protein phosphatase 1 regulatory inhibitor subunit 16B and Serine/threonine-protein kinase D.

According to some embodiments, the subject at risk is a smoker, a former smoker, or a non-smoker that is chemonaive; the subject at risk has been treated for lung cancer and is in remission; the subject at risk is at risk for a recurrence of lung cancer; or the subject at risk is at risk for progression of lung cancer.

According to some embodiments, a method for detecting lung colonizing cells derived from a primary tumor during early development of a secondary lung cancer comprises:
(a) identifying a unique cancer surface protein derived from the primary tumor in a first subject by:
(i) obtaining an exhaled breath sample from the first subject with a primary tumor, wherein the primary tumor has not metastasized and from a healthy control;
(ii) condensing the exhaled breath sample in a cooling chamber and collecting the exhaled breath condensate (EBC);
(iii) purifying EVs derived from the primary tumor and contained in the EBC obtained from the first subject and the healthy control;
(iv) evaluating a level of expression of miRNAs, mRNAs, surface proteins or a ratio of any two thereof contained in the EVs contained in the EBC sample from the first subject and in the EVs contained in the EBC sample from the healthy control;
(v) identifying the unique cancer protein derived from the primary tumor in the first subject;
(b) using the unique cancer protein derived from the primary tumor in (a), obtaining an exhaled breath sample from a second subject, wherein the second subject is at risk for a secondary lung tumor derived from primary tumor in (a);
(c) condensing the exhaled breath from the second subject in a cooling chamber and collecting the exhaled breath condensate;
(d) purifying a population of EVs contained in the EBC from the second subject,
(e) identifying a therapeutic biosignature for the secondary lung cancer in the EVs contained in the EBCs comprising expression of one or more of miRNAs, mRNAs and surface proteins derived from the primary tumor; and
(f) identifying the patient as one that can benefit therapeutically from being treated for the secondary lung cancer at an early stage.

According to some embodiments of the method,

The first subject is an orthotopic animal model and the second subject is an orthotopic model.

The first subject is a orthotopic animal model and the second subject is a PDX animal model.

The first subject is a PDX animal model and the second subject is an orthotopic animal model.

The first subject is a PDX animal model and the second subject is a PDX animal model; and
control animal colonies are established with primary human small airway epithelial cells (HSAECs).

According to some embodiments of the method, the orthotopic human tumor NOD/SCID mouse model bearing a human lung tumor comprises mutations including EGFR and KRAS mutations, wherein human NSCLC lung cancer cell lines are transduced for ex vivo bioluminescence, expanded in vitro and administered into the lungs of the NOD/SCID nude mice by tracheal instillation for lung uptake,

According to some embodiments, the PDX model is a human tumor NOD/SCID mouse model established using NSCLC patient-derived tumor cells comprising mutations including EGFR and KRAS mutations.

According to some embodiments of the method, the primary tumor is a colorectal cancer, a breast cancer or a bladder cancer.

According to some embodiments, using the orthotopic and PDX animal models, exh-EV biomarkers will be identified for early detection of secondary lung cancer EVs derived from primary colorectal, breast or bladder cancer. According to some embodiments, a unique colorectal cancer surface protein will be used to purify secondary lung cancer EVs derived from a primary colorectal cancer. According to some embodiments, a unique breast cancer surface protein will be used to purify secondary lung cancer EVs derived from a primary breast cancer. According to some embodiments, a unique bladder cancer surface protein will be used to purify secondary lung cancer EVs derived from a primary bladder cancer.

According to some embodiments, using the orthotopic and PDX animal models, exh-EV biomarkers will be identified for early detection of tuberculosis (TB) in households where specific individuals from TB+ households are susceptible to contracting TB as well as those who have active disease even though treatment was administered.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise. The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Materials and Methods

### Extracellular Vesicle Capture by AnTibody of CHoice and Enzymatic Release (EV-CATCHER) Assay

The release protocol described by Löf, et al. (2017) was further optimized and modified as follows; HPLC-purified uracilated oligonucleotides (Integrative DNA Technology) for the 5′-Azide (5′Az-AAAAACGAUUCGAGAACGUGACUGCCAUGCCAGCUCGUACUAU CGAA (SEQ ID NO: 1)) and 3′-Biotin (5′Bio-CGAUAGUACGAGCUGGCAUGGCAGUCACGUUCUCGAA UCGUUUU (SEQ ID NO: 2)), adapted from Löf et al. (2017) were resuspended in RNase-free water at a concentration of 250 ng/μl. Equimolar amounts (1:1 ratios) of each oligo were annealed (90° C. for 2 min, 90-42° C. for 40 min, 42° C. for 120 min) in 1×RNA annealing buffer (60 mM KCl, 6 mM HEPES (pH 7.5), 0.2 mM MgCl₂), prior to separation on a 15% non-denaturing polyacrylamide (PAGE) gel (0.5×TBE (ThermoFisher, #15581044) at 450 volts for 90 min). The double stranded (ds) DNA linker was visualized on a blue light box with SYBR® Gold™ dye (ThermoFisher, #S11494), excised, centrifugally crushed using a gel breaker tube (IST Engineering, #3388-100) and resuspended in 400 mM NaCl and shaken overnight (O/N) on a thermomixer set to 4° C. and 1,100 RPM. The solution was filtered, and the dsDNA linker was purified using the QIAEX® II gel extraction kit (Qiagen, #20021) according to manufacturer instructions. Purified dsDNA linker was evaluated on a NanoDrop 2000 and diluted to 250 ng/μl. Antibodies (1 mg/ml) used for exosome pulls (anti-CD63 (Abcam, #ab59479; RRID:AB_940915), anti-CD81 (Abcam, #ab233692) and anti-CD9 (Abcam, #ab263023)) were activated using 5 μl of freshly prepared 4 mM DBCO—S-S-NHS ester (Sigma Aldrich, #761532) and incubated for 30 min at room temperature (RT) in the dark, reactions were stopped by adding 2.5 μl of 1M Tris-Cl (pH 8.0) at RT for 5 min in the dark. DBCO-activated antibodies were desalted onto pre-equilibrated Zeba desalting columns (ThermoFisher, #89882) by incubation for 1 min and centrifugation at 1,500×g for 2 min. Antibodies were quantified on a Nanodrop 2000 instrument using protein A280 and antibody-dsDNA (Ab-dsDNA) stock solutions were prepared by conjugating 50 μg of activated antibody with 25 μg of purified DNA linker, O/N at 4° C. on a rotator. Validation of Ab-dsDNA binding was performed by PAGE where the Ab-dsDNA (1 μg) product was run under non-denaturing and non-reducing conditions, followed by Coomassie (Bio-Rad #1610786) staining to visualize the shift in Ab-dsDNA migration. The next day, Ab-dsDNA conjugates were bound to streptavidin coated 96-well plates (Pierce, #15120). Either single anti-CD63 antibody (1 μg) or a combination of anti-CD63, -CD81 and -CD9 (1 μg of each antibody) (linker bound) was added to single wells in 100 μl 1×PBS. Streptavidin coated 96-well plates with Ab-dsDNA conjugates were placed on a plate shaker at 300 RPM at 4° C., for 8 h to allow for binding to the plate. Solutions were carefully removed, and wells were washed three times with cold 1×PBS solution, prior to addition of RNase-A (12.5 μg/ml) treated samples (100 μl). Plates were sealed using microAMP optical adhesive film (Applied Biosystems, #4311971) and placed on a shaker at 300 RPM at 4° C., overnight. Samples were carefully removed, wells were washed 3 times with cold 1×PBS and 100 μl of freshly prepared uracil glycosylase (UNG) enzyme (ThermoFisher, #EN0362) in 1×PBS (1×UNG buffer (200 mM Tris-Cl (pH 8.0), 10 mM EDTA and 100 mM NaCl), with 1 unit of enzyme) was added to each well. Plates were incubated at 37° C. for 2 h on a shaker at 300 RPM for UNG digest of the dsDNA linker, and exosomes were recovered in this solution for further characterization and downstream analyses.

### Western Blot Analyses

Western blot analyses were conducted to evaluate presence of exosome surface protein biomarkers from purified exosome fractions. Purified exosomes were lysed and denatured in 1× Laemmli buffer (Bio-Rad, #161-0747) containing 355 mM β-mercaptoethanol, and heated at 95° C. for 5 min. Denatured lysates were pulse centrifuged and separated on 4-12% polyacrylamide precast mini-PROTEAN TGX gel (Bio-Rad, #4561086) by sodium dodecyl sulphate polyacrylamide gel electrophoresis (SDS-PAGE). Five μl of Precision Plus Protein™ Dual Xtra Prestained Protein Standard (Bio-Rad, #1610377) was loaded and used for gel orientation and determination of molecular weights of separated proteins. Samples were loaded and gels were run at 100 V and 400 mA for 90 min (Power Pac 300, Bio-Rad) in 1× Tris/Glycine/SDS buffer (Bio-Rad, #1610732). After the SDS-PAGE run, proteins were transferred to 0.2 μm polyvinylidene fluoride (PVDF) membranes (Bio-Rad, #1704156) using a semidry electro-transfer system (TransBlot Turbo™ v1.02, Bio-Rad) for 30 min at 25 V. Membranes were visualized using the stain-free blot protocol provided on a Chemi-Doc™ MP (Bio-Rad) system to evaluate protein transfer and non-specific binding was prevented by blocking membranes in EveryBlot™ blocking buffer (Bio-Rad, #12010020) for 30 min. Membranes were incubated at 4° C. overnight with TBS-T (1×TBS, pH 6.8, 0.1% Tween® 20) diluted anti-mouse primary antibodies (1:1000) targeted against Alix (Life Technologies, #MA183977) and CD63 (Abcam, #ab59479) or with TBS-T diluted anti-rabbit primary antibodies (1:1000) targeted against CD81 (Abcam, #ab233692) and CD9 (Abcam, #ab263023). Membranes were washed with TBS-T (3×5 min) before incubation in either anti-mouse or anti-rabbit IgG horseradish peroxidase conjugated secondary antibodies (1:10000) for 1 h, with gentle agitation at RT. Membranes were washed with TBS-T (3×5 min) before proteins were detected using SuperSignal™ West Femto Maximum Sensitivity Substrate (Pierce, #34095) and protein bands were visualized using ImageLab 4.0 software on a Chemi-Doc MP (Bio-Rad) imaging system.

### Transmission Electron Microscopy

Transmission electron microscopy (TEM) of purified exosomes was performed by Charles River Laboratories (Pathology Associates (PAI), Durham NC). Briefly, purified exosomes were fixed using 2% Glutaraldehyde in phosphate buffer (Electron Microscopy Services, #6536-05) and stored at 4° C. 300 mesh formvar-coated grids were inverted onto 20 μl of fixed exosome suspensions for approximately 2 min and wicked dry. Grids were then inverted onto 40 μl of 2% aqueous uranyl acetate for approximately 1 min, and wicked dry. Samples were imaged on a JEOL JEM-1400+ transmission electron microscope (JEOL Ltd.; Tokyo, Japan) operating at an accelerating voltage of 80 kV. High resolution TIFF images were acquired and saved using an AMT 16 MP digital camera system (Advanced Microscopy Techniques Corp.; Woburn, MA).

### Nanoparticle Tracking Analysis (NTA)

Size and particle concentration of purified exosomes were assessed using a Spectradyne nCS1 instrument. Briefly, 2 μl of purified exosomes were loaded onto TS400 microfluidic cartridges (Spectradyne, #TS400), which allows for the analysis of particles between 65-400 nm. Loaded cartridges were primed in the instrument using 0.2 μm filtered buffer (1×PBS containing 1% Tween® 20) and particle acquisition was carried out. Acquired stats files were analyzed for particle concentration and size distribution using Spectradyne Viewer software, where peak-filtered CSD graphs were generated.

Super Resolution Microscopy (ONI). Using super-resolution microscopy, fluorescence from labeled EVs in two fluorescent channels at the same time can be imaged and combined with two-color NTA.

Characterizing biomarkers on individual EVs posses a significant challenge to researches in the field. While traditional TEM techniques have the capability to resolve individual EVs, they do not easily allow for the detection of multiple markers at the same time and are limited to fixed cells. With conventional light microscopy techniques, a number of proteins can be labeled, but the small size of EVs means that the majority fall well below the resolution limit of light microscopy, restricting the usefulness of these techniques in identifying different subpopulations of vesicles.

Super-resolution microscopy surpasses the resolution barrier of conventional light microscopes and enables detection and quantification of single proteins and nucleic acids at the sub-vesicular level. Recently developed super-resolution techniques have overcome the diffraction limit of light and allow for EVs and their contents to be investigated at molecular resolution.

For example, a population of exh-EVs in EBC can be immunostained with a combination of three fluorescent labeled antibodies against EV biomarkers of interest, each tagged with a different dSTORM-compatible fluorescent flurophore, such as Alexa Fluor 488, ATTO488, Alexa Fluor 555, Cy3B, CF568, DyLight 550, Alexa Fluor 568, Cy5, CF647 and Alexa Fluor 647. Cell specific protein markers include but are not limited to CCSP, SFTPC, Goblet cell Protein Clcal (Alias mClca3 or Gob-5), PGP9.5 (also known as UCHL1), CGRP, Advillin, etc. Fluorescently labeled lectins, which specifically bind terminal carbohydrate moieties on the vesicle membrane surface, can be used as an internal standard. Only dual-labeled EVs are followed and and the unwanted population is excluded during analysis.

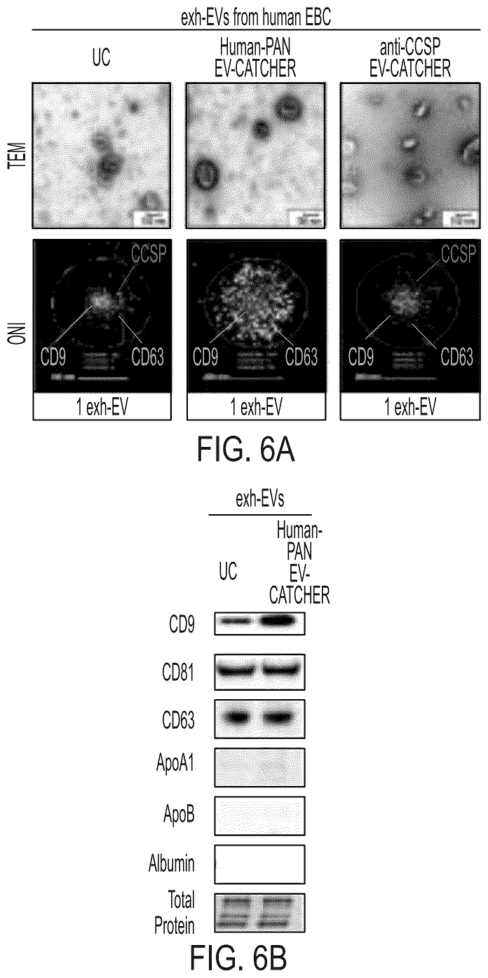

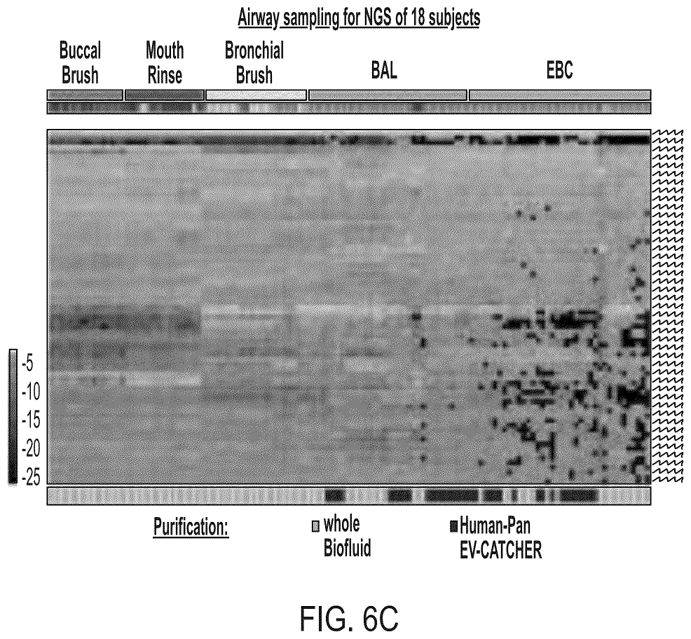

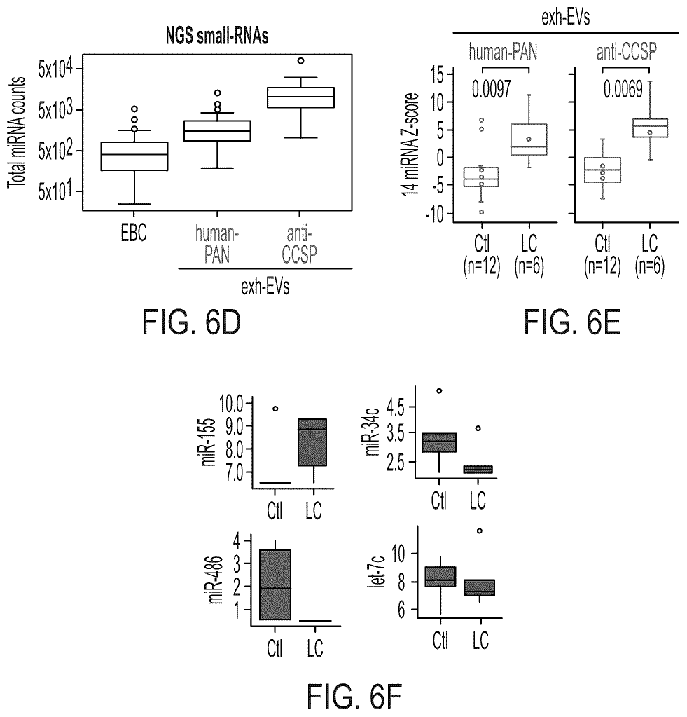

Super-resolution microscopy also allows for direct localization of surface proteins on EVs. FIG. 6a shows images of CCSP proteins on EVs purified from human exh-EVs, validating that exh-EVs indeed are coming directly from the deep lung.

### Quantitative PCR Experiments

Quantitative PCR (qPCR) experiments were performed using TaqMan® microRNA reverse-transcription kits, TaqMan® microRNA master mix PCR kits, and individual TaqMan® microRNA assays following manufacturer's instructions (ThermoFisher). For evaluation of non-specific exosome binding to Dynabeads™ and streptavidin coated 96-well plates, an ath-miR-159a RNA oligo (Integrated DNA Technologies) was used. One picogram of ath-miR-159a was also added during RNA extractions of exosomes used as a technical normalization control. For optimization experiments we quantified hsa-miR-21 (ThermoFisher, #000397) and hsa-miR-200c (ThermoFisher, #000505) with RNA extracted from MCF-7 exosomes. For validation of differentially expressed miRNAs identified by sequencing of exosomal RNA, TaqMan® miRNA primer assays for hsa-miR-126-3p (#002228), hsa-miR-146a (#002163), hsa-miR-126-5p (#000451), hsa-miR-205 (#000509) were used. For these qPCR reactions, reverse transcriptions were set up using 10% (2 μl out of 20 μl) of RNA extracted from combined CD63/CD81/CD9 exosome purifications from 100 μl of serum. For individual transcript quantifications, 1/3 of the RT reactions (5 μl) was used to set up the three individual qPCR experiments. Quantitative PCR measurements were performed on a Step-One-Plus instrument using manufacturer's recommended streptavidin coated 96-well plates and covers. Quantitative data was transferred to Excel sheet for statistical analyses, as described below. Exemplary miRNA sequences are listed in Table 5 below.

TABLE 5
Exemplary miRNA sequences

| Description | Sequence | SEQ ID NO: |
|---|---|---|
| ath-miR-159a | UUUGGAUUGAAGGGAGCUCUA | SEQ ID NO: 3 |
| hsa-miR-21 | UAGCUUAUCAGACUGAUGUUGA | SEQ ID NO: 4 |
| hsa-miR-200c | UAAUACUGCCGGGUAAUGAUGG | SEQ ID NO: 5 |
| hsa-miR-126-3p | UCGUACCGUGAGUAAUAAUGCG | SEQ ID NO: 6 |
| hsa-miR-146a | CCUCUGAAAUUCAGUUCUUCAG | SEQ ID NO: 7 |
| hsa-miR-126-5p | CAUUAUUACUUUUGGUACGCG | SEQ ID NO: 8 |
| hsa-miR-205 | UCCUUCAUUCCACCGGAGUCUG | SEQ ID NO: 9 |
| hsa-miR-125a | UCCCUGAGACCCUUUAACCUGUGA | SEQ ID NO: 10 |
| hsa-miR-10b | UACCCUGUAGAACCGAAUUUGUG | SEQ ID NO: 11 |
| hsa-miR-451-DICER 1 | AAACCGUUACCAUUACUGAGUU | SEQ ID NO: 12 |
| hsa-miR-126* | CAUUAUUACUUUUGGUACGCG | SEQ ID NO: 13 |
| hsa-miR-30d | UGUAAACAUCCCCGACUGGAAG | SEQ ID NO: 14 |
| hsa-let-7g | UGAGGUAGUAGUUUGUACAGUU | SEQ ID NO: 15 |
| hsa-miR-30e | UGUAAACAUCCUUGACUGGAAG | SEQ ID NO: 16 |
| hsa-miR-221 | ACCUGGCAUACAAUGUAGAUUU | SEQ ID NO: 17 |
| hsa-let-7f(2) | UGAGGUAGUAGAUUGUAUAGUU | SEQ ID NO: 18 |
| hsa-miR-24(2) | UGCCUACUGAGCUGAAACACAG | SEQ ID NO: 19 |
| hsa-miR-26a | UUCAAGUAAUCCAGGAUAGGCU | SEQ ID NO: 20 |
| hsa-miR-423-Sp | UGAGGGGCAGAGAGCGAGACUUU | SEQ ID NO: 21 |
| hsa-miR-22 | AGUUCUUCAGUGGCAAGCUUUA | SEQ ID NO: 22 |
| hsa-miR-320-RNASEN | GCCUUCUCUUCCCGGUUCUUCC | SEQ ID NO: 23 |
| hsa-let-7b | UGAGGUAGUAGGUUGUGUGGUU | SEQ ID NO: 24 |
| hsa-miR-126 | CAUUAUUACUUUUGGUACGCG | SEQ ID NO: 25 |
| hsa-let-7 a(3) | UGAGGUAGUAGGUUGUAUAGUU | SEQ ID NO: 26 |
| hsa-miR-203 | AGUGGUUCUUAACAGUUCAACAGUU | SEQ ID NO: 27 |
| hsa-miR-486 | UCCUGUACUGAGCUGCCCCGAG | SEQ ID NO: 28 |
| hsa-miR-92a(2) | GGGUGGGGAUUUGUUGCAUUAC | SEQ ID NO: 29 |
| hsa-miR-29a | ACUGAUUUCUUUUGGUGUUCAG | SEQ ID NO: 30 |
| hsa-miR-27a | AGGGCUUAGCUGCUUGUGAGca | SEQ ID NO: 31 |
| hsa-miR-140-3p | CAGUGGUUUUACCCUAUGGUAG | SEQ ID NO: 32 |
| hsa-miR-10a | UACCCUGUAGAUCCGAAUUUGUG | SEQ ID NO: 33 |
| hsa-miR-23a | GGGGUUCCUGGGGAUGGGAUUU | SEQ ID NO: 34 |
| hsa-miR-148a | AAAGUUCUGAGACACUCCGACU | SEQ ID NO: 35 |
| hsa-miR-103(2) | AGCUUCUUUACAGUGCUGCCUUG | SEQ ID NO: 36 |
| hsa-miR-93 | CAAAGUGCUGUUCGUGCAGGUAG | SEQ ID NO: 37 |
| hsa-miR-16(2) | UAGCAGCACGUAAAUAUUGGCG | SEQ ID NO: 38 |
| hsa-miR-125b | UCCCUGAGACCCUAACUUGUGA | SEQ ID NO: 39 |
| hsa-miR-423-5p | UGAGGGGCAGAGAGCGAGACUUU | SEQ ID NO: 40 |
| hsa-miR-191 | CAACGGAAUCCCAAAAGCAGCUG | SEQ ID NO: 41 |
| hsa-miR-30a | UGUAAACAUCCUCGACUGGAAG | SEQ ID NO: 42 |
| hsa-let-7i | UGAGGUAGUAGUUUGUGCUGUU | SEQ ID NO: 43 |
| hsa-miR-101(2) | UCGGUUAUCAUGGUACCGAUGC | SEQ ID NO: 44 |
| hsa-miR-25 | AGGCGGAGACUUGGGCAAUUG | SEQ ID NO: 45 |
| hsa-miR-484-RNASEN | UCAGGCUCAGUCCCCUCCCGAU | SEQ ID NO: 46 |
| hsa-miR-181 a(2) | AACAUUCAACGCUGUCGGUGAGU | SEQ ID NO: 47 |
| hsa-miR-378 | CUCCUGACUCCAGGUCCUGUGU | SEQ ID NO: 48 |
| hsa-miR-30c(2) | UGUAAACAUCCUACACUCUCAGC | SEQ ID NO: 49 |
| hsa-miR-27b | AGAGCUUAGCUGAUUGGUGAAC | SEQ ID NO: 50 |
| hsa-miR-122 | UGGAGUGUGACAAUGGUGUUUG | SEQ ID NO: 51 |
| hsa-let-7d* | AGAGGUAGUAGGUUGCAUAGUU | SEQ ID NO: 52 |
| hsa-miR-19b(2) | AGUUUUGCAGGUUUGCAUUUCA | SEQ ID NO: 53 |
| hsa-miR-26b | UUCAAGUAAUUCAGGAUAGGU | SEQ ID NO: 54 |
| hsa-miR-505 | GGGAGCCAGGAAGUAUUGAUGU | SEQ ID NO: 55 |
| hsa-miR-7(3) | UGGAAGACUAGUGAUUUUGUUGUU | SEQ ID NO: 56 |
| miR-423-3p | AGCUCGGUCUGAGGCCCCUCAGU | SEQ ID NO: 57 |
| miR-186 | CAAAGAAUUCUCCUUUUGGGCU | SEQ ID NO: 58 |
| let-7b | UGAGGUAGUAGGUUGUGUGGUU | SEQ ID NO: 59 |
| miR-99a | AACCCGUAGAUCCGAUCUUGUG | SEQ ID NO: 60 |
| miR-99b | CACCCGUAGAACCGACCUUGCG | SEQ ID NO: 61 |
| miR-122 | UGGAGUGUGACAAUGGUGUUUG | SEQ ID NO: 62 |
| miR-378 | CUCCUGACUCCAGGUCCUGUGU | SEQ ID NO: 63 |
| miR-93 | CUCCUGACUCCAGGUCCUGUGU | SEQ ID NO: 64 |
| miR-185 | UGGAGAGAAAGGCAGUUCCUGA | SEQ ID NO: 65 |
| miR-184 | UAUGGAGGUCUCUGUCUGACU | SEQ ID NO: 66 |
| miR-183 | UAUGGCACUGGUAGAAUUCACU | SEQ ID NO: 67 |
| miR-34a | UGGCAGUGUCUUAGCUGGUUGU | SEQ ID NO: 68 |
| miR-132 | AACCGUGGCUUUCGAUUGUUAC | SEQ ID NO: 69 |
| miR-7 | UGGAAGACUAGUGAUUUUGUUGU | SEQ ID NO: 70 |
| let-7c | UGAGGUAGUAGGUUGUAUGGUU | SEQ ID NO: 71 |
| miR-18a | UAAGGUGCAUCUAGUGCAGAUAG | SEQ ID NO: 72 |
| miR-13Ob | ACUCUUUCCCUGUUGCACUAC | SEQ ID NO: 73 |
| miR-210 | AGCCACUGCCCACCGCACACUG | SEQ ID NO: 74 |
| miR-146b | UGAGAACUGAAUUCCAUAGGCUG | SEQ ID NO: 75 |
| miR-29b | GCUGGUUUCAUAUGGUGGUUUAGA | SEQ ID NO: 76 |
| miR-20a | GCUGGUUUCAUAUGGUGGUUUAGA | SEQ ID NO: 77 |
| miR-194 | UAAAGUGCUUAUAGUGCAGGUAG | SEQ ID NO: 78 |
| miR-30e* | CUUUCAGUCGGAUGUUUACAGC | SEQ ID NO: 79 |
| miR-374b | AUAUAAUACAACCUGCUAAGUG | SEQ ID NO: 80 |
| miR-203 | AGUGGUUCUUAACAGUUCAACAGUU | SEQ ID NO: 81 |
| miR-144* | GGAUAUCAUCAUAUACUGUAAG | SEQ ID NO: 82 |
| miR-374a | UUAUAAUACAACCUGAUAAGUG | SEQ ID NO: 83 |
| miR-584 | UUAUGGUUUGCCUGGGACUGAG | SEQ ID NO: 84 |
| miR-143 | GGUGCAGUGCUGCAUCUCUGGU | SEQ ID NO: 85 |
| miR-144 | GGAUAUCAUCAUAUACUGUAAG | SEQ ID NO: 86 |
| let-7d* | CUAUACGACCUGCUGCCUUUCU | SEQ ID NO: 87 |
| miR-30b | UGUAAACAUCCUACACUCAGCU | SEQ ID NO: 88 |
| miR-100 | AACCCGUAGAUCCGAACUUGUG | SEQ ID NO: 89 |
| miR-199a-5p | CCCAGUGUUCAGACUACCUGUUC | SEQ ID NO: 90 |
| miR-192 | CUGACCUAUGAAUUGACAGCC | SEQ ID NO: 91 |
| miR-148a | AAAGUUCUGAGACACUCCGACU | SEQ ID NO: 92 |
| miR-342 | AGGGGUGCUAUCUGUGAUUGA | SEQ ID NO: 93 |
| miR-375 | GCGACGAGCCCCUCGCACAAACC | SEQ ID NO: 94 |
| miR-409-3p | GAAUGUUGCUCGGUGAACCCCU | SEQ ID NO: 95 |
| miR-10b | ACCCUGUAGAACCGAAUUUGUG | SEQ ID NO: 96 |
| miR-130a | GCUCUUUUCACAUUGUGCUACU | SEQ ID NO: 97 |
| miR-126-5p | CAUUAUUACUUUUGGUACGCG | SEQ ID NO: 98 |
| miR-150 | UCUCCCAACCCUUGUACCAGUG | SEQ ID NO: 99 |
| miR-155 | UUAAUGCUAAUCGUGAUAGGGGUU | SEQ ID NO: 100 |
| miR-32 | UAUUGCACAUUACUAAGUUGCA | SEQ ID NO: 101 |
| miR-34c | AGGCAGUGUAGUUAGCUGAUUGC | SEQ ID NO: 102 |
| miR-193a-3p | AACUGGCCUACAAAGUCCCAGU | SEQ ID NO: 103 |
| miR-636* | UGUGCUUGCUCGUCCCGCCCGCA | SEQ ID NO: 104 |
| miR-1 | ACAUACUUCUUUAUAUGCCCAU | SEQ ID NO: 105 |
| miR-133a | GCUGGUAAAAUGGAACCAAAU | SEQ ID NO: 106 |
| miR-205 | UCCUUCAUUCCACCGGAGUCUG | SEQ ID NO: 107 |
| miR-141 | CAUCUUCCAGUGCAGUGUUGGA | SEQ ID NO: 109 |
| miR-200a | CAUCUUACCGGACAGUGCUGG | SEQ ID NO: 110 |
| miR-200b | CAUCUUACUGGGCAGCAUUGGA | SEQ ID NO: 111 |
| miR-145* | GGAUUCCUGGAAAUACUGUUCU | SEQ ID NO: 112 |
| miR-486 | UCCUGUACUGAGCUGCCCCGAG | SEQ ID NO: 113 |
| miR-126-3p | UCGUACCGUGAGUAAUAAUGCG | SEQ ID NO: 114 |
| miR-223 | CGUGUAUUUGACAAGCUGAGUUG | SEQ ID NO: 115 |
| miR-451 | AAACCGUUACCAUUACUGAGUU | SEQ ID NO: 116 |
| miR486 | UCCUGUACUGAGCUGCCCCGAG | SEQ ID NO: 117 |
| miR-146a | CCUCUGAAAUUCAGUUCUUCAG | SEQ ID NO: 118 |
| miR-222 | CUCAGUAGCCAGUGUAGAUCCU | SEQ ID NO: 119 |

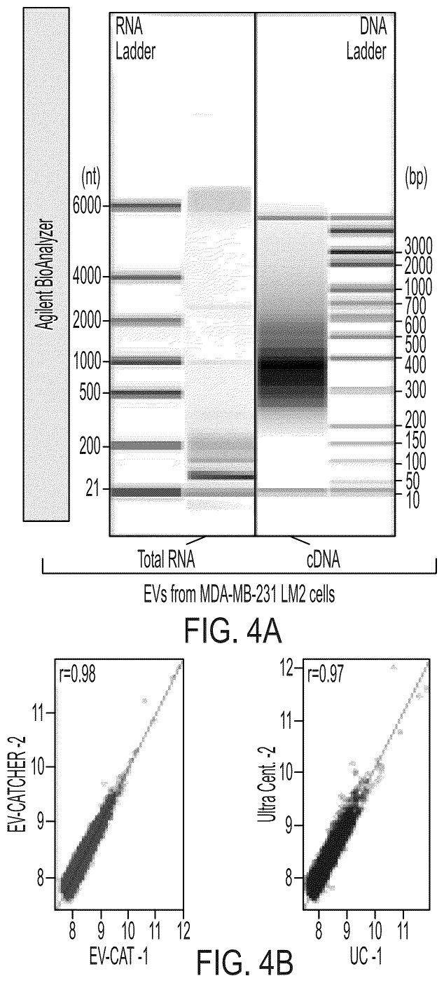

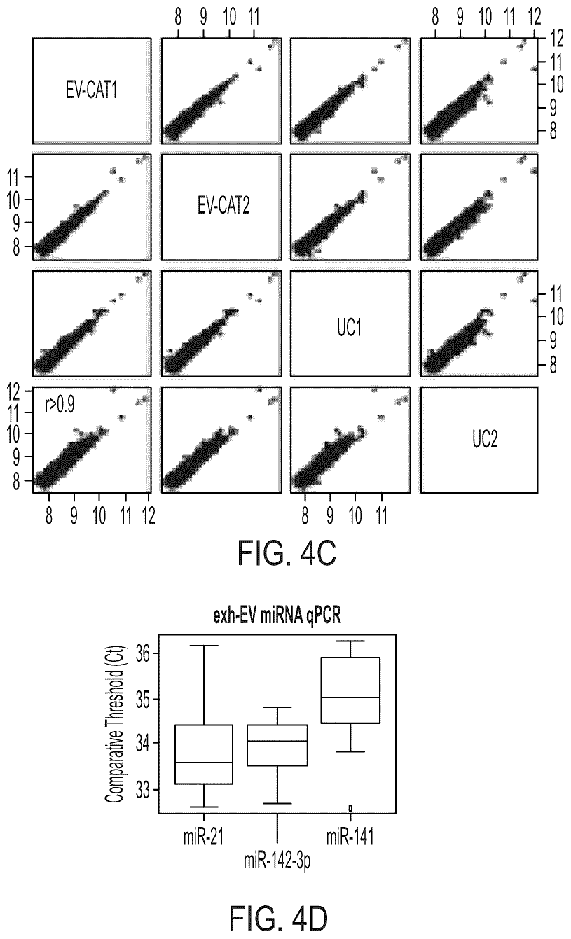

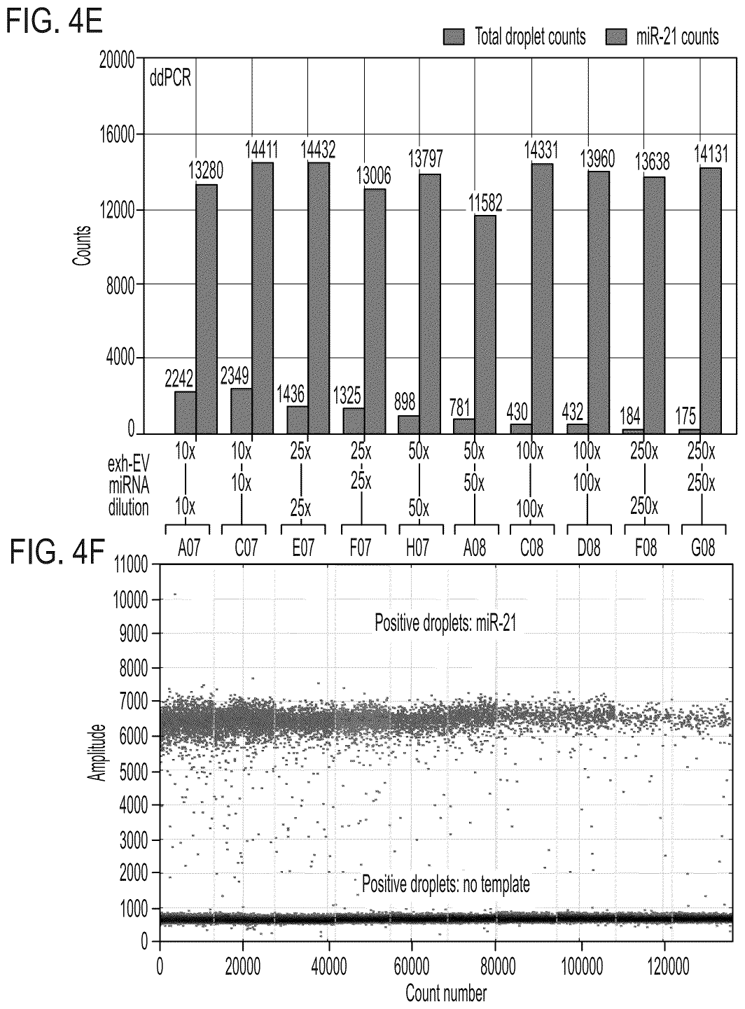

Droplet Digital Polymerase Chain Reaction (ddPCR). ddPCR is based on the partitioning of a sample into thousands of micro-reactions of defined volume using water-in-oil emulsions to create droplets from which nucleic acid targets within a sample can be identified and quantified.under the assumption of a Poisson distribution. Typically, results are expressed as target copies per microliter of reaction. This technique can detect miRNAs (see, e.g., FIGS. 4e and 4f) as well as unique mRNA mutations on EGFR or other lung oncogenes for early detection of disease.

According to some embodiments, lung tumor mutations also may be detected by deep sequencing, a next generation sequencing method for sequencing a genomic region multiple times, sometimes hundreds, or even thousands of times in order to detect rar clonal types or cells.

### Example 1. Preliminary Studies

a. Collection Device and Optimized Molecular Assays:

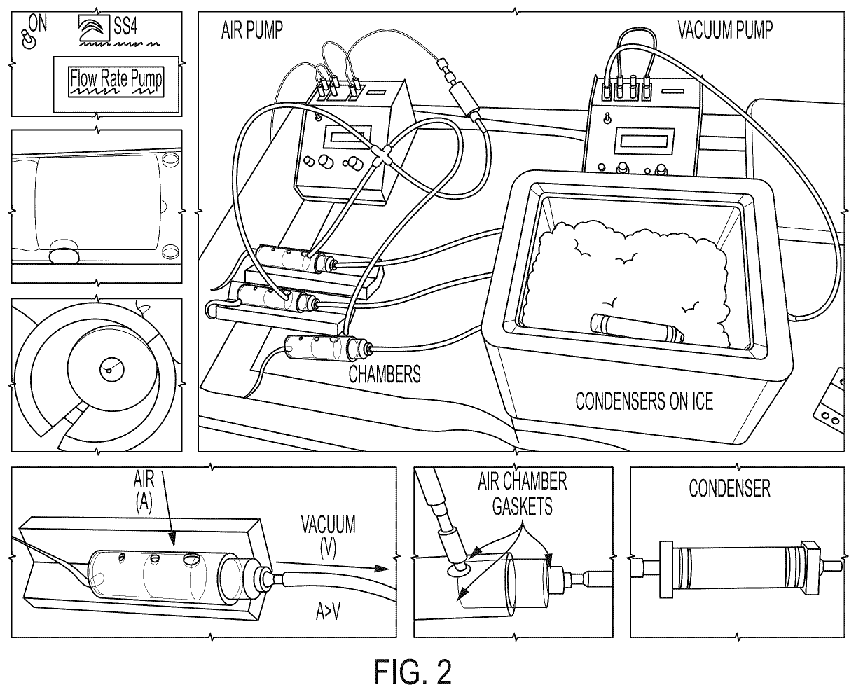

a.1. Mouse EBC collection system: In collaboration with Sables Systems International a single animal EBC collection system capable of handling one to six separate animals was engineered in parallel (FIG. 2). With this system, each animal is restrained inside a PVC cylinder for 2 hours while breathing into an air chamber. An airflow regulator pumps air unidirectionally into the sterile air-tight air chamber (sealed with gaskets) where the animal's nose exchanges air. The exhaled breath of the animal is pumped out of the air chamber by a second airflow regulator in vacuum mode to improve airflow (air in>air out in chamber) and directs it into a condenser that is sitting on ice. EBC accumulates on the surface of the airtight glass cylinder of the condenser. After 2 hours EBC is collected by placing the glass cylinder into a 50 ml conical tube that is centrifuged at 1,000 rpm for 3 minutes. The EBC is then transferred to a barcoded 1.5 mL tube and stored at −80° C. Based on pilot studies, at least 20 μL EBC per animal can be consistently collected in 2 hours. One system can accommodate up to 3 control and 3 tumor-bearing animals (with separate air tubing in parallel). Three systems side-by-side can thus handle 18 single animals in parallel (9 control and 9 tumor-bearing animals) for the 2 hours EBC collections. After each collection, the glass, the plastic chambers, the condensers, and caps are disinfected with Peroxyguard™, rinsed (ddH2O), and UV treated to prevent cross-contamination.

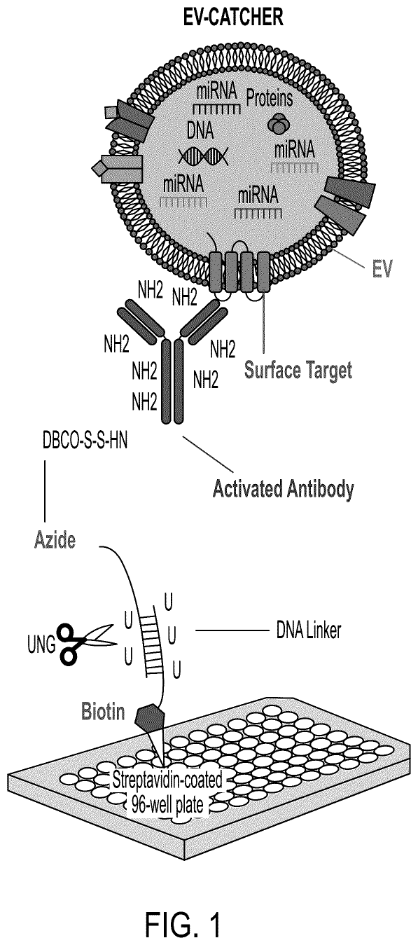

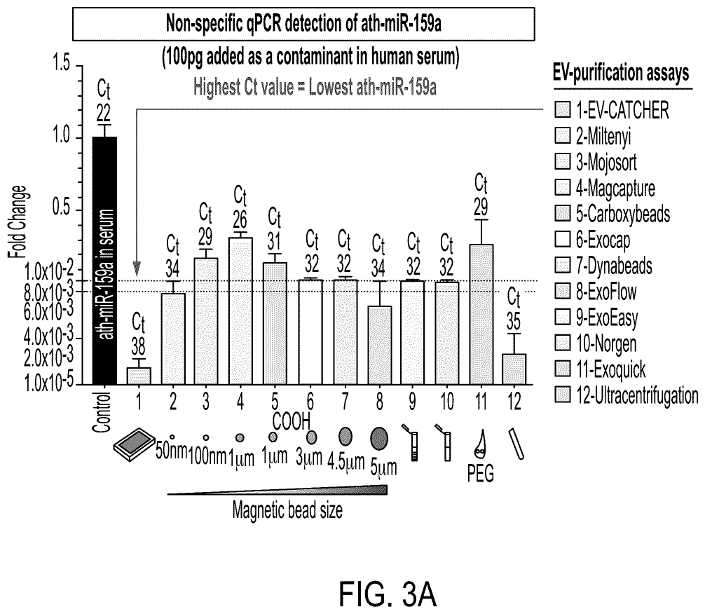

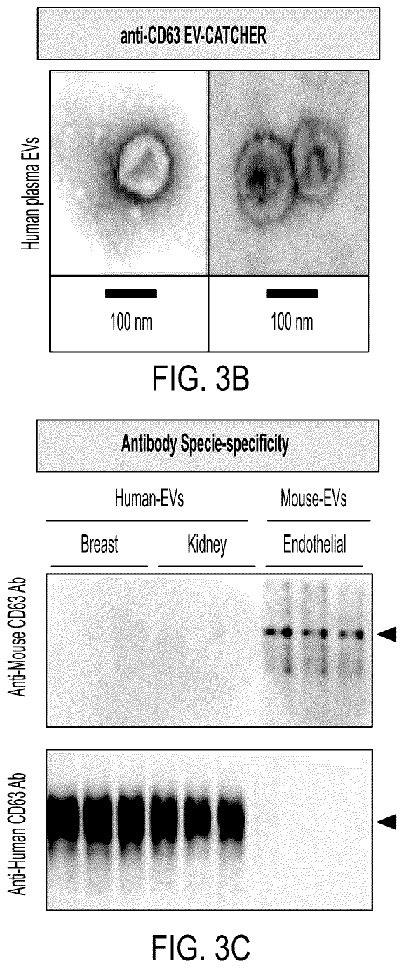

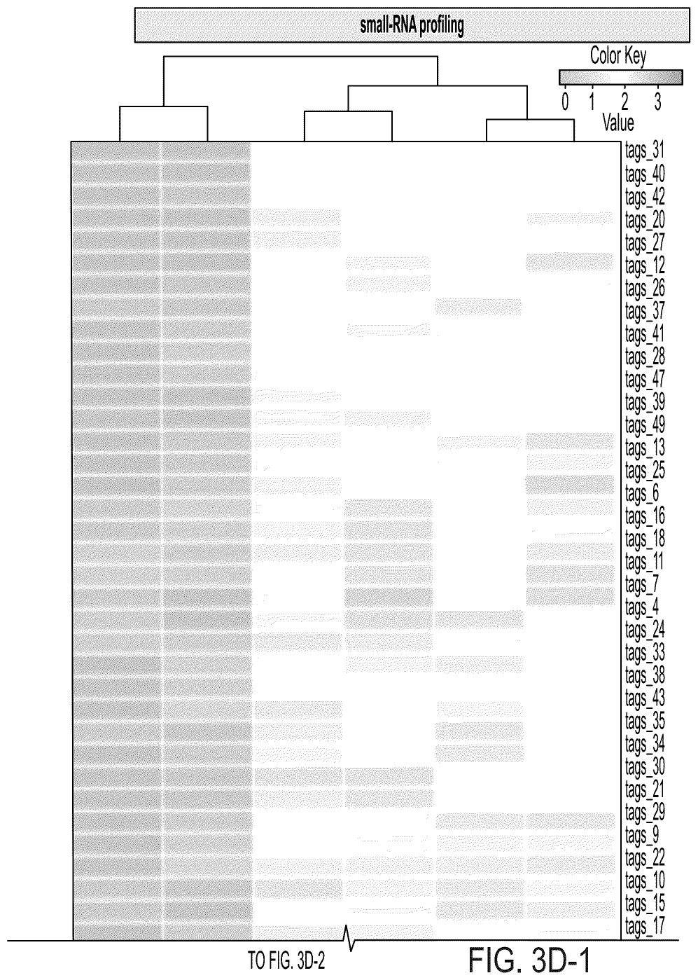

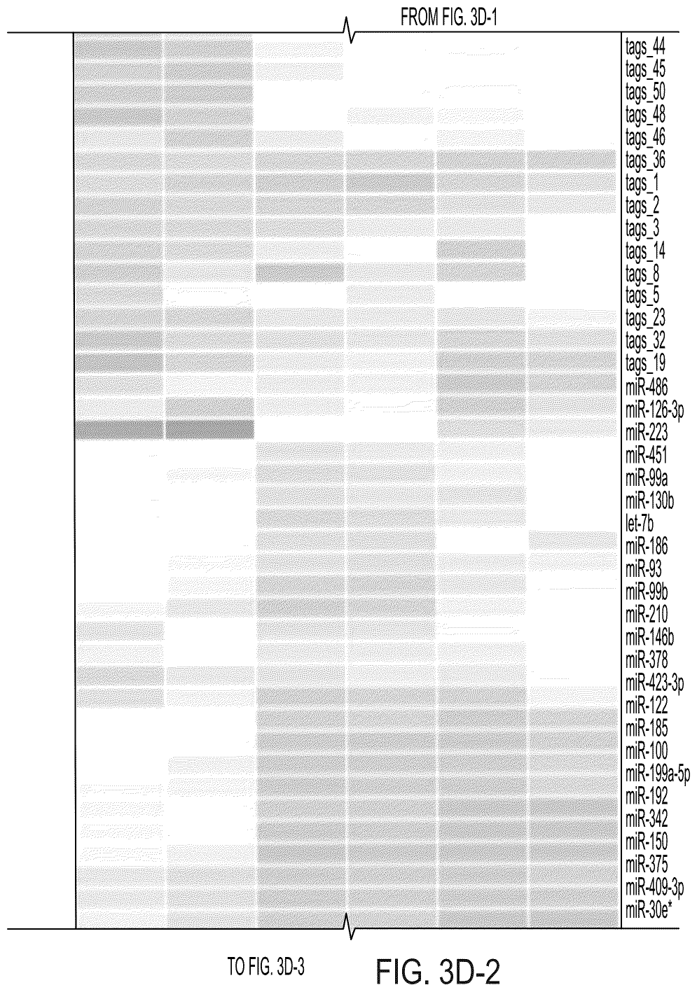

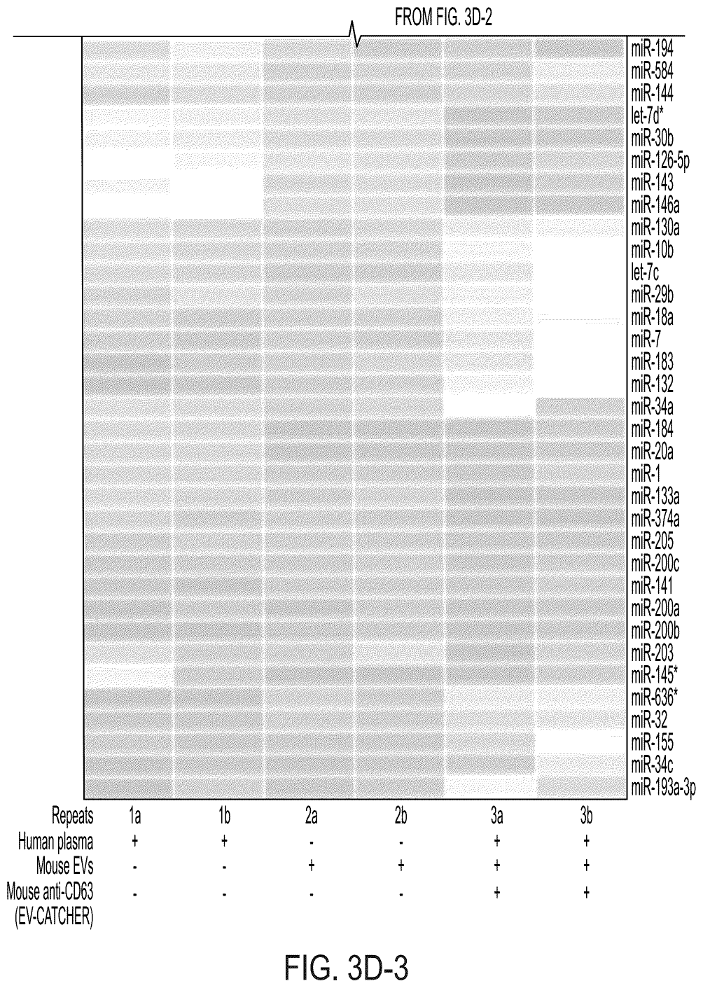

a.2. EV-CATCHER assay: This assay (FIG. 1) was developed to address limitations of commercial assays, particularly the very high level of noise acquired by non-specific binding of RNA molecules and EVs to the magnetic beads, which are widely used with those types of assays [42]. This improved assay prevents this non-specific binding, as demonstrated by comparison to 11 commercially available and laboratory-based EV purification assays (FIG. 3a). As most commercial antibody-based assays only allow for bulk purification of EVs by targeting tetraspanins (CD9, CD81, and CD63), which are specifically present on the surface of EVs, this assay uses click-chemistry to activate any antibody of choice. Thus, the assay can be customized to target any unique surface protein particular to a desired cell type for selection of specific EVs from biofluids. By adding an enzymatically degradable uracilated double-stranded DNA linker between the immobilizing platform and the activated antibody, intact release of functional EVs is enabled (FIG. 3b) by incubation with uracil glycosylase (UNG) [42]. As it is relevant to this project, the robustness of EV-CATCHER for specie-specific selection of EVs has already been validated [42]. In particular, we showed that it allows recovery of small-RNA biomarkers encapsulated in mouse-EVs that are spiked and purified from human plasma using EV-CATCHER customized with an anti-mouse anti-CD63 antibody (FIG. 3c & FIGS. 3D-1 to 3D-3). For this project, when purifying exh-EVs from EBC of orthotopic and PDX animal models, EV-CATCHER will be customized with anti-human anti-CD63/CD9/CD81 antibodies (human-PAN) to direct selective capture of human tumor exh-EVs from mouse EBC. For transgenic animals, as tumor exh-EVs will be secreted by specific lung cells expressing EGFRL858R or KRASG12D proteins, their purification will be targeted by customizing EV-CATCHER with antibodies targeting surface proteins unique to these lung cells.

a.3. RNA extractions from exh-EVs: We have demonstrated that miRNA extraction from exh-EVs, purified with EV-CATCHER from EBC, can be performed reproducibly using the miRNeasy serum/plasma kit (Qiagen). By adding carrier oligonucleotides that are used during the preparation of miRNA libraries (sequencing) or the ath-miR-159a exogenous plant miRNA that is used as a normalization control during qPCR analyses, miRNA recovery from exh-EVs is increased by over two-fold when compared with extractions performed without these carriers [42]. For total mRNA extraction from exh-EVs purified from EBC with EV-CATCHER, the Norgen plasma/serum exosome kit was found to provide sufficient RNA for the preparation of mRNA libraries (i.e., amplification), as it can be evaluated and quantified on an Agilent Bioanalyzer pico RNA chip (FIG. 4a).

a.4. Exh-EV miRNA and mRNA Discovery and Validation:

i. miRNA analyses-Using the optimized miRNA library preparation protocol for multiplexed analysis of 18 samples [42,45,46] by Next Generation Sequencing (NGS), miRNA profiles have been reproducibly generated from exh-EVs purified with EV-CATCHER from human EBC (See b.1 and b.2 below).

ii. mRNA analyses—For preparation of mRNA libraries using total RNA extracted from human EVs, we and others have successfully used the SMARTer® Stranded Total RNA-Seq library preparation Kit v2-Pico Input from Takara [80,81] to generate both properly sized libraries (FIG. 4a) and reproducible mRNA NGS data (FIG. 4b). For pilot analyses, total RNA was extracted from exh-EVs purified by ultracentrifugation [RCF of UC please?] or with our human-PAN EV-CATCHER assay from a healthy EBC donor. We observed high reproducibility (r>0.9) even when comparing mRNA NGS data between the two EV purification methods (FIG. 4b).

iii. qPCR validation assays-We confirmed that miRNAs extracted from exh-EVs (i.e., purified from the EBC of healthy humans), can be reproducibly detected by NGS analyses. When selecting three miRNAs (miR-21, miR 142-3p, and miR-141) displaying different ranges of expression in these exh-EVs, we also found that we can use Taqman® miRNA qPCR assays to validate their expression. Our recently published data also demonstrates that miRNAs can be detected by a different PCR method from human whole EBC samples [47]. However, these miRNAs all display high comparative thresholds, indicative of their low abundance (˜33-36 cycles in FIG. 4d), similarly to what we recently described for miRNAs detectable from EVs purified with EV-CATCHER from scrum [42].

iv. Droplet digital PCR (ddPCR) analyses-A ddPCR approach was optimized for ultra-sensitive quantification of miRNAs, using a BioRad QX200 ddPCR instrument. As BioRad does not supply miRNA ddPCR assays, the use of Taqman® miRNA assays has been optimized as also shown by others [82]. Its sensitivity was tested by serially diluting miRNAs extracted from exh-EVs of a healthy donor (collected using the FDA-approved R-Tube™ EBC collector) up to 250-fold and a robust linear detection of miR-21 demonstrated (FIG. 4.e and f). For this proposal, ddPCR assays will be used for early detection of the top differentially expressed miRNA (Taqman®) and mRNA transcripts (using BioRad mRNA assays) present in tumor exh-EVs purified from mouse EBC. To normalize the samples, Spectradyne Nanoparticle tracker will be used to quantify exh-EVs purified with EV-CATCHER prior to the analyses. Additionally, as a miRNA qPCR and ddPCR normalization control, 100 pg of plant ath-miR-159a will be spiked with the purified exh-EVs prior to RNA extractions. Finally, the most stably expressed exh-EV miRNAs and mRNAs will be identified from the discovery datasets (Examples, Aims 1a and 2a), which will be used as biological qPCR and ddPCR normalization controls.

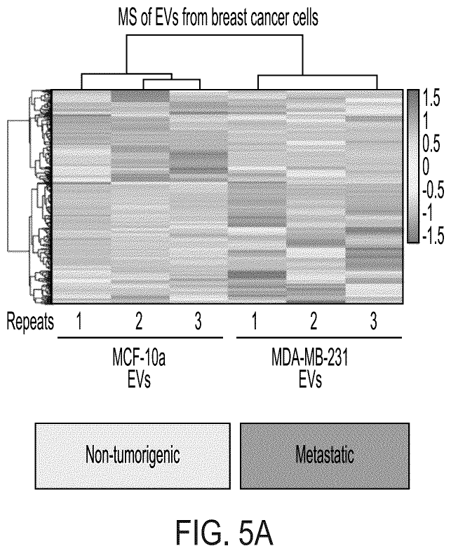

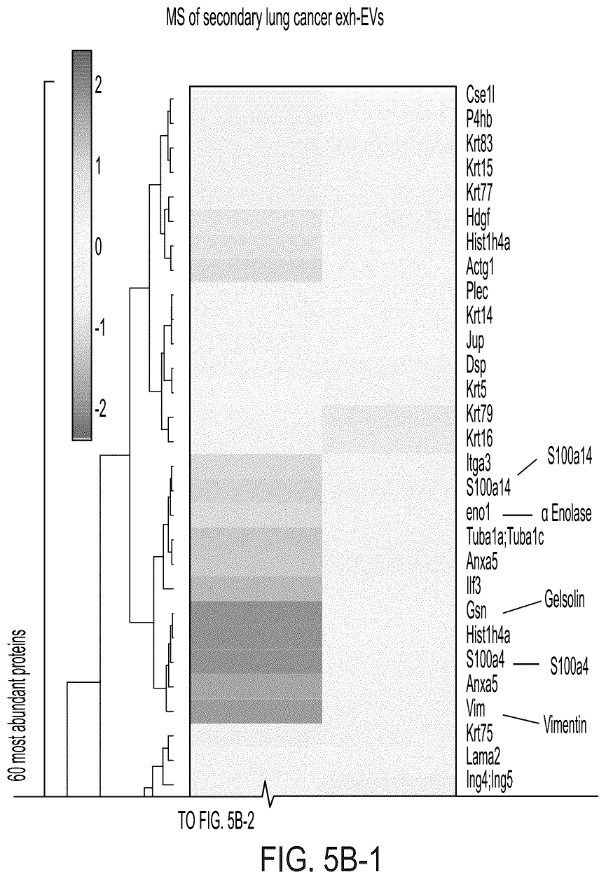

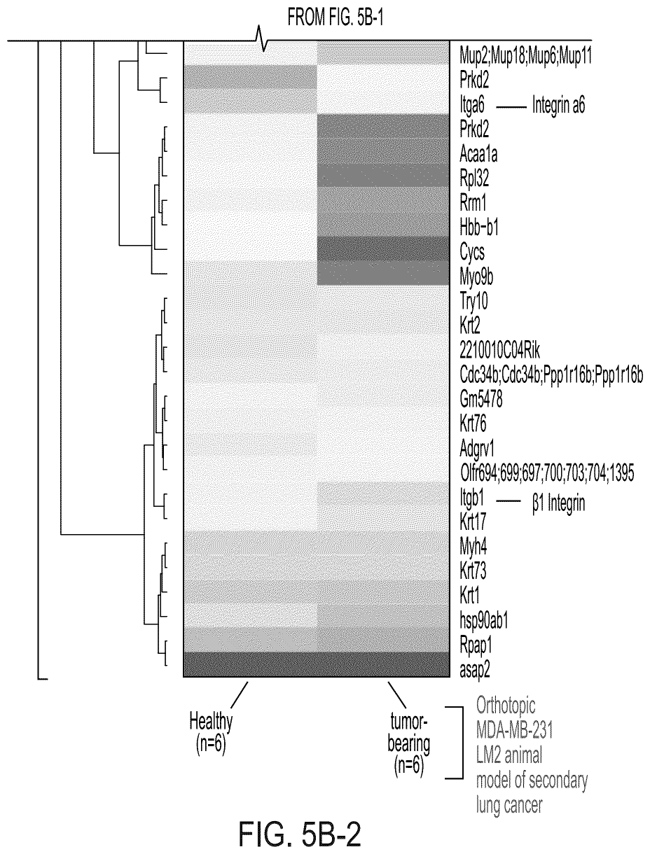

a.5 Proteomic analysis of EVs: Proteomic analyses of exh-EVs purified from EBC of our different lung cancer animal models will be performed. For this purpose, an ultra-sensitive and quantitative proteomic workflow was recently developed in collaboration with Dr. Junfeng Ma (co-director of Georgetown Proteomics Core) [74]. Our recently published approach was optimized using decreasing amounts of EVs purified from non-tumorigenic (MCF-10a) and metastatic breast cancer (MDA-MB-231) cell lines [74]. By integrating sample preparation by Suspension Trapping (S-Trap) and label-free data-independent acquisition (DIA) Mass Spectrometry (MS), the superiority of this procedure to other known MS sample preparation workflows was demonstrated by detecting a higher number of proteins (˜2,000), and obtaining higher reproducibility and overall sensitivity. When comparing the EV proteomic profiles of non-tumorigenic and metastatic breast cancer cells, we identified differentially expressed proteins known to be associated with metastatic processes (e.g., B1 integrin and a enolase [84]) unique to MDA-MB-231 cell EVs (FIG. 5a) and detectable when using as low as 5 ng of total EV proteins. During pilot analyses, it was determined that when using exh-EVs purified with the human-PAN EV-CATCHER assay from 100 μL human EBC or from 100 μL mouse EBC (orthotopic lung tumor model), an average of ˜1.8-3.4 μg total EV protein can be purified. Applicability of this MS approach to exh-EVs was also confirmed by obtaining protcomic profiles of tumor exh-EVs (FIG. 5b-1 and FIG. 5b-2) purified with the human-PAN EV-CATCHER assay from mouse EBC (n=6) collected from the orthotopic animal model (i.e., following tail vein injection of highly metastatic MDA-MD-231 LM2 breast cancer cells). The MS analyses showed that tumor exh-EVs from lung tumor-bearing animals contain metastasis-related proteins, which we identified in our published study using similar cells [83,84,74] (FIG. 5B-1 and FIG. 5B-2, see β1 integrin & α enolase). For this proposal, the protein validations and early detection analyses will be performed using commercial ELISA assays (Invitrogen).

b. Preliminary Studies of Human and Mouse Exh-EVs:

b.1 exh-EVs purified from human EBC contain lung cancer biomarkers: The identification, purification, and evaluation of exh-EVs was initiated using human EBC samples collected with the R-tube™ device (FDA #3004852415) from healthy individuals in the context of an ongoing study. Using Nanoparticle Tracking, TEM (FIG. 6a upper; cup-shaped EV), super resolution microscopy (FIG. 6a lower; ONi) and western blot analyses (FIG. 6b, anti-tetraspanins (anti-CD9, -CD81, and -CD63) antibodies), the presence of exh-EVs in human EBC was validated. Then, the miRNA profiles of exh-EVs purified with our human-Pan EV-CATCHER assay from EBC was compared to those of four other airway biofluids collected from the same 18 subjects (e.g., mouth rinse, buccal brush, bronchial alveolar lavage (BAL), and bronchial brushing). We found that miRNA profiles of exh-EVs clustered closer to those of BAL than those of upper respiratory miRNA profiles (FIG. 6c), confirming the anatomic surrogacy of exh-EVs for the deep lung. A 10-fold increase in the miRNA sequencing depth of exh-EVs was also observed when compared to whole EBC (FIG. 6d). To further confirm deep-lung surrogacy of exh-EVs, EV-CATCHER was customized with an antibody against the lung bronchiolar epithelial Clara Cell Specific Protein (CCSP), a surface protein unique to Clara cells that is inherited by EVs secreted by these cells. Using super-resolution microscopy (ONi), the presence of CCSP on the surface of these exh-EVs was confirmed and a further 10-fold increase in miRNA reads (n=18) when compared to global exh-EVs purified with our human-PAN EV-CATCHER assay was confirmed. These analyses further confirmed that exh-EVs originate from the deep lung and suggests that Clara cells may contribute to the bulk of exh-EV miRNAs. Finally, to investigate the potential diagnostic value of exh-EVs, a lung cancer study for collection of human EBC was established at Hackensack Meridian Health. The miRNA profiles of exh-EVs purified with human-PAN and anti-CCSP EV-CATCHER assays from EBC of healthy controls (Ctl; n=12) and age-matched patients with stage IV lung cancer (LC; n=6) were obtained. Fourteen differentially expressed miRNAs (pval<0.05) were identified in exh-EVs of patients with advanced lung cancer (FIG. 6f), some of which were identically deregulated to those previously identified in circulating EVs purified from the plasma of patients diagnosed with lung cancer (FIG. 6g; miR-486, miR-155) [85,86], which suggests that exh-EVs contain lung cancer biomarkers.

b.2 Utility of human tumor exh-EVs purified from human tumor-bearing models: Considering that it will take time to assemble a cohort of asymptomatic subjects to evaluate the utility of exh-EV biomarkers for early detection of primary and secondary lung cancers, animal models bearing human tumors were developed to accelerate identification of multi-omic tumor exh-EV biomarkers.

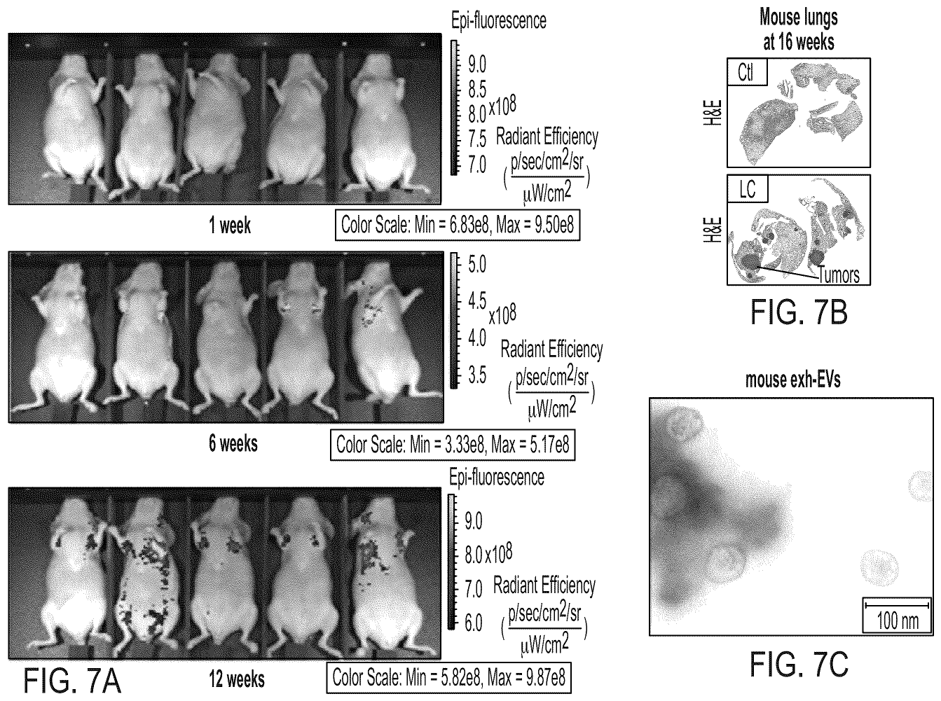

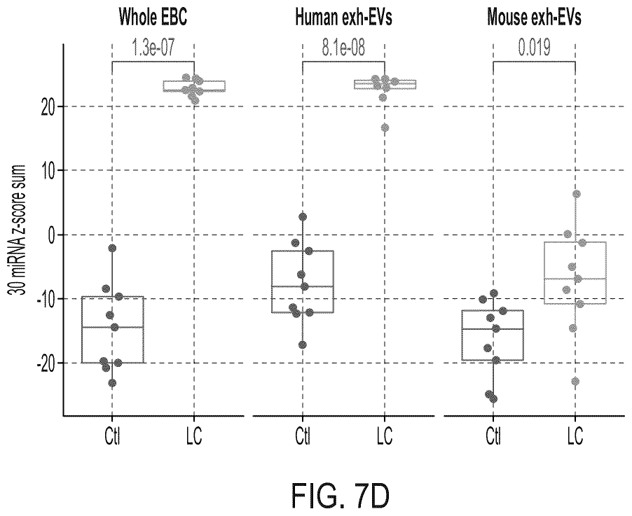

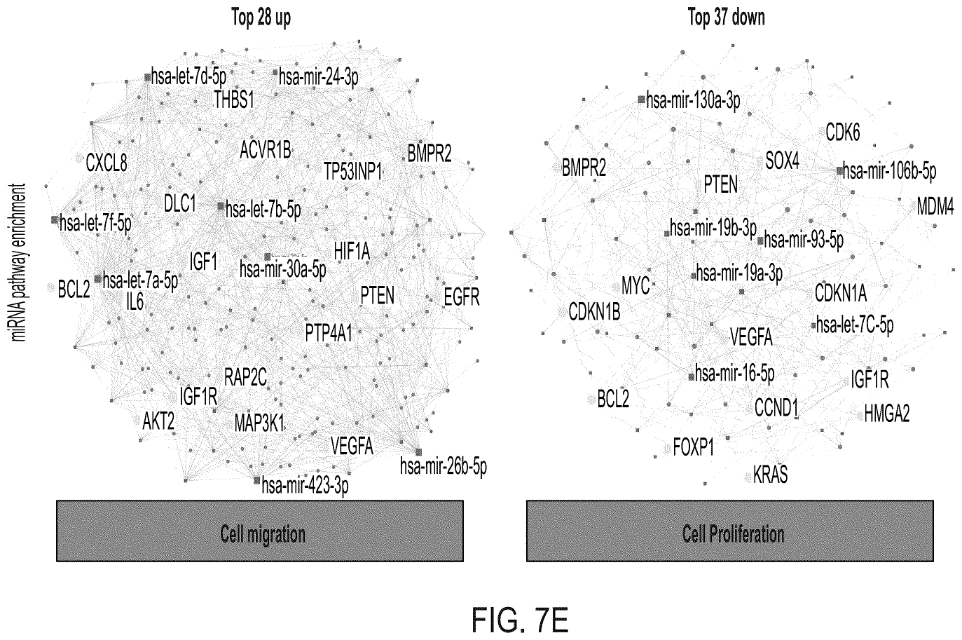

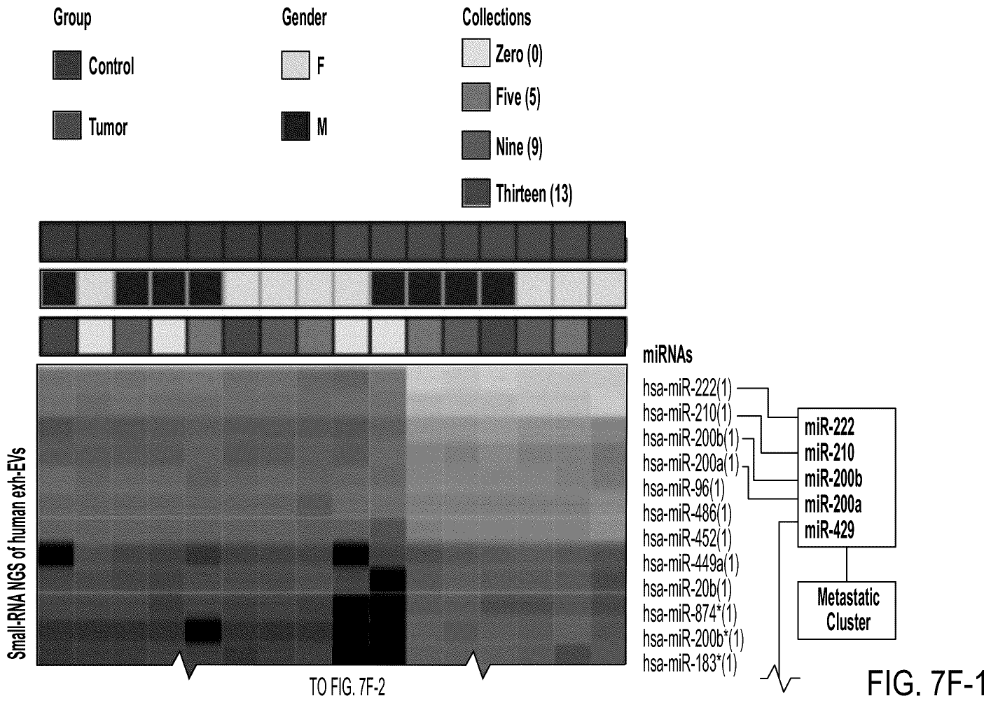

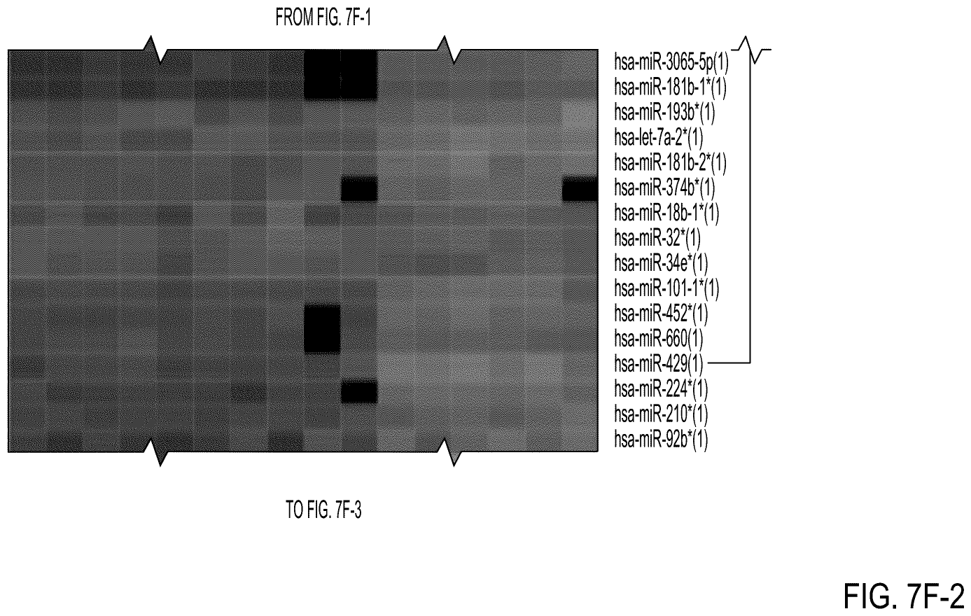

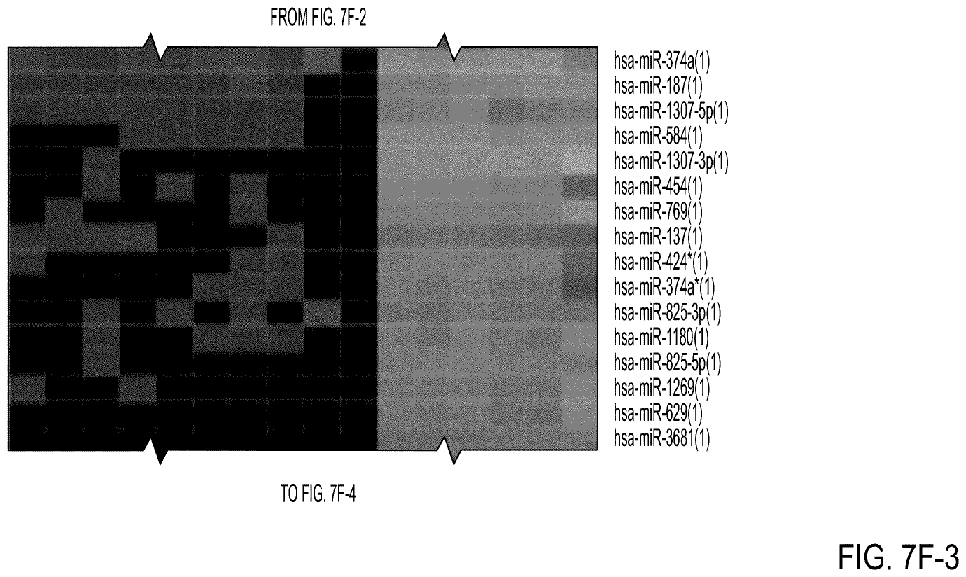

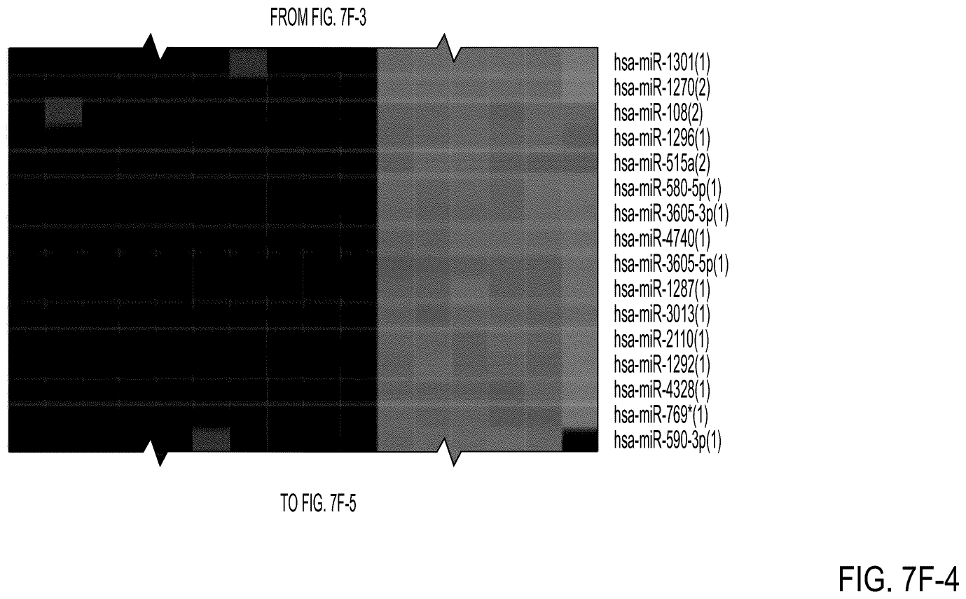

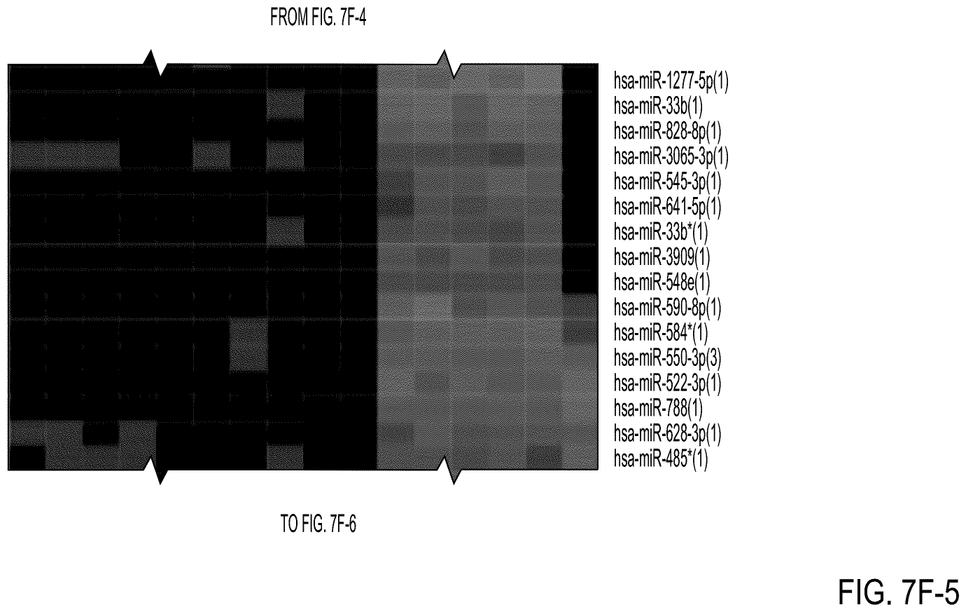

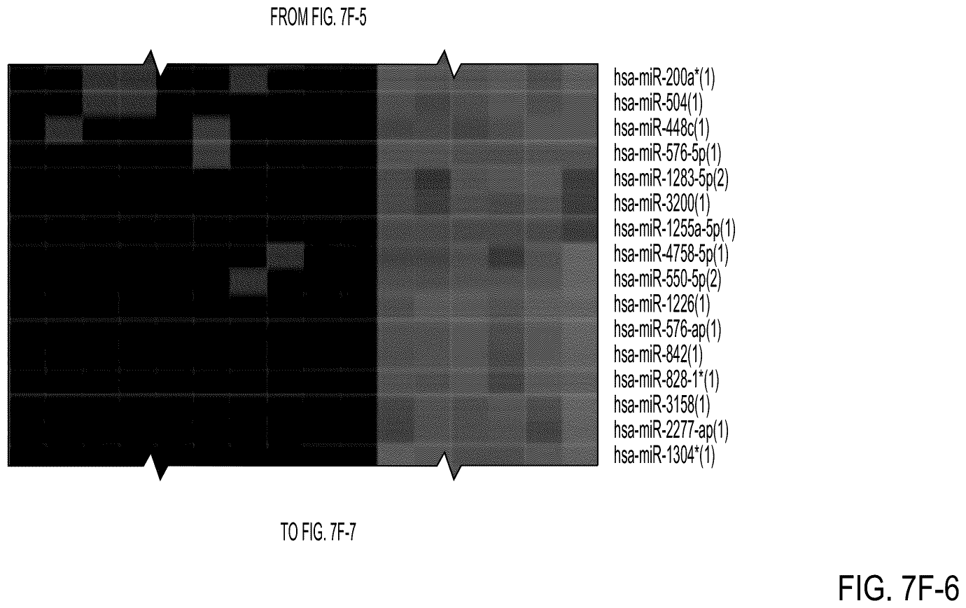

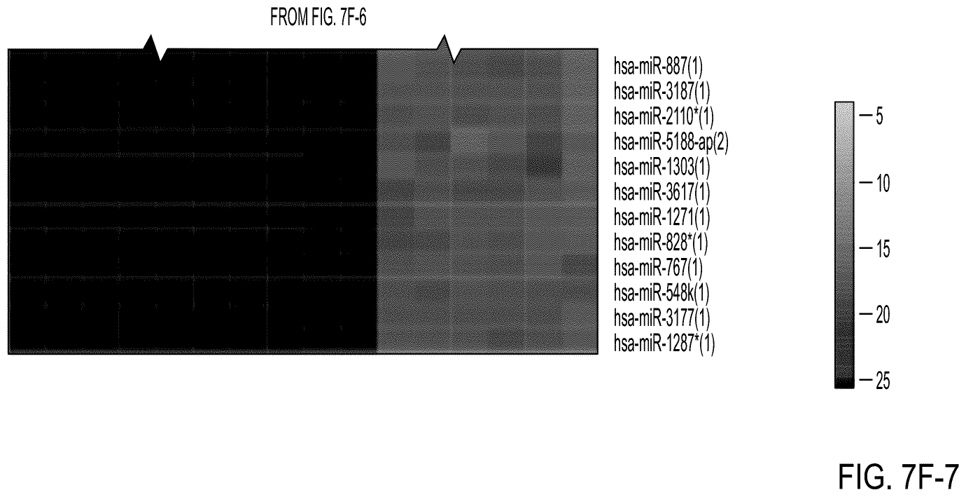

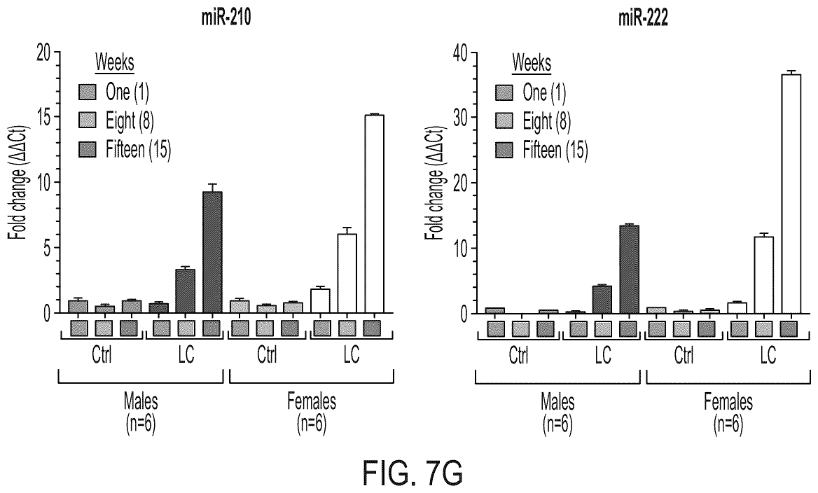

Our mouse model of secondary lung cancer was developed by tail-vein injection of 10⁶ highly metastatic MDA-MB-231 LM2 triple negative breast cancer cells seeding in the lungs of NOD/SCID mice, and transduced with a dual Td-Tomato/luciferase reporter (TdT-Luc) for in vivo bioluminescence (FIG. 7a). These cells were previously studied for their enhanced capacity to colonize the lungs [88,89]. Mouse EBC was collected thrice weekly from single animals for 16 weeks and stored at −80° C. until all samples were collected. At 16 weeks (study term), large tumors were identified by histopathology in the lungs of animals that received the MDA-MB-231 LM2 cells (n=12; (6 males/6 females)), but none in control animals (n=12; (6 males/6 females)) (FIG. 7b). By ultracentrifuging mouse EBC, the presence of exh-EVs in mouse EBC was confirmed by TEM (FIG. 7c).

We then sought to determine if there were human tumor exh-EVs in the EBC of our orthotopic animal model. For these analyses weekly EBC collections of two animals (˜120 μl) from the same group (control or tumor-bearing) were combined and miRNA NGS analyses of whole EBC, of human tumor exh-EVs purified with our human-PAN EV-CATCHER assay, and mouse lung exh-EVs purified with our mouse-PAN (anti-mouse CD9/CD63/CD81 antibodies) EV-CATCHER assay were conducted. miRNA data from control (n=3 pairs) and tumor-bearing (n=3 pairs) animals were compared at three time points. Our analyses revealed that whole EBC of tumor-bearing animals contained highly differentially expressed miRNAs (p<0.05) when compared to whole EBC of controls (FIG. 7d, left). Furthermore, when comparing the miRNA profiles of human and mouse exh-EVs purified from mouse EBC (FIG. 7d, center and right), that the tumor miRNA signal originates from human tumor-derived exh-EVs was confirmed. Our miRNA pathway enrichment analyses of the top 28 upregulated miRNAs in human tumor exh-EVs reveal their role in the control of cellular migration, while the top 37 downregulated miRNAs are associated with the control of cellular proliferation (FIG. 7e). For upregulated miRNAs, the miR-200a/200b/429 metastatic miRNA cluster (FIGS. 7F-1 to 7F-7), was detected as well as mi-210 and miR-222 that have been described during breast cancer metastasis for their enhanced metastatic role upon delivery by circulating EVs [91-93]. Using qPCR, their upregulation over time in human tumor-derived exh-EVs was validated, noting a significantly higher expression in female mice, where increased expression of miR-210 could be detected only 1 week after tail-vein injection of the metastatic cells (FIG. 7g). Our data suggests that qPCR detection of tumor exh-EV biomarkers may be more sensitive than imaging, as bioluminescence in FIG. 7a was inconsistent between mice at 6 weeks.

### Example 2. Aim 1. To Identify and Validate Multi-Omic Tumor Exh-EV Biomarkers and Evaluate their Sensitivity for Early Detection of Human Primary Lung Tumors in Multiple Complementary Animal Models

Rationale: Because assembling a large cohort of asymptomatic individuals for prospective collection of EBC will take a long time, we propose to accelerate early lung cancer biomarker discovery by analyzing the multi-omic contents (miRNAs, mRNAs, and proteins) of tumor exh-EVs purified from the EBC of several complementary animal models of human primary lung cancers. Human primary lung tumors carry mutations in key signaling pathways [94], including TP53 (50-70%) [95], EGFR (20-30%) [96], and KRAS (15-25%) in Non-Small Cell Lung Carcinomas (NSCLC), and RB1 (65%) and TP53 (90%) in Small Cell Lung Carcinomas (SCLC) [94,98]. An estimated ˜85% of all human lung cancers are NSCLC and ˜15% are SCLC [99]. For this first study we propose to establish complementary animal models of human NSCLC carrying EGFR and KRAS mutations (Table 6).

TABLE 6
Primary lung cancer animal models

| | Primary | | | | | |
|---|---|---|---|---|---|---|
| Lung | Model | Species | Mutation | Mouse Models | Induction | Imaging |
| Cancer | Transgenic | Mouse | EGFR | B6; CBA-Tg- | Doxycycline | X-Ray |
| | | | (L858R) | (tetO- | | |
| | | | | EGFR^{L858R})56Hev | | |
| | | Mouse | KRAS | B6.129S4- | AdenoCre | |
| | | | (G12D) | Kras^{tm4Tj}/J | | |
| NSCLC | Orthotopic | Human | EGFR | NOD/SCID | Intra- | Bioluminescence |
| | | H3255 | (L858R) | | Tracheal | (TdT-Luc) |
| | | cells | | | Instillation | |
| | | Human | KRAS | NOD/SCID | | |
| | | A549 | (G12S) | | | |
| | | cerlls | | | | |
| | PDX | Human | EGFR | NOD/SCID | | X-Ray |
| | | tumor | (L858R) | | | |
| | | Human | KRAS | NOD/SCID | | |
| | | tumor | G12 mut | | | |

The transgenic animal models were carefully selected because the production of mutant EGFR or KRAS proteins can be induced in specific lung cells, which then undergo progressive changes leading to NSCLC. By customizing EV-CATCHER to purify tumor exh-EVs originating from these specific lung cells, the sensitivity of detecting early mouse tumor exh-EV biomarkers within mouse EBC will be evaluated (thus mimicking the purification of human tumor exh-EVs contained within the EBC of patients carrying early lung tumors).

Additionally, expression of KRAS, p53 and other oncogenes in Clara cells and/or AT2 cells will be evaluated in the TA-CCSP transgenic animal model. Upon expression of the oncogene in this model, the exh-EVs will be evaluated early, e.g., at the stages of hyperplasia, and during non-invasive disease.

The orthotopic animal models were selected because the human cancer cells that will be instilled through the trachea of the animals will carry the same mutations as those of the transgenic animal models but will also carry a bioluminescent construct to enhance lung tissue imaging of human tumor cells. This will enable thresholds of early biomarker detection to be correlated with respect to tumor load using in vivo and ex-vivo imaging, histology, and immunohistochemistry (i.e., IHC) to track human tumor cells in mouse lungs. The human-PAN EV-CATCHER assay will be customized for direct purification of these human tumor exh-EVs from mouse EBC.

The PDX animal models will be established using NSCLC patient-derived tumor cells carrying the same key mutations as those of the two other animal models. These tumors will be carefully identified a surgical thoracic oncologist and Co-PI on this project. The human-PAN EV-CATCHER assay will be used to capture human tumor-derived exh-EVs from mouse EBC and identify real lung cancer patient tumor-derived exh-EV biomarkers. Control animal colonies will be established with primary human small airway epithelial cells (HSAEC), one for each model, to collect and analyse the content of normal lung exh-EVs. Histology and IHC will be used to precisely quantify tumor formation.

As all three animal models will contain the same oncogene mutations, exh-EV biomarkers will be identified that will enable consistent early detection of these tumors, while establishing a pipeline applicable for identification of exh-EV biomarkers for other primary lung cancer types (i.e., NSCLC containing different mutations or SCLCs).

b. Selection of Human Primary Lung Cancer Models:

i. Transgenic NSCLC mouse models-Transgenic inducible EGFRL858R (C57BL/6J) and KRASG12D (B6CBA) NSCLC mouse strains have been selected so the capture of human tumor exh-EVs from mouse EBC100-102 can be directed (Table 1). Indeed, the double transgenic tetO-EGFRL858R and CCSP-rtTA animal model (acquired from Dr. Jerry Wu) will receive dietary doxycycline to produce tetracyclin and induce expression of the EGFRL858R protein in bronchiolar secretory epithelial Clara cells, where adenocarcinoma will progress [100,103]. The other transgenic model will express the KRASG12D protein upon intranasal introduction of the AdenoCre virus (with modulation of lung tumor load by use of 5×10⁶ to 5×10⁴ PFUs), and activation of the Lox-stop-lox-k-ras G12D allele to lox-k-ras G12D functional mutant allele in both Clara cells and alveolar type II (AT2) cells [104]. Thus, EV-CATCHER will be customized with our anti-mouse anti-Clara Cell Specific Protein (CCSP) antibody in combination with the anti-mouse anti-Surfactant Protein C (SFTPC is highly expressed in AT2 cells [105]) antibodies to purify mouse tumor exh-EVs from mouse EBC (FIG. 6d).

ii. Orthotopic animal models-Human NSCLC lung cancer cell lines will be transduced with the Td-Tomato-Luc lentiviral vector for weekly in vivo and end-point ex vivo bioluminescence [106]. The selected human cancer cells will be expanded prior to tracheal instillation into the lungs of immuno-deficient NOD/SCID nude mice [107,108].

iii. PDX animal models-Human NSCLC lung tumors will be resected and transported within 2 hours on ice to the laboratory of Dr. Loudig. Tumors will then be dissociated using the human tumor dissociation kit (Miltenyi, 130-095-929) and the GentleMACS™ Octo Dissociator following manufacturer's instructions [109]. After dissociation, the cells will be expanded in tissue culture, quantified, and instilled in the trachea of NOD/SCID mice for lung uptake [107-109]. EGFR and KRAS mutation profiles will be obtained from biopsies prior to patient surgery to select the right tumors for the study. For these PDX animal models, animal controls will be established by tracheal instillation of HSAECs, one type per model, to collect normal human exh-EVs from mouse EBC (i.e., normal human lung biomarker background for our analyses).

The human-PAN EV-CATCHER assay will be used to purify human exh-EVs from mouse EBC of orthotopic (i.e., tumor bearing) and PDX (i.e., control and tumor-bearing) animal models.

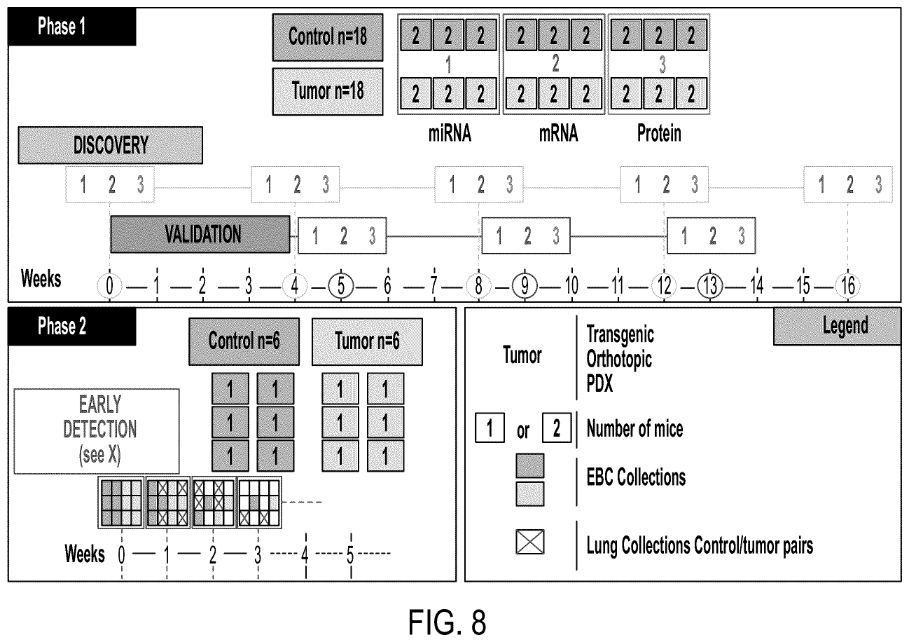

c. EBC collections: For transgenic, orthotopic, and PDX animal models, EBC will be collected from each individual animal. All collection devices, filters, and condensers will be cleaned between collections (see supra, Example 1 section a. 1) and the EBC samples will be transferred to barcoded tubes and stored at −80° C. until all experiments can be run. For discovery and validation (Phase 1), EBC will be collected thrice-weekly for 16 weeks from control (n=18) and tumor-bearing (n=18) animals for each animal model (FIG. 8). Pilot analyses revealed that 20 μL EBC can be collected from a single animal within 2 hours. EBC from two animals (set animal pairs for Phase 1) of the same group (control or tumor-bearing) will be pooled to collect ˜40 μL EBC per day or 120 μL per week, per animal pair. Because we seek to generate triplicate measures for miRNA, mRNA, proteomic analyses and we have 18 animals per group, 9 EBC samples of 120 μL will be generated weekly per group (FIG. 8). Multi-omic biomarker discovery will be done with exh-EVs purified from EBC on even (i.e., 0, 4, 8, 12, and 16) weeks. Validation analyses will be performed with exh-EVs purified from EBC on odd (i.e., 5, 9, and 13) weeks. For early detection of primary NSCLC lung cancers (Phase 2), new animal colonies will be established, including 6 control and 6 tumor-bearing animals, per model. EBC will be collected thrice weekly and 60 μl per animal (i.e., 3×20 μl) will be obtained, from which exh-EVs will be purified using our EV-CATCHER assay. The purified exh-EVs will be split into three aliquots, to undergo early biomarker detection (i.e., ddPCR, ELISA). Upon detection of one, two, or the three types of biomarkers the animals will be euthanized, the lungs will be imaged and undergo histological and IHC reviews to establish a correlation between the early biomarker detection and tumor burden.

d. Imaging and Histology: Bioluminescence and X-ray analyses will be conducted on live animals and on lung tissues of euthanized animals. Histological tumor reviews will be performed on resected lung tissues obtained at the end of Phase 1 for all animals for all models, and during Phase 2, upon detection of the selected biomarkers. Lung tissues will be formalin-fixed paraffin-embedded (FFPE), sectioned (5 μm), and hematoxylin and eosin (H&E) stained for histological evaluations. IHC evaluations of lung tissues from our transgenic animal models will be done using antibodies targeting the mouse EGFRL858R (ABclonal Cat #A5031) and KRASG12D (GeneTEX Cat #HL10) proteins on unstained 5 μm FFPE sections. Antibodies targeting the Td-Tomato luciferase protein will be used for detection of lung tumor cells for orthotopic and anti-human anti-EGFRL858R (Abcam Cat #ab192263) and anti-KRASG12D (Abcam Cat #ab192263) antibodies for PDX animal models.

Additionally, a microCT (e.g., SkyScan 1276, Microphotonics.com) may be used for imaging lung tissue in live animals to enable visualization of the progression of disease compared to biomarker level before it is visible by stanrd imaging methods.

e. Experimental Design:

Subaim 1a (Phase 1): Using the mouse EBC collection systems and optimized molecular assays, we propose to identify miRNA, mRNA, and protein biomarkers from lung tumor exh-EVs of the transgenic (T), orthotopic (O), and PDX animal models (P), which will be referred to as T.O.P. throughout this aim.

For multi-omic biomarker discovery, exh-EVs will be purified from mouse EBC samples collected from paired-animals on even (i.e., 0, 4, 8, 12, and 16) weeks from both controls and tumor-bearing T.O.P. animal models (FIG. 8). For transgenic animal models the EV-CATCHER assay (customized with anti-mouse anti-CCSP and anti-SFTPC antibodies) will be used to capture mouse tumor exh-EVs from mouse EBC [100, 101]. For orthotopic and PDX animal models, our human-PAN EV-CATCHER assay will be used to capture human tumor exh-EVs from mouse EBC samples (i.e., for our PDX models, normal human exh-EVs will also be purified from the controls). miRNA and mRNA will be extracted in triplicate from the purified exh-EVs and NGS libraries produced using our optimized and validated methods [42-46]. For proteomics discovery, the S-Trap/DIA mass spectrometric pipeline that was developed in collaboration with our partner at Georgetown University will be used [74]. When NGS and MS data (i.e., triplicate measures) become available for all the T.O.P. animal models, statistical analyses (i.e., as described in ¶ C.iii.3) will be completed, to establish a list of our top differentially expressed 5 miRNAs, 5 mRNAs, and 5 proteins unique to EGFRL858R or KRASG12D mutations, but also possibly including common biomarkers to both mutations. For multi-omic biomarker validations, qPCR (miRNA, mRNA) and ELISA (protein) assays will be used to validate detection of the top biomarkers using miRNA, mRNA, or mildly collagenase digested proteins [108, 109] purified from exh-EVs captured from mouse EBC collected on odd (i.e., 5, 9, and 13) weeks from the T.O.P. animal models (i.e., control and tumor-bearing animals) of human primary lung NSCLC.

Subaim 1b (Phase 2): To establish a correlation between the lowest biomarker detection threshold and lung tumor burden (i.e., imaging and histology), we propose performing analyses at early stages of disease in the T.O.P. animal models by testing EBC from single animals. The control and tumor-bearing animal colonies will each include 6 animals (FIG. 8). As each mutation (i.e., EGFR or KRAS) may result in different detection thresholds, early detection of the validated biomarkers for each of our T.O.P. animal models will be tested. The analyses will be started with the orthotopic model by decreasing initial tumor load (i.e., tracheal instillation of 10⁵ and 10⁴ cells). Three weekly collections per single animal (i.e., 60 μl) will be collected and combined, exh-EVs will be purified using the appropriate EV-CATCHER assay, samples will be separated into three fractions to undergo miRNA and mRNA extractions and protein pre-processing [108,109] prior to conducting ddPCR (i.e., ultra-sensitive for low input material) and ELISA analyses of the top 5 validated miRNA, mRNA, and protein biomarkers. Upon detecting the lowest amount of tumor cells at the earliest time-point, the same amount of human patient tumor cells will be used to establish the PDX models and conduct the same early detection analyses. Finally, as we wish to evaluate early detection of primary lung NSCLC at early neoplastic stages, tumor load in the transgenic animal models will be modulated by decreasing doxycycline-dosage (i.e., EGFRL858R) and AdenoCre intranasal exposure (i.e., KRASG12D; using 5×10⁴, and 5×10³ PFUs). Upon significant detection of one of the biomarkers (miRNA, mRNA, or proteins) by comparison to control animals, the lungs will be collected for pathology and immunohistochemistry (IHC) (anti-Td-Tomato-Luc for orthotopic; anti-EGFRL858R, and anti-KRASG12D for transgenic and PDX models) to correlate the findings.

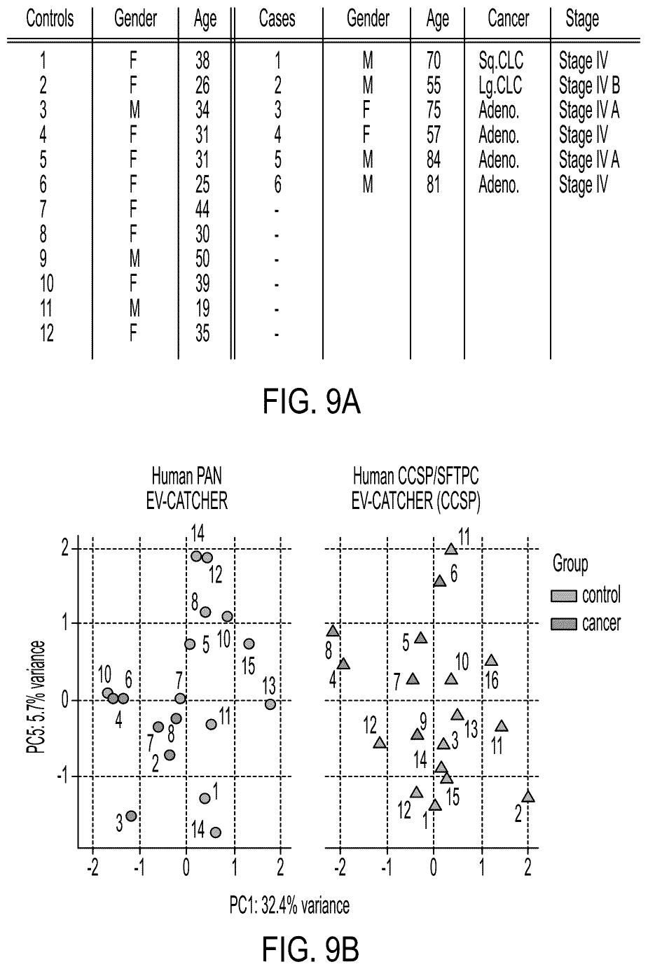

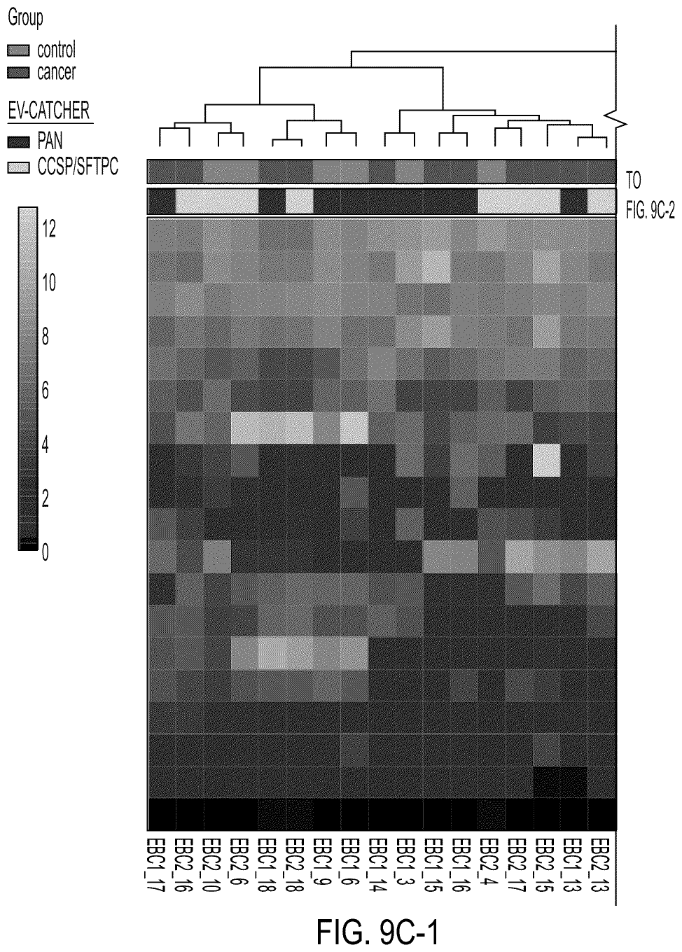

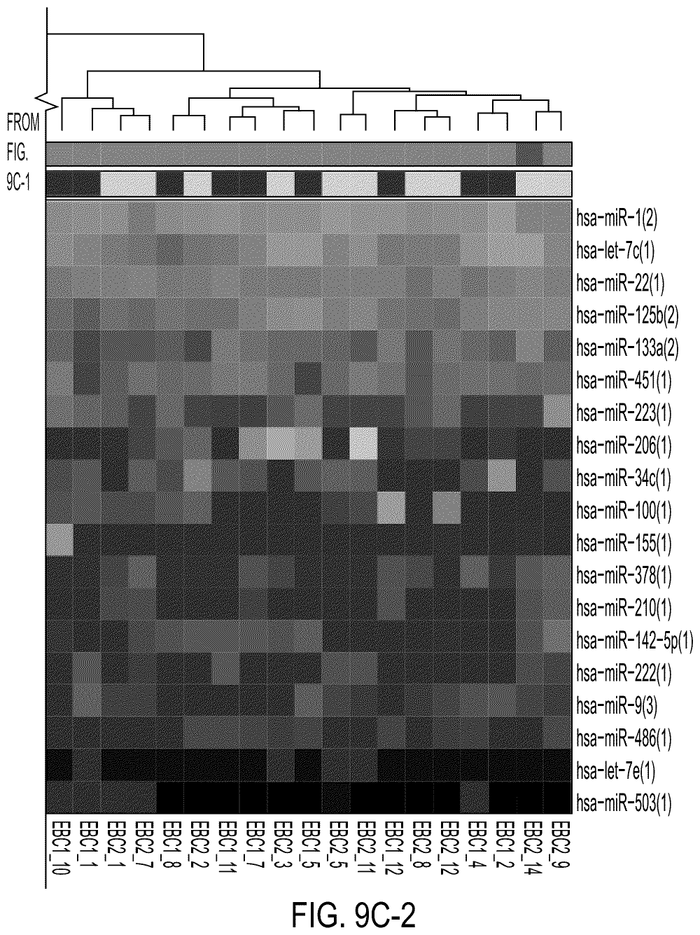

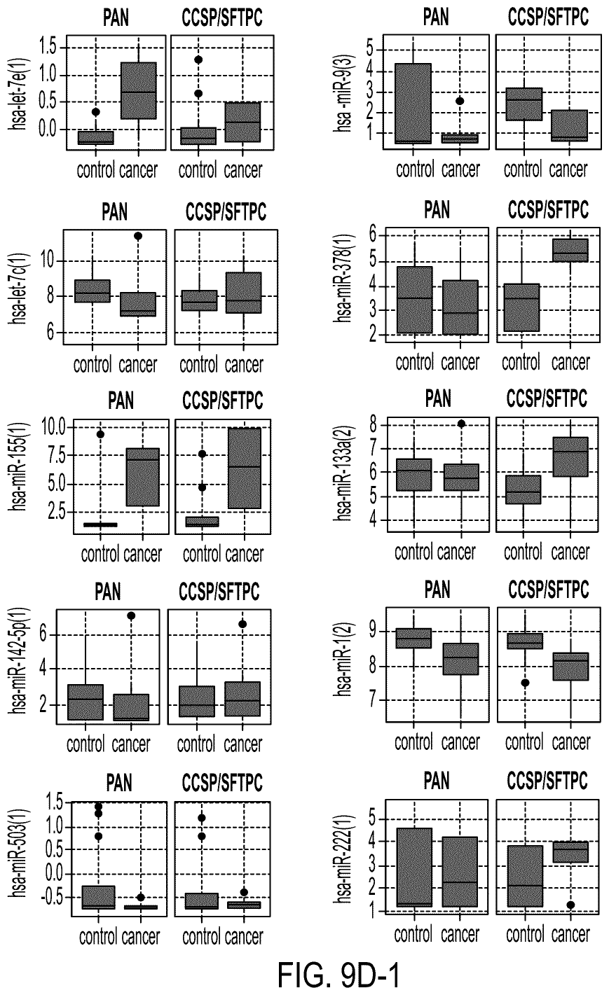

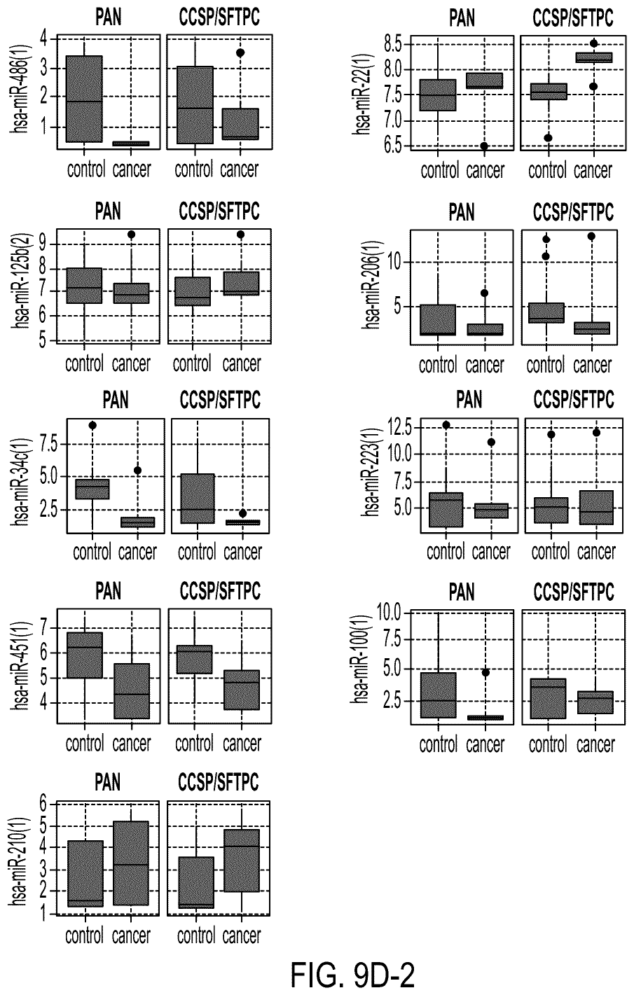

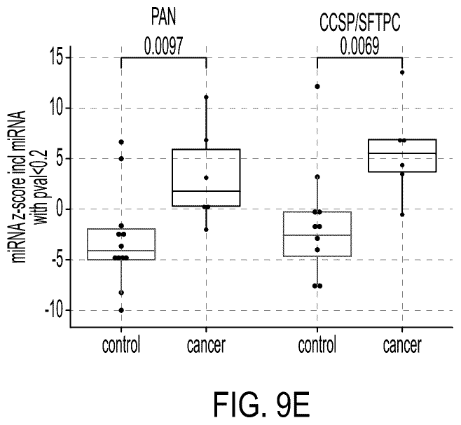

FIGS. 9A-9E shows MiRNA expression profiling of exh-EVs purified from EBC of healthy controls and patients with advanced lung cancer. FIG. 9a shows a table detailing sample ID, gender, and age of the subjects involved in this comparative analysis, which includes 12 controls and 6 treatment naïve patients diagnosed with stage IV lung cancer. The first patient was diagnosed with Squamous Cell lung Carcinoma, the second with Large Cell Lung Carcinoma, and the third, fourth, fifth, and sixth patients were diagnosed with adenocarcinoma (Non-small Cell Lung Carcinoma). FIG. 9b shows a PCA plot displaying the similarities in miRNA expression between the 12 controls (Healthy) and the 6 cases (Lung cancer) for exh-EVs that were purified from EBC with the PAN (CD63/CD9/CD81) EV-CATCHER assay or with the CCSP/SFTPC EV-CATCHER assay from the same samples. FIG. 9c-1 and FIG. 9C-2 shows Heatmap expression analysis of the top 19 most differentially expressed miRNAs between the 12 controls (Healthy) and the 6 cases (Lung cancer) for exh-EVs that were purified from EBC using with the PAN EV-CATCHER assay or the CCSP/SFTPC EV-CATCHER assays. FIG. 9d-1 and FIG. 9D-2 shows Box plot analyses of the top 15 most differentially expressed miRNAs between the 12 controls (Healthy) and the 6 cases (Lung cancer), displaying significant log₂ fold expression differences, as detected by small-RNA sequencing analyses. FIG. 9e shows Box plot analyses of the Z-score obtained from the top 14 most differentially expressed miRNAs between the 12 controls (left; healthy) and the 6 cases (right; lung cancer), for miRNA profiles after small-RNA sequencing of exh-EVs purified with the PAN EV-CATCHER assay (PAN; left) or the CCSP/SFTPC EV-CATCHER assay (CCSP/SFTPC; right). The p-value for differences between controls (n=12) and the cases (lung cancer; n=6) is displayed in the panels. This data shows that the purification of EVs with CCSP/SFTPC from human EBC specimens provides a more sensitive detection of the common biomarkers, as well as additional ones (MiR-22 and miR-378, and miR-133a).

### Example 3. Aim 2. To Identify and Validate Multi-Omic Tumor Exh-EV Biomarkers that Allow the Detection of Secondary Human Lung Cancers Earlier than by Imaging Thresholds

a. Rationale: It is estimated that up to 30% of patients diagnosed with a malignant disease will develop secondary cancer in the lungs (pulmonary metastases), the second most frequent site of metastatic growth [20,111,112]. Lung metastases most often originate from primary lung, colorectal, breast, urogenital cancers, and melanoma [112]. In this aim, multi-omic tumor exh-EV biomarkers will be identified, validated and sensitivity evaluated for early detection of the most frequent secondary lung cancers, including those derived from human colorectal, breast, and bladder cancers using orthotopic and PDX animal models (Table 7).

TABLE 7
Human cancer cell lines selected for Aim 2.

| | | | Primary | | | |
|---|---|---|---|---|---|---|
| | | | Cancer | Animal | | Tumor |
| | | Species | Origin | Model | Induction | Detection |
| Cell Lines | HT29 cells | Human | Colorectal | NOD/SCID | Tail vein | Bioluminescence |
| | MDA-MB- | | Breast | | injection | Td-Tomato- |
| | 231 LM2 | | | | | Luc |
| | cells | | | | | |
| | T24T cells | | Bladder | | | |
| Tumors | Patient | Human | Colorectal | NOD/SCID | Tracheal | X-Ray |
| | Derived | | Breast | | installation | |
| | Xenograft | | Bladder | | | |

Our preliminary study of human tumor exh-EVs purified from EBC of mice developing secondary lung cancer, upon tail-vein injection of human lung metastatic breast cancer cells, indicates that miRNA (FIGS. 7F-1 to 7F-7) and protein (FIG. 5A, FIG. 5B-1 and FIG. 5B-2) biomarkers of human tumor exh-EVs are detectable and quantifiable at early disease stages (i.e., increased expression of miR-210 in females, 1 week after injection of tumor cells without bioluminescent detection in vivo). Thus, we hypothesize that human secondary lung tumors secrete EVs with highly different multi-omic biomarker contents, which are specific to their metastasizing tissue of origin (i.e., foreign to the lung), which become aerosolized during normal tidal respiration as exh-EVs, and that can be detected at early stages of cellular invasion, before lesions become detectable by imaging. To demonstrate this, matched secondary cancers will be carefully established in orthotopic and PDX animal models, to capture and identify unique (i.e., specific of the primary tumor-tissue origin) and potentially common (i.e., metastatic molecular pathways) multi-omic human tumor exh-EV biomarkers from mouse EBC. By decreasing tumor cell input, the sensitivity of our approach for early and non-invasive exh-EV biomarker detection of secondary human lung cancers will also be evaluated.

b. Mouse models of human secondary lung cancers: Orthotopic animal models of human secondary lung cancers will be developed by tail-vein injection of metastatic colorectal (HT29) 113, breast (MDA-MB-231 LM2) [87-89] and bladder (T24T) cancer cells known to preferentially colonize the lungs (Table 3). Each of the cell lines will be transduced with the TdTomato-Luc lentiviral vector for in vivo (i.e., monitoring of disease progression) and ex vivo (study endpoint) bioluminescence of lung tissues [106]. For development of PDX animal models, matching the orthotopic secondary lung cancer animal models, human patient secondary lung tumors metastasized from primary colorectal, breast, and bladder cancers (i.e., clinically validated by cytokeratin profiling of the biopsies) will be harvested. The resected tumors (>1 cm³) will be dissociated, expanded in tissue culture, and about 1×10⁶ cells will be transplanted in the lungs of tumor-bearing NOD/SCID nude mice animals [107-108] via tracheal instillation. Control mice will receive tracheal instillation of primary Human Small Airway Epithelial Cells (HSAECs) to account for normal human exh-EVs. The human-PAN EV-CATCHER assay will be used to purify human exh-EVs from mouse EBC.

c. Sample collections: For both the orthotopic and PDX animal models, the experiments will be completed in two phases (FIG. 8). During discovery and validation (Phase 1), for all lung cancer animal models, EBC will be collected from 18 control (9 pairs) and 18 tumor-bearing (9 pairs) animals, using the single animal EBC collection systems (FIG. 2 left). We anticipate collecting about 20 μl EBC/animal within two hours, thrice weekly, to obtain 60 μl per animal. However, to collect sufficient EBC for our multi-omic analyses, EBC from two animals (i.e., set pairs throughout our study) will be combined to obtain about 120 μl EBC sample. With 18 animals or 9 pairs per group, 9 EBC samples of about 120 μl per control and 9 per tumor-bearing animal groups will be collected per week for 16 weeks. Tumor formation/size will be measured by X-Ray (PDX models) and bioluminescence (orthotopic models). EBC samples will be barcoded and stored at −80° C. For early detection analyses (Phase 2) EBC will be collected thrice weekly from single animals (60 μl/animal/week) for ddPCR (miRNA, mRNA) and ELISA (protein) analyses.

d. Imaging and Histology: For Phase 1, histology of the lungs from matched control and tumor-bearing animals will be evaluated at study endpoint (16 weeks) for both PDX and orthotopic animal models. Bioluminescent imaging (orthotopic models) and X-Ray imaging will be obtained weekly. For Phase 2, when at least one of the multi-omic biomarkers is reproducibly detected, lungs from matched controls and tumor-bearing animals will be collected for histological evaluation. As human tumor cells may be disseminated in mouse lung tissues at early disease stages, which we will detect with our biomarkers, IHC (anti-human anti-cytokeratin 7 and/or anti-Ki67 antibodies) will be used to localize tumor cells.

e. Experimental design of Aim 2: For all animal models (i.e., PDX and orthotopic), human tumor exh-EVs will be purified from mouse EBC using our human-PAN EV-CATCHER assay prior to multi-omic analyses.

Subaim 2a: For all (i.e., 3 PDX and 3 orthotopic) animal models that will be established for this aim, multi-omic biomarker discovery that will include NGS (miRNA and mRNA) and MS (proteomic) analyses will be performed in triplicate on human exh-EVs purified from EBC samples of paired mice collected on even (i.e., 0, 4, 8, 12, and 16) weeks (FIG. 8). Once data from all models is available, the statistical analytical approaches described below in (3) will be used to identify the top 5 most detectable (i.e., high reads or peptide counts) miRNAs, mRNAs, and proteins specific to human tumor exh-EVs for each secondary tumor model type (i.e., from primary colorectal, breast, and bladder cancers/tumors), which are differentially expressed between lung tumor-bearing and control animals but that are common to matched PDX and orthotopic models. Because early detection may vary between the different biomarkers, validation analyses will be conducted using qPCR (miRNAs and mRNAs) and ELISA (proteins) assays to detect the top 5 miRNA, 5 mRNA, and 5 proteins biomarkers (i.e., common between PDX and orthotopic secondary lung cancer models) and using human tumor exh-EVs purified from mouse EBC samples collected from paired-mice on odd (i.e., 5, 9, and 13) weeks (FIG. 8). Imaging and histological validations of tumor formation will be performed as described above.

Subaim 2b: First, we propose to assess the reproducibility and sensitivity of detecting our top multi-omic human tumor exh-EV biomarkers identified in subaim 2a, at the earliest possible stage of metastasis. To this end, EBC from single animals (orthotopic models) will be evaluated when decreasing tumor loads by tail-vein injection of 10⁵, 10⁴, or 10³ cells in three different groups of 6 animals (i.e., one group per concentration) and a group of 6 controls. For these analyses EBC samples from single animals will be collected thrice weekly and combined prior to human tumor exh-EV purifications, miRNA and mRNA ddPCR (i.e., more sensitive than qPCR for low input) and ELISA (protein) analyses. Upon positive detection of at least one type of biomarkers, the positive animals and matched controls will be sacrificed, the lungs will be collected, and perform histological and IHC analyses will be performed to establish a correlation between tumor burden and biomarker detection. Second, we will seek to improve customization and selectivity of EV-CATCHER based on tissue origin of the secondary lung tumors. To this end, proteomics data from our matched orthotopic and PDX animal models will be analyzed to identify surface proteins unique to the tissue origin of the primary cancers (i.e., colorectal, breast, and bladder), to improve capture of their respective exh-EVs. Because we will have banked EBC samples collected at 2, 6, 10 and 14 weeks from the orthotopic animal models, the newly customized EV-CATCHER assays will be tested to purify human tumor exh-EVs and allow qPCR (miRNA, mRNA) and ELISA (proteins) detection of the multi-omic biomarkers identified in subaim 2a. Upon validation of the customized assays (i.e., one per type of primary cancer), detection of validated biomarkers will be evaluated using exh-EVs purified from EBC collected at 2, 6, 10 and 14 weeks from our PDX animal models. These analyses will show that the clinical detection of secondary lung cancers originating from primary colorectal, breast, and bladder cancers can be specifically customized.

Subaim 2c: Based on our preliminary data showing that miRNAs deregulated in human tumor exh-EVs purified from the EBC of our secondary lung cancer animal model may regulate genes involved in both cellular proliferation and migration (FIG. 7e), we propose to evaluate how tumor exh-EVs from secondary lung tumors may facilitate tumor cell growth and dissemination in the lung tissues. For this exploratory subaim, we have chosen to work with an expert in the modelling of colorectal carcinogenesis [115,116], who will guide us when we will conduct in vitro transwell migration assays. For these experiments, Human Pulmonary Microvascular Endothelial Cells (HPMECs) will be seeded as a monolayer into the upper compartment of our Transwell chambers. For these assays the non-metastatic GFP-labelled SW480 colorectal cells will be seeded on HPMECSs in the upper transwell chambers, which will be not treated (control) or treated with 1×10⁹ tumor exh-EVs (i.e., quantified with our Spectradyne nanoparticle tracker) purified with our human-PAN EV-CATCHER from banked EBC collected at weeks 3, 7, and 13 (in triplicate) from our orthotopic and then PDX models of secondary lung tumor originating from primary colorectal cancer (subaim 2a). If no changes in migration of SW480 cells are detectable by bioluminescence, two additional non-metastatic colorectal cancer cell lines will be tested. However, based on our RNAseq analyses we anticipate that the migratory properties of these cells will be enhanced by exh-EVs. The NGS transcriptomic (miRNA, mRNA) profiles GFP-SW480 cells treated with exh-EVs and migrated to the lower compartment of the chamber, GFP-SW480 cells from the upper compartment (i.e., tumor exh-EV-treated but non-migrated), and untreated GFP-SW480 cells will be obtained and analyzed. In addition to the transwell migration assays MTT cell viability & proliferation assays will be performed, where exh-EV-treated and untreated GFP-SW480 cells will be analyzed for the reduction of tetrazolium salts to colored formazan compounds, which provides a sensitive and accurate method for the measurement of cell viability and proliferation [118]. These experiments will lay the groundwork for identification of cancer cell pathways that are enhanced by metastatic tumor exh-EVs at early disease stages.

3. Statistical analysis, power considerations: MiRNA and mRNA sequencing data will be processed by our established pipeline [42,46], with sequencing counts (Discovery phase) normalized using R package DESeq2 [120]. Proteomic data will be transformed using natural logarithm and scaled to a mean of 0 and a standard deviation (SD) of 1. First, principal component analysis (PCA) and heatmap will be applied to examine biologic and assay variability between samples collected at different times (weeks), between triplicate measures, and between different tumor-bearing animal models. Assay variability (batch effects) will be adjusted and technical outliers will be identified using robust PCA implemented in the rrcov package in R. To account for multiple testing, discovery analyses will be corrected using a false discovery rate with the threshold of 0.05. Subsequent analyses are designed to effectively address the specific aims of this study, as detailed below:

Discovery of top differentially expressed EV, exh-EV and circ-EV miRNA, mRNA, and protein biomarkers: At each time point, differentially expressed miRNAs and mRNAs between control (3 animal pairs per RNA type) and tumor-bearing animal models (3 animal pairs per RNA type), processed together to reduce bias, will be analyzed using DESeq2 package in R, while proteomic data will be analyzed by linear regression. In addition, linear mixed model (LLM) framework will be applied to integrate repeated measures of the longitudinal data, while adjusting for within-subject correlation. Let Yij be the normalized and transformed level of a miRNA, mRNA and protein for pooling animal index i and week j. The general form of the LMM model will be specified as: E(Yij)=αi+βXi+θTj+λXiTj, in which αi is the random effect for pooling sample i, Xi denotes group, β is the corresponding regression coefficient for Xi, Tj is a vector of weeks, θ is the corresponding regression coefficients, λ is the interaction effect between group and week, and εij is a random error. When comparing control and tumor-bearing (or treated and untreated for assessment of response to therapy) animal groups, the difference in the change of biomarker (miRNA, mRNA, or protein) between different weeks will be assessed by λ.

Validation/detection of miRNA, mRNA, and protein biomarkers: The average of triplicate qPCR/ddPCR and ELISA quantifications after normalization will be calculated using the same approaches described for Discovery.

Power consideration: The power of was estimated by PASS sample size software (v15, www.ncss.com/software/pass/). Based on preliminary data, it was assumed that 20% of miRNAs, mRNAs, and proteins are truly different between EVs of control and tumour-bearing groups. Given the sample size n=3/group and time, the study will have 80% power at FDR=0.05 to identify a miRNA, mRNA or protein with Fold Change (FC)≥2.5 when the coefficient of variation is 0.2. Of note, because longitudinal repeated measures are available, this study would be expected to have an even higher power than that estimated above. Our preliminary data have suggested many signature markers have much higher FCs.

Tables 8-11, which follow, list miRNAs compiled based on the human miRNA data from primary lung cancer and exh-EVs in the secondary human lung cancer animal models.

TABLE 8
Human Primary lung Cancer miRNA biomarkers
Human PAN (using human anti-CD63/CD9/CD81)

| Human Primary lung Cancer miRNA | |
|---|---|
| biomarkers Human Pan (using human | Deregulation in |
| anti-CD63/CD9/CD81) | cancer exh-EVs |
| Let-7e | Upregulated |
| miR-9 | Downregulated |
| miR-486 | Downregulated |
| Let-7c | Downregulated |
| miR-206 | Downregulated |
| miR-155 | Upregulated |
| miR-34c | Downregulated |
| MiR-1 | Downregulated |
| MR-451 | Downregulated |
| miR-100 | Downregulated |
| miR-503 | Downregulated |

TABLE 9
Human Primary Lung Cancer miRNA biomarkers
(using Human anti-CCSP/SFTPC)1

| Human Primary Lung Cancer miRNA | Deregulation in |
|---|---|
| biomarkers (Human anti-CCSP/SFTPC)l | cancer exh-EVs |
| Let-7e | Upregulated |
| miR-9 | Downregulated |
| miR-22 | Upregulated |
| miR-378 | Upregulated |
| miR-206 | Downregulated |
| miR-155 | Upregulated |
| miR-133a | Upregulated |
| miR-34c | Downregulated |
| miR-1 | Downregulated |
| miR-451 | Downregulated |
| miR-503 | Downregulated |
| miR-222 | Upregulated |
| miR-210 | Upregulated |

TABLE 10
Mouse model of human secondary lung cancer human Exh-Ev
miRNA biomarkers using Human PAN (anti-CD63/CD81/CD9)

| Mouse model of human secondary lung | |
|---|---|
| cancer human Exh-Ev miRNA biomarkers | Deregulation in |
| using human anti-CD63/CD81/CD9 | cancer exh-EVs |
| hsa-miR-30c(2) | Upregulated |
| hsa-let-7(d)(1) | Upregulated |
| hsa-miR-151-3p(1) | Upregulated |
| hsa-miR-222(1) | Upregulated |
| hsa-miR-210(1) | Upregulated |
| hsa-miR-30a(1) | Upregulated |
| hsa-let-7g(1) | Upregulated |
| hsa-miR-125b(2) | Upregulated |
| hsa-miR-29b(2) | Upregulated |
| hsa-miR-27a(1) | Upregulated |
| hsa-miR-181a(2) | Upregulated |
| hsa-miR-100(1) | Upregulated |
| hsa-let-7a(3) | Upregulated |
| hsa-miR-29a(1) | Upregulated |
| hsa-miR-423-3p(1) | Upregulated |
| hsa-miR-27b(1) | Upregulated |
| hsa-miR-200b(1) | Upregulated |
| hsa-miR-24(2) | Upregulated |
| hsa-miR-26a(2) | Upregulated |
| hsa-let-7b(1) | Upregulated |
| hsa-miR-30e1 | Upregulated |

TABLE 11
Mouse model of human secondary lung Cancer Mouse Exh-EV
miRNA biomarkers using Mouse PAN (anti-CD63/CD9)

| Mouse model of human secondary lung | Deregulation in |
|---|---|
| Cancer Mouse Exh-EV miRNA biomarkers | mouse tissue |
| using mouse Pan (anti-CD63/CD9) | exh-EVs |
| Hsa-miR-22(1)A82 | Downregulated |
| Hsa-miR-451(1) | Downregulated |
| Hsa-miR-221(1) | Downregulated |
| Hsa-miR-30e(1) | Downregulated |
| Hsa-miR-30d(1) | Downregulated |
| Hsa-miR-126-3p(1) | Downregulated |
| Hsa-miR-125b(2) | Downregulated |
| Hsa-miR-34c(1) | Downregulated |
| Hsa-miR-203(1) | Downregulated |
| Other_miRNA | Downregulated |
| Hsa-miR-92a(2) | Downregulated |
| Hsa-miR-23a(1) | Downregulated |
| Hsa-miR-21*(1) | Downregulated |
| Hsa-miR-148a(1) | Downregulated |
| Hsa-miR-30c(2) | Downregulated |
| Hsa-miR-103(2) | Downregulated |
| Hsa-miR-206(1) | Downregulated |
| Hsa-miR-143(1) | Downregulated |
| Hsa-miR-16(20 | Downregulated |
| Hsa-miR-200c(1) | Downregulated |
| Hsa-miR-101(2) | Downregulated |
| Hsa-miR-140(1) | Downregulated |
| Hsa-let-7c(1) | Downregulated |
| Hsa-miR-30a*(1) | Downregulated |
| Hsa-miR-184(1) | Downregulated |
| Hsa-miR-1(2) | Downregulated |
| Hsa-miR-19b(2) | Downregulated |
| Hsa-miR-141(1) | Downregulated |
| Hsa-miR-190b(1) | Downregulated |
| Hsa-miR-148b(1) | Downregulated |
| Hsa-miR-10a(1) | Downregulated |
| Hsa-miR-7(3) | Downregulated |

### REFERENCES

1. Sung H, Ferlay J, Siegel R L, Laversanne M, Soerjomataram I, Jemal A, Bray F. Global Cancer Statistics 2020: GLOBOCAN Estimatesof Incidence and Mortality World-wide for 36 Cancers in 185 Countries. CA Cancer J Clin. 2021; 71 (3): 209-249. PMID: 33538338.
2. Siegel R L, Miller K D, Fuchs H E, Jemal A. Cancer statistics, 2021. CA Cancer J Clin. 2021; 71 (1): 7-33. PMID: 33433946.
3. US Preventive Services Task Force, Krist A H, Davidson K W, Mangione C M, Barry M J, Cabana M, Caughey A B, Davis E M, Donahue K E, Doubeni C A, Kubik M, Landefeld C S, Li L, Ogedegbe G, Owens D K, Pbert L, Silverstein M, Stevermer J, Tseng C W, Wong J B. Screening for Lung Cancer: US Preventive Services Task Force Recommendation Statement. JAMA. 2021; 325 (10): 962-970. PMID: 33687470.
4. Henschke C I, McCauley D I, Yankelevitz D F, Naidich D P, McGuinness G, Miettinen O S, Libby D M, Pasmantier M W, Koizumi J, Altorki N K, Smith J P. Early Lung Cancer Action Project: overall design and findings from baseline screening. Lancet. 1999; 354 (9173): 99-105. PMID: 10408484.
5 International Early Lung Cancer Action Program Investigators, Henschke C I, Yankelevitz D F, Libby D M, Pasmantier M W, Smith J P, Miettinen O S. Survival of patients with stage I lung cancer detected on CT screening. N Engl J Med. 2006 Oct. 26; 355 (17): 1763-71. PMID: 17065637.
6. Bach P B, Mirkin J N, Oliver T K, Azzoli C G, Berry D A, Brawley O W, Byers T, Colditz G A, Gould M K, Jett J R, Sabichi A L, Smith-Bindman R, Wood D E, Qascem A, Detterbeck F C. Benefits and harms of CT screening for lung cancer: a systematic review. JAMA. 2012; 307 (22): 2418-29. PMID: 22610500.
7. Sands J, Tammemagi M C, Couraud S, Baldwin D R, Borondy-Kitts A, Yankelevitz D, Lewis J, Grannis F, Kauczor H U, von Stackelberg O, Sequist L, Pastorino U, McKee B. J Lung Screening Benefits and Challenges: A Review of The Data and Outline for Implementation. Thorac Oncol. 2021; 16 (1): 37-53. PMID: 33188913.
8. Jonas D E, Reuland D S, Reddy S M, Nagle M, Clark S D, Weber R P, Enyioha C, Malo T L, Brenner A T, Armstrong C, Coker-Schwimmer M, Middleton J C, Voisin C, Harris R P. Screening for lung cancer with low-dose computed tomography: Updated Evidence Report and Systematic Review for the US Preventive Services Task Force. JAMA. 2021; 325 (10): 971-987. PMID: 33687468.
9. Caparica R, Mak M P, Rocha C H, Velho P H I, Viana P, Moura M R L, Menezes M R, Amato M B P, Feher O. Pulmonary Nodules in Patients With Non-pulmonary Cancer: Not Always Metastases. J Glob Oncol. 2016; 2 (3): 138-144. PMID: 28717693.
10. Haddad D N, Sandler K L, Henderson L M, Rivera M P, Aldrich M C. Disparities in Lung Cancer Screening: A Review. Ann Am Thorac Soc. 2020; 17 (4): 399-405. PMID: 32017612.
11. Blandin Knight S, Crosbie P A, Balata H, Chudziak J, Hussell T, Dive C. Progress and prospects of early detection in lung cancer. Open Biol. 2017; 7 (9): 170070. PMID: 28878044.
12. Corner J, Hopkinson J, Fitzsimmons D, Barclay S, Muers M. Is late diagnosis of lung cancer inevitable? Interview study of patients' recollections of symptoms before diagnosis. Thorax. 2005; 60 (4): 314-9. PMID: 15790987.
13. Van Hal G, Diab Garcia P. Lung cancer screening: targeting the hard to reach-A review. Transl Lung Cancer Res. 2021; 10 (5): 2309-2322. PMID: 34164279.
14. Chen H, Stoltzfus K C, Lehrer E J, Horn S R, Siva S, Trifiletti D M, Meng M B, Verma V, Louic A V, Zaorsky N G. The epidemiology of lung metastases. Front Med (Lausanne). 2021; 8:723396. PMID: 34616754.
15. Ha D, Choi H, Chevalier C, Zell K, Wang X F, Mazzone P J. Survival in patients with metachronous second primary lung cancer. Ann Am Thorac Soc. 2015; 12 (1): 79-84. PMID: 25494009.
16. Jamil A. and Kasi A. Lung Metastasis. Stat Pearls Publishing. 2022, NCBI bookshelf ID NBK553111. PMID: 31971751.
17. Wang R, Yin Z, Liu L, Gao W, Li W, Shu Y, Xu J. Second Primary Lung Cancer After Breast Cancer: A Population-Based Study of 6,269 Women. Front Oncol. 2018; 8:427. PMID: 30356721.
18. Del Ciello A, Franchi P, Contegiacomo A, Cicchetti G, Bonomo L, Larici A R. Missed lung cancer: when, where, and why? Diagn Interv Radiol. 2017; 23 (2): 118-126. PMID: 28206951.
19. Antonoff M B, Sofocleous C T, Callstrom M R, Nguyen Q N. The roles of surgery, stereotactic radiation, and ablation for treatment of pulmonary metastases. J Thorac Cardiovasc Surg. 2022; 163 (2): 495-502. PMID: 33838914.
20. Mangiameli G, Cioffi U, Alloisio M, Testori A. Lung Metastases: Current Surgical Indications and New Perspectives. Front Surg. 2022; 9:884915. PMID: 35574534.
21. Dama E, Colangelo T, Fina E, Cremonesi M, Kallikourdis M, Veronesi G, Bianchi F. Biomarkers and lung cancer early detection: State-of-the-art. Cancers (Basel). 2021; 13 (15): 3919. PMID: 34359818.
22. Haranguş A, Berindan-Neagoe I, Todea D A, Şimon I, Şimon M. Noncoding RNAs and Liquid Biopsy in Lung Cancer: A Literature Review. Diagnostics (Basel). 2019; 9 (4): 216. PMID: 31818027.
23. De Fraipont F, Gazzeri S, Cho W C, Eymin B. Circular RNAs and RNA Splice Variants as Biomarkers for Prognosis and Therapeutic Response in the Liquid Biopsies of Lung Cancer Patients. Front Genet. 2019; 10:390. PMID: 31134126.
24. Zhong S, Golpon H, Zardo P, Borlak J. A systematic review identifies predictive and prognostic miRNA candidates for precision medicine in lung cancer. Transl Res. 2021; 230:164-196. PMID: 33253979.
25. Seijo L M, Peled N, Ajona D, Boeri M, Field J K, Sozzi G, Pio R, Zulueta J J, Spira A, Massion P P, Mazzone P J, Montuenga L M. Biomarkers in Lung Cancer Screening: Achievements, Promises, and Challenges. J Thorac Oncol. 2019; 14 (3): 343-357. PMID: 30529598.
26 Yendamuri S, Vaporciyan A A, Zaidi T, Feng L, Fernandez R, Bekele N B, Hofstetter W L, Jiang F, Mehran R J, Rice D C, Spitz M R, Swisher S G, Walsh G L, Roth J A, Katz R L. 3p22.1 and 10q22.3 deletions detected by fluorescence in situ hybridization (FISH): a potential new tool for early detection of non-small cell lung Cancer (NSCLC). J Thorac Oncol. 2008; 3 (9): 979-84. PMID: 18758299.
27. AEGIS Study Team. Shared Gene Expression Alterations in Nasal and Bronchial Epithelium for Lung Cancer Detection. J Natl Cancer Inst. 2017; 109 (7). PMID: 28376173.
28. Chen C C, Bai C H, Lec K Y, Chou Y T, Pan S T, Wang Y H. Evaluation of the diagnostic accuracy of bronchial brushing cytology in lung cancer: A meta-analysis. Cancer Cytopathol. 2021; 129 (9): 739-749. PMID: 33886162.
29. Pavel A B, Campbell J D, Liu G, Elashoff D, Dubinctt S, Smith K, Whitney D, Lenburg M E, Spira A; AEGIS Study Team. Alterations in Bronchial Airway miRNA Expression for Lung Cancer Detection. Cancer Prev Res (Phila). 2017; 10 (11): 651-659. PMID: 28877936.
30. Matthiesen R. MS-Based Biomarker Discovery in Bronchoalveolar Lavage Fluid for Lung Cancer. Proteomics Clin Appl. 2020; 14 (1): e1900077. PMID: 31631581.
31. Engel E, Schmidt B, Carstensen T, Weickmann S, Jandrig B, Witt C, Fleischhacker M. Detection of tumor-specific mRNA in cell-free bronchial lavage supernatant in patients with lung cancer. Ann N Y Acad Sci. 2004; 1022:140-6. PMID: 15251953.
32. Anglim P P, Alonzo T A, Laird-Offringa I A. DNA methylation-based biomarkers for early detection of non-small cell lung cancer: an update. Mol Cancer. 2008; 7:81. PMID: 18947422.
33. Schmidt B, Rchbein G, Fleischhacker M. Liquid Profiling in Lung Cancer-Quantification of Extracellular miRNAs in Bronchial Lavage. Adv Exp Med Biol. 2016; 924:33-37. PMID: 27753015.
34. Mutlu G M, Garcy K W, Robbins R A, Danziger L H, Rubinstein I. Collection and analysis of exhaled breath condensate in humans. Am J Respir Crit Care Med. 2001; 164 (5): 731-7. PMID: 11549524.
35. Rahimpour E, Khoubnasabjafari M, Jouyban-Gharamaleki V, Jouyban A. Non-volatile compounds in exhaled breath condensate: review of methodological aspects. Anal Bioanal Chem. 2018; 410 (25): 6411-6440. PMID: 30046867.
36. Papineni R S, Rosenthal F S. The size distribution of droplets in the exhaled breath of healthy human subjects. J Acrosol Med. 1997; 10 (2): 105-16. PMID: 10168531.
37. Horváth I, Hunt J, Barnes P J, Alving K, Antczak A, Baraldi E, Becher G, van Beurden W J, Corradi M, Dekhuijzen R, Dweik R A, Dwyer T, Effros R, Erzurum S, Gaston B, Gessner C, Greening A, Ho L P, Hohlfeld J, Jobsis Q, Laskowski D, Loukides S, Marlin D, Montuschi P, Olin A C, Redington A E, Reinhold P, van Rensen E L, Rubinstein I, Silkoff P, Toren K, Vass G, Vogelberg C, Wirtz H; ATS/ERS Task Force on Exhaled Breath Condensate. Exhaled breath condensate: methodological recommendations and unresolved questions. Eur Respir J. 2005; 26 (3): 523-48. PMID: 16135737.
38. Boots A W, Bos L D, van der Schee M P, van Schooten F J, Sterk P J. Exhaled Molecular Fingerprinting in Diagnosis and Monitoring: Validating Volatile Promises. Trends Mol Med. 2015; 21 (10): 633-644. PMID: 26432020.

39 Horváth I, Lázár Z, Gyulai N, Kollai M, Losonczy G. Exhaled biomarkers in lung cancer. Eur Respir J. 2009; 34 (1): 261-75. PMID: 19567608.
40. Khoubnasabjafari M, Mogaddam M R A, Rahimpour E, Soleymani J, Saci A A, Jouyban A. Breathomics: Review of Sample Collection and Analysis, Data Modeling and Clinical Applications. Crit Rev Anal Chem. 2021; 11:1-27. PMID: 33691552.
41. Campanella A, De Summa S, Tommasi S. Exhaled breath condensate biomarkers for lung cancer. J Breath Res. 2019; 13 (4): 044002. PMID: 31282387.
42. Mitchell M. I., Ben-Dov I. Z., Liu C., Ye K., Chow K., Kramer Y., Gangadharan A., Park S., Fitzgerald S., Ramnauth A., Perlin D. S., Donato M., Bhoy E., Manouchehri Doulabi E., Poulos M., Kamali-Moghaddam M., Loudig O. Extracellular Vesicle Capture by AnTibody of CHoice and Enzymatic Release (EV-CATCHER): A customizable purification assay designed for small-RNA biomarker identification and evaluation of circulating small-EVs. J Extracell Vesicles. 2021; 10 (8):e12110. PMID: 34122779.
43. Giricz O, Reynolds P A, Ramnauth A, Liu C, Wang T, Stead L, Childs G, Rohan T, Shapiro N, Fineberg S, Kenny P A, Loudig O. Hsa-miR-375 is differentially expressed during breast lobular neoplasia and promotes loss of mammary acinar polarity. J Pathol. 2012; 226 (1): 108-19. PMID: 21953071.
44. Kotorashvili A, Ramnauth A, Liu C, Lin J, Ye K, Kim R, Hazan R, Rohan T, Fineberg S, Loudig O. Effective DNA/RNA Co-Extraction for Analysis of MicroRNAs, mRNAs, and Genomic DNA from Formalin-Fixed Paraffin-Embedded Specimens. PLOS One. 2012; 7:034683. PMID: 22514653.
45. Loudig O, Liu C, Rohan T, and Ben-Dov I Z. Retrospective MicroRNA Sequencing: Complementary DNA Library Preparation Protocol Using Formalin-fixed Paraffin-embedded RNA Specimens. Journal of Visualized Experiments, 2018, (135): 57471. PMID: 29781987.
46. Loudig O, Wang T, Ye K, Lin J, Wang Y, Ramnauth A, Liu C, Stark A, Chitale D, Greenlee R, Multerer D, Honda S, Daida Y, Spencer Feigelson H, Glass A, Couch F J, Rohan T, Ben-Dov I Z. Evaluation and Adaptation of a Laboratory-Based cDNA Library Preparation Protocol for Retrospective Sequencing of Archived MicroRNAs from up to 35-Year-Old Clinical FFPE Specimens. Int J Mol Sci. 2017; 18 (3). pii: E627. PMID: 28335433.
47. Shi M, Han W, Loudig O, Shah C D, Dobkin J B, Keller S, Sadoughi A, Zhu C, Siegel R E, Fernandez M K, DeLaRosa L, Patel D, Desai A, Siddiqui T, Gombar S, Suh Y, Wang T, Hosgood H D, Pradhan K, Ye K, Spivack S D. Initial development and testing of an exhaled microRNA detection strategy for lung cancer case-control discrimination. Sci Rep. 2023 Apr. 24; 13 (1): 6620. doi: 10.1038/s41598-023-33698-8.PMID: 37095155.
48. O'Brien J, Hayder H, Zayed Y, Peng C. Overview of MicroRNA Biogenesis, Mechanisms of Actions, and Circulation. Front Endocrinol (Lausanne). 2018; 9:402. PMID: 30123182.
49. Acunzo M, Romano G, Wernicke D, Croce C M. MicroRNA and cancer—a brief overview. Adv Biol Regul. 2015; 57:1-9. PMID: 25294678.
50. Santos R M, Moreno C, Zhang W C. Non-Coding RNAs in Lung Tumor Initiation and Progression. Int J Mol Sci. 2020; 21 (8): 2774. PMID: 32316322.
51. Yanaihara N, Caplen N, Bowman E, Seikc M, Kumamoto K, Yi M, Stephens R M, Okamoto A, Yokota J, Tanaka T, Calin G A, Liu C G, Croce C M, Harris C C. Unique microRNA molecular profiles in lung cancer diagnosis and prognosis. Cancer Cell. 2006; 9 (3): 189-98. PMID: 16530703.
52. Asakura K, Kadota T, Matsuzaki J, Yoshida Y, Yamamoto Y, Nakagawa K, Takizawa S, Aoki Y, Nakamura E, Miura J, Sakamoto H, Kato K, Watanabe S I, Ochiya T. A miRNA-based diagnostic model predicts resectable lung cancer in humans with high accuracy. Commun Biol. 2020; 3 (1): 134. PMID: 32193503.
53. Zhu X, Kudo M, Huang X, Sui H, Tian H, Croce C M, Cui R. Frontiers of MicroRNA Signature in Non-small Cell Lung Cancer. Front Cell Dev Biol. 2021 Apr. 7; 9:643942. PMID: 33898432.
54. Kasinski A L, Slack F J. MiRNA-34 prevents cancer initiation and progression in a therapeutically resistant K-ras and p53-induced mouse model of lung adenocarcinoma. Cancer Res. 2012; 72 (21): 5576-87. PMID: 22964582.
55. Chen X, Ba Y, Ma L, Cai X, Yin Y, Wang K, Guo J, Zhang Y, Chen J, Guo X, Li Q, Li X, Wang W, Zhang Y, Wang J, Jiang X, Xiang Y, Xu C, Zheng P, Zhang J, Li R, Zhang H, Shang X, Gong T, Ning G, Wang J, Zen K, Zhang J, Zhang C Y. Characterization of microRNAs in serum: a novel class of biomarkers for diagnosis of cancer and other diseases. Cell Res. 2008; 18 (10): 997-1006. PMID: 18766170.
56. Chen X, Hu Z, Wang W, Ba Y, Ma L, Zhang C, Wang C, Ren Z, Zhao Y, Wu S, Zhuang R, Zhang Y, Hu H, Liu C, Xu L, Wang J, Shen H, Zhang J, Zen K, Zhang C Y. Identification of ten serum microRNAs from a genome-wide serum microRNA expression profile as novel noninvasive biomarkers for non-small cell lung cancer diagnosis. Int J Cancer. 2012; 130 (7): 1620-8. PMID: 21557218.
57. Sohel M H Extracellular/circulating microRNAs: release mechanisms, functions and challenges. Achiev. Life Sci. 2016; 10:175-186. PMID: 33430399.
58. Cui M, Wang H, Yao X, Zhang D, Xic Y, Cui R, Zhang X. Circulating MicroRNAs in Cancer: Potential and Challenge. Front Genet. 2019; 10:626. PMID: 31379918.
59. Gao S, Guo W, Liu T, Liang N, Ma Q, Gao Y, Tan F, Xue Q, He J. Plasma extracellular vesicle microRNA profiling and the identification of a diagnostic signature for stage I lung adenocarcinoma. Cancer Sci. 2022; 113 (2): 648-659. PMID: 34837453.
60. Théry C., Zitvogel L, Amigorena S. Exosomes: composition, biogenesis and function. Nat Rev Immunol. 2002; 2 (8): 569-79. PMID: 12154376.
61. Zhong Y, Ding X, Bian Y, Wang J, Zhou W, Wang X, Li P, Shen Y, Wang J J, Li J, Zhang C, Wang C. Discovery and validation of extracellular vesicle-associated miRNAs as noninvasive detection biomarkers for early-stage non-small-cell lung cancer. Mol Oncol. 2021; 15 (9): 2439-2452. PMID: 33340250.
62. Nawaz M, Camussi G, Valadi H, Nazarenko I, Ekström K, Wang X, Principe S, Shah N, Ashraf N M, Fatima F, Neder L, Kislinger T. The emerging role of extracellular vesicles as biomarkers for urogenital cancers. Nat Rev Urol. 2014; 11 (12): 688-701. PMID: 25403245.
63. Kalluri R, LeBleu V S. The biology, function, and biomedical applications of exosomes. Science. 2020; 367 (6478): caau6977. PMID: 32029601.
64. Wortzel I, Dror S, Kenific C M, Lyden D. Exosome-Mediated Metastasis: Communication from a Distance. Dev Cell. 2019; 49 (3): 347-360. PMID: 31063754.
65. Mao S, Lu Z, Zheng S, Zhang H, Zhang G, Wang F, Huang J, Lei Y, Wang X, Liu C, Sun N, He J. Exosomal miR-141 promotes tumor angiogenesis via KLF12 in small cell lung cancer. J Exp Clin Cancer Res. 2020; 39 (1): 193. PMID: 32958011.
66. He S, Li Z, Yu Y, Zeng Q, Cheng Y, Ji W, Xia W, Lu S. Exosomal miR-499a-5p promotes cell proliferation, migration and EMT via mTOR signaling pathway in lung adenocarcinoma. Exp Cell Res. 2019 Jun. 15; 379 (2): 203-213. PMID: 30978341.
67. Fabbri M, Paone A, Calore F, Galli R, Gaudio E, Santhanam R, Lovat F, Fadda P, Mao C, Nuovo G J, Zanesi N, Crawford M, Ozer G H, Wernicke D, Alder H, Caligiuri M A, Nana-Sinkam P, Perrotti D, Croce C M. MicroRNAs bind to Toll-like receptors to induce prometastatic inflammatory response. Proc Natl Acad Sci USA. 2012; 109 (31): E2110-6. PMID: 22753494.
68. Zeng Z, Li Y, Pan Y, Lan X, Song F, Sun J, Zhou K, Liu X, Ren X, Wang F, Hu J, Zhu X, Yang W, Liao W, Li G, Ding Y, Liang L. Cancer-derived exosomal miR-25-3p promotes pre-metastatic niche formation by inducing vascular permeability and angiogenesis. Nat Commun. 2018; 9 (1): 5395. PMID: 30568162.
69. Valadi, H., Ekström, K., Bossios, A., Sjöstrand, M., Lec, J. J., and Lötvall, J. O. Exosome-mediated transfer of mRNAs and microRNAs is a novel mechanism of genetic exchange between cells. Nat. Cell Biol. 2007; 9, 654-659.
70. Yokoi A., Yoshioka Y., Yamamoto Y., Ishikawa M., Ikeda S. I., Kato T., Kiyono T., Takeshita F., Kajiyama H., Kikkawa F., et al. Malignant extracellular vesicles carrying MMP1 mRNA facilitate peritoneal dissemination in ovarian cancer. Nat. Commun. 2017; 8:14470. PMID: 28262727.
71. Liu Y, Fan J, Xu T, Ahmadinejad N, Hess K, Lin S H, Zhang J, Liu X, Liu L, Ning B, Liao Z, Hu T Y. Extracellular vesicle tetraspanin-8 level predicts distant metastasis in non-small cell lung cancer after concurrent chemoradiation. Sci Adv. 2020; 6 (11):eaaz6162. PMID: 32195353.

72 Sandfeld-Paulsen B, Jakobsen K R, Bæk R, Folkersen B H, Rasmussen T R, Meldgaard P, Varming K, Jørgensen MM, Sorensen B S. Exosomal Proteins as Diagnostic Biomarkers in Lung Cancer. J Thorac Oncol. 2016; 11 (10): 1701-10. PMID: 27343445.
73. Choi E S, Faruque H A, Kim J H, Kim K J, Choi J E, Kim B A, Kim B, Kim Y J, Woo M H, Park J Y, Hur K, Lec M Y, Kim D S, Lec S Y, Kim E. CD5L as an Extracellular Vesicle-Derived Biomarker for Liquid Biopsy of Lung Cancer. Diagnostics (Basel). 2021; 11 (4): 620. PMID: 33808296.
74. Wu C, Zhou S, Mitchell M, Hou C, Byers S, Loudig O, Ma J. Coupling Suspension Trapping-based Sample Preparation and Data-Independent Acquisition Mass Spectrometry for Sensitive Exosomal Protcomic Analysis. Anal Bioanal Chem. 2022; 414 (8): 2585-2595. PMID: 35181835.
75. Mitchell M I, Ma J, Carter C L, Loudig O. Circulating Exosome Cargoes Contain Functionally Diverse Cancer Biomarkers: From Biogenesis and Function to Purification and Potential Translational Utility. Cancers (Basel). 2022; 14 (14): 3350. PMID: 35884411.
76. Pinkerton M, Chinchilli V, Banta E, Craig T, August A, Bascom R, Cantorna M, Harvill E, Ishmael F T. Differential expression of microRNAs in exhaled breath condensates of patients with asthma, patients with chronic obstructive pulmonary disease, and healthy adults. J Allergy Clin Immunol. 2013; 132 (1): 217-9. PMID: 23628339.
77. Pérez-Sánchez C, Barbarroja N, Pantaleão LC, López-Sánchez LM, Ozanne S E, Jurado-Gámez B, Aranda E, Lopez-Pedrera C, Rodríguez-Ariza A. Clinical Utility of microRNAs in Exhaled Breath Condensate as Biomarkers for Lung Cancer. J Pers Med. 2021; 11 (2): 111. PMID: 33572343.
78. Mendes F C, Paciência I, Ferreira A C, Martins C, Rufo J C, Silva D, Cunha P, Farraia M, Moreira P, Delgado L, Soares M L, Moreira A. Development and validation of exhaled breath condensate microRNAs to identify and endotype asthma in children. PLOS One. 2019; 14 (11):e0224983. PMID: 31703106.
79. Faversani A, Favero C, Dioni L, Pesatori A C, Bollati V, Montoli M, Musso V, Terrasi A, Fusco N, Nosotti M, Vaira V, Palleschi A. An EBC/Plasma miRNA Signature Discriminates Lung adenocarcinomas from pleural mesothelioma and healthy controls. Front Oncol. 2021; 11:643280. PMID: 34211838.

80 Shi A, Kasumova G G, Michaud W A, Cintolo-Gonzalez J, Díaz-Martínez M, Ohmura J, Mehta A, Chien I, Frederick D T, Cohen S, Plana D, Johnson D, Flaherty K T, Sullivan R J, Kellis M, Boland G M. Plasma-derived extracellular vesicle analysis and deconvolution enable prediction and tracking of melanoma checkpoint blockade outcome. Sci Adv. 2020; 6 (46): cabb3461. PMID: 33188016.
81. Rodosthenous R S, Hutchins E, Reiman R, Yeri A S, Srinivasan S, Whitsett T G, Ghiran I, Silverman M G, Laurent L C, Van Keuren-Jensen K, Das S. Profiling Extracellular Long RNA Transcriptome in Human Plasma and Extracellular Vesicles for Biomarker Discovery. iScience. 2020; 23 (6): 101182. PMID: 32512385.
82. Campomenosi P, Gini E, Noonan D M, Poli A, D′Antona P, Rotolo N, Dominioni L, Imperatori A. A comparison between quantitative PCR and droplet digital PCR technologies for circulating microRNA quantification in human lung cancer. BMC Biotechnol. 2016 Aug. 18; 16 (1): 60. doi: 10.1186/s12896-016-0292-7. PMID: 27538962; PMCID: PMC4991011.
83. Hoshino A, Costa-Silva B, Shen T-L, Rodrigues G, Hashimoto A, Tesic Mark M, et al. Tumour exosome integrins determine organotropic metastasis. Nature. 2015; 527 (7578): 329-35. PMID: 26524530.
84. Hsiao K-C, Shih N-Y, Fang H-L, Huang T-S, Kuo C-C, Chu P-Y, et al. Surface α-enolase promotes extracellular matrix degrada-tion and tumor metastasis and represents a new therapeutic target. Karamanos N K, editor. PLOS ONE. 2013; 8 (7):e69354. PMID: 23894455.
85. Zheng D, Haddadin S, Wang Y, Gu L Q, Perry M C, Freter C E, Wang M X. Plasma microRNAs as novel biomarkers for early detection of lung cancer. Int J Clin Exp Pathol. 2011; 4 (6): 575-86. PMID: 21904633.
86 Li W, Wang Y, Zhang Q, Tang L, Liu X, Dai Y, Xiao L, Huang S, Chen L, Guo Z, Lu J, Yuan K. MicroRNA-486 as a Biomarker for Early Diagnosis and Recurrence of Non-Small Cell Lung Cancer. PLOS Onc. 2015; 10 (8):e0134220. PMID: 26237047.
87. Minn A. J., Gupta G. P., Siegel P. M., Bos P. D., Shu W., Giri D. D., Viale A., Olshen A. B., Gerald W. L., Massagué J. Genes that mediate breast cancer metastasis to lung. Naturc. 2005; 436:518-524. PMID: 16049480.
88. Agiostratidou G, Li M, Suyama K, Badano I, Keren R, Chung S, Anzovino A, Hulit J, Qian B, Bouzahzah B, Eugenin E, Loudig O, Phillips G R, Locker J, Hazan R B. Cancer Rcs. 2009; 69 (12): 5030-8. PMID: 19491271.
89. Kim S, Yao J, Suyama K, Qian X, Qian B Z, Bandyopadhyay S, Loudig O, De Leon-Rodriguez C, Zhou Z N, Segall J, Macian F, Norton L, Hazan R B. Slug promotes survival during metastasis through suppression of Puma-mediated apoptosis. Cancer Res. 2014; 74 (14): 3695-706. PMID: 24830722.
90. Cavallari I, Ciccarese F, Sharova E, Urso L, Raimondi V, Silic-Benussi M, D'Agostino D M, Ciminale V. The miR-200 Family of microRNAs: Fine Tuners of Epithelial-Mesenchymal Transition and Circulating Cancer Biomarkers. Cancers (Basel). 2021 Nov. 23; 13 (23): 5874. PMID: 34884985.
91. Fan, G. Xu, Z. Chang, L. Zhu, J. Yao. miR-210 transferred by lung cancer cell-derived exosomes may act as proangiogenic factor in cancer-associated fibroblasts by modulating JAK2/STAT3 pathway. Clin. Sci. (Lond). 2020; 134:807-825. PMID: 32219336.
92. Song Q, An Q, Niu B, Lu X, Zhang N, Cao X. Role of miR-221/222 in Tumor Development and the Underlying Mechanism. J Oncol. 2019; 2019:7252013. PMID: 31929798.
93. Ding J, Xu Z, Zhang Y, Tan C, Hu W, Wang M, Xu Y, Tang J. Exosome-mediated miR-222 transferring: An insight into NF-κB-mediated breast cancer metastasis. Exp Cell Res. 2018; 369 (1): 129-138. PMID: 29778754.
94. Ding L, Getz G, Wheeler D A, et al. Somatic mutations affect key pathways in lung adenocarcinoma. Nature. 2008; 455:1069-75. PMID: 18948947.
95. Mogi A, Kuwano H. TP53 mutations in non-small cell lung cancer. J Biomed Biotechnol. 2011; 2011:583929.PMID: 21331359.
96. Midha A, Dearden S, McCormack R. EGFR mutation incidence in non-small-cell lung cancer of adenocarcinoma histology: a systematic review and global map by ethnicity (mutMapII). Am J Cancer Res. 2015; 5 (9): 2892-911. eCollection. PMID: 26609494.
97. Ricly G J, Kris M G, Rosenbaum D, Marks J, Li A, Chitale D A, Nafa K, Riedel E R, Hsu M, Pao W, Miller V A, Ladanyi M. Frequency and distinctive spectrum of KRAS mutations in never smokers with lung adenocarcinoma. Clin Cancer Res. 2008; 14 (18): 5731-4. PMID: 18794081.
98. D'Amico D, Carbone D, Mitsudomi T, Nau M, Fedorko J, Russell E, Johnson B, Buchhagen D, Bodner S, Phelps R, et al. High frequency of somatically acquired p53 mutations in small-cell lung cancer cell lines and tumors. Oncogene 1992; 7:339-46. PMID: 1312696.
99. Duma N, Santana-Davila R, Molina J R. Non-Small Cell Lung Cancer: Epidemiology, Screening, Diagnosis, and Treatment. Mayo Clin Proc. 2019; 94 (8): 1623-1640. PMID: 31378236.
100. Politi K, Zakowski M F, Fan P D, Schonfeld E A, Pao W, Varmus H E. Lung adenocarcinomas induced in mice by mutant EGF receptors found in human lung cancers respond to a tyrosine kinase inhibitor or to down-regulation of the receptors. Genes Dev. 2006; 20 (11): 1496-510. PMID: 16705038.
101. Jackson E L, Willis N, Mercer K, Bronson R T, Crowley D, Montoya R, Jacks T, Tuveson D A. Analysis of lung tumor initiation and progression using conditional expression of oncogenic K-ras. Genes Dev. 2001; 15 (24): 3243-8. PMID: 11751630.
102. Schneeberger V E, Ren Y, Luetteke N, Huang Q, Chen L, Lawrence H R, Lawrence N J, Haura E B, Koomen J M, Coppola D, Wu J. Inhibition of Shp2 suppresses mutant EGFR-induced lung tumors in transgenic mouse model of lung adenocarcinoma. Oncotarget. 2015; 6 (8): 6191-202. PMID: 25730908.
103. Rokicki W, Rokicki M, Wojtacha J, Dżeljijli A. The role and importance of club cells (Clara cells) in the pathogenesis of some respiratory diseases. Kardiochir Torakochirurgia Pol. 2016; 13 (1): 26-30. PMID: 27212975.
104. Xu X, Rock J R, Lu Y, Futtner C, Schwab B, Guinncy J, Hogan B L, Onaitis M W. Evidence for type II cells as cells of origin of K-Ras-induced distal lung adenocarcinoma.Proc Natl Acad Sci USA. 2012; 109 (13): 4910-5. PMID: 22411819.
105. Barkauskas C E, Cronce M J, Rackley C R, Bowic E J, Keene D R, Stripp B R, Randell S H, Noble P W, Hogan B L. Type 2 alveolar cells are stem cells in adult lung. J Clin Invest. 2013; 123 (7): 3025-36. PMID: 23921127
106. Liu P, Zhao L, Senovilla L, Kepp O, Kroemer G. In Vivo Imaging of Orthotopic Lung Cancer Models in Mice. Methods Mol Biol. 2021; 2279:199-212. PMID: 33683696.
107. Buckle T, van Leeuwen F W. Validation of intratracheal instillation of lung tumour cells in mice using single photon emission computed tomography/computed tomography imaging. Lab Anim. 2010; 44 (1): 40-5. PMID: 19854758.
108. Limjunyawong N, Mock J, Mitzner W. Instillation and Fixation Methods Useful in Mouse Lung Cancer Research. J Vis Exp. 2015; (102):e52964. PMID: 26381993.
109. Janik K, Popeda M, Peciak J, Rosiak K, Smolarz M, Treda C, Rieske P, Stoczynska-Fidelus E, Ksiazkiewicz M. Efficient and simple approach to in vitro culture of primary epithelial cancer cells. Biosci Rep. 2016; 36 (6):e00423. PMID: 27803125.
110. Iha K, Tsurusawa N, Tsai H Y, Lin M W, Sonoda H, Watabe S, Yoshimura T, Ito E. Ultrasensitive ELISA detection of proteins in separated lumen and membrane fractions of cancer cell exosomes. Anal Biochem. 2022 Oct. 1; 654:114831. doi: 10.1016/j.ab.2022.114831. Epub 2022 Jul. 31. PMID: 35921878.
111. Stella G M, Kolling S, Benvenuti S, Bortolotto C. Lung-seeking metastases. Cancers (Basel). 2019; 11 (7): 1010. PMID: 31330946.
112. Gerull W D, Puri V, Kozower B D. The epidemiology and biology of pulmonary metastases. J Thorac Dis. 2021; 13 (4): 2585-2589. PMID: 34012606.
113. Wang M, Vogel I, Kalthoff H. Correlation between metastatic potential and variants from colorectal tumor cell line HT-29. World J Gastroenterol. 2003; 9 (11): 2627-31. PMID: 14606113.
114. Jin H, Yu Y, Hu Y, Lu C, Li J, Gu J, Zhang L, Huang H, Zhang D, Wu X R, Gao J, Huang C. Divergent behaviors and underlying mechanisms of cell migration and invasion in non-metastatic T24 and its metastatic derivative T24T bladder cancer cell lines. Oncotarget. 2015; 6 (1): 522-36.PMID: 25402510.
115. Tong K, Pellon-Cardenas O, Sirihorachai V R, Warder B N, Kothari O A, Perckatt A O, Fokas E E, Fullem R L, Zhou A, Thackray J K, Tran H, Zhang L, Xing J, Verzi M P.Degree of Tissue Differentiation Dictates Susceptibility to BRAF-Driven Colorectal Cancer. Cell Rep. 2017; 21 (13): 3833-3845. PMID: 29281831.
116. Tong K, Kothari O A, Haro K S, Panda A, Bandari M M, Carrick J N, Hur J J, Zhang L, Chan C S, Xing J, Gatza M L, Ganesan S, Verzi M P. SMAD4 is critical in suppression of BRAF-V600E serrated tumorigenesis. Oncogene. 2021; 40 (41): 6034-6048. PMID: 34453124.
117. Mc Donnell S, Chaudhry V, Mansilla-Soto J, Zeng Z S, Shu W P, Guillem J G. Metastatic and non-metastatic colorectal cancer (CRC) cells induce host metalloproteinase production in vivo. Clin Exp Metastasis. 1999; 17 (4): 341-9. PMID: 10545021.
118. Kumar P, Nagarajan A, Uchil P D. Analysis of Cell Viability by the MTT Assay. Cold Spring Harb Protoc. 2018; 2018 (6). PMID: 29858338.
119. Mazumdar A, Urdincz J, Boro A, Migliavacca J, Arlt M J E, Muff R, Fuchs B, Snedeker J G, Gvozdenovic A. Osteosarcoma-Derived Extracellular Vesicles Induce Lung Fibroblast Reprogramming. Int J Mol Sci. 2020 Jul. 30; 21 (15): 5451. doi: 10.3390/ijms 21155451. PMID: 32751693.
120. Love M I, Huber W, Anders S. Moderated estimation of fold change and dispersion for RNA-seq data with DEScq2. Genome Biol. 2014; 15 (12): 550. PMID: 25516281.

### Example 4. Noninvasive Detection of Orthotopic Human Lung Tumors by microRNA Analysis Profiling of Mouse Exhaled Breath Condensates and Exhaled Extracellular Vesicles

Background Lung cancer is the second leading cause of cancer incidence (2.1 million cases per year) and mortality (1.8 million deaths per year) globally [1, 2]. However, the lung is also the second most frequent site of metastatic growth for extra-thoracic malignancies [3]. It is recognized that because of its role in blood circulation the lung offers optimal conditions for the development of secondary cancers that arise from primary colorectal (˜25.8%), head and neck (˜19.4%), urologic (i.e., bladder, kidney, and testicular; ˜14.7%), breast (˜10.5%), melanoma (˜6.5%), gynecological, blood, and other cancers (˜6.1%) [4]. Currently, it is estimated that ˜5-10% of patients with a malignant cancer, will at some point develop pulmonary metastatic lesions that are either synchronous (i.e., found at the time of primary cancer diagnosis) or metachronous (i.e., found as a recurrent lesion or after primary cancer diagnosis) [5, 6]. The 5-year survival rate for patients who develop secondary lung metastases is very low; for example, it is estimated at ˜21% for patients diagnosed with primary breast cancer and <10% for those diagnosed with primary colorectal cancer [7, 8]. Clinically, the early detection of micro-metastases developing in the lung is extremely difficult as early invading cells are disseminated within the lung tissue, thus preventing individual cell detection when using x-ray or computed tomography (CT) [9]. Although late detection of secondary lung cancer has a very poor prognosis, early radiation therapy, chemotherapy, and metastasectomy have been shown to significantly increase patient survival [10, 11]. Therefore, for optimal sequence and timing of local interventions, it is imperative to improve early detection of metastatic disease.

Thus, to improve the clinical detection of secondary lung cancers and to complement current imaging strategies, recent research efforts have led to the development of robust non-symptom-driven molecular screening assays. These assays have been designed to detect circulating primary tumor bioproducts such as circulating tumor cells (CTCs), circulating tumor DNA (ctDNA), circulating RNA transcripts (i.e., circular RNAs, messenger RNAs (mRNA), microRNAs (miRNA) and long non-coding RNAs), and more recently circulating tumor extracellular vesicles (EVs) [12-18]. Although the detection of CTCs can be used prognostically, it is not foolproof and a significant subset of patients who develop metastatic disease are not identified using this approach [19-22]. Currently there are no blood-based biomarker assays (e.g., AFP+CEA+CA125 for breast cancer or PSA for prostate cancer) that can predict the development of secondary lung cancer [23]. Although direct airway collection strategies (i.e., nasal epithelial brushing, sputum, bronchial brushing, bronchioalveolar lavage (BAL), and exhaled breath condensates (EBC)) are being investigated for the detection of primary lung cancer, they have not been evaluated for the detection of secondary lung cancer [24-32].

The growing field of breath biopsy (i.e., the collection and analysis of exhaled breath) is showing great promise for the detection of lung tumors [32-34]. The analysis of volatile organic compounds (VOCs) (i.e., ammonia, nitric oxide, hydrogen sulphide, acetone, aldehyde, methane, ethane, propane and carbon dioxide) [35, 36] and non-volatile organic compounds (non-VOCs) (e.g., urea, amino-acids, RNA, DNA, proteins, lipids, surfactants) has been shown to reflect the metabolic and biologic effects of lung tumors [37,38]. Ongoing studies on VOCs are aimed at identifying metabolic changes associated with disease, whereas non-VOC studies are aimed at identifying and measuring biological compounds originating from tumor cells [39,40]. The collection of VOCs requires air-tight equipment for instant electronic analysis of exhaled gasses, whereas non-VOCs are collected by the condensation of exhaled vapors and provide an exhaled breath condensate (EBC) biofluid that can be stored and analyzed using multiple assay types (i.e., NGS, qPCR, methylation assays, etc.,) [41,42]. Studies already conducted on EBC have revealed that it contains miRNAs, including those deregulated in tumors, with unique expression ratios that may be quantifiable for the detection lung tumors [43-47]. Interestingly, recent investigations have also revealed that EBC contains extracellular vesicles (EVs), whose miRNA cargoes can be analyzed and may allow for detection of lung disease [48-50].

It is well documented that miRNAs are involved in the regulation of all biological processes [51,52] and a large body of research has demonstrated that the deregulation of miRNA expression is associated with the initiation, the development, and metastatic dissemination of human tumor cells [53-58]. Recent studies have also shown that miRNA profiles of tumor cells can provide both diagnostic and prognostic information on cancer progression [59-62]. Importantly, tumor cells can exchange miRNAs with neighboring and long distant cells, by means of EVs. The molecular cargoes of tumor EVs, particularly miRNAs, have been shown to play important roles in the transcriptomic re-programming of target cells [63-65]. For example, tumor EV miRNAs of breast, lung, and other types of human tumors have been associated with the modulation of angiogenesis [60,65], cellular proliferation [67, 68], immune response [69, 70] and the establishment of distal pre-metastatic niches [71-75].

EVs represent a large family of robust phospholipid bi-layered membrane-bound nanoparticles that are secreted by all human cells and that can diffuse within tissues, circulate in the bloodstream, and be found in all biofluids [76, 77]. EVs share common surface protein markers (i.e., tetraspanins including CD9, CD81, CD83, Flotilin, etc.), as well as unique surface protein markers acquired from their cell of origin, which can be targeted by antibodies for their purification [78-83]. Studies of the MiRNA content of circulating tumor EVs, specifically those purified from the circulation or from other biofluids, have identified unique profiles, which can be associated with their tumor cells of origin [84]. Therefore, it is well accepted that the isolation of tumor EVs from biofluids and the analysis of their miRNA cargos has the potential to enable the development of non-invasive tumor detection assays for diagnostic and prognostic applications [85-91].

In this study we sought to explore the potential of utilizing exhaled miRNAs for non-invasive detection of secondary lung cancer in animal models. Therefore, we inoculated a highly metastatic breast cancer cell line well documented to establish significant pathological lung tumor burden in athymic nude mice to conduct these exhaled breath analyses [92-95].

### Materials and Methods

### Cell Culture

MDA-MB-231 subline 3475 triple negative breast cancer cells expressing both TdTomato-Luc and CD63-GFP were cultured in a standard growth media comprised of Dulbecco's Modified Eagles Medium (DMEM) supplemented with 10% EV depleted fetal bovine serum (FBS) and 1% Penicillin Streptomycin. Cells were maintained at an atmosphere of 37° C. and a humidity 5% CO2 and regularly sub cultured once a confluency of 70-80% was reached. Upon reaching 80% confluency, cells were split with fresh media and allowed to undergo two rounds of passaging after cracking vials. The day prior to tumor inoculations in mice, cells were transported to the tissue culture facility housed within the CDI research animal facility to allow cells to acclimatize.

### Animals

All animal husbandry and procedures involving mice in this study were conducted under the Center for Discovery and Innovation IACUC approved protocol (#288.00) in an Association for Assessment and Accreditation of Laboratory Animal Care International (AAALAC) accredited research animal facility in accordance with all NIH guidelines for the use and care of experimental animals.

### Tumor Inoculations for Lung Metastasis

On the day of tumor inoculations TdTomato-Luc+/CD63-GFP+ MDA-MB-231 subline 3475 cells were trypsinized and counted prior to being resuspended in warm 1× sterile PBS at a concentration of 1×10⁶ cells per 200 μl. Immediately after cell preparations athymic BALB/C mice were “pre-warmed” in a cage by placing a heating pad under the cage in order to dilate veins, animals were restrained, and the tail grasped at the mid length and slight tension applied. The lateral tail vein was located, and the needle inserted parallel into the vein. Once the needle is inserted into the vein cells were slowly injected. Any bleeding at the injection site was stopped by applying gentle compression and animals were returned to their cage and monitored.

### In Vivo Bioluminescence Imaging

Animals were anesthetized using isoflurane inhalation prior to receiving an intraperitoneal (I.P.) injection of D-luciferin (150 mg/kg). 15 min after D-luciferin administration animals were then placed onto the warming pad in the imaging box of an IVIS instrument, oriented to expose the front of their body to the camera, so that the tumors located in the lungs are well within the imaging area. The light box was then closed, and anterior images were acquired using the auto-exposure feature. Animals were imaged once a week for the duration of the study to i) determine the site of cancer cell growth and ii) monitor tumor burden.

### Exhaled Breath Condensate (EBC) Collection Using the RC3 Dual Mouse Chamber

EBC collection was achieved through the collection of EBC from two mice placed together into a sterile glass RC3 respirometer chamber (Sable Systems International) attached to a SS4 flow pump/meter set to a rate of ˜20 ml/min. The flow pump/meter sets the rate of inlet air into the chamber, with the ˜20 ml/min setting being the recommended flow rate needed to allow for enough air flow into the chamber to not have animals suffocate during the collection period. The RC3 respiratory chamber is large enough that it easily allows two mice to be placed in the chamber and does not lead to significant restraint of the animals, as animals still have freedom to move around with normal postural movement (i.e., walk and turn around freely) within the chamber (length=10″ and the diameter=3″). Mice were kept in the chamber for one hour to allow for adequate volumes of EBC to be collected. EBC collection was performed every week for the duration of this 16-week study.

### Exhaled Breath Condensate (EBC) Collection Using a Single Mouse Nose-Mouth Device

In order to collect EBC from single mice without the risk of contamination from urine, skin and feces we modified the design and setup of the single breath collection device described in Liu et al., 2019 [96]. For this method of EBC collection single mice were placed into the modified mouse restrainer which has been designed to only expose the nose-mouth of mice allowing for uncontaminated collection of EBC. The design of our single mouse EBC collection device involves placing the mice into a restrainer that does not allow the mice to turn around so that maximum EBC volumes can be obtained. Since the use of full body restraint of animals can lead to enhanced stress, we designed the restraint in such a way that the head and torso of the animals are held within a dark chamber allows for animals to be within a dark enclosed space to reduce anxiety throughout the collection time period. Our single mouse EBC collection device utilizes the same flow pump/meter described above for the collection of EBC from a dual mouse chamber and rate of inlet air into the chamber will be set to ˜20 ml/min. Additionally, in order to ensure one-way air flow a second flow meter will be attached to the other end of our collection (condensation) chamber and will be set to a flow rate of ˜0.2 ml/min. Since, our single collection device is smaller in size and ensures the collection of EBC only from the nose-mouse of animals EBC collections were performed for 2 hours three times a week for the duration of this study.

### Lung Tissue Collection and H&E Staining

At the end of the study period mice were sacrificed and lungs were perfused and inflated for tumor histological examination by inserting a 3 ml syringe with a 22 g needle attached into the trachea and slowly inflating the lungs with 10% formalin at a rate of approximately 200 ul/see until the lungs have fully inflated. Following inflation, the trachea was severed, and the lungs removed from the respiratory cavity and placed into a tube containing 10% buffered formalin and were fixed for 24 hours. Following fixation, lungs were processed by histological sectioning and H&E staining at the histology & comparative pathology core facility at the Albert Einstein College of Medicine. Briefly, formalin fixed tissue samples were placed into clean individually labelled cassettes, where samples were orientated in such a way as to achieve a longitudinal section through the sample. Cassettes were then filled with hot paraffin wax and allowed to cool on a cold block, before being sectioned into 5 μm sections using a microtome. One section of each sample was placed onto a slide and slides were placed to on a pre-warmed heating plate to dry. Prior to any staining procedures samples were deparaffinized and rehydrated and underwent a series of incubation steps in xylene followed by incubation in decreasing concentrations of alcohol (i.e., 100% for 3 min twice, 95% for 2 min, 80% for 2 min then 70% for 1 min). Samples were washed for 2 min and then incubated in a hematoxylin solution for 8 min followed by a 10 sec wash step and a second 5 min wash step. Tissue specimens were then acid washed in 1% alcohol solution for 5 seconds followed by a 3 min wash step, followed directly by a 10 second ammonia dip and another 4 min wash step. Slides were incubated in 80% alcohol for 1 min followed by a 5 min incubation step in a solution of eosin. Finally, slides underwent three 1 min incubation steps in 100% alcohol followed by one 2 min incubation step in xylene followed by a final two 5 min incubation steps in xylene.

### Spectradyne Microfluidic Resistive Pulse Sensing (MRPS)

Particle size distribution of exhaled EVs isolated from mouse EBC was performed using MRPS measurements on a nCS1 instrument (Spectradyne LLC, Signal Hill CA). Initially the microfluidic system was primed using a solution of 0.2 μm filtered PBS containing 1% Tween 20 (v/v). For each purified exhaled EV sample 2 μl was loaded onto a C-400 cartridge (i.e., analysis of particles between 65 and 400 nm), the instrument pressure and voltage parameters were automatically determined using the instrument software. Acquisition of data from >6,000 particle detection events was collected for each sample, and all data were combined into a single stats file and using the nCS1 Data Viewer software and (i) peak filters and (ii) background subtraction were applied, according to manufacturer recommendations. Peak filters set were (i) transit time <60 μs, (ii) diameter >65 nm, and signal to noise ratio (S/N)>10. Additionally, combined stats files were analysed for size distribution and particle concentration and peak-filtered CSD graphs were generated.

### EV-CATCHER Isolation of the Extracellular Vesicle.

The isolation of exhaled EVs was performed using the EV-CATCHER isolation protocol described by Mitchell et al., 2021 and in PCT/US2021/065060 and PCT/US2024/028979, customized with either human-specific CD63 or mouse-specific as the capture antibodies [97]. Briefly, equimolar amounts of 5′-Azide modified and 3′-Biotin modified oligonucleotides (Integrated DNA Technologies) were annealed in 1×RNA annealing buffer, prior to separation on a 15% non-denaturing polyacrylamide (PAGE) gel. The annealed double stranded (ds) DNA product was visualized using SYBR® Gold™ dye (ThermoFisher, #S11494), excised, crushed using a gel breaker tube (IST Engineering, #3388-100), resuspended in 400 mM NaCl and placed on a thermomixer set to 4° C. and 1,100 RPM overnight. The solution was filtered, and the dsDNA linker was purified using the QIAEX® II gel extraction kit (Qiagen, #20021) according to manufacturer instructions. Capture antibodies (1 mg/ml) used for exh-EV pulls, were activated using 5 μl of freshly prepared 4 mM DBCO-NHS ester (Lumiprobe, #94720) and incubated for 30 min at room temperature (RT) in the dark, reactions were stopped by adding 2.5 μl of 1M Tris-Cl (pH 8.0) at RT for 5 min in the dark. DBCO-activated antibodies were then desalted using Zeba desalting columns (ThermoFisher, #89882). and quantified on a Nanodrop 2000 instrument prior to the preparation of antibody-dsDNA (Ab-dsDNA) stock solutions (i.e., 100 μg of activated antibody conjugated to 50 μg of purified DNA linker. The Ab-dsDNA conjugates were then bound to streptavidin coated 96-well plates (Pierce, #15120) by incubating 1 μg of each Ab-dsDNA in 100 μl PBS per well (2 wells were prepared per sample). Solutions were carefully removed, and wells were washed three times with cold 1×PBS solution, prior to addition of RNase-A (12.5 μg/ml) treated samples (100 μl). Plates were sealed using microAMP optical adhesive film (Applied Biosystems, #4311971) and placed on a shaker at 300 RPM at 4° C., O/N. Samples were carefully removed, wells were washed 3 times with cold 1×PBS and 100 μl of freshly prepared uracil glycosylase (UNG) enzyme (ThermoFisher, #EN0362) in 1×PBS (1×UNG buffer (200 mM Tris-Cl (pH 8.0), 10 mM EDTA and 100 mM NaCl), with 1 unit of enzyme) was added to each well. Plates were incubated at 37° C. for 2 h on a shaker at 300 RPM for UNG digestion of the dsDNA linker, and isolated EVs were collected for downstream analyses.

### Transmission Electron Microscopy

Transmission electron microscopy (TEM) of exhaled EVs purified by ultracentrifugation and the EV-CATCHER assay was performed at the analytical imaging facility at the Albert Einstein College of Medicine. Briefly, ultracentrifugation isolated exhaled EVs, obtained from 3 ml of mouse EBC (i.e., collected from six mice over a period of three weeks) were fixed using 2% Glutaraldehyde in phosphate buffer (Electron Microscopy Services, #6536-05) and stored at 4° C. 300 mesh formvar-coated grids were inverted onto 20 μl of fixed exhaled EV suspensions for 2 min and wicked dry. Grids were then inverted onto 40 μl of 2% aqueous uranyl acetate for 1 min, and wicked dry. Samples were imaged on a JEOL JEM-1400+ transmission electron microscope (JEOL Ltd.; Tokyo, Japan) operating at an accelerating voltage of 80 kV. High resolution TIFF images were acquired and saved using an AMT 16 MP digital camera system (Advanced Microscopy Techniques Corp.; Woburn, MA).

### ONi Super Resolution Nanoimaging

Purified exhaled EVs were processed for imaging on the highly sensitive ONi super resolution Nanoimager using the ONi EV Profiler kit v2.0 customized for the capture and assessment of mouse-specific EVs. Briefly, the surface of the assay capture chip was prepared by applying 5 μl of S3 buffer to each lane and incubated at room temperature for 10 min. 30 μl of W I was then applied to each lane to remove excess S3, after which 10 μl of biotinylated mouse CD63 (Novus Biologicals, #FAB5417B) was slowly pipetted to each lane ensuring that no bubbles were introduced into lane and incubated for a period of 10-minute at room temperature. All lanes were again washed by slowly applying 30 μl of W1 buffer to each lane. EV capture was then performed by immediately applying 10 μL of ultracentrifugation purified exhaled EVs and allowing binding to occur for 15 min. Lanes were then washed using 30 μl of W1 buffer and captured EVs were fixed by applying 20 μl of F1 to each lane and incubating the chip at room temperature for 10-minutes. The staining of captured exhaled EVs was performed by firstly preparing a two-antibody working solution comprised of CD9-Alexa Fluor® 488 (Novus Biologicals, #FAB5218G) and CD81-Alexa Fluor® 647 (Novus Biologicals, #FAB4865R) antibodies combined together in W1 buffer so that each antibody is at a working dilution of 1:20. The final staining solution was prepared by combining 1 μl of the prepared working solution with 9 μl of N1 buffer to each lane, gently pipetting to mix the solution and applying 10 μl to each lane of the EV profiler chip and allowed to incubate for 50 minutes at room temperature in the dark. Immediately following antibody incubation lanes were washed with 30 μl of W1 buffer followed by a 20-minute incubation with 20 μl of F1 buffer for 10 minutes. A final wash step was performed and BCubed™ dSTORM imaging buffer added to each well immediately before EV profiler chips were imaged. Image acquisition on the ONi super resolution Nanoimager was performed in the NimOS Light program with a 640 dichroic split using the following parameters: 640 nm laser set to 20-30% laser power, the 560 nm laser at 35% laser power and the 473/488 nm laser set to 70% laser power. The number of runs (frames) for all laser lines was set to 1000 and all image analyses were performed using CODI software.

### Small-RNA Extractions

Small-RNA extractions from exhaled EVs were performed using the miRNeasy Serum/Plasma kit (Qiagen, Cat #217184) according to manufacturer's instructions with some modifications to improve total RNA yield. Briefly, QIAzol was added to 100 μl of purified exh-Evs, vortexed and incubated at RT for 3 min, after which chloroform was added to each sample. Samples were vortexed again and incubated at RT for 3 min. Samples were then centrifuged at 12,000×g, at 40° C. for 15 min and the upper aqueous phase was carefully removed and transferred into new siliconized 1.5 ml eppendorf tubes, to which 100% ethanol and 2 μl of miRNA-Seq 19nt/24nt (1.5 ng+1.5 ng) size marker required for NGS library preparations was added to each sample. Samples were incubated on ice for 40 min prior to undergoing column purification, where each sample was passed twice through RNeasy minElute columns, followed by a working solution of RPE wash buffer, and finally ice cold 80% ethanol. Columns were spun to remove residual ethanol and total RNA was eluted with 50 μl of RNase-free water. Samples were then speed-vacuumed to 9.5 μl prior to small-RNA sequencing.

Small-RNA cDNA Library Preparations

Small-RNA sequencing from EVs purified from mouse exhaled breath condensates (EBC), were performed using the cDNA library preparation protocol described by Loudig et al. (2017), with modifications for low input RNA from purified small-EVs [98]. small-RNA cDNA library preparations were performed using total RNA recovered from exhaled EVs purified from either whole EBC or exh-EVs purified from mouse EBC using the EV-CATCHER assay customized for the isolation of either human-specific or mouse-specific exhaled EVs. 18 individual ligations were set up for each library by combining 9.5 μl of total RNA, 8.5 μl of the master-mix and 1 μl of 50 μM adenylated barcoded 3′ adapter (Integrated DNA Technologies). A master-mix using 0.0052 nM calibrator cocktail (40 μl 10×RNA ligase-2 buffer, 120 μl 50% DMSO, and 10 μl calibrator cocktail) was prepared and each reaction was heated at 90° C. for 1 min, incubated on ice for 2 min, and 1 μl ( 1/10 diluted) truncated K227Q T4 RNA Ligase 2 (New England Biolabs, #M0351L) was added, samples were then incubated on ice in a cold room overnight. The next day, ligations were heat inactivated at 90° C. for 1 min, and individually precipitated by addition of 1.2 μl of Glycoblue mix (1 μl Glycoblue™ Co-precipitant (15 mg/ml; ThermoFisher, #AM9516) in 26 μl 5 M NaCl (ThermoFisher, #AM9579)) and 63 μl of 100% ethanol was added to each tube. Reactions were combined, precipitated on ice and centrifuged for 1 h at 14,000 RPM, at 4° C. The pellet was dried and resuspended in 20 μl nuclease-free water and 20 μl denaturing PAA gel loading solution and separated on a 15% Urca-PAGE gel. Size marker RNA oligonucleotides (IDT) were used to guide gel excision. The excised gel piece was crushed using a gel-breaker tube (IST Engineering, #3388-100) and incubated in 400 mM NaCl O/N at 4° C., at 1,100 RPM on a thermomixer. The next day the solution was filtered, precipitated and a RNA pellet was obtained by centrifugation at 14,000 RPM for 1 h at 4° C. The 5′ adapter was added to the resuspended pellet and T4 RNA Ligase 1 (New England Biolabs, #M0204L) was added for 1 h at 37° C. The ligated product was separated on a 12% Urea-PAGE gel in the presence of 5′ ligated size markers, as guide for size selection. Again, the excised gel piece was crushed, resuspended in 300 mM NaCl solution with lul 100 M 3′ PCR primer, and incubated O/N on a thermomixer at 1,100 RPM at 4° C. Subsequently, the solution was filtered, precipitated with 100% ethanol, incubated on ice for 1 h and pelleted by centrifugation at 14,000 RPM for 1 h at 4° C. The RNA pellet was resuspended in nuclease free water for reverse transcription (3 μl 5× first strand buffer, 1.5 μl of 0.1M DTT, and 4.2 μl dNTP Mix (2 mM each; ThermoFisher, #R0241)) with 0.75 μl SuperScript® III Reverse Transcriptase (ThermoFisher, #18080-093) and incubated at 50° C. for 30 min. Reverse transcription was deactivated at 95° C. for 1 min, followed by addition of 95 μl nuclease-free water. A pilot PCR reaction was set up (10 μl 10×PCR buffer, 10 μl dNTP Mix (2 mM each), 10 μl cDNA, 67 μl nuclease-free water, 0.5 μl 3′ PCR primer, 0.5 μl 5′ PCR primer, and 2 μl Titanium® Taq DNA Polymerase (Clontech Laboratories, #639208)). 12 μl aliquots were withdrawn at cycles 10, 12, 14, 16, 18, 20 and 22 for analysis on a 2.5% agarose gel, and identification of the optimal PCR amplification cycle. Six PCR reactions were then set up, run for the optimal number of amplification cycles, a portion (10 μl) was evaluated on a 2.5% agarose gel. The remaining solution was combined, precipitated, digested with Pmel for removal of size markers, and separated on a 2.5% gel. The 100 nt PCR library product was excised, purified with QIAquick Gel Extraction Kit (Qiagen, #28704) and quantified using the Qubit® dsDNA HS Assay Kit (ThermoFisher, #Q32854). cDNA libraries were then sequenced (single-read 50 cycles) on a HiSeq 2500 Sequencing System (Illumina, #SY-401-2501), after which FASTQ files containing raw sequencing data were processed for adapter trimming and small-RNA alignment to the hg-19 genome. Read counts were normalized to total counts and subjected to statistical analyses (see below).

In Silico miRNA Enrichment and Gene Ontology Analyses

Based on the list of the top differentially expressed miRNAs between identified to be differentially expressed in human EVs isolated from tumor-bearing mice and control mice, miRNAs were stratified by disregarding any miRNAs where the baseMean (readcount). The resulting lists of the top 33 upregulated and the top 11 downregulated miRNAs in human pulled exhaled EVs were then subjected to both miRNA enrichment analysis (miRNet). Assessment of the top 33 upregulated and top 11 downregulated miRNAs were assessed separately in miRNet (www.mirnct.ca/upload/MirUploadView.xhtml), where each of the upregulated and downregulated miRNA lists were imputed using their miRBase IDs and selecting miTRarBase v8.0 as target. The interaction networks for both the upregulated and downregulated miRNAs were filtered using a degree filter of 2.0 for all network nodes and the minimum network selection was selected to visualize the predicted miRNA-gene interactions.

miRNA Data Analysis

Raw FASTQ data files obtained on an Illumina HiSeq2500 sequencer were processed using the RNAworld server from the Tuschl Laboratory at the Rockefeller University, including adapter trimming and read alignments and annotation. MiRNA counts were exported to spreadsheets for data analysis. Statistical analyses of miRNA counts were performed using dedicated Bioconductor packages in the R platform, as detailed below. Heat maps were generated from transformed counts using the ‘NMF’ package (aheatmap function). Differential expression was assessed using ‘DESeq2’ and ‘edgeR’. Differential expression models included a batch variable (library) to reduce batch biases. To maximize the discrimination ability of miRNA we computed a score for each sample (‘miRNA score’, [99]), assembled by summing the standardized levels (z-values) of all significantly upregulated miRNA, and the negative of the z-values of all significantly downregulated miRNA.

### Proteomic Data Analysis

Proteins present in mouse EBC were analyzed by a workflow integrating suspension trapping (S-Trap)-based sample processing and data-independent acquisition mass spectrometry (DIA-MS) [100]. In brief, proteins were extracted with 5% SDS, reduced with DTT, alkylated witth iodoacetamide, and then digested on a S-Trap column (ProtiFi, LLC) with sequencing-grade trypsin/Lys-C (Promega). The resulting peptides were analyzed with a nanoAcquity UPLC system (Waters) coupling with an Orbitrap Fusion Lumos mass spectrometer (Thermo Fisher) in DIA mode, with parameters similar as described previously [100]. The DIA data files were processed by Spectronaut (Biognosys) with default settings.

### Results

### Orthotopic Tumor-Bearing Mouse Model of Secondary Lung Cancer

The goal of our study was to determine whether secondary lung cancer can be non-invasively detected at an early stage through the analysis of exhaled breath condensates, and for this purpose we selected a human cancer cell line that when delivered via tail vein injection unequivocally migrates and colonizes the lungs. The group of Dr. Massagué developed the highly metastatic breast cancer MDA-MB-231 subline 3475 (lung metastatic (LM)-3475 cell line) to form lung tumor foci that expand to form large tumors within 16 to 20 weeks [101]. Here we also demonstrated that these cells preferentially colonize the lungs of athymic BALB/C mice upon their tail vein injection [102,103].

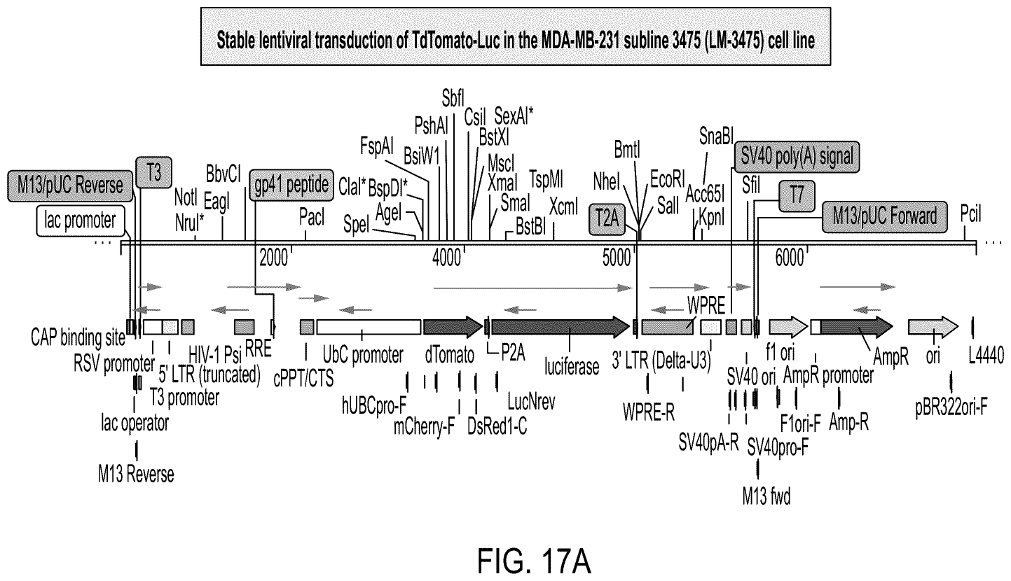

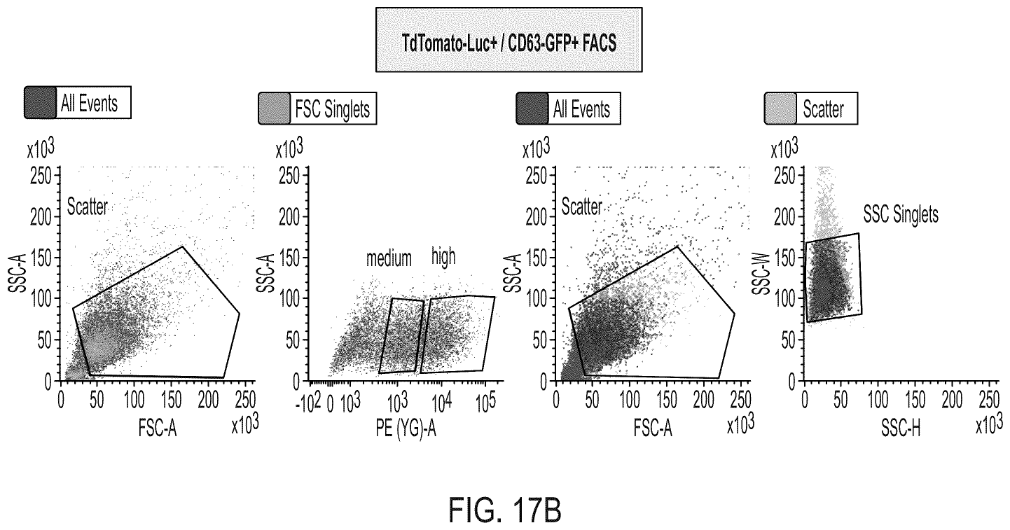

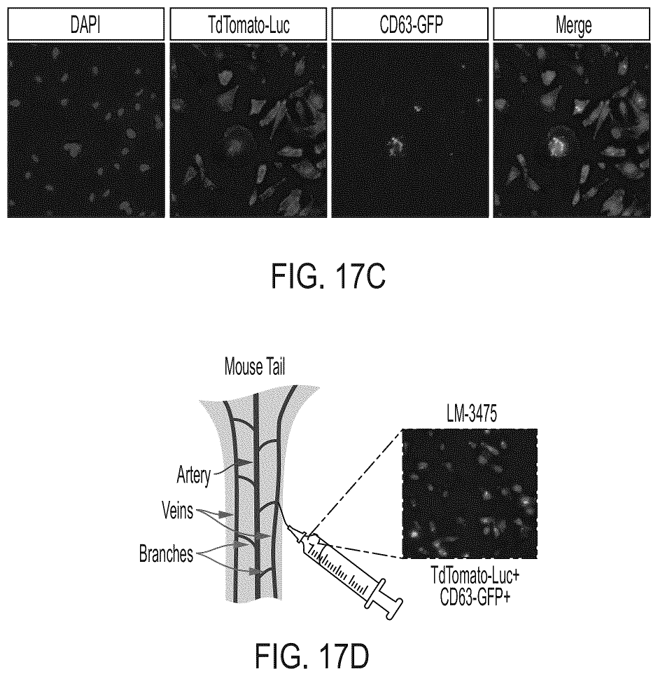

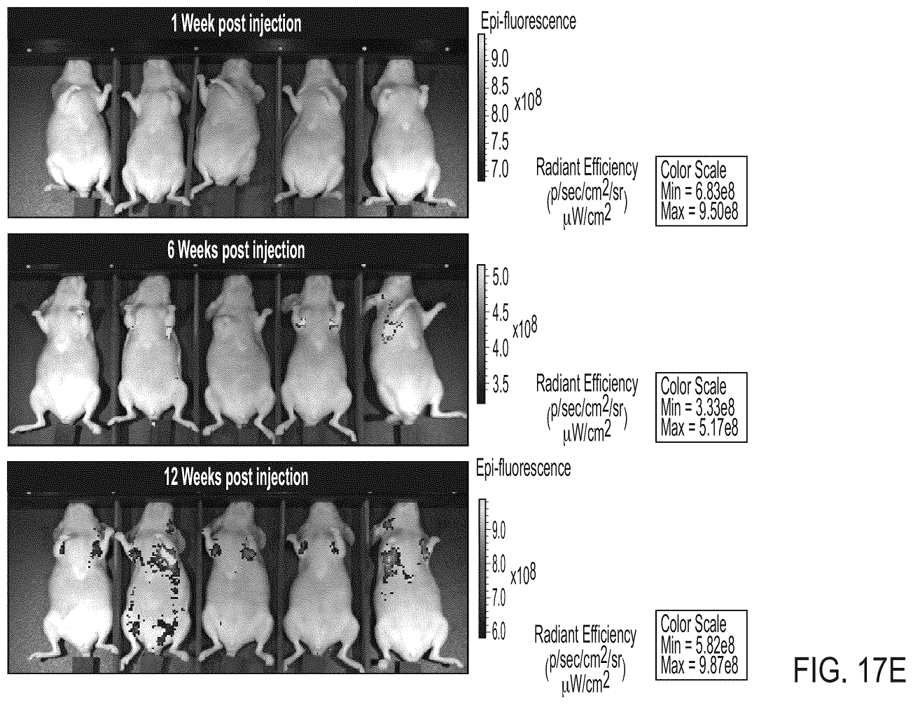

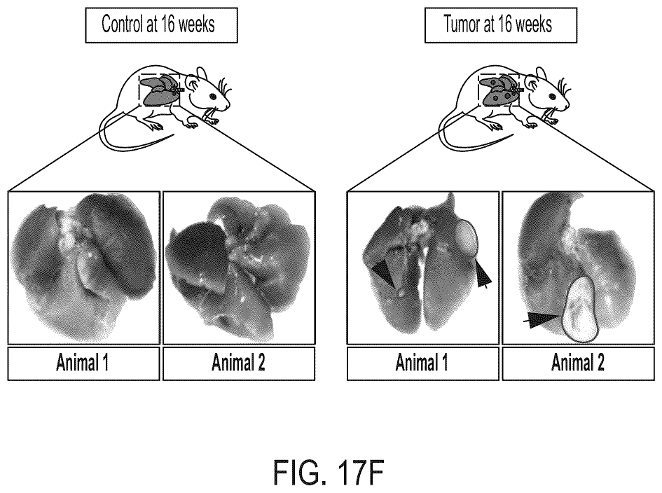

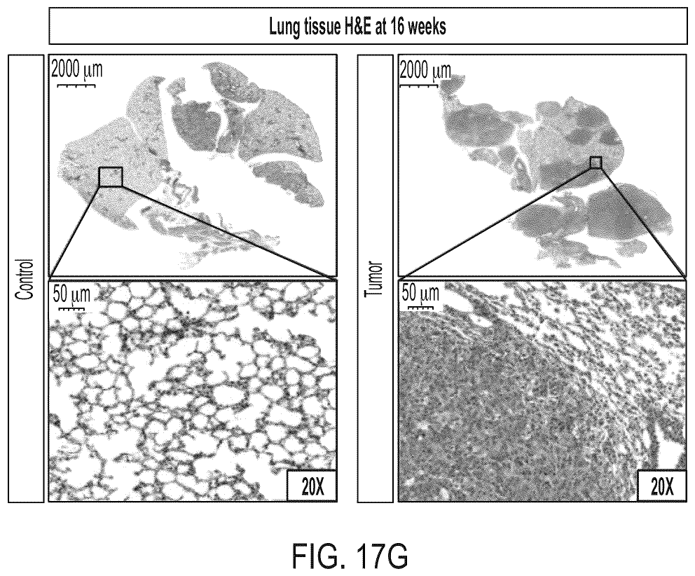

In this study and to enable tumor growth evaluation in vivo, we stably transduced the LM-3475 cells with the TdTomato-Luciferase (pUltra-Chili-Luc (9947 bp)) construct, which allows for bioluminescent imaging of tumor burden in mice (FIG. 17A). These cells were also stably transduced with the CD63-GFP construct which allows for the tracking of EV biogenesis through the visualization of CD63 within these cells. Following transduction, cells were sorted by means of fluorescent activated cell sorting (FACS) and single-clones expressing high intensity levels of both TdTomato and CD63-GFP were selected (i.e., TdTomato-Luc/CD63-GFP+ double positive cells) (FIG. 17B). Clonal expansion of double positive cells was performed, and the expression and co-localization of fluorescent signals was confirmed by confocal imaging (See FIG. 17C, DAPI nuclei staining (blue), Td-Tomato-Luc (red) and CD63-GFP (green)). The selected double positive fluorescent LM-3475 clone was expanded in tissue culture and for our in vivo animal experiments 1×10⁶ LM-3475 cells were delivered via tail vein injection into male and female athymic BALB/C mice (FIG. 17D). We conducted in vivo bioluminescent imaging, weekly, to monitor tumor localization and detection of tumor burden in individual mice. Consistent with previous studies, we determined that luciferin bioluminescent signal could be detected within the thoracic area of animals by week 6 (FIG. 1e). Using bioluminescent in vivo imaging we observed that signal intensity increased and became more consistent across all animals by week 12 (FIG. 17E). At week 16, the animals were sacrificed, and whole lungs were collected from both lung tumor-bearing and healthy control animals and we visually observed large macroscopic tumor lesions in the lungs of tumor-bearing animals (FIG. 17F). Lungs were formalin-fixed and paraffin embedded (FFPE) and Hematoxylin and Eosin (H&E) stained 5 m sections were evaluated by our study pathologist (FIG. 17G). As expected, the pathological evaluations revealed the presence of both microscopic tumor foci as well as large tumors within lung tissue (FIG. 17G). We did not observe significant differences in the growth or number of tumors between males and females.

### Evaluation of a Whole Mouse EBC Collection System (Version 1.0)

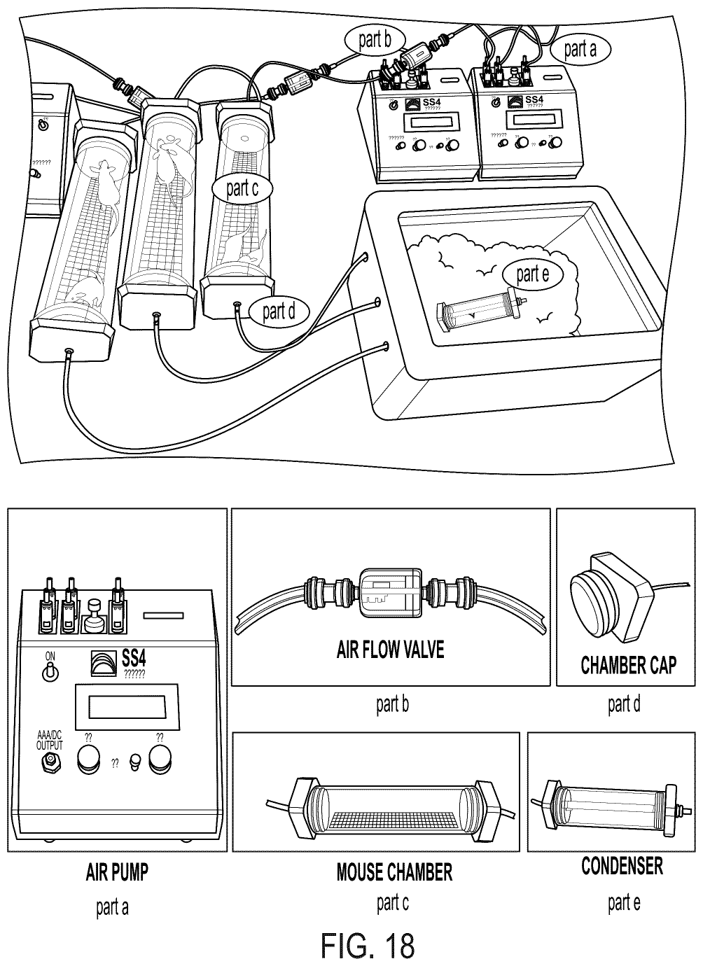

For these experiments, we sought to determine whether EBC could be collected from unrestrained lung tumor-bearing and control athymic BALB/C mice. Utilizing the Sable Systems International (SSI) classic instrumentation line, we combined devices to enable their system to collect exhaled breath condensates (FIG. 18). In this airtight system, air is circulated unidirectionally to collect vaporized exhaled breath from free-roaming animals by condensation (part a). Using the SS4 pump (SSI), which is set at a flow rate of 2 ml per minute, compressed breathing-grade air is circulated through a one-way Balston air flow filter (i.e., thus preventing air back flow) and directed by ¼ inch tubes into the successive chambers (part b). The first is a glass chamber (part c), which accommodates up to two mice, is sealed on both ends by caps with double gaskets to prevent air leakage (part d). This chamber is fitted with a removable metal grate to allow the mice to move freely and to allow urine and feces to drop in the lower section of the glass chamber. As the air is continually pushed through the mouse glass chamber, it is directed toward a small glass chamber (i.e., condenser), which is surrounded with ice to enable condensation of exhaled breath (part e). This glass condenser is also sealed on both ends with tight fitting endcaps equipped with double gaskets that prevent air leakage. For our experiments, we collected condensates from animal pairs of the same sex, weekly, for 16 weeks. Our weekly collections revealed that we could collect an average volume of ˜62.5 μl of biofluid in each condenser within one hour (i.e., average collected over 96 collections (3 animal pairs×1 collection per week ×16 weeks×2 groups)). We set up our system to allow for the collection of three separate condensates from three dedicated pairs of animals. For our analyses, we collected condensates from males (n=6; 3 pairs) and from females (n=6; 3 pairs) separately, both from our control (6 males and 6 females) and lung tumor-bearing (6 males and 6 females) groups.

### MiRNA Expression Analyses of Exhaled Breath Condensates

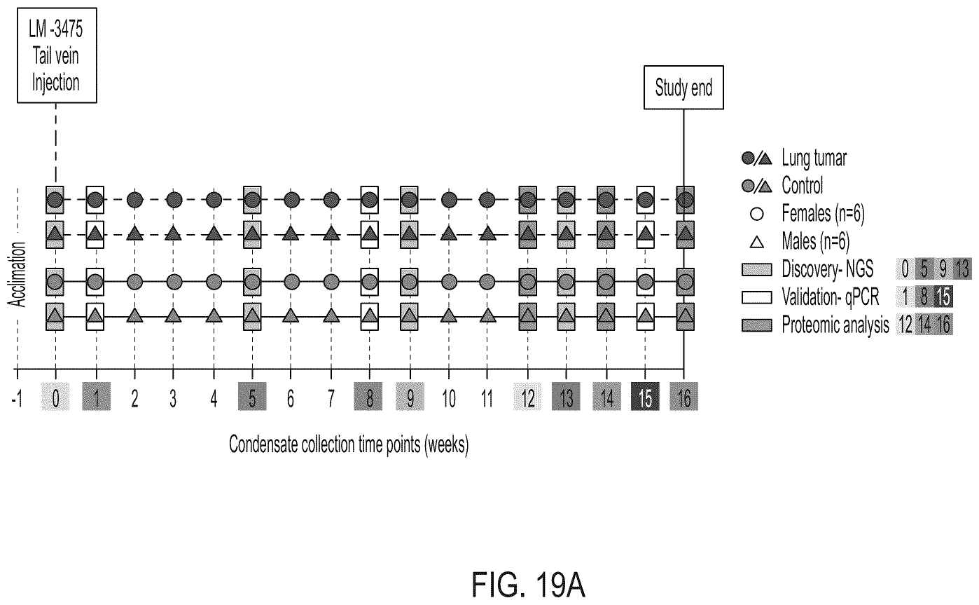

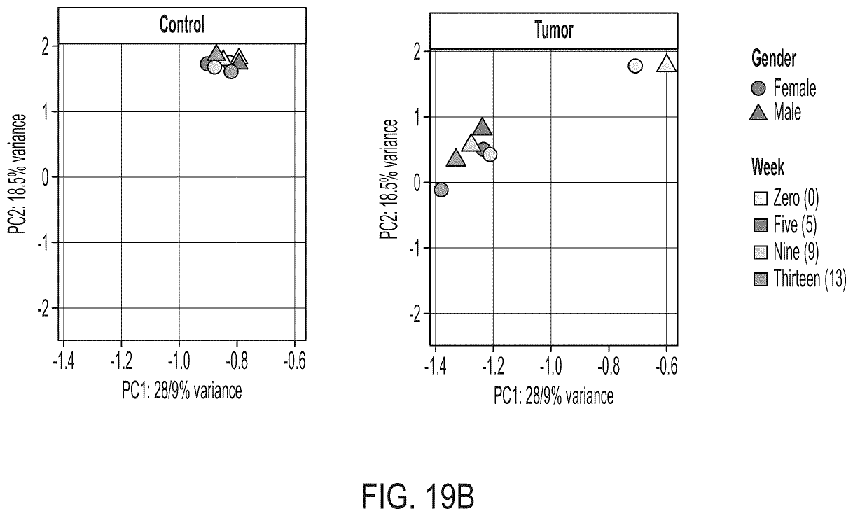

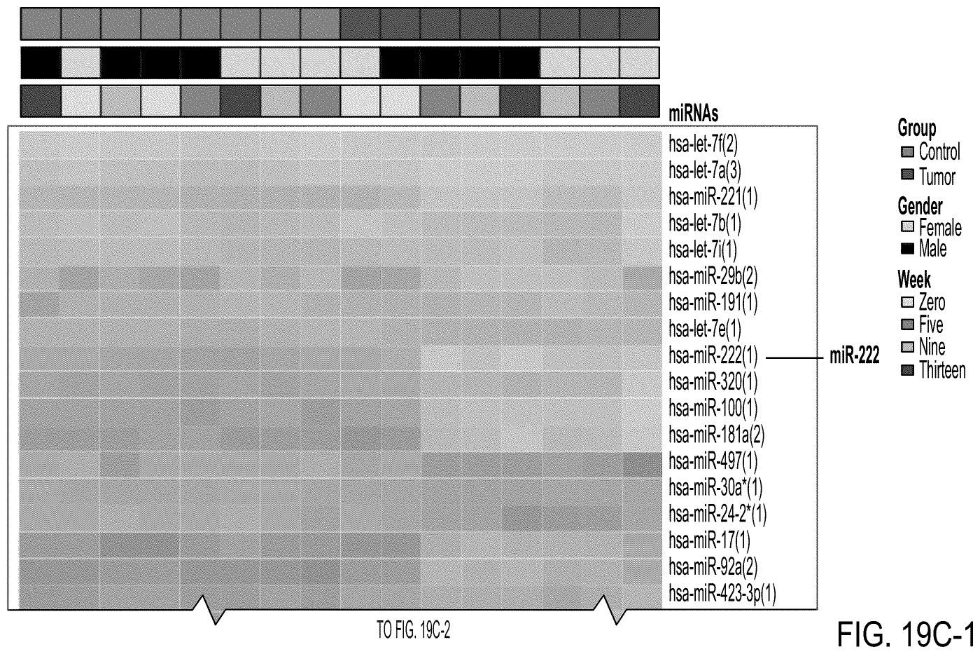

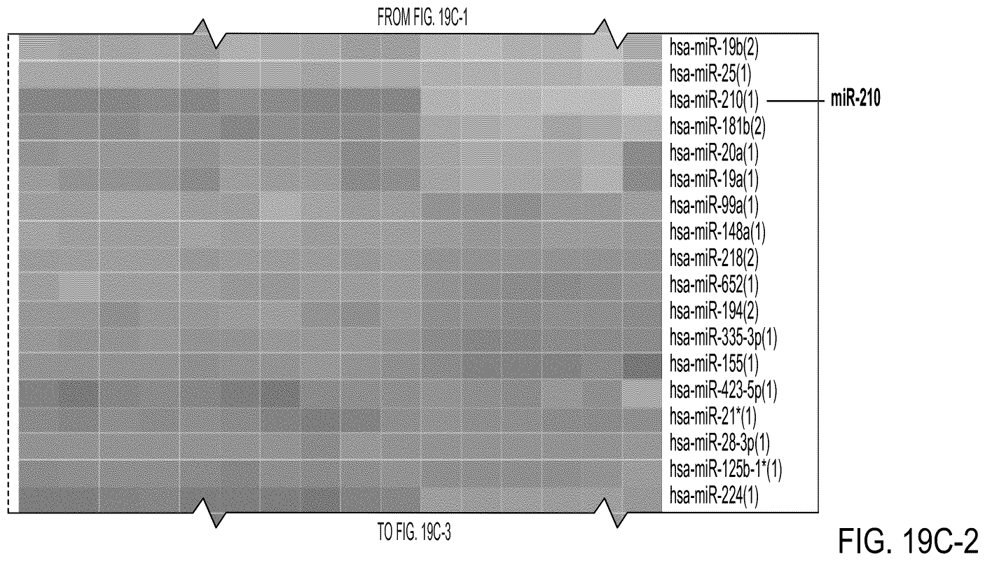

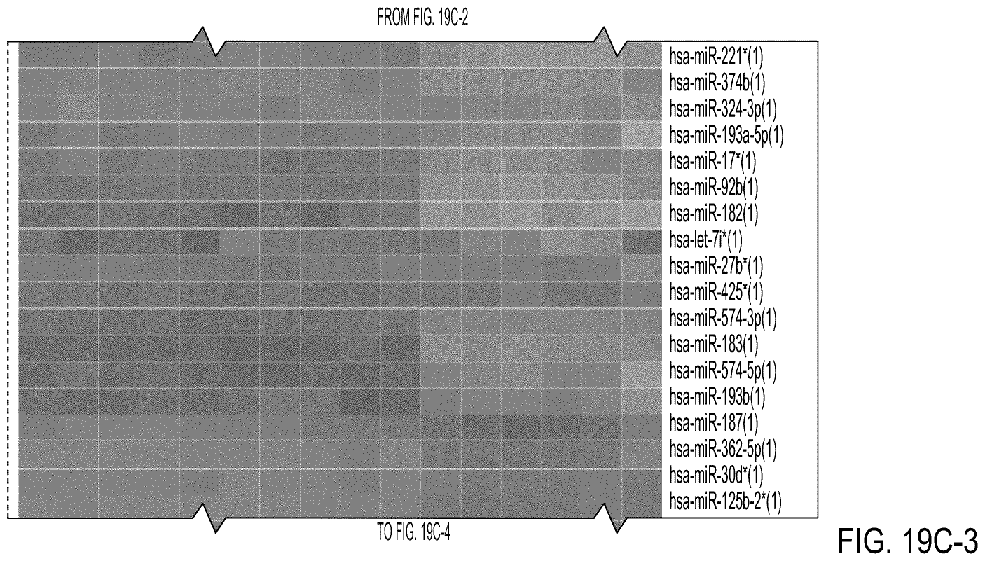

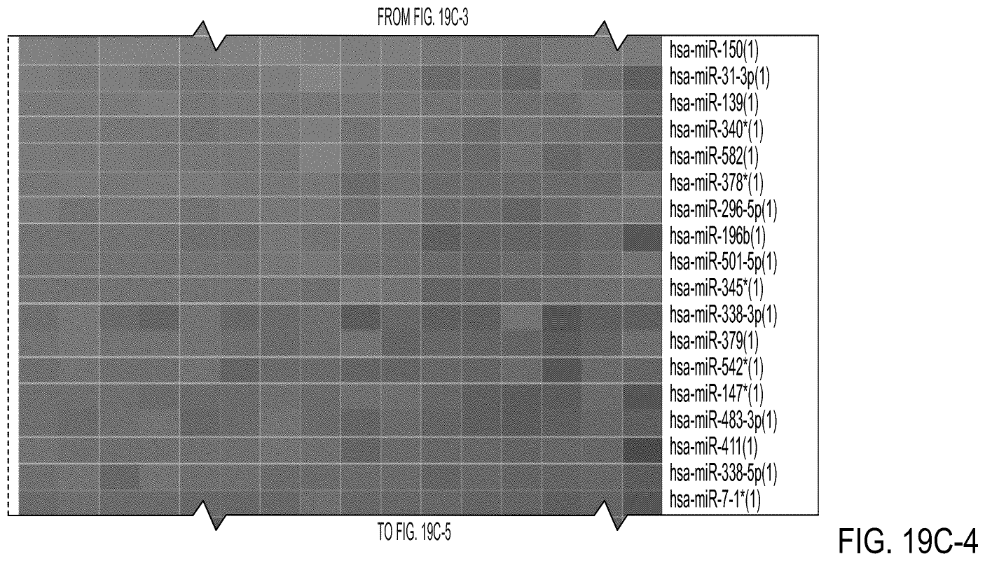

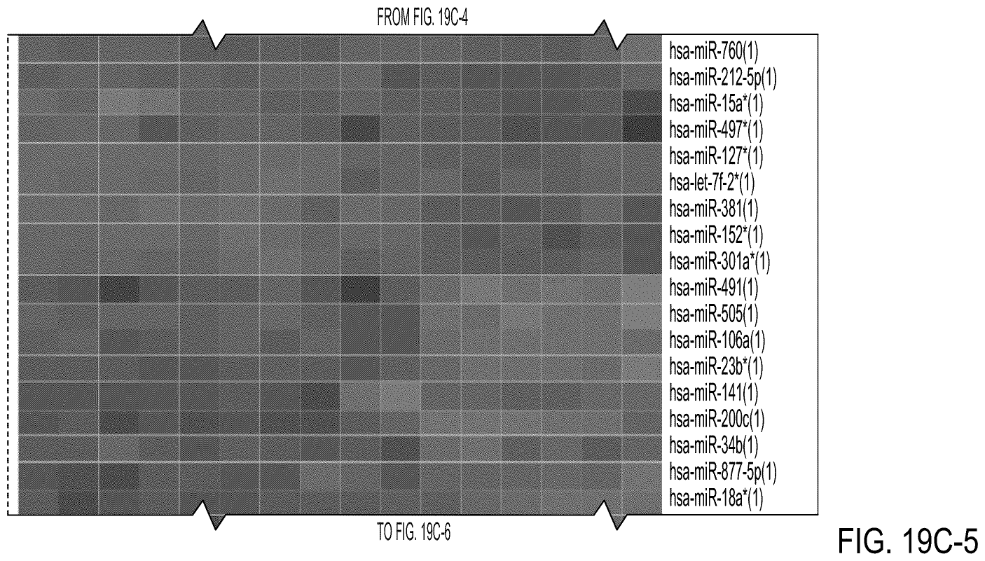

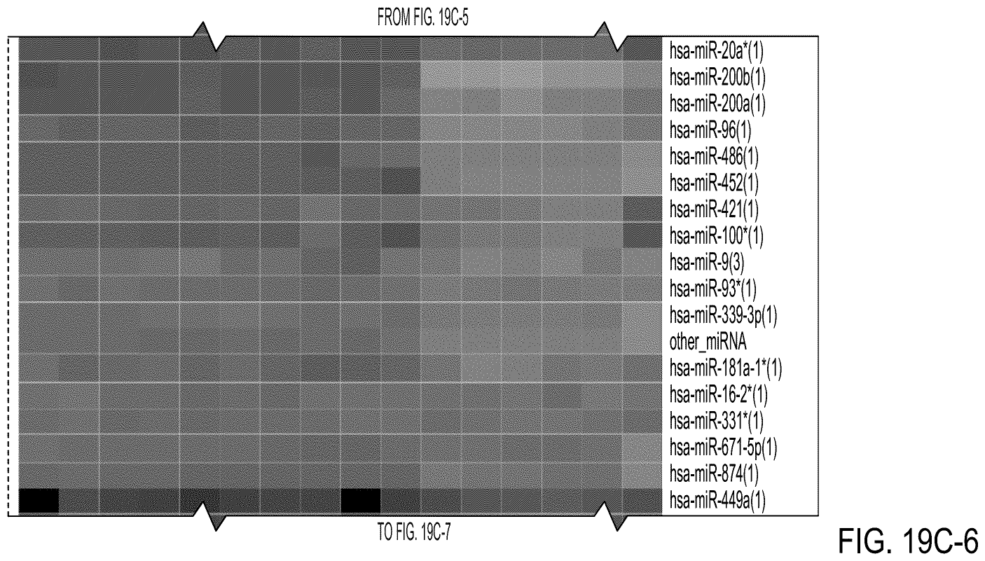

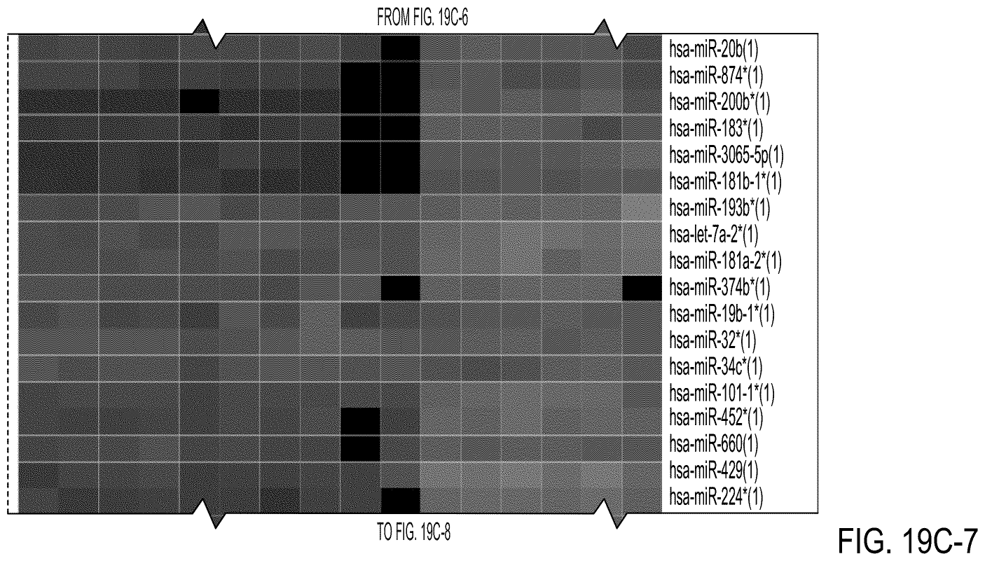

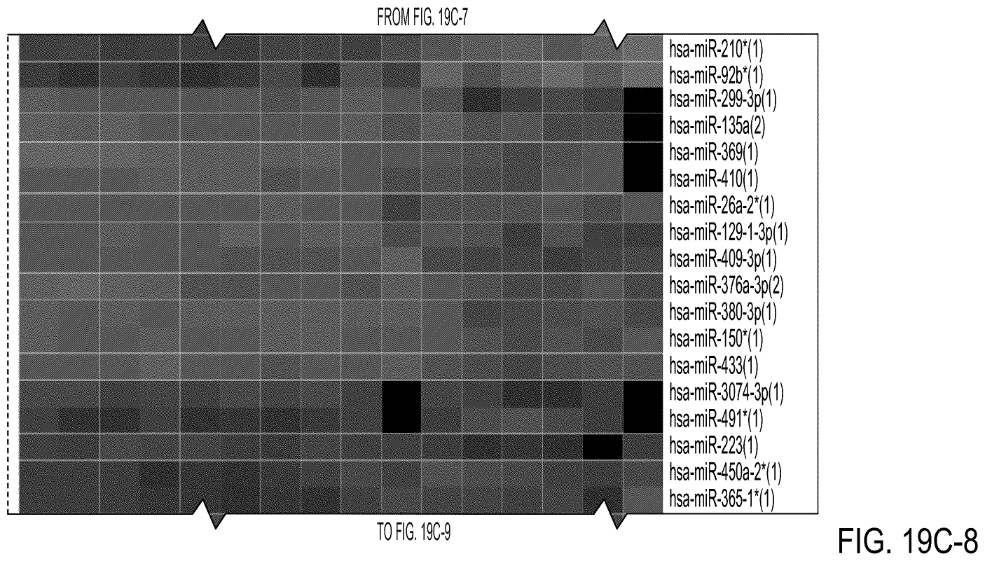

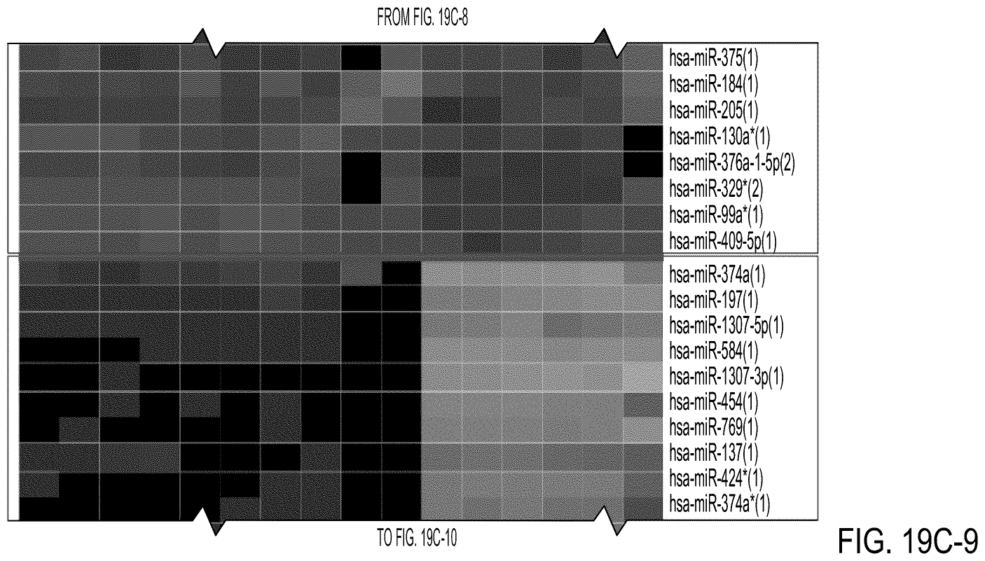

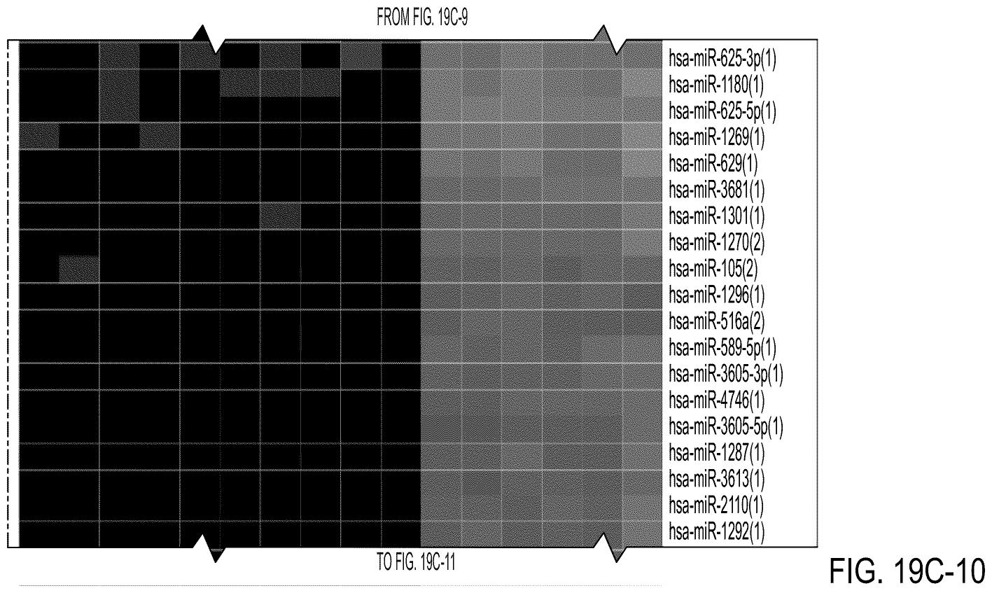

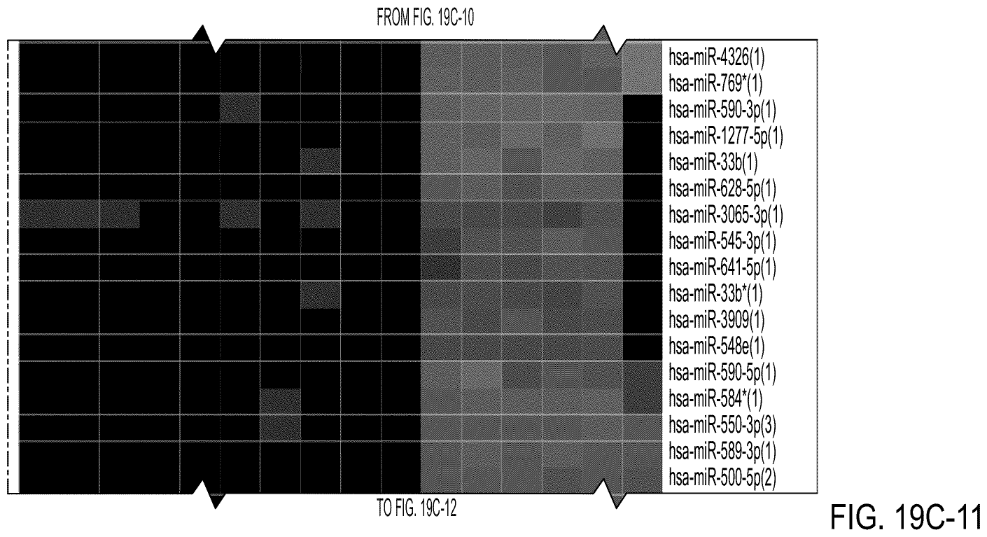

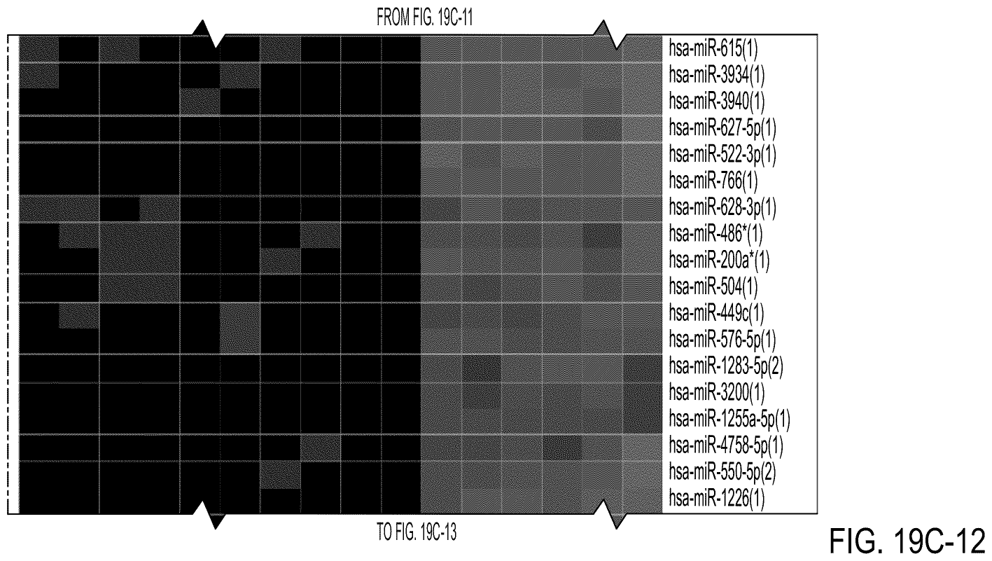

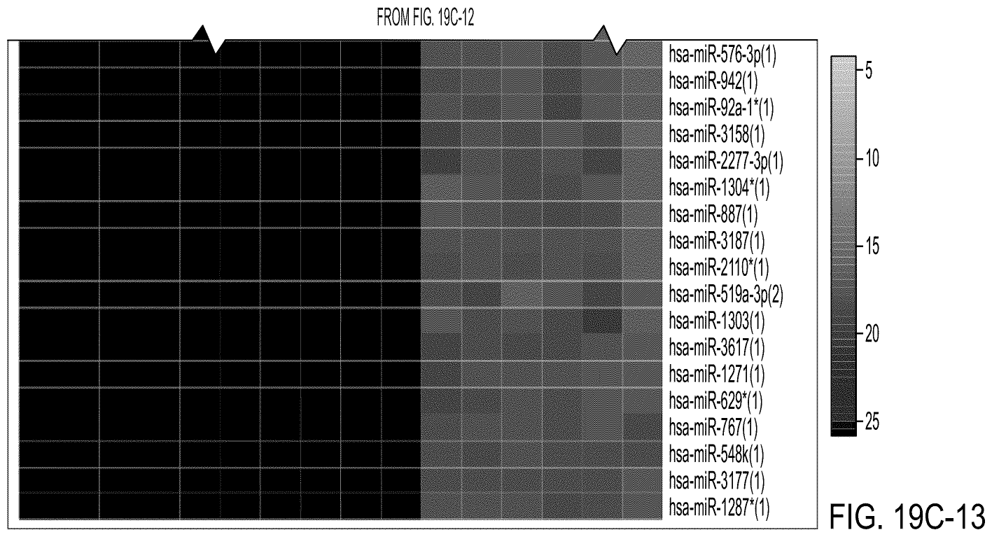

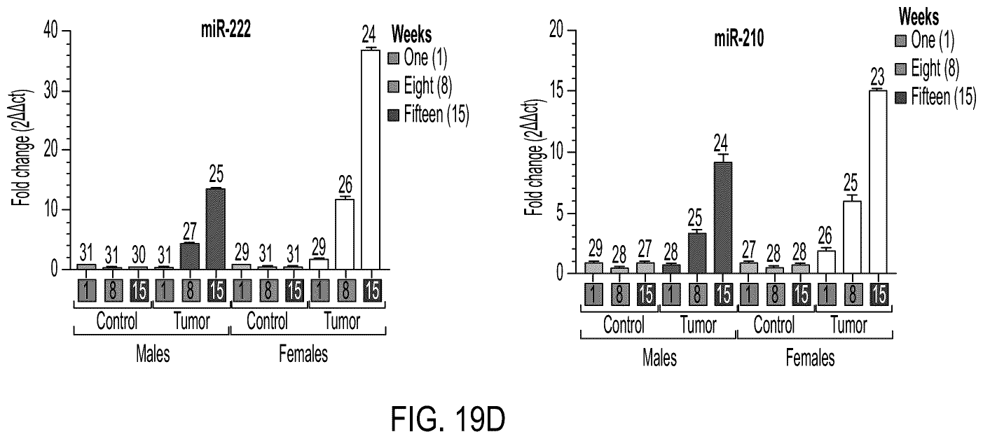

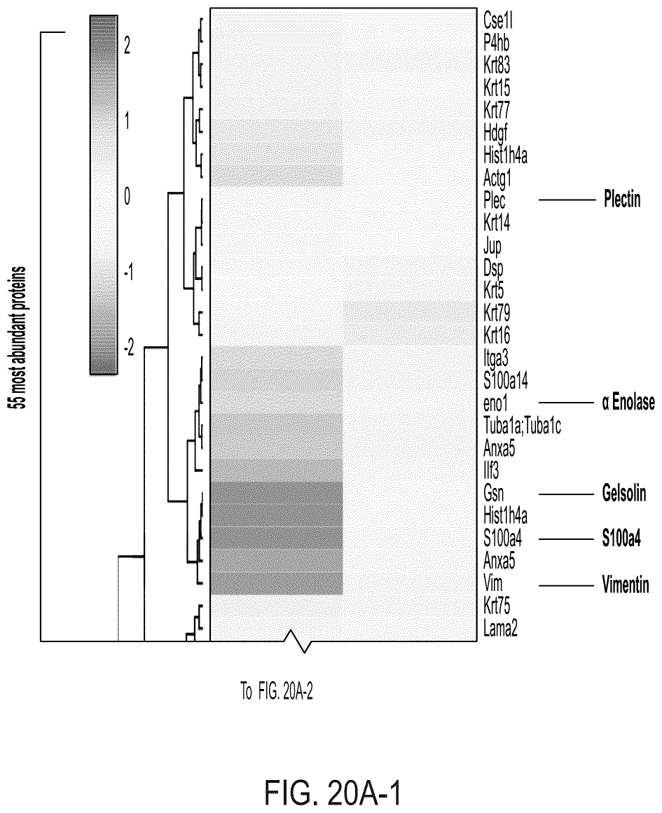

Considering that we recently demonstrated that miRNAs are detectable in human EBC and that they may have diagnostic value for the detection of lung cancer [42], we investigated whether secondary lung tumors could likewise be non-invasively detected in the EBC of lung tumor bearing mice by microRNA expression analyses. To this end, we conducted small-RNA extractions from EBC collected at weeks 0, 5, 9 and 13 followed by small-RNA next generation sequencing (NGS) analyses (FIG. 19) [97,98]. For these analyses, we separately collected condensates from control females (FIG. 19A, blue circles (n=6)), control males (FIG. 19A, blue triangle (n=6)), and from lung tumor-bearing females (FIG. 19A, red circle (n=6)), and lung tumor-bearing males (FIG. 19A, red triangle (n=6)), which had received 1×10E6 LM-3475 cells delivered via tail vein injection at week 0 (FIG. 19A). Control animals received a mock injection of 1×PBS at the same time as tumor-bearing animals received LM-3475 cells, to eliminate experimental biases due to biofluid injection. We separately combined condensates from male pairs and from female pairs, for each group, at each timepoint to enable RNA extractions from at least 100 μl biofluid. Principle Component Analysis (PCA) plots revealed that the miRNA profiles captured from control animals were consistent across the 16 weeks of collection (FIG. 19B, control (blue)), but revealed differences between week 0 and weeks 5, 9, and 13 for lung tumor-bearing animals (FIG. 19B, tumor (red)). Furthermore, the miRNA profiles of condensates collected at weeks 5, 9, and 13 from lung tumor-bearing animals clustered together and appeared consistent between males and females, but highly different from those of controls (FIGS. 19C-1 to FIG. 19C-13). We observed that the miRNA profiles of EBC collected at week 0, right after tail-vein injections from lung tumor-bearing animals clustered together with those of control animals (FIGS. 19C-1 to 19C-13). Overall, the miRNA profiles displayed on the heatmap identified a set of common miRNAs that between the two groups but differentially expressed (FIGS. 19C-1 to 19C-13, purple rectangle) and a set of tumor-associated miRNAs highly detectable in the tumor group at weeks 5, 9 and 13 (FIGS. 19C-1 to 19C-13, red rectangle; ˜80 miRNAs). Although we identified miRNA expression differences between the condensates of control and tumor groups, we could not fully determine whether all these miRNAs were of human (tumor cell) or mouse (mouse lung) origin due to strong sequence homology between these two species. As we sought to evaluate the early detection of secondary lung tumors by analysis, we selected miR-222 and miR-210, which displayed high read counts by NGS, and has been reported in literature to be increased in circulation during breast cancer metastasis [104,105], for qPCR analyses. Our qPCR analyses revealed an increase in both miRNAs between weeks 1, 5, and 8 in EBC from lung tumor-bearing animals, for both males and females, when compared to respective male and female control animals. [FIG. 19D] We also observed that comparatively, the increase in expression of both miR-222 and miR-210 was higher in the EBC from females than that of males, and that their increased expression was already detectable at week 1 in female tumor-bearing animals [FIG. 19D].

### Proteomic Analyses of Whole Animal EBC

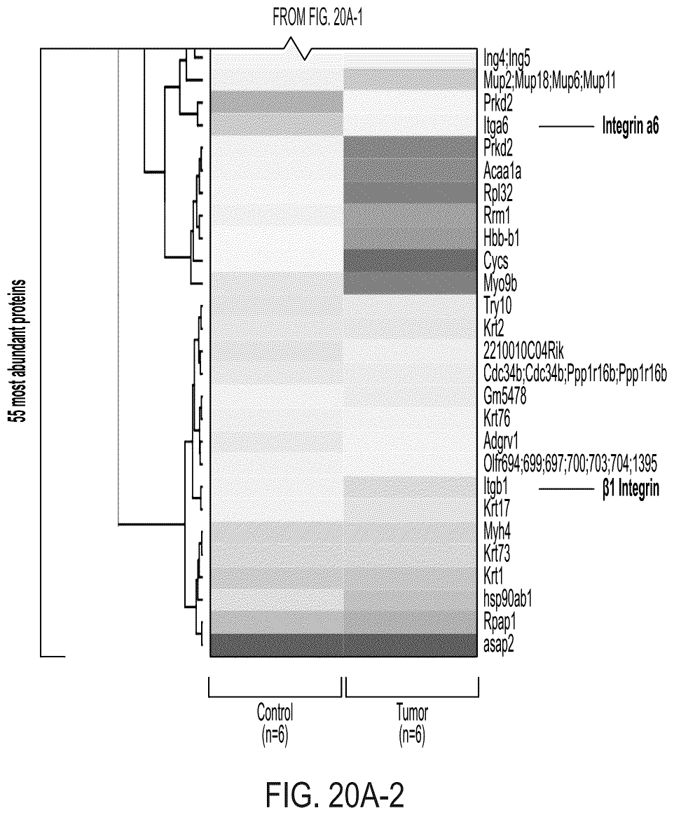

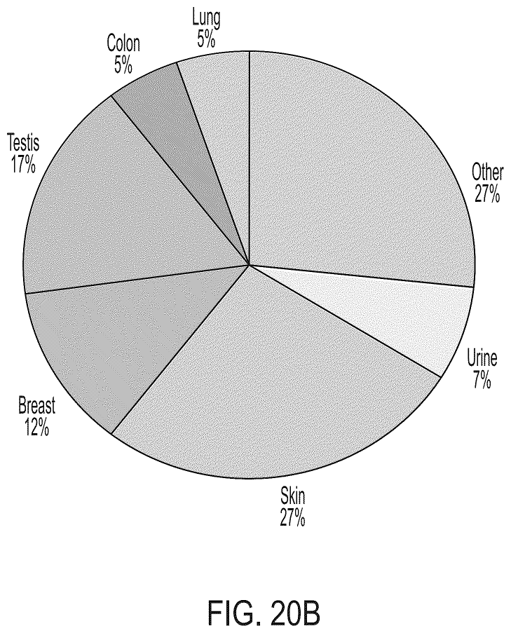

Our experiments indicated that whole animal EBCs contain miRNAs that are differentially expressed between control and lung tumor-bearing animals, however our NGS analyses could not demonstrate that the condensed biofluid was solely obtained by condensation of exhaled breath. Thus, we investigated the protein content of these condensates by using S-trap-based sample processing coupled with DIA-MS, a workflow that was developed for the robust and ultra-sensitive proteomic analysis of low input proteins and EVs [100]. We performed proteomic analyses on whole condensates combined separately from 6 control and 6 lung tumor-bearing animals, collected at weeks 12, 14, and 16 (FIG. 20A-1, FIG. 20A-2, and FIG. 20B). We identified a total of 289 proteins that appeared to be represented in whole EBC from both control and lung tumor-bearing animals. When evaluating the 55 most differentially expressed proteins (FIG. 20A-1 and FIG. 20A-2), we were able to identify several proteins that we previously identified as differentially expressed between EVs from MDA-MB-231 cells (i.e., parental cells for the LM-3475 cells used in this study) and EVs from non-tumorigenic MCF-10A cells. Some of these differentially expressed proteins identified in this study have been correlated with pro-metastatic properties in previous studies (i.e., Plectin, Gelsolin, Vimentin, B1 integrin, Integrin a6, a enolase, S100A4) [100, 106-107]. The identification of these proteins suggested that the EBC contained proteins of tumor origin. Upon further analysis of the proteomic data (FIG. 20B), we determined that murine proteins other than those associated predominantly with lung origin (i.e., skin, urine, upper digestive tract, colon, testes, and breast) could be detected (i.e., as categorized by prominent tissue of expression in the ProteinAtlas). Altogether these analyses revealed that although our miRNA analyses identified consistent expression differences between control and tumor groups, our proteomic data suggested tissue contributions other than the lung, and therefore a potential for other tissue miRNA sources.

### Evaluation of a Nose/Mouth Targeted EBC Collection System (Version 2.0)

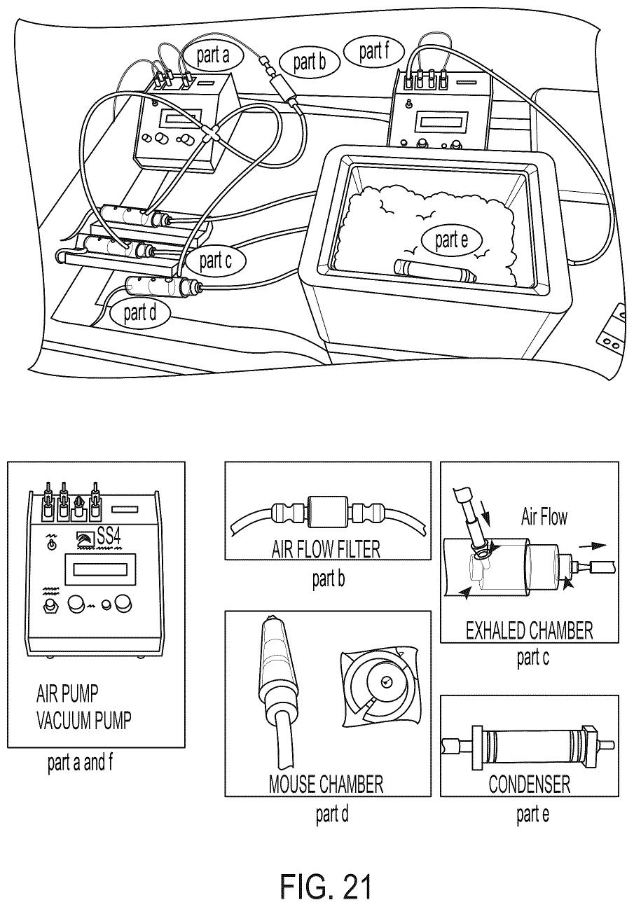

In order to target our EBC collection more precisely to organic compounds originating from the respiratory system, we modified the design of our collection system and included a mouse restraining device (i.e., described by Liu et al., 2019 [96]), to improve the direct collection of EBC from the nose/mouth of individual animals (FIG. 21). Similarly to that of our system v1.0, this system includes an air pump (part a; 2 ml per minute) supplying compressed breathing-grade air, a one-way airflow Balston filter (part b) to enable unidirectional air flow, an exhaled chamber (part c; see blue arrows for airflow) that is sealed by a gasket at its connection at the end of the mouse restrainer (part d), and a glass condenser scaled on both ends with caps with double gaskets (part e). Importantly, to enhance airflow we added an air pump to gently pull air from the end of the condenser (part f; 1 ml per minute). We determined that within a 2-hour collection period, we can collect an average of ˜29 μl of EBC from individual animals. Although single animal analysis would be preferable to evaluate disease burden, for our analyses we chose to evaluate combined EBC samples to reach a total of working volume of 100 μl. Since the LM-3475 metastatic cell line is of female breast tumor origin, and because our qPCR miRNA validations determined higher differential expression between control and lung tumor-bearing females, we chose to focus the rest of our analyses on female mice.

### Detection of Human Tumor EVs in Mouse EBC

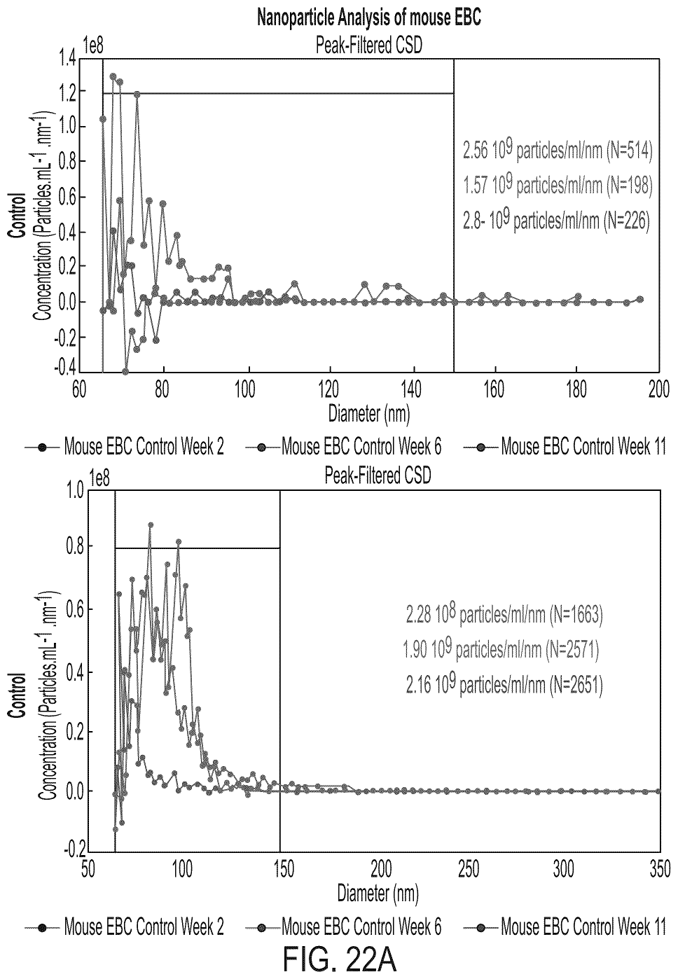

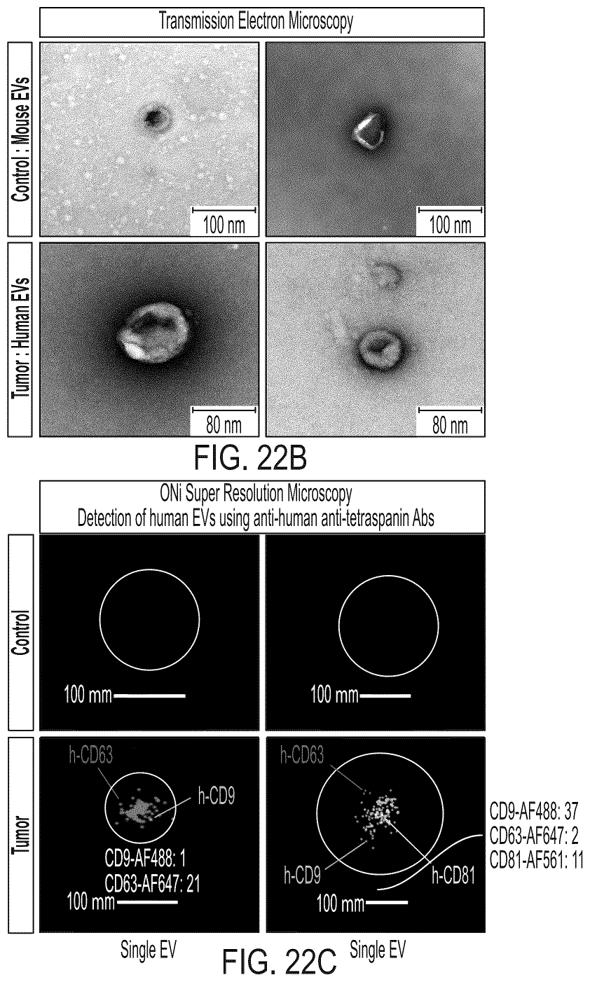

As determined in previous studies, human EBC contains EVs [48-50] and our recent study, based on the miRNA analysis of their content, also demonstrated that exhaled EVs from human subjects are of deep lung tissue origin [108, manuscript under review]. In this lung-tumor bearing animal study, we sought to determine that the EBC collected from restrained animals also contains EVs. Therefore, we conducted nanoparticle analyses of ultracentrifuged EBC pellets from both control and lung tumor-bearing animals using the Spectradyne nCS1 instrument (FIG. 22A). Our analyses of pooled EBC collected from three animals at weeks 2, 6, and 11 revealed that small particles of 65-150 nm in diameter were detected in EBC pellets from both control (n>57 particles) and lung tumor-bearing groups (n>1663 particles) but were present at much greater concentrations in EBC pellets from the tumor group. We also noted a steep increase in the number of particles detected upon disease progression from week 2 to week 11 for the lung tumor-bearing group (FIG. 22A; from 1,663 at week 2 to 2,651 particles at week 11, detected after background subtraction) . . . . Next, we performed Transmission Electron Microscopy (TEM) on EVs obtained from 1 ml ultracentrifugation pelleted EBC collected from multiple lung tumor-bearing animals between 19 and 22 weeks (FIG. 22B; bottom panels). Because our Spectradyne analyses showed that EVs from control animals were in much lower concentrations we collected a total of 3 ml from multiple control animals over a period of four weeks before performing ultracentrifugation and TEM analysis (FIG. 22B; top panels). EVs detected from the EBC of lung tumor-bearing and control mice ranged in sizes of ˜80-100 nm in diameter. Because our analyses suggested that mouse EBC from lung tumor-bearing mice contained a higher number of particles (FIG. 22A; bottom panel), we sought to determine if these particles were human tumor EVs and used super resolution microscopy to confirm both the particle size and the presence of human tetraspanins (ONi; FIG. 22C). Using the commercially available ONi human EV profiler kit and their super resolution microscopy platform, we captured and confirmed the presence of human CD9, CD63, and CD81 proteins on the surface of EV pelleted by ultracentrifugation of EBC from lung tumor-bearing mice (FIG. 22C, bottom panels). We could not capture and detect human EVs from the EBC collected from control animals (FIG. 22C; top panels). These analyses indicate that athymic BALB/c mice inoculated with the highly metastatic human LM-3475 breast cancer cell line exhaled human lung tumor EVs, which could be collected using our EBC collection system (FIG. 21; v2.0 system).

MiRNA Analyses of EBC and Exhaled EVs Collected Directly from Nose/Mouth

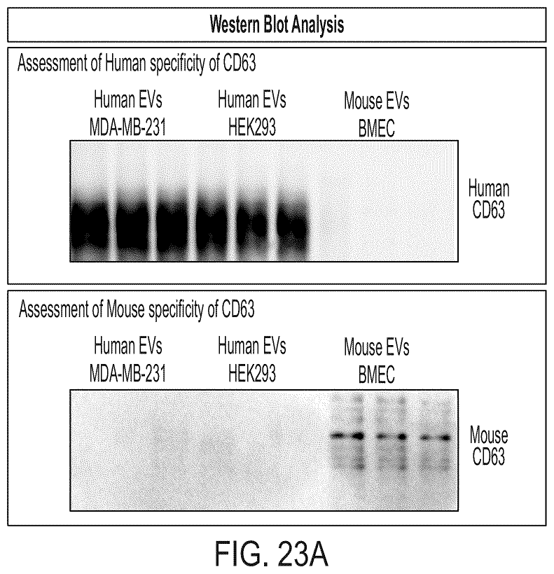

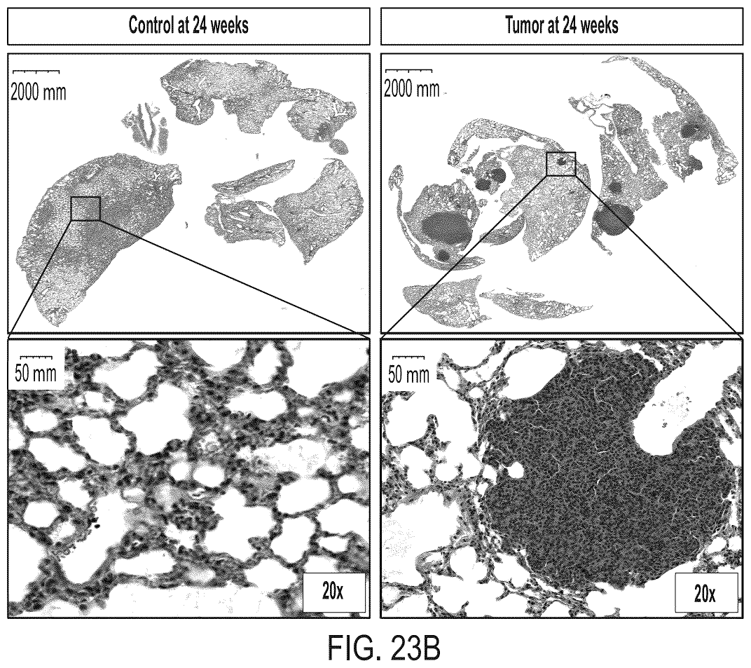

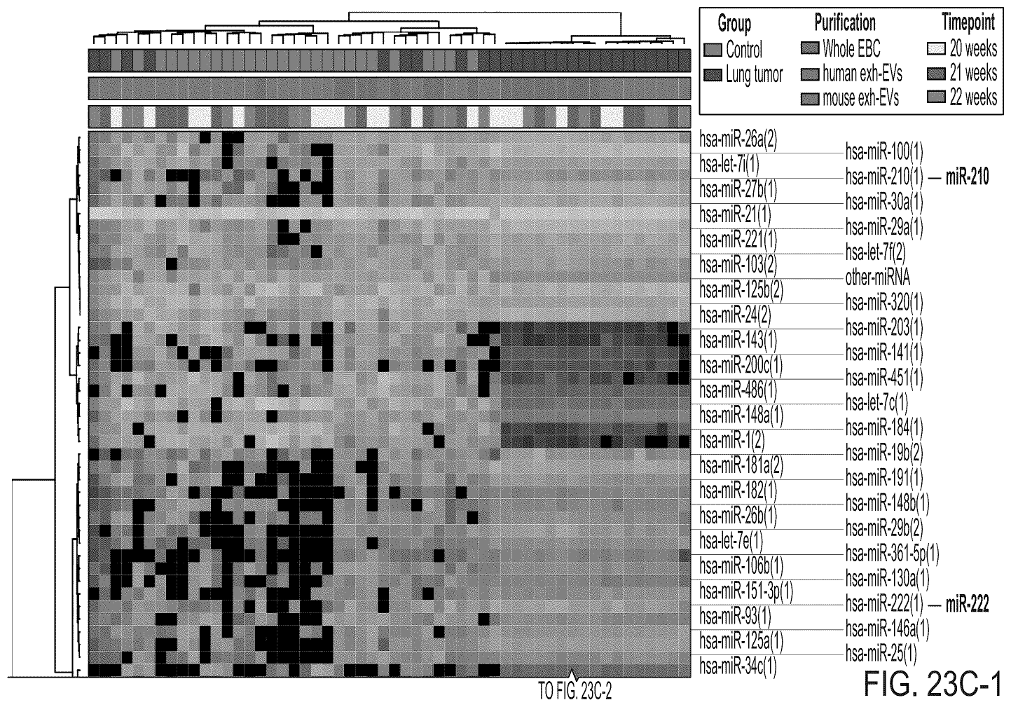

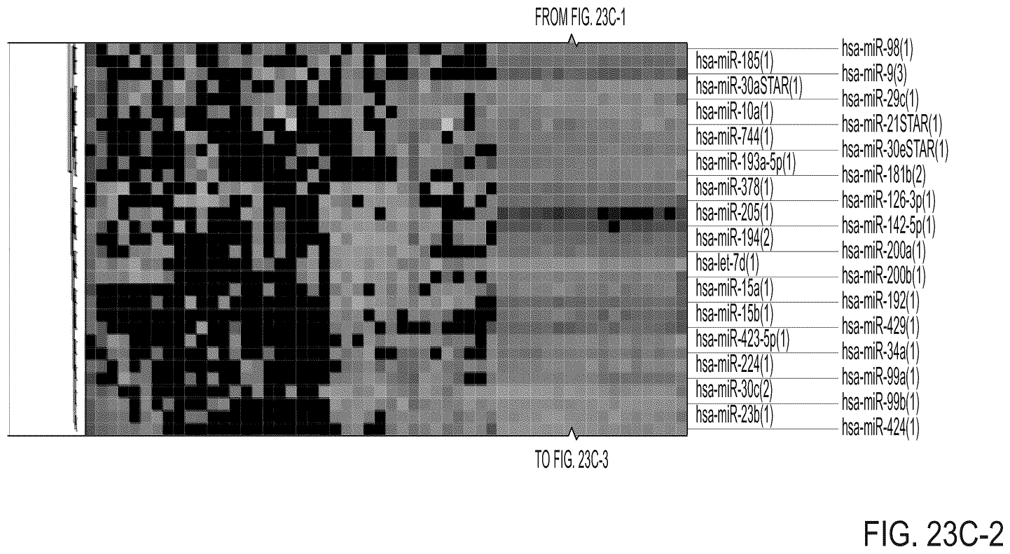

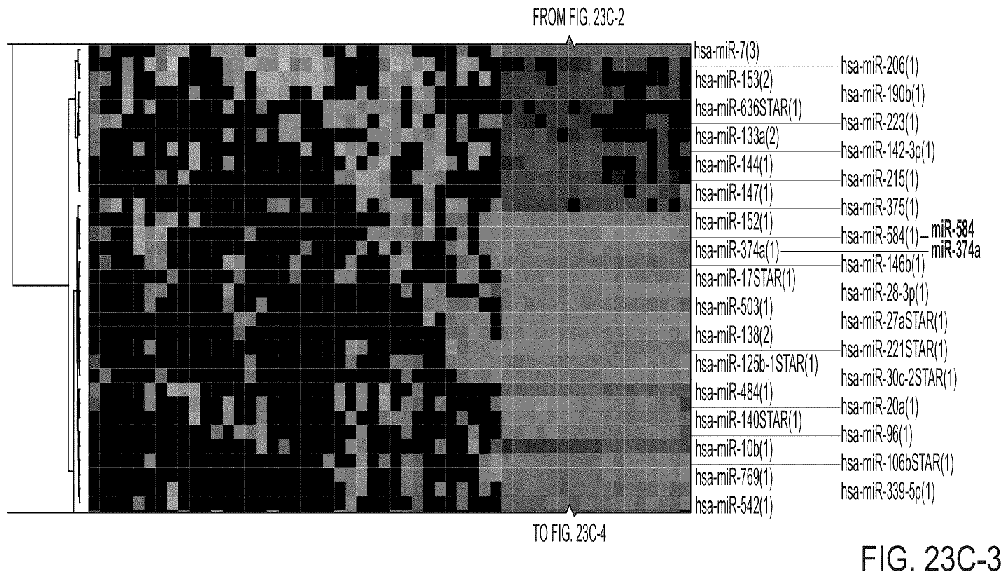

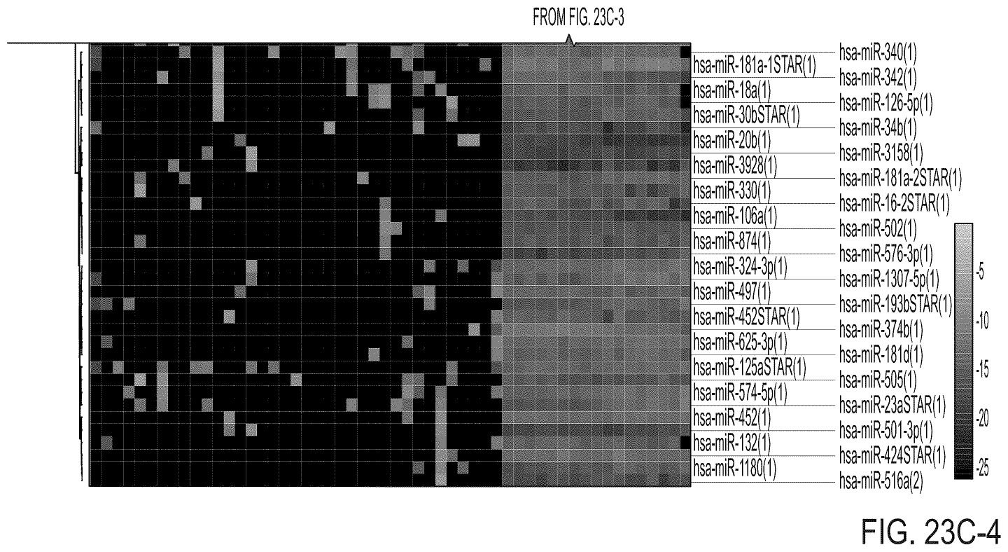

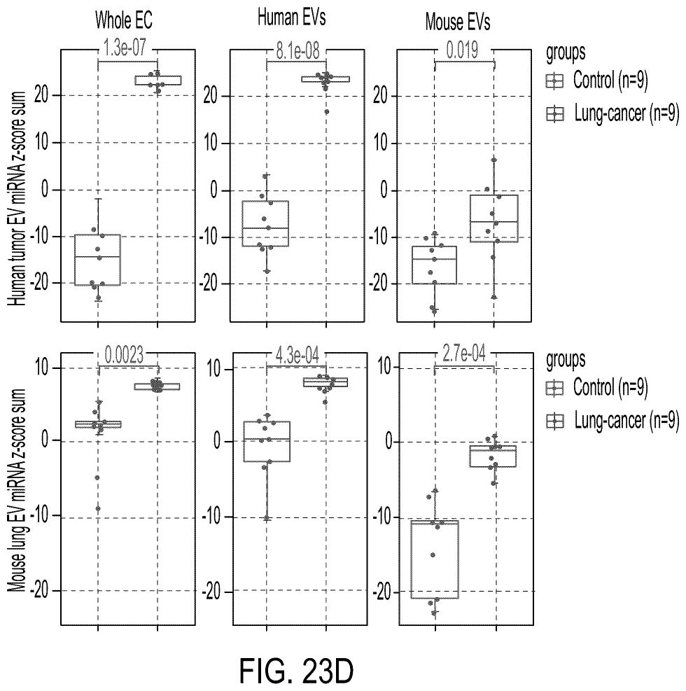

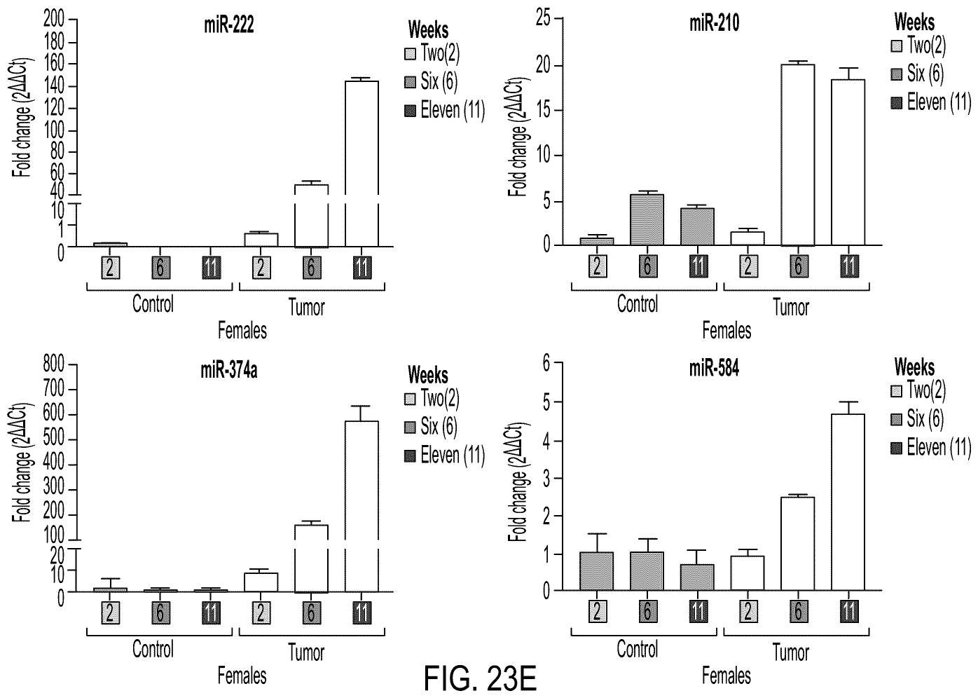

Because we recently developed EV-CATCHER, a customizable EV purification assay designed for the selection of circulating EVs and the analysis of their miRNA cargo [97], we sought to use it for the purification of human exhaled tumor EVs from EBC of lung tumor-bearing animals. For these analyses, we selected a human-specific anti-CD63 antibody and a mouse-specific anti-CD63 antibody for customization of EV-CATCHER, and for comparative analysis of human and mouse EVs purified from mouse EBC. We validated the species specificity of each CD63 antibody by Western blot analyses of EVs ultracentrifuged from the tissue culture-derived media of human MDA-MB-231 breast cancer cell, human HEK293 cells, and primary mouse bone marrow derived endothelial cells (BMEC) (FIG. 23A). Our semi-quantitative Western blot analyses revealed high species-specific recognition of both anti-human and anti-mouse anti-CD63 antibodies (FIG. 23A). Considering the high tumor burden observed at 24 weeks (FIG. 23B; see H&E), we chose to conduct miRNA NGS analyses using EBC collected at weeks 20, 21, and 22 to ensure similar disease burden among the animals and high detectability. Prior to NGS analyses, we performed species-specific sequential isolations of human and then mouse EVs using our respective anti-human and anti-mouse CD63-EV-CATCHER assays, respectively from whole EBC. Our NGS data of whole EBC (FIGS. 23C-1 to 23C-4, blue and green squares) identified ˜70 miRNAs unique to lung tumor-bearing animals when compared to whole EBC from controls (FIGS. 23C-1 to 23C-4, red and green squares). In contrast, the EV-CATCHER guided purification of human tumor EVs from EBC of lung tumor-bearing animals (FIGS. 23C-1 to 23C-4, red and orange squares) identified over ˜120 miRNAs uniquely detected in human EVs when compared to control EBC (FIGS. 23C-1 to 23C-4, blue and red squares). When using the mouse-specific EV-CATCHER assay, we generated miRNA profiles (FIGS. 23C-1 to 23C-4, blue and purple squares), which clustered closely to that of whole EBC from control mice (FIGS. 23C-1 to 23C-4, blue and green squares). It is important to note that when using the anti-human EV-CATCHER assay with EBC of control animals (FIGS. 23C-1 to 23C-4 blue and orange squares), due to the minimal cross-reactivity of the human anti-CD63 antibody against mouse CD63, we were able to detect a background miRNA profile. However, when comparing miRNA profiles obtained from whole EBC with that of anti-human CD63-purified tumor EVs from lung tumor-bearing animals, we confirmed that the bulk of differentially expressed miRNAs originated from human tumor exhaled EVs.

When selecting the top differentially expressed miRNAs in the EBC of lung tumor-bearing animals, when compared with EBC of control animals, we generated z-scores for whole EBC, human tumor EVs and mouse EVs (FIG. 23D). The box plot analyses confirmed the strong overlap in detectability of human tumor miRNAs from whole EBC (p<1.3×10E-7) and human tumor EVs (p<8.1×10E-8) (FIG. 23D, top three panels) further supporting the notion that the bulk of the miRNA signal detected in whole EBC of lung tumor-bearing animals came from exhaled human tumor EVs. When evaluating these miRNAs in mouse EVs, we found that they were not significantly detectable (FIG. 23D, top right panel, p<0.19). Although the overall mouse EV miRNA signal was weaker (i.e., lower read counts) than that of human EV signal, we identified a subset of miRNAs that discriminated mouse EVs from human EVs in mouse EBC (FIG. 23D, bottom three panels). Our miRNA z-score indicated that although there was an overlap in miRNA expression between human-(p<4.3×10E-4) and mouse-(p<2.7×10E-4) EVs, some of the selected miRNAs were preferentially expressed in mouse EVs, especially in comparison with whole EBC (p<2.3×10E-3). together, these results indicate that human tumor EVs can be selectively purified and profiled from mouse EBC, allowing for the non-invasive identification of human tumor-specific miRNAs.

Next, we sought to retrospectively validate our findings by qPCR using EBC samples collected at earlier stages of disease (i.e., weeks 2, 6 and 11) for animals that developed significant disease (FIG. 23E). Similarly, to NGS data obtained from EBC collected with our v1.0 collection system, we found miR-222 and miR-210 to be increased in whole EBC of lung tumor-bearing animals collected with our v2.0 collection system. We also identified two additional miRNAs (miR-374a and miR-584) that were primarily detected in whole EBC of lung tumor-bearing animals but not detected in EBC of controls by NGS, which were similarly detected in EBC collected from animals using our v1.0 collection system (see FIGS. 19C-1 to 19C-13 and FIGS. 23C-1 to 23C-4). Our qPCR analyses validated increased expression of all 4 miRNAs, with miR-222 and miR-374a providing greater sensitivity for earlier detection of disease than miR-210 and miR-584. These results confirm that exhaled lung tumor EVs provide molecular surrogates for early non-invasive detection of secondary lung tumors in mice.

### Proteomic Analyses of Individual Animal Condensates

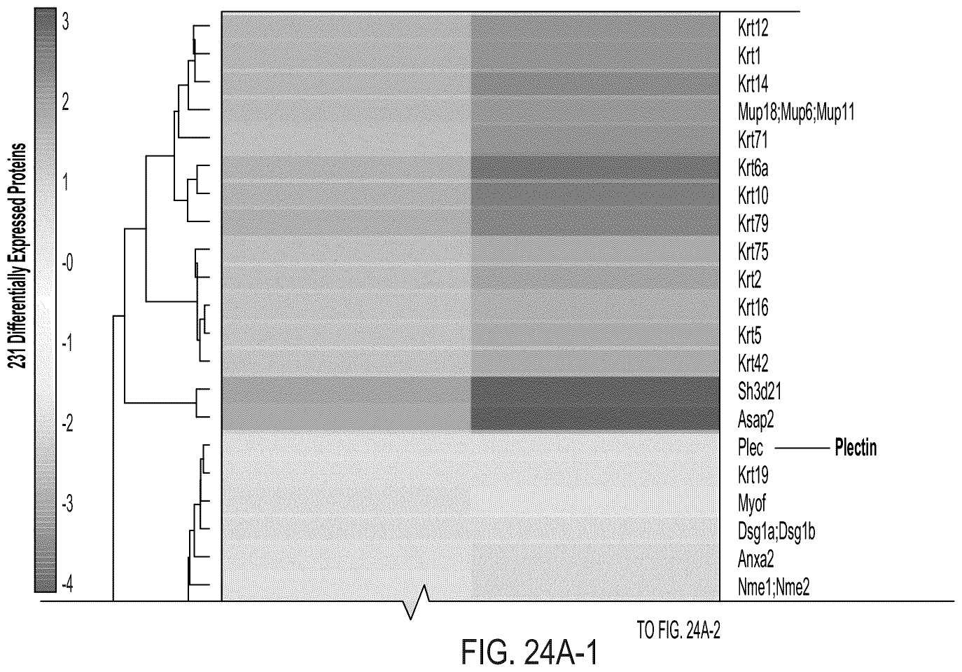

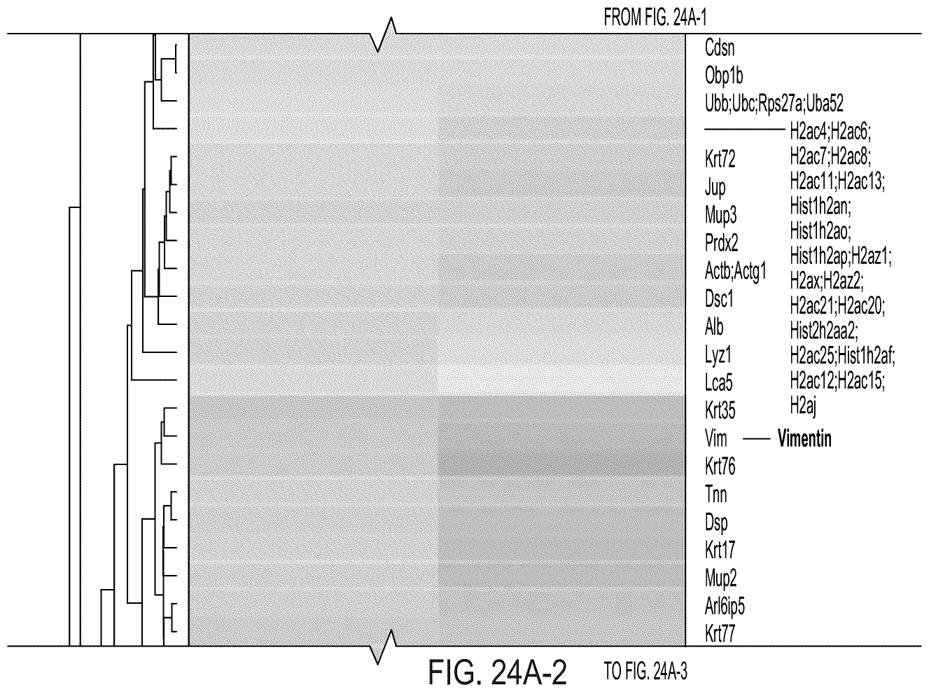

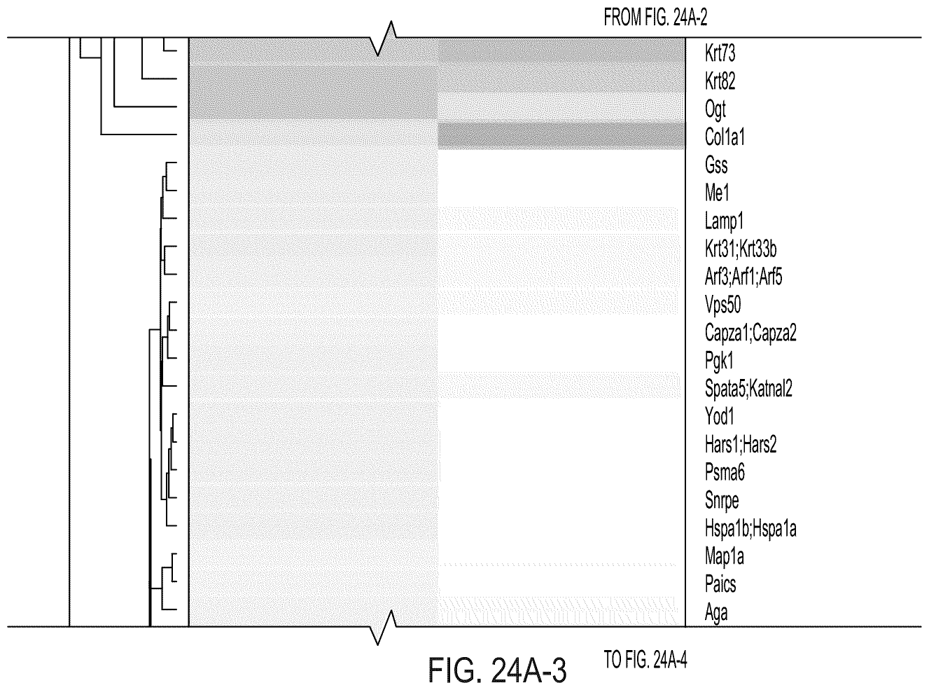

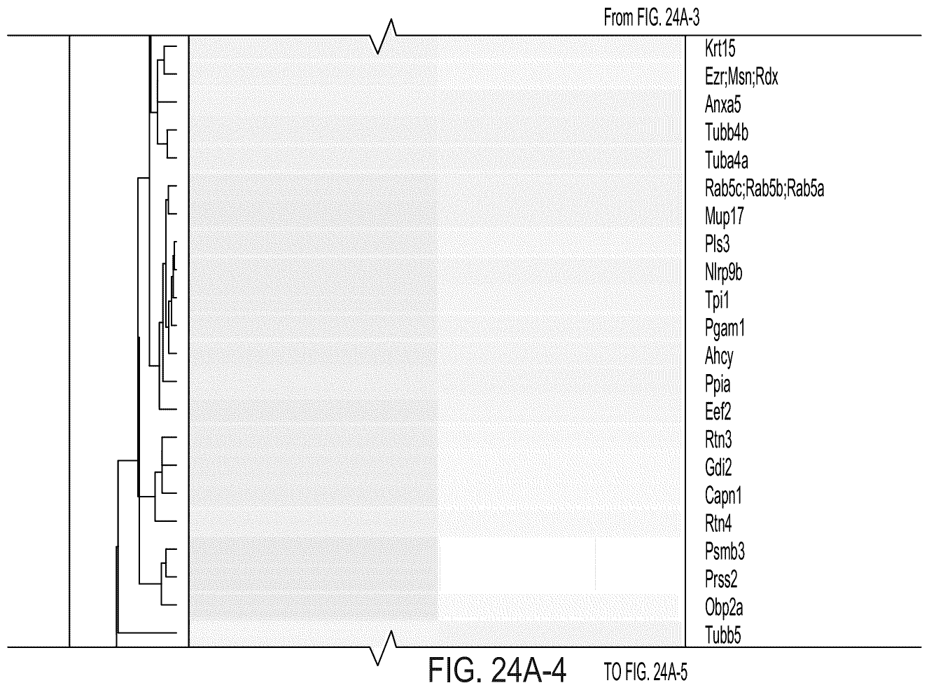

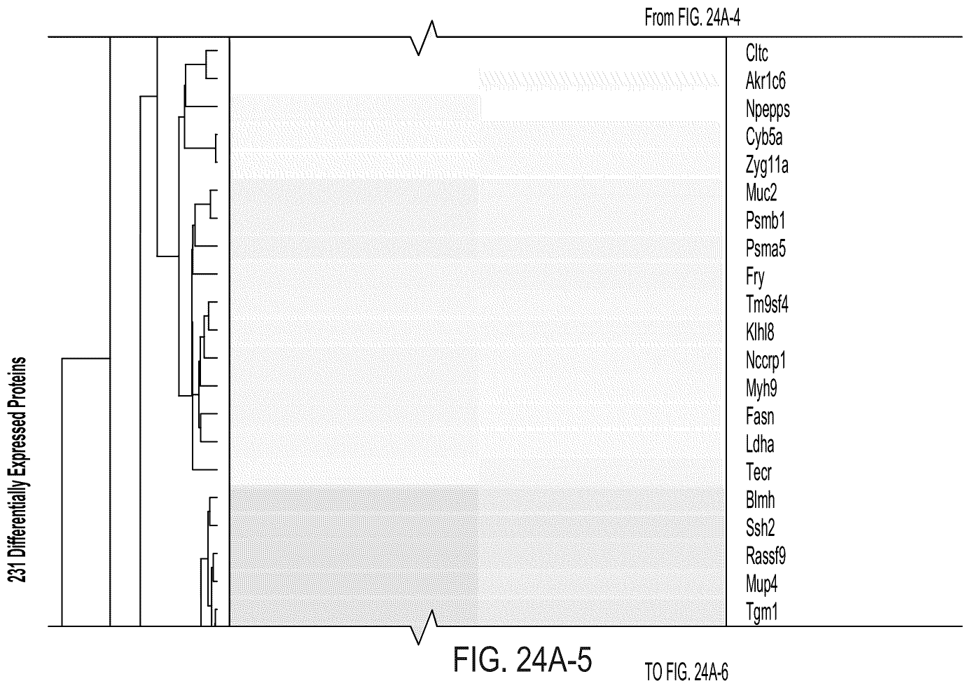

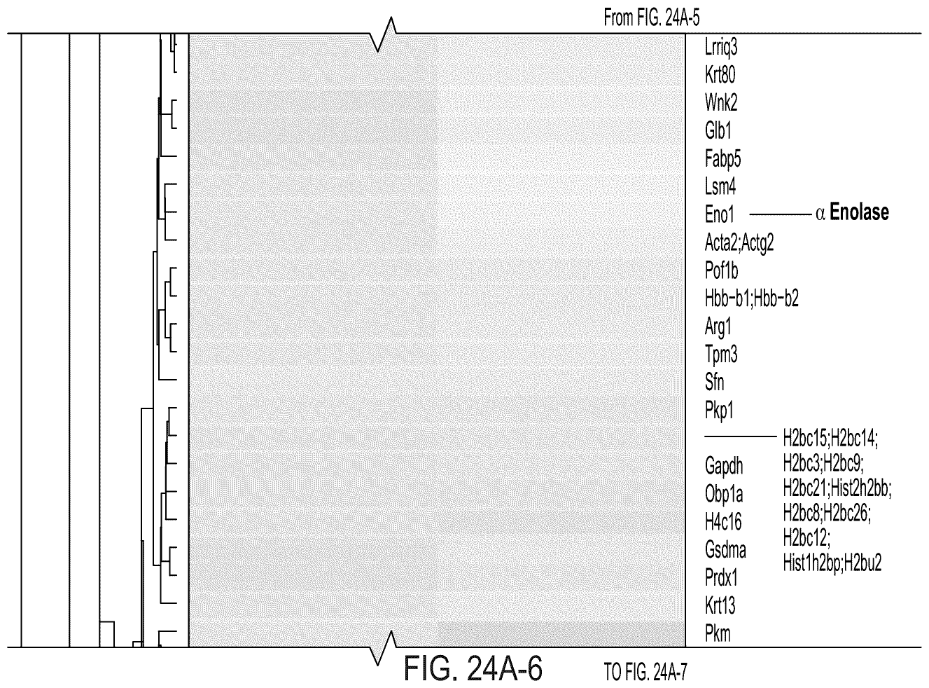

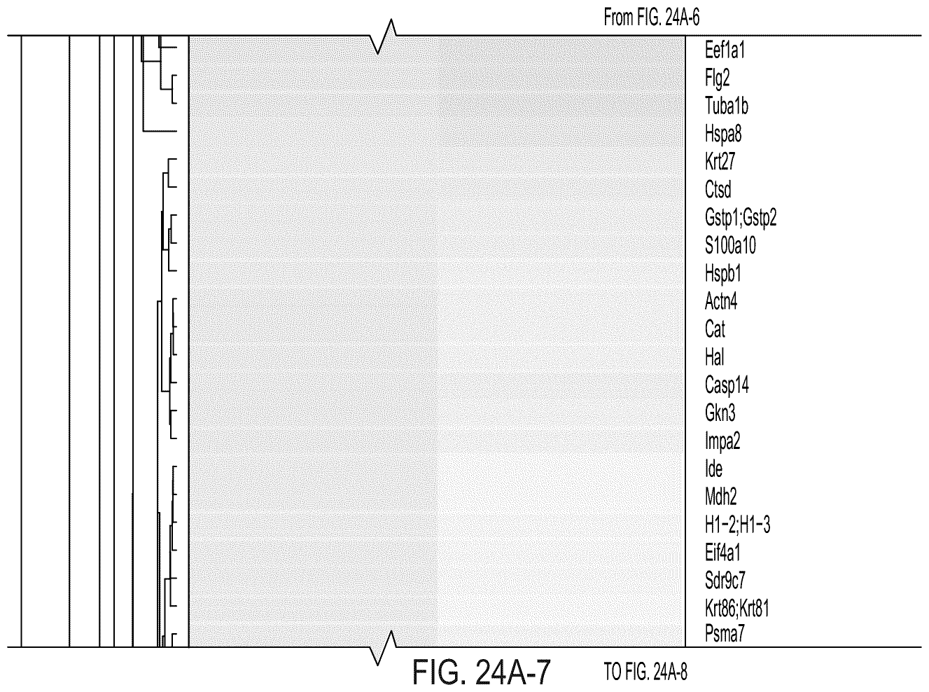

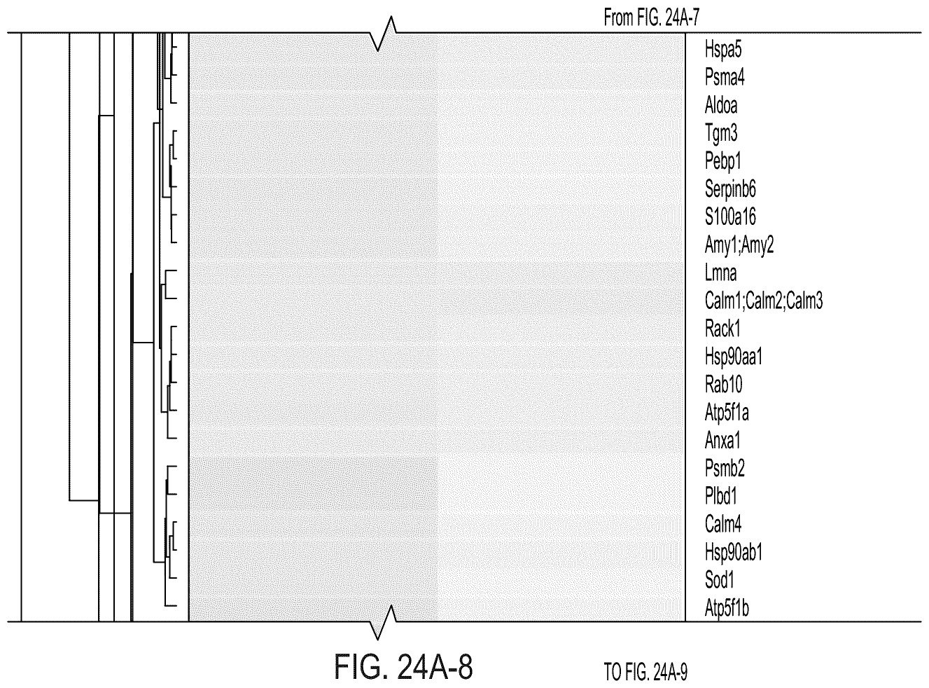

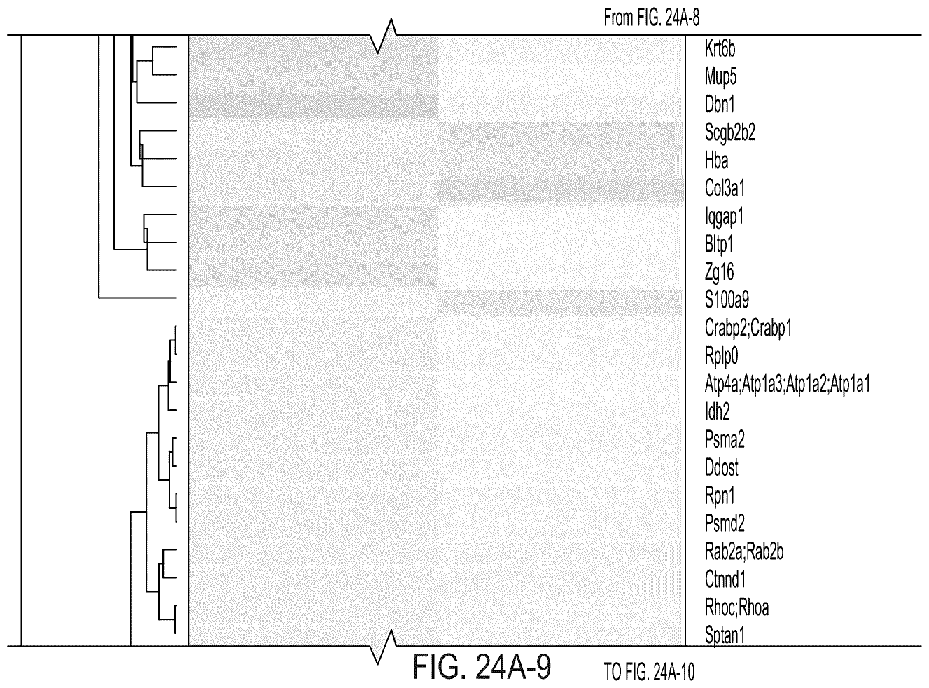

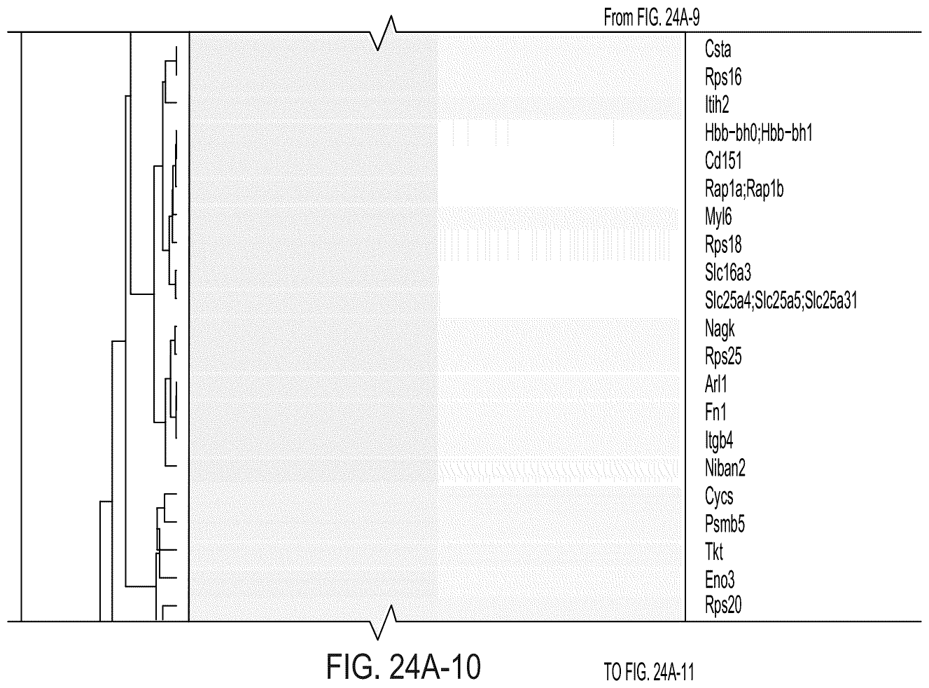

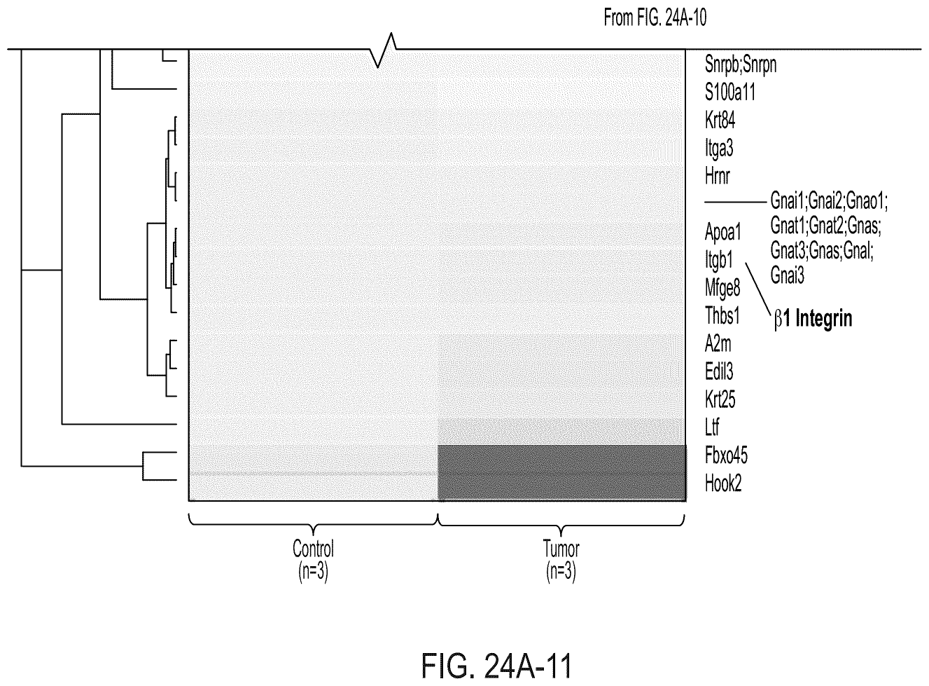

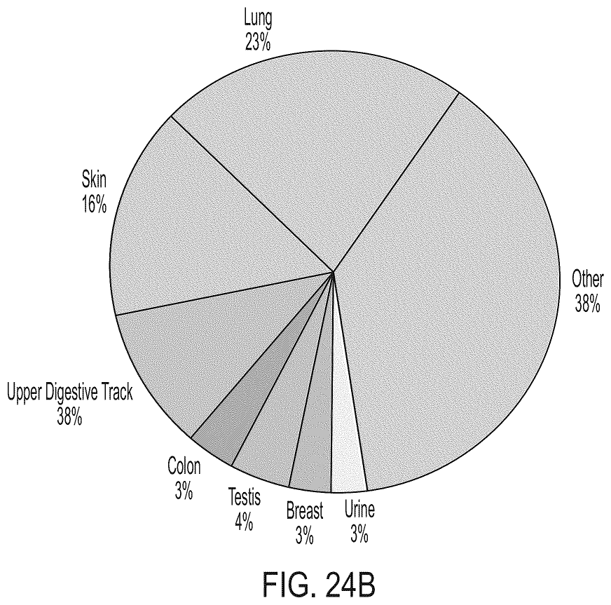

Using our EV proteomic analytical approach, we investigated the protein content of EBC collected with our v2.0 system for nose/mouth EBC collection from single animals. These analyses identified a total of 231 detectable proteins, of which we selected the top 60 most differentially expressed between control and lung tumor-bearing animals (FIGS. 24A-1 to 24A-11). Interestingly, we determined that several of the metastatic associated proteins identified from EBC collected with our v1.0 system were also present in EBC collected with our v2.0 system, but with greater differential expression (Vimentin, α-enolase, integrin B1, Plectin). Furthermore, when we analyzed the most likely tissue distribution/origin of these proteins using ProteinAtlas, we determined that our v2.0 collection device enabled the collection of EBC with a higher proportion of lung proteins (FIG. 24B). These analyses further illustrate the improved performance of our v2.0 system for the collection of mouse EBC samples. In addition, our results demonstrate the sensitivity of our proteomics workflow for the analysis of EBC proteins, and also revealed the potential of exhaled EV proteins for detection of lung tumors.

### Human Tumor Exhaled EV MiRNA Enrichment Pathway Analyses

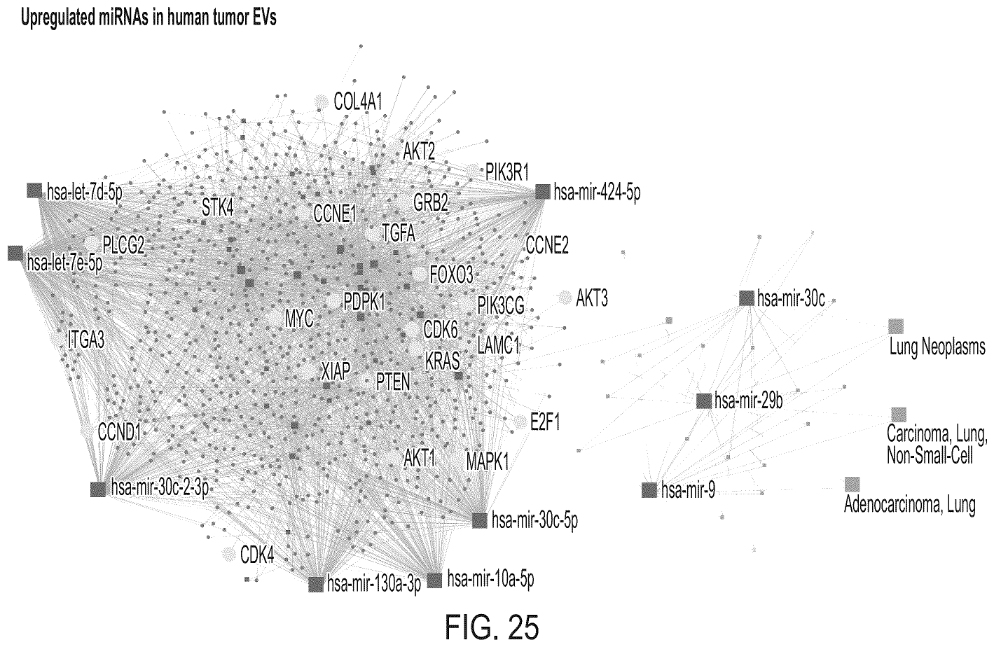

Considering that we identified miRNAs that were preferentially upregulated in whole EBC and in human-derived exhaled EVs selectively purified from EBC of individual animals using our v2.0 collection system, we sought to conduct in silico miRNA enrichment analyses on the top 33 miRNAs upregulated in human tumor EVs compared to mouse EVs. Using the web-based platform miRNET, we assessed any potential miRNA-gene interactions within biological processes of cancer development and progression that may be associated with the upregulation of these 33 miRNAs (FIG. 25). This broad proof-of-concept enrichment analysis revealed that miRNAs that were highly upregulated in human tumor EVs (i.e., hsa-miR-9, hsa-miR-10a, hsa-miR-29b, hsa-miR-30c hsa-miR-130a, hsa-miR-424, hsa-let-7d and hsa-let-7c) were associated with published regulatory roles of genes known to be involved in lung cancer, and more precisely, metastatic progression. Several of the putative miRNA-gene interactions involved degregulation of PI3K, AKTI and PTEN, which are known to be directly involved in the regulation of PI3K/AKT/mTOR dependent cell migration, invasion, and cell cycle regulation in lung cancer progression [109-112]. Additional interactions were identified for KRAS, MAPK, and FOXO, which have been shown to regulate cell survival during lung cancer development and progression [113-119]. Together, these results further supported the notion that miRNA cargoes from exhaled human tumor EVs purified from mouse EBC may play important roles in the progression of metastatic disease in the lungs.

### Discussion

In this proof-of-principle study we investigated how the collection and analysis of EBC from a lung tumor-bearing animal model may enable the non-invasive detection of secondary lung tumors. Because we and others have previously demonstrated that miRNAs can be detected in human exhaled breath [43-47] and that they hold both diagnostic and prognostic potential for the detection of lung cancer, we focused our analyses on the detection and quantification of exhaled miRNAs in relation to the presence of lung tumors using an orthotopic tumor-bearing mouse model of lung cancer.

As we sought to evaluate the potential of mouse EBC for detection of exhaled lung tumor miRNAs, we fine-tuned the design of two different mouse EBC collection systems: one that allowed for the collection of EBC from free-roaming animals (v1.0 system), and one that enabled direct nose and mouth collection of EBC from restrained animals (v2.0 system). Although our results demonstrate that the unrestrained v1.0 system does not allow for the collection of pure EBC from animals (because our proteomic data suggested the presence of urine, skin, and other contaminants), the collection chamber accommodated two animals and allowed for identification of miRNAs and proteins that were of human lung-tumor origin (i.e., as confirmed with our second set of analyses). When using PCR to quantify two miRNAs (i.e., miR-222 and miR-210), which displayed high read counts by NGS analyses of whole EBC from lung tumor-bearing animals, we confirmed their increased expression when compared to controls, and validated their detectability by means of qPCR. We found that as early as one week post tail-vein injection of the highly metastatic LM-3475 cells, we could differentiate EBC of lung tumor-bearing from control animals by quantifying both miR-222 and miR-210 in female mice. Our results suggest that the collection of EBC from unrestrained mice, contained in an enclosed chamber under regulated air flow, allowed for the capture of miRNAs that allowed detection of lung tumors. Despite our ability to detect differences in miRNA profiles of EBC collected from lung tumor-bearing and control animals, our proteomic analyses of the EBC samples determined that a large number of the differentially expressed proteins were likely to be predominantly associated with tissue/organ origins outside that of the respiratory system (i.e., skin, urogenital (i.e., urine), reproductive (i.e., testis and breast), lower (i.e., the colon) and upper digestive tracts (i.e., esophagus and mouth)), which precluded the determination that the miRNAs detected as differentially expressed were solely from lung tumor origin. Although the LM-3475 cell line was established in order to allow for the enhanced colonization of these cells in the lungs, their inoculation into the circulatory system (i.e., tail-vein injection) it is likely that some of these metastatic cells could still colonize and develop tumor foci in other organs [101,119,120]. Despite these limitations, our v1.0 system still allowed for the collection of important biological information contained within collected EBC during the progression of the lung tumors and without animal restraint, which may not be possible when studying animals with advanced lung cancer, or lung disease/lung injuries that would cause difficultly with restraint or shallow breathing. However, in an attempt to minimize the representation of non-respiratory system tumor profiles, we improved the design of our EBC collection system to enhance the nose and mouth only collection from individually immobilized animals (i.e., v2.0 system). We found that although the use our individually restrained mouse system decreased the volume of EBC collected (i.e., from ˜63 μl for unrestrained animal pairs/hour to ˜29 μl for restrained animal/2 hours), our analyses unequivocally demonstrated that lung tumor burden could be detected by the analysis of miRNAs present in condensed exhaled breath of lung tumor-bearing mice. Finally, we found that there was a significant overlap in the identity and expression of the miRNAs and proteins detectable in EBC from both collection systems, which further validated the utility of our v1.0 system.

Considering that the highly metastatic LM-3475 cell line was human in origin, we sought to determine if we could specifically isolate tumor (human) from normal (mouse) lung miRNA signals contained in EBC samples of lung tumor-bearing animals. Since several human studies have found that EBC contains exhaled extracellular vesicles (EVs) [121, 122], we chose to investigate both the human tumor-derived and mouse-derived EV content of mouse EBC samples. Our nanoparticle distribution analyses revealed that while we were able to detect a low number of nanoparticles in the size range of EVs (65-150 nm) in EBC collected from control animals, these nanoparticles were in a significantly higher number in the EBC of lung tumor-bearing animals. Furthermore, we found that the number of these nanoparticles increased over time as disease burden progressed. When using TEM, we confirmed that EBC collected from both control and lung-tumor bearing animals contained EVs. Additionally, when using the ONi super resolution microscopy platform with their commercially available human EV-profiler (CD63/CD9/CD81) kit, we confirmed that mouse EBC collected from lung tumor-bearing animals contained EVs that are of human and thus tumor origin. While we were able to detect EVs in the EBC collected from our mouse models, significantly large volumes of EBC combined with ultracentrifugation to concentrate EVs are required to overcome the sensitivities of these traditional EV characterization techniques. Collectively, these EV characterization results strongly suggest that metastatic tumor cells proliferating in the lungs released EVs that became aerosolized during normal murine tidal respiration, and these EVs could be non-invasively captured through the condensation of exhaled breath in the form of EBC. Furthermore, when using our EV-CATCHER assay customized with an anti-human anti-CD63 antibody and conducting miRNA NGS analyses of the purified EVs, we determined that we could specifically capture human EVs from mouse EBC and that the bulk of the miRNA signal detectable in whole mouse EBC was of human lung tumor origin. Furthermore, using miRNet to perform crude miRNA-target pathway enrichment analyses of the top upregulated miRNAs found in EBC of lung tumor-bearing by comparison to control animals, we found that they have known published interactions with genes involved in several pathways that regulate cancer cell proliferation and survival, cellular migration, and metastatic dissemination, which further supports our data suggesting that EV-miRNAs isolated from mouse EBC were of tumor origin.

Our findings are highly relevant as the lung represents a central organ for the circulation of blood, the exchange of oxygen/carbon dioxide, and is an opportunistic site for metastatic colonization of circulating tumor cells (CTCs), which can originate from several primary tumor organ sites that include breast, colorectal, head-and-neck, urogenital, gynecological and lymphatic cancers [123-128]. Considering that these biologically different cell types have the potential to colonize the lung, the analysis of EBC from lung tumor-bearing animal models established with metastatic cells originating from these different tissue types may help identify common metastatic miRNAs (i.e., in both our mouse EBC datasets the miR-200 metastatic cluster was detected) as well as potential primary tumor site-specific miRNAs, which may help with the development of molecular assays for early non-invasive detection of metastatic disease in the lungs. Our qPCR data using mouse EBC collected with both systems indicated that metastatic EV miRNAs are detectable within 1 to 2 weeks upon tail-vein injection of the highly metastatic LM-3475 cell line in mice, while in vivo bioluminescent imaging of tumor cells was only capable of detecting tumor burden after 6 weeks.

In conclusion, to our knowledge this study is the first of its kind, as we demonstrated that exhaled lung tumor-derived EVs and EV miRNAs can be purified and quantified from EBC of lung tumor-bearing animal models and enable the non-invasive exhaled breath detection of secondary lung cancer.

### REFERENCES

1. Sung H, Ferlay J, Siegel R L, Laversanne M, Soerjomataram I, Jemal A, Bray F. Global Cancer Statistics 2020: GLOBOCAN Estimatesof Incidence and Mortality World-wide for 36 Cancers in 185 Countries. CA Cancer J Clin. 2021; 71 (3): 209-249. PMID: 33538338.
2. Siegel R L, Miller K D, Fuchs H E, Jemal A. Cancer statistics, 2021. CA Cancer J Clin. 2021; 71 (1): 7-33. PMID: 33433946.
3. Stella G M, Kolling S, Benvenuti S, Bortolotto C. Lung-Seeking Metastases. Cancers (Basel). 2019 Jul. 19; 11 (7): 1010. doi: 10.3390/cancers11071010. PMID: 31330946; PMCID: PMC6678078.
4. Caparica R, Mak M P, Rocha C H, Velho P H I, Viana P, Moura M R L, Menezes M R, Amato M B P, Feher O. Pulmonary Nodules in Patients With Non-pulmonary Cancer: Not Always Metastases. J Glob Oncol. 2016; 2 (3): 138-144. PMID: 34616754.
5. Chen H, Stoltzfus K C, Lehrer E J, Horn S R, Siva S, Trifiletti D M, Meng M B, Verma V, Louie A V, Zaorsky N G. The epidemiology of lung metastases. Front Med (Lausanne). 2021; 8:723396. PMID: 34616754.
6 Ha D, Choi H, Chevalier C, Zell K, Wang X F, Mazzone P J. Survival in patients with metachronous second primary lung cancer. Ann Am Thorac Soc. 2015; 12 (1): 79-84. PMID: 25494009.
7. Jamil A. and Kasi A. Lung Metastasis. Stat Pearls Publishing. 2022, NCBI bookshelf ID NBK553111. PMID: 31971751.
8. Wang R, Yin Z, Liu L, Gao W, Li W, Shu Y, Xu J. Second Primary Lung Cancer After Breast Cancer: A Population-Based Study of 6,269 Women. Front Oncol. 2018; 8:427. PMID: 30356721.
9. Medeiros B, Allan A L. Molecular mechanisms of breast cancer metastasis to the lung: clinical and experimental perspectives. Int J Mol Sci. 2019; 20:2272. PMID: 31071959.
10. Mangiameli G, Cioffi U, Alloisio M, Testori A. Lung Metastases: Current Surgical Indications and New Perspectives. Front Surg. 2022; 9:884915. PMID: 35574534.
11. Antonoff M B, Sofocleous C T, Callstrom M R, Nguyen Q N. The roles of surgery, stereotactic radiation, and ablation for treatment of pulmonary metastases. J Thorac Cardiovasc Surg. 2022; 163 (2): 495-502. PMID: 33838914.
12. Dama E, Colangelo T, Fina E, Cremonesi M, Kallikourdis M, Veronesi G, Bianchi F. Biomarkers and lung cancer early detection: State-of-the-art. Cancers (Basel). 2021; 13 (15): 3919. PMID: 34359818.
13. Haranguş A, Berindan-Neagoe I, Todea D A, Şimon I, Şimon M. Noncoding RNAs and Liquid Biopsy in Lung Cancer: A Literature Review. Diagnostics (Basel). 2019; 9 (4): 216. PMID: 31818027.
14. De Fraipont F, Gazzeri S, Cho W C, Eymin B. Circular RNAs and RNA Splice Variants as Biomarkers for Prognosis and Therapeutic Response in the Liquid Biopsies of Lung Cancer Patients. Front Genet. 2019; 10:390. PMID: 31134126.
15. Zhong S, Golpon H, Zardo P, Borlak J. A systematic review identifies predictive and prognostic miRNA candidates for precision medicine in lung cancer. Transl Res. 2021; 230:164-196. PMID: 33253979.
16. Patel U, Susman D, Allan A L. Influence of Extracellular Vesicles on Lung Stromal Cells during Breast Cancer Metastasis. Int J Mol Sci. 2023 Jul. 22; 24 (14): 11801. doi: 10.3390/ijms241411801. PMID: 37511559; PMCID: PMC10380344.
17. Hinestrosa J P, Kurzrock R, Lewis J M, Schork N J, Schroeder G, Kamat A M, Lowy A M, Eskander R N, Perrera O, Searson D, Rastegar K, Hughes J R, Ortiz V, Clark I, Balcer H I, Arakelyan L, Turner R, Billings P R, Adler M J, Lippman S M, Krishnan R. Early-stage multi-cancer detection using an extracellular vesicle protein-based blood test. Commun Med (Lond). 2022 Mar. 17; 2:29. doi: 10.1038/s43856-022-00088-6. PMID: 35603292; PMCID: PMC9053211.
18. Liu Z, Kong Y, Dang Q, Weng S, Zheng Y, Ren Y, Lv J, Li N, Han Y, Han X. Liquid Biopsy in Pre-Metastatic Niche: From Molecular Mechanism to Clinical Application. Front Immunol. 2022 Jul. 15; 13:958360. doi: 10.3389/fimmu.2022.958360. PMID: 35911705; PMCID: PMC9334814.
19. Habli Z, AlChamaa W, Saab R, Kadara H, Khraiche M L. Circulating Tumor Cell Detection Technologies and Clinical Utility: Challenges and Opportunities. Cancers (Basel). 2020 Jul. 17; 12 (7): 1930. doi: 10.3390/cancers12071930. PMID: 32708837; PMCID: PMC7409125.
20. Agashe R, Kurzrock R. Circulating Tumor Cells: From the Laboratory to the Cancer Clinic. Cancers (Basel). 2020 Aug. 21; 12 (9): 2361. doi: 10.3390/cancers12092361. PMID: 32825548; PMCID: PMC7564158.
21. Pantel K, Alix-Panabières C. Circulating tumour cells in cancer patients: challenges and perspectives. Trends Mol Med. 2010 September; 16 (9): 398-406. doi: 10.1016/j.molmed.2010.07.001. Epub 2010 Jul. 29. PMID: 20667783.
22. Ricthdorf S, Fritsche H, Müller V, Rau T, Schindlbeck C, Rack B, Janni W, Coith C, Beck K, Janicke F, Jackson S, Gornet T, Cristofanilli M, Pantel K. Detection of circulating tumor cells in peripheral blood of patients with metastatic breast cancer: a validation study of the CellSearch system. Clin Cancer Res. 2007 Feb. 1; 13 (3): 920-8. doi: 10.1158/1078-0432.CCR-06-1695. PMID: 17289886.
23. Scijo L M, Peled N, Ajona D, Bocri M, Field J K, Sozzi G, Pio R, Zulueta J J, Spira A, Massion P P, Mazzone P J, Montuenga L M. Biomarkers in Lung Cancer Screening: Achievements, Promises, and Challenges. J Thorac Oncol. 2019 March; 14 (3): 343-357. doi: 10.1016/j.jtho.2018.11.023. Epub 2018 Dec. 4. PMID: 30529598; PMCID: PMC6494979.
24. Yendamuri S, Vaporciyan A A, Zaidi T, Feng L, Fernandez R, Bekele N B, Hofstetter W L, Jiang F, Mehran R J, Rice D C, Spitz M R, Swisher S G, Walsh G L, Roth J A, Katz R L. 3p22.1 and 10q22.3 deletions detected by fluorescence in situ hybridization (FISH): a potential new tool for early detection of non-small cell lung Cancer (NSCLC). J Thorac Oncol. 2008 September; 3 (9): 979-84. doi: 10.1097/JTO.0b013e3181834f3a. PMID: 18758299; PMCID: PMC3370669.
25. Chen C C, Bai C H, Lec K Y, Chou Y T, Pan S T, Wang Y H. Evaluation of the diagnostic accuracy of bronchial brushing cytology in lung cancer: A meta-analysis. Cancer Cytopathol. 2021 Scp; 129 (9): 739-749. doi: 10.1002/cncy.22436. Epub 2021 Apr. 22. PMID: 33886162.
26. Pavel A B, Campbell J D, Liu G, Elashoff D, Dubinett S, Smith K, Whitney D, Lenburg M E, Spira A; AEGIS Study Team. Alterations in Bronchial Airway miRNA Expression for Lung Cancer Detection. Cancer Prev Res (Phila). 2017 November; 10 (11): 651-659. doi: 10.1158/1940-6207.CAPR-17-0098. Epub 2017 Scp 6. PMID: 28877936; PMCID: PMC6758560.
27. Matthiesen R. MS-Based Biomarker Discovery in Bronchoalveolar Lavage Fluid for Lung Cancer. Proteomics Clin Appl. 2020 January; 14 (1):e1900077. doi: 10.1002/prca.201900077. Epub 2019 Nov. 12. PMID: 31631581.
28. Engel E, Schmidt B, Carstensen T, Weickmann S, Jandrig B, Witt C, Fleischhacker M. Detection of tumor-specific mRNA in cell-free bronchial lavage supernatant in patients with lung cancer. Ann N Y Acad Sci. 2004 June; 1022:140-6. doi: 10.1196/annals.1318.023. PMID: 15251953.
29. Anglim P P, Alonzo T A, Laird-Offringa I A. DNA methylation-based biomarkers for early detection of non-small cell lung cancer: an update. Mol Cancer. 2008 Oct. 23; 7:81. doi: 10.1186/1476-4598-7-81. PMID: 18947422; PMCID: PMC2585582.
30. Schmidt B, Rehbein G, Fleischhacker M. Liquid Profiling in Lung Cancer-Quantification of Extracellular miRNAs in Bronchial Lavage. Adv Exp Med Biol. 2016; 924:33-37. doi: 10.1007/978-3-319-42044-8_7. PMID: 27753015.
31. Mutlu G M, Garey K W, Robbins R A, Danziger L H, Rubinstein I. Collection and analysis of exhaled breath condensate in humans. Am J Respir Crit Care Med. 2001 Sep. 1; 164 (5): 731-7. doi: 10.1164/ajrccm.164.5.2101032. PMID: 11549524.
32 Keogh R J, Riches J C. The Use of Breath Analysis in the Management of Lung Cancer: Is It Ready for Primetime? Curr Oncol. 2022 Sep. 30; 29 (10): 7355-7378. doi: 10.3390/curroncol29100578. PMID: 36290855; PMCID: PMC9600994.
33. Wang P, Huang Q, Meng S, Mu T, Liu Z, He M, Li Q, Zhao S, Wang S, Qiu M. Identification of lung cancer breath biomarkers based on perioperative breathomics testing: A prospective observational study. EClinicalMedicine. 2022 Apr. 16; 47:101384. doi: 10.1016/j.eclinm.2022.101384. PMID: 35480076; PMCID: PMC9035731.
34. van der Schee M, Pinheiro H, Gaude E. Breath biopsy for early detection and precision medicine in cancer. Ecancermedicalscience. 2018 Jul. 19; 12:ed84. doi: 10.3332/ecancer.2018.cd84. PMID: 30093919; PMCID: PMC6070367.
35 Marzorati D, Mainardi L, Sedda G, Gasparri R, Spaggiari L, Cerveri P. A review of exhaled breath: a key role in lung cancer diagnosis. J Breath Res. 2019 Apr. 1; 13 (3): 034001. doi: 10.1088/1752-7163/ab0684. PMID: 30754033.
36 Jia Z, Patra A, Kutty V K, Venkatesan T. Critical Review of Volatile Organic Compound Analysis in Breath and In Vitro Cell Culture for Detection of Lung Cancer. Metabolites. 2019 Mar. 18; 9 (3): 52. doi: 10.3390/mctabo9030052. PMID: 30889835; PMCID: PMC6468373.
37. Kubáň P, Foret F. Exhaled breath condensate: determination of non-volatile compounds and their potential for clinical diagnosis and monitoring. A review. Anal Chim Acta. 2013 Dec. 17; 805:1-18. doi: 10.1016/j.aca.2013.07.049. Epub 2013 Jul. 31. PMID: 24296139.
38. Rahimpour E, Khoubnasabjafari M, Jouyban-Gharamaleki V, Jouyban A. Non-volatile compounds in exhaled breath condensate: review of methodological aspects. Anal Bioanal Chem. 2018 October; 410 (25): 6411-6440. doi: 10.1007/s00216-018-1259-4. Epub 2018 Jul. 25. PMID: 30046867.
39. Campanella A, De Summa S, Tommasi S. Exhaled breath condensate biomarkers for lung cancer. J Breath Res. 2019 Aug. 20; 13 (4): 044002. doi: 10.1088/1752-7163/ab2f9f. PMID: 31282387.
40. Effros R M, Peterson B, Casaburi R, Su J, Dunning M, Torday J, Biller J, Shaker R. Epithelial lining fluid solute concentrations in chronic obstructive lung disease patients and normal subjects. J Appl Physiol (1985). 2005 October; 99 (4): 1286-92. doi: 10.1152/japplphysiol.00362.2005. Epub 2005 May 26. PMID: 15920100.
41. Horváth I, Hunt J, Barnes P J, Alving K, Antczak A, Baraldi E, Becher G, van Beurden W J, Corradi M, Dekhuijzen R, Dweik R A, Dwyer T, Effros R, Erzurum S, Gaston B, Gessner C, Greening A, Ho L P, Hohlfeld J, Jobsis Q, Laskowski D, Loukides S, Marlin D, Montuschi P, Olin A C, Redington A E, Reinhold P, van Rensen E L, Rubinstein I, Silkoff P, Toren K, Vass G, Vogelberg C, Wirtz H; ATS/ERS Task Force on Exhaled Breath Condensate. Exhaled breath condensate: methodological recommendations and unresolved questions. Eur Respir J. 2005 September; 26 (3): 523-48. doi: 10.1183/09031936.05.00029705. PMID: 16135737.
42. Kuo T C, Tan C E, Wang S Y, Lin O A, Su B H, Hsu M T, Lin J, Cheng Y Y, Chen C S, Yang Y C, Chen K H, Lin S W, Ho C C, Kuo C H, Tseng Y J. Human Breathomics Database. Database (Oxford). 2020 Jan. 1; 2020:baz139. doi: 10.1093/database/baz139. PMID: 31976536; PMCID: PMC6978997.
43. Shi M, Han W, Loudig O, Shah C D, Dobkin J B, Keller S, Sadoughi A, Zhu C, Siegel R E, Fernandez M K, DeLaRosa L, Patel D, Desai A, Siddiqui T, Gombar S, Suh Y, Wang T, Hosgood H D, Pradhan K, Ye K, Spivack S D. Initial development and testing of an exhaled microRNA detection strategy for lung cancer case-control discrimination. Sci Rep. 2023 Apr. 24; 13 (1): 6620. doi: 10.1038/s41598-023-33698-8. PMID: 37095155; PMCID: PMC10126132.
44. Pinkerton M, Chinchilli V, Banta E, Craig T, August A, Bascom R, Cantorna M, Harvill E, Ishmael F T. Differential expression of microRNAs in exhaled breath condensates of patients with asthma, patients with chronic obstructive pulmonary disease, and healthy adults. J Allergy Clin Immunol. 2013 July; 132 (1): 217-9. doi: 10.1016/j.jaci.2013.03.006. Epub 2013 Apr. 28. PMID: 23628339.
45. Pérez-Sánchez C, Barbarroja N, Pantaleão LC, López-Sánchez LM, Ozanne S E, Jurado-Gámez B, Aranda E, Lopez-Pedrera C, Rodríguez-Ariza A. Clinical Utility of microRNAs in Exhaled Breath Condensate as Biomarkers for Lung Cancer. J Pers Med. 2021 Feb. 9; 11 (2): 111. doi: 10.3390/jpm11020111. PMID: 33572343; PMCID: PMC7916163.
46. Mendes F C, Paciência I, Ferreira A C, Martins C, Rufo J C, Silva D, Cunha P, Farraia M, Moreira P, Delgado L, Soares M L, Moreira A. Development and validation of exhaled breath condensate microRNAs to identify and endotype asthma in children. PLOS Onc. 2019 Nov. 8; 14 (11):e0224983. doi: 10.1371/journal.pone.0224983. PMID: 31703106; PMCID: PMC6839869.
47. Faversani A, Favero C, Dioni L, Pesatori A C, Bollati V, Montoli M, Musso V, Terrasi A, Fusco N, Nosotti M, Vaira V, Palleschi A. An EBC/Plasma miRNA Signature Discriminates Lung Adenocarcinomas From Pleural Mesothelioma and Healthy Controls. Front Oncol. 2021 Jun. 15; 11:643280. doi: 10.3389/fonc.2021.643280. PMID: 34211838; PMCID: PMC8239300.
48. Lucchetti D, Santini G, Perelli L, Ricciardi-Tenore C, Colella F, Mores N, Macis G, Bush A, Sgambato A, Montuschi P. Detection and characterisation of extracellular vesicles in exhaled breath condensate and sputum of COPD and severe asthma patients. Eur Respir J. 2021 Aug. 5; 58 (2): 2003024. doi: 10.1183/13993003.03024-2020. PMID: 33795323.
49. Dobhal G, Datta A, Ayupova D, Teesdale-Spittle P, Gorcham R V. Isolation, characterisation and detection of breath-derived extracellular vesicles. Sci Rep. 2020 Oct. 15; 10 (1): 17381. doi: 10.1038/s41598-020-73243-5. PMID: 33060613; PMCID: PMC7566616.
50. Sinha A, Yadav A K, Chakraborty S, Kabra S K, Lodha R, Kumar M, Kulshreshtha A, Scthi T, Pandey R, Malik G, Laddha S, Mukhopadhyay A, Dash D, Ghosh B, Agrawal A. Exosome-enclosed microRNAs in exhaled breath hold potential for biomarker discovery in patients with pulmonary diseases. J Allergy Clin Immunol. 2013 July; 132 (1): 219-22. doi: 10.1016/j.jaci.2013.03.035. Epub 2013 May 14. PMID: 23683467.
51. O'Brien J, Hayder H, Zayed Y, Peng C. Overview of MicroRNA Biogenesis, Mechanisms of Actions, and Circulation. Front Endocrinol (Lausanne). 2018 Aug. 3; 9:402. doi: 10.3389/fendo.2018.00402. PMID: 30123182; PMCID: PMC6085463.
52. Ketting R F. microRNA Biogenesis and Function: An overview. Adv Exp Med Biol. 2011; 700:1-14. doi: 10.1007/978-1-4419-7823-3_1. PMID: 21755468.
53. Ardekani A M, Nacini M M. The Role of MicroRNAs in Human Diseases. Avicenna J Med Biotechnol. 2010 October; 2 (4): 161-79. PMID: 23407304; PMCID: PMC3558168.
54. Acunzo M, Romano G, Wernicke D, Croce C M. MicroRNA and cancer—a brief overview. Adv Biol Regul. 2015 January; 57:1-9. doi: 10.1016/j.jbior.2014.09.013. Epub 2014 Sep. 28. Erratum in: Adv Biol Regul. 2015 May; 58:53. PMID: 25294678.
55. Santos R M, Moreno C, Zhang W C. Non-Coding RNAs in Lung Tumor Initiation and Progression. Int J Mol Sci. 2020 Apr. 16; 21 (8): 2774. doi: 10.3390/ijms21082774. PMID: 32316322; PMCID: PMC7215285.
56 Rohan T E, Wang T, Weinmann S, Wang Y, Lin J, Ginsberg M, Loudig O. A miRNA Expression Signature in Breast Tumor Tissue Is Associated with Risk of Distant Metastasis. Cancer Res. 2019 Apr. 1; 79 (7): 1705-1713. doi: 10.1158/0008-5472.CAN-18-2779. Epub 2019 Feb. 13. PMID: 30760517.
57. Yanaihara N, Caplen N, Bowman E, Seike M, Kumamoto K, Yi M, Stephens R M, Okamoto A, Yokota J, Tanaka T, Calin G A, Liu C G, Croce C M, Harris C C. Unique microRNA molecular profiles in lung cancer diagnosis and prognosis. Cancer Cell. 2006 March; 9 (3): 189-98. doi: 10.1016/j.ccr.2006.01.025. PMID: 16530703.
58. Zhu X, Kudo M, Huang X, Sui H, Tian H, Croce C M, Cui R. Frontiers of MicroRNA Signature in Non-small Cell Lung Cancer. Front Cell Dev Biol. 2021 Apr. 7; 9:643942. doi: 10.3389/fcell.2021.643942. PMID: 33898432; PMCID: PMC8058364.
59. Chakrabortty A, Patton D J, Smith B F, Agarwal P. miRNAs: Potential as Biomarkers and Therapeutic Targets for Cancer. Genes (Basel). 2023 Jun. 29; 14 (7): 1375. doi: 10.3390/genes 14071375. PMID: 37510280; PMCID: PMC10378777.
60. Warc A P, Kabekkodu S P, Chawla A, Paul B, Satyamoorthy K. Diagnostic and prognostic potential clustered miRNAs in bladder cancer. 3 Biotech. 2022 August; 12 (8): 173. doi: 10.1007/s13205-022-03225-z. Epub 2022 Jul. 13. PMID: 35845108; PMCID: PMC9279521.
61. Quirico L, Orso F. The power of microRNAs as diagnostic and prognostic biomarkers in liquid biopsies. Cancer Drug Resist. 2020 Feb. 21; 3 (2): 117-139. doi: Mao S, Lu Z, Zheng S, Zhang H, Zhang G, Wang F, Huang J, Lei Y, Wang X, Liu C, Sun N, He J. Exosomal miR-141 promotes tumor angiogenesis via KLF12 in small cell lung cancer. J Exp Clin Cancer Res. 2020 Sep. 21; 39 (1): 193. doi: 10.1186/s13046-020-01680-1. PMID: 32958011; PMCID: PMC7504642.
62. Quirico L, Orso F. The power of microRNAs as diagnostic and prognostic biomarkers in liquid biopsies. Cancer Drug Resist. 2020 Feb. 21; 3 (2): 117-139. doi: 10.20517/cdr.2019.103. PMID: 35582611; PMCID: PMC9090592.
63. Théry C, Zitvogel L, Amigorena S. Exosomes: composition, biogenesis and function. Nat Rev Immunol. 2002 August; 2 (8): 569-79. doi: 10.1038/nri855. PMID: 12154376.
64. Pitt J M, Kroemer G, Zitvogel L. Extracellular vesicles: masters of intercellular communication and potential clinical interventions. J Clin Invest. 2016 Apr. 1; 126 (4): 1139-43. doi: 10.1172/JCI87316. Epub 2016 Apr. 1. PMID: 27035805; PMCID: PMC4811136.
65. Mitchell M I, Loudig O. Communicator Extraordinaire: Extracellular Vesicles in the Tumor Microenvironment Are Essential Local and Long-Distance Mediators of Cancer Metastasis. Biomedicines. 2023 Sep. 14; 11 (9): 2534. doi: 10.3390/biomedicines 11092534. PMID: 37760975; PMCID: PMC10526527.
66. Brena D, Huang M B, Bond V. Extracellular vesicle-mediated transport: Reprogramming a tumor microenvironment conducive with breast cancer progression and metastasis. Transl Oncol. 2022 January; 15 (1): 101286. doi: 10.1016/j.tranon.2021.101286. Epub 2021 Nov. 25. PMID: 34839106; PMCID: PMC8636863.
67. Ozawa P M M, Alkhilaiwi F, Cavalli I J, Malheiros D, de Souza Fonseca Ribeiro E M, Cavalli L R. Extracellular vesicles from triple-negative breast cancer cells promote proliferation and drug resistance in non-tumorigenic breast cells. Breast Cancer Res Treat. 2018 Dcc; 172 (3): 713-723. doi: 10.1007/s10549-018-4925-5. Epub 2018 Sep. 1. PMID: 30173296; PMCID: PMC6245099.
68. He S, Li Z, Yu Y, Zeng Q, Cheng Y, Ji W, Xia W, Lu S. Exosomal miR-499a-5p promotes cell proliferation, migration and EMT via mTOR signaling pathway in lung adenocarcinoma. Exp Cell Res. 2019 Jun. 15; 379 (2): 203-213. doi: 10.1016/j.yexcr.2019.03.035. Epub 2019 Apr. 10. PMID: 30978341.
69. Yang M, Xiao R, Wang X, Xiong Y, Duan Z, Li D, Kan Q. MiR-93-5p regulates tumorigenesis and tumor immunity by targeting PD-L1/CCND1 in breast cancer. Ann Transl Med. 2022 February; 10 (4): 203. doi: 10.21037/atm-22-97. PMID: 35280383; PMCID: PMC8908185.
70. Fabbri M, Paone A, Calore F, Galli R, Gaudio E, Santhanam R, Lovat F, Fadda P, Mao C, Nuovo G J, Zanesi N, Crawford M, Ozer G H, Wernicke D, Alder H, Caligiuri M A, Nana-Sinkam P, Perrotti D, Croce C M. MicroRNAs bind to Toll-like receptors to induce prometastatic inflammatory response. Proc Natl Acad Sci USA. 2012 Jul. 31; 109 (31):E2110-6. doi: 10.1073/pnas. 1209414109. Epub 2012 Jul. 2. PMID: 22753494; PMCID: PMC3412003.
71. Wong G L, Abu Jalboush S, Lo H W. Exosomal MicroRNAs and Organotropism in Breast Cancer Metastasis. Cancers (Basel). 2020 Jul. 7; 12 (7): 1827. doi: 10.3390/cancers 12071827. PMID: 32646059; PMCID: PMC7408921.
72. Zeng Z, Li Y, Pan Y, Lan X, Song F, Sun J, Zhou K, Liu X, Ren X, Wang F, Hu J, Zhu X, Yang W, Liao W, Li G, Ding Y, Liang L. Cancer-derived exosomal miR-25-3p promotes pre-metastatic niche formation by inducing vascular permeability and angiogenesis. Nat Commun. 2018 Dec. 19; 9 (1): 5395. doi: 10.1038/s41467-018-07810-w. PMID: 30568162; PMCID: PMC6300604.
73. Zhong Y, Ding X, Bian Y, Wang J, Zhou W, Wang X, Li P, Shen Y, Wang J J, Li J, Zhang C, Wang C. Discovery and validation of extracellular vesicle-associated miRNAs as noninvasive detection biomarkers for early-stage non-small-cell lung cancer. Mol Oncol. 2021 Scp; 15 (9): 2439-2452. doi: 10.1002/1878-0261.12889. Epub 2021 Jan. 6. PMID: 33340250; PMCID: PMC8410569.
74. Gao S, Guo W, Liu T, Liang N, Ma Q, Gao Y, Tan F, Xuc Q, He J. Plasma extracellular vesicle microRNA profiling and the identification of a diagnostic signature for stage I lung adenocarcinoma. Cancer Sci. 2022 February; 113 (2): 648-659. doi: 10.1111/cas. 15222. Epub 2021 Dec. 6. PMID: 34837453; PMCID: PMC8819331.
75. Wortzel I, Dror S, Kenific C M, Lyden D. Exosome-Mediated Metastasis: Communication from a Distance. Dev Cell. 2019 May 6; 49 (3): 347-360. doi: 10.1016/j.devcel.2019.04.011. PMID: 31063754.
76. Mitchell M I, Ma J, Carter C L, Loudig O. Circulating Exosome Cargoes Contain Functionally Diverse Cancer Biomarkers: From Biogenesis and Function to Purification and Potential Translational Utility. Cancers (Basel). 2022 Jul. 10; 14 (14): 3350. doi: 10.3390/cancers 14143350. PMID: 35884411; PMCID: PMC9318395.
77. Couch Y, Buzas E I, Di Vizio D, Gho Y S, Harrison P, Hill A F, Lötvall J, Raposo G, Stahl P D, Thery C, Witwer K W, Carter D R F. A brief history of nearly EV-erything—The rise and rise of extracellular vesicles. J Extracell Vesicles. 2021 December; 10 (14):e12144. doi: 10.1002/jev2.12144. PMID: 34919343; PMCID: PMC8681215.
78. Logozzi M, Angelini D F, Giuliani A, Mizzoni D, Di Raimo R, Maggi M, Gentilucci A, Marzio V, Salciccia S, Borsellino G, Battistini L, Sciarra A, Fais S. Increased Plasmatic Levels of PSA-Expressing Exosomes Distinguish Prostate Cancer Patients from Benign Prostatic Hyperplasia: A Prospective Study. Cancers (Basel). 2019 Sep. 27; 11 (10): 1449. doi: 10.3390/cancers11101449. PMID: 31569672; PMCID: PMC6826376.
79. Newman L A, Useckaite Z, Johnson J, Sorich M J, Hopkins A M, Rowland A. Selective Isolation of Liver-Derived Extracellular Vesicles Redefines Performance of miRNA Biomarkers for Non-Alcoholic Fatty Liver Disease. Biomedicines. 2022 Jan. 17; 10 (1): 195. doi: 10.3390/biomedicines 10010195. PMID: 35052873; PMCID: PMC8773667.
80 Chiasserini D, van Wecring J R, Piersma S R, Pham T V, Malekzadch A, Teunissen C E, de Wit H, Jiménez CR. Proteomic analysis of cerebrospinal fluid extracellular vesicles: a comprehensive dataset. J Protcomics. 2014 Jun. 25; 106:191-204. doi: 10.1016/j.jprot.2014.04.028. Epub 2014 Apr. 24. PMID: 24769233.

81. Choudhary I, Vo T, Paudel K, Wen X, Gupta R, Kesimer M, Patial S, Saini Y. Vesicular and extravesicular protein analyses from the airspaces of ozone-exposed mice revealed signatures associated with mucoinflammatory lung disease. Sci Rep. 2021 Dec. 1; 11 (1): 23203. doi: 10.1038/s41598-021-02256-5. PMID: 34853335; PMCID: PMC8636509.
82. Mimmi S, Zimbo A M, Rotundo S, Cione E, Nisticò N, Aloisio A, Maisano D, Tolomeo A M, Dattilo V, Lionello R, Fioravanti A, Di Loria A, Quirino A, Marascio N, Russo A, Trecarichi E M, Matera G, Quinto I, Torti C, Iaccino E. SARS COV-2 spike protein-guided exosome isolation facilitates detection of potential miRNA biomarkers in COVID-19 infections. Clin Chem Lab Med. 2023 Mar. 27; 61 (8): 1518-1524. doi: 10.1515/cclm-2022-1286. PMID: 36972680.
83. Miranda J, Paules C, Nair S, Lai A, Palma C, Scholz-Romero K, Rice G E, Gratacos E, Crispi F, Salomon C. Placental exosomes profile in maternal and fetal circulation in intrauterine growth restriction-Liquid biopsies to monitoring fetal growth. Placenta. 2018 April; 64:34-43. doi: 10.1016/j.placenta.2018.02.006. Epub 2018 Mar. 2. PMID: 29626979.
84 Than U T T, Guanzon D, Broadbent J A, Parker T J, Leavesley D I. Decp Sequencing MicroRNAs from Extracellular Membrane Vesicles Revealed the Association of the Vesicle Cargo with Cellular Origin. Int J Mol Sci. 2020 Feb. 8; 21 (3): 1141. doi: 10.3390/ijms21031141. PMID: 32046334; PMCID: PMC7036882.
85. Yu H, Guan Z, Cuk K, Brenner H, Zhang Y. Circulating microRNA biomarkers for lung cancer detection in Western populations. Cancer Med. 2018 October; 7 (10): 4849-4862. doi: 10.1002/cam4.1782. Epub 2018 Scp 27. PMID: 30259714; PMCID: PMC6198213.
86. Asakura K, Kadota T, Matsuzaki J, Yoshida Y, Yamamoto Y, Nakagawa K, Takizawa S, Aoki Y, Nakamura E, Miura J, Sakamoto H, Kato K, Watanabe S I, Ochiya T. A miRNA-based diagnostic model predicts resectable lung cancer in humans with high accuracy. Commun Biol. 2020 Mar. 19; 3 (1): 134. doi: 10.1038/s42003-020-0863-y. PMID: 32193503; PMCID: PMC7081195.
87. Vykoukal J, Fahrmann J F, Patel N, Shimizu M, Ostrin E J, Dennison J B, Ivan C, Goodman G E, Thornquist M D, Barnett M J, Feng Z, Calin G A, Hanash S M. Contributions of Circulating microRNAs for Early Detection of Lung Cancer. Cancers (Basel). 2022 Aug. 30; 14 (17): 4221. doi: 10.3390/cancers14174221. PMID: 36077759; PMCID: PMC9454665.
88. Wang X, Tian L, Lu J, Ng I O. Exosomes and cancer-Diagnostic and prognostic biomarkers and therapeutic vehicle. Oncogenesis. 2022 Sep. 15; 11 (1): 54. doi: 10.1038/s41389-022-00431-5. PMID: 36109501; PMCID: PMC9477829.
89. He X, Park S, Chen Y, Lee H. Extracellular Vesicle-Associated miRNAs as a Biomarker for Lung Cancer in Liquid Biopsy. Front Mol Biosci. 2021 Feb. 24; 8:630718. doi: 10.3389/fmolb.2021.630718. PMID: 33718435; PMCID: PMC7943919.
90. Zhang C, Yang J, Chen Y, Jiang F, Liao H, Liu X, Wang Y, Kong G, Zhang X, Li J, Gao J, Shen L. miRNAs derived from plasma small extracellular vesicles predict organo-tropic metastasis of gastric cancer. Gastric Cancer. 2022 March; 25 (2): 360-374. doi: 10.1007/s10120-021-01267-5. Epub 2022 Jan. 15. PMID: 35031872.
91. Geng N, Qi Y, Qin W, Li S, Jin H, Jiang Y, Wang X, Wei S, Wang P. Two microRNAs of plasma-derived small extracellular vesicles as biomarkers for metastatic non-small cell lung cancer. BMC Pulm Med. 2023 Jul. 14; 23 (1): 259. doi: 10.1186/s12890-023-02538-w. PMID: 37452310; PMCID: PMC10347730.
92. Tanaka R, Yoshinouchi S, Karouji K, Tanaka Y, Tominari T, Hirata M, Matsumoto C, Itoh Y, Miyaura C, Inada M. A mouse model of lung cancer induced via intranasal injection for anticancer drug screening and evaluation of pathology. FEBS Open Bio. 2023 January; 13 (1): 51-59. doi: 10.1002/2211-5463.13486. Epub 2022 Nov. 29. PMID: 36102619; PMCID: PMC9810119.
93. Kang Y, Omura M, Suzuki A, Thecraladanon C, Oka T, Nakagami Y, Suzuki A, Nagashima Y, Inoue T. Proliferation of human lung cancer in an orthotopic transplantation mouse model. Exp Ther Med. 2010 May; 1 (3): 471-475. doi: 10.3892/ctm_00000073. Epub 2010 May 1. PMID: 22993563; PMCID: PMC3445926.
94. Madero-Visbal R A, Colon J F, Hernandez I C, Limaye A, Smith J, Lec C M, Arlen P A, Herrera L, Baker C H. Bioluminescence imaging correlates with tumor progression in an orthotopic mouse model of lung cancer. Surg Oncol. 2012 March; 21 (1): 23-9. doi: 10.1016/j.suronc.2010.07.008. Epub 2010 Aug. 30. PMID: 20801643.
95 Stevens L E, Arnal-Estapé A, Nguyen D X. Pre-Conditioning the Airways of Mice with Bleomycin Increases the Efficiency of Orthotopic Lung Cancer Cell Engraftment. J Vis Exp. 2018 Jun. 28; (136): 56650. doi: 10.3791/56650. PMID: 30010648; PMCID: PMC6102009.
96. Liu Y, Hou Y, Hua Y, Gao Y. A device to collect exhaled breath to study biomarkers in small animal models. bioRxiv 511931; doi: doi.org/10.1101/511931.
97. Mitchell M I, Ben-Dov I Z, Liu C, Ye K, Chow K, Kramer Y, Gangadharan A, Park S, Fitzgerald S, Ramnauth A, Perlin D S, Donato M, Bhoy E, Manouchehri Doulabi E, Poulos M, Kamali-Moghaddam M, Loudig O. Extracellular Vesicle Capture by AnTibody of CHoice and Enzymatic Release (EV-CATCHER): A customizable purification assay designed for small-RNA biomarker identification and evaluation of circulating small-EVs. J Extracell Vesicles. 2021 June; 10 (8):e12110. doi: 10.1002/jev2.12110. Epub 2021 Jun. 3. PMID: 34122779; PMCID: PMC8173589.
98. Loudig O, Liu C, Rohan T, Ben-Dov I Z. Retrospective MicroRNA Sequencing: Complementary DNA Library Preparation Protocol Using Formalin-fixed Paraffin-embedded RNA Specimens. J Vis Exp. 2018 May 5; (135): 57471. doi: 10.3791/57471. PMID: 29781987; PMCID: PMC6101110.
99. Mensà E, Giuliani A, Matacchione G, Gurău F, Bonfigli A R, Romagnoli F, De Luca M, Sabbatinelli J, Olivieri F. Circulating miR-146a in healthy aging and type 2 diabetes: Age- and gender-specific trajectories. Mech Ageing Dev. 2019 June; 180:1-10. doi: 10.1016/j.mad.2019.03.001. Epub 2019 Mar. 14. PMID: 30880174.

100. Wu C, Zhou S, Mitchell M I, Hou C, Byers S, Loudig O, Ma J. Coupling suspension trapping-based sample preparation and data-independent acquisition mass spectrometry for sensitive exosomal proteomic analysis. Anal Bioanal Chem. 2022 March; 414 (8): 2585-2595. doi: 10.1007/s00216-022-03920-z. Epub 2022 Feb. 18. PMID: 35181835; PMCID: PMC9101639.
101. Minn A J, Gupta G P, Siegel P M, Bos P D, Shu W, Giri D D, Viale A, Olshen A B, Gerald W L, Massagué J. Genes that mediate breast cancer metastasis to lung. Nature. 2005 Jul. 28; 436 (7050): 518-24. doi: 10.1038/nature03799. PMID: 16049480; PMCID: PMC1283098.
102. Agiostratidou G, Li M, Suyama K, Badano I, Keren R, Chung S, Anzovino A, Hulit J, Qian B, Bouzahzah B, Eugenin E, Loudig O, Phillips G R, Locker J, Hazan R B. Loss of retinal cadherin facilitates mammary tumor progression and metastasis. Cancer Res. 2009 Jun. 15; 69 (12): 5030-8. doi: 10.1158/0008-5472.CAN-08-4007. Epub 2009 Jun. 2. PMID: 19491271; PMCID: PMC4382672.
103. Kim S, Yao J, Suyama K, Qian X, Qian B Z, Bandyopadhyay S, Loudig O, De Leon-Rodriguez C, Zhou Z N, Segall J, Macian F, Norton L, Hazan R B. Slug promotes survival during metastasis through suppression of Puma-mediated apoptosis. Cancer Res. 2014 Jul. 15; 74 (14): 3695-706. doi: 10.1158/0008-5472.CAN-13-2591. Epub 2014 May 15. PMID: 24830722; PMCID: PMC4437462.
104. Fan J, Xu G, Chang Z, Zhu L, Yao J. miR-210 transferred by lung cancer cell-derived exosomes may act as proangiogenic factor in cancer-associated fibroblasts by modulating JAK2/STAT3 pathway. Clin Sci (Lond). 2020 Apr. 17; 134 (7): 807-825. doi: 10.1042/CS20200039. Erratum in: Clin Sci (Lond). 2020 Jul. 17; 134 (13): 1801-1804. PMID: 32219336.
105. Ding J, Xu Z, Zhang Y, Tan C, Hu W, Wang M, Xu Y, Tang J. Exosome-mediated miR-222 transferring: An insight into NF-κB-mediated breast cancer metastasis. Exp Cell Res. 2018 Aug. 1; 369 (1): 129-138. doi: 10.1016/j.yexcr.2018.05.014. Epub 2018 May 17. PMID: 29778754.
106. Perez S M, Brinton L T, Kelly K A. Plectin in Cancer: From Biomarker to Therapeutic Target. Cells. 2021 Aug. 30; 10 (9): 2246. doi: 10.3390/cells 10092246. PMID: 34571895; PMCID: PMC8469460.
107. Fei F, Qu J, Zhang M, Li Y, Zhang S. S100A4 in cancer progression and metastasis: A systematic review. Oncotarget. 2017 May 19; 8 (42): 73219-73239. doi: 10.18632/oncotarget.18016. PMID: 29069865; PMCID: PMC5641208.

108. Mitchell M I, Ben-Dov I Z, Ye K, Liu C, Shi M, Sadoughi A, Shah C, Siddiqui T, Okorozo A, Gutierrez M, Unawane R, Biamonte L, Parihk K, Spivack S, Loudig O. Exhaled breath condensate contains extracellular vesicles that carry surrogate miRNA biomarker cargo of bronchoalveolar epithelial cells from the deep lung.
109. Jouida A, O'Callaghan M, Mc Carthy C, Fabre A, Nadarajan P, Keane M P. Exosomes from EGFR-Mutated Adenocarcinoma Induce a Hybrid E M T and MMP9-Dependant Tumor Invasion. Cancers (Basel). 2022 Aug. 3; 14 (15): 3776. doi: 10.3390/cancers14153776. PMID: 35954442; PMCID: PMC9367273.
110. Tan A C. Targeting the PI3K/Akt/mTOR pathway in non-small cell lung cancer (NSCLC). Thorac Cancer. 2020 March; 11 (3): 511-518. doi: 10.1111/1759-7714.13328. Epub 2020 Jan. 27. PMID: 31989769; PMCID: PMC7049515.
111. Fumarola C, Bonelli M A, Petronini P G, Alfieri R R. Targeting P I3K/AKT/mTOR pathway in non small cell lung cancer. Biochem Pharmacol. 2014 Aug. 1; 90 (3): 197-207. doi: 10.1016/j.bcp.2014.05.011. Epub 2014 May 24. PMID: 24863259.
112. Aredo J V, Padda S K, Kunder C A, Han S S, Neal J W, Shrager J B, Wakelec H A. Impact of KRAS mutation subtype and concurrent pathogenic mutations on non-small cell lung cancer outcomes. Lung Cancer. 2019 July; 133:144-150. doi: 10.1016/j.lungcan.2019.05.015. Epub 2019 May 15. PMID: 31200821; PMCID: PMC9348589.
113. Li K, Liu Y, Ding Y, Zhang Z, Feng J, Hu J, Chen J, Lian Z, Chen Y, Hu K, Chen Z, Cai Z, Liu M, Pang X. BCL6 is regulated by the MAPK/ELKI axis and promotes KRAS-driven lung cancer. J Clin Invest. 2022 Nov. 15; 132 (22):e161308. doi: 10.1172/JCI161308. PMID: 36377663; PMCID: PMC9663163.
114. Sun T, Zhang J, Deng B, Fan X, Long T, Jin H, Tao S, Kang P, Tan Q. FOXO1 and FOXO3a sensitize non-small-cell lung cancer cells to cisplatin-induced apoptosis independent of Bim. Acta Biochim Biophys Sin (Shanghai). 2020 Dec. 29; 52 (12): 1348-1359. doi: 10.1093/abbs/gmaa129. PMID: 33167006.
115. Jiramongkol Y, Lam E W. FOXO transcription factor family in cancer and metastasis. Cancer Metastasis Rev. 2020 September; 39 (3): 681-709. doi: 10.1007/s10555-020-09883-w. PMID: 32372224; PMCID: PMC7497309.
116. Ebrahimnezhad M, Natami M, Bakhtiari G H, Tabnak P, Ebrahimnezhad N, Yousefi B, Majidinia M. FOXO1, a tiny protein with intricate interactions: Promising therapeutic candidate in lung cancer. Biomed Pharmacother. 2023 Dec. 31; 169:115900. doi: 10.1016/j.biopha.2023.115900. Epub 2023 Nov. 18. PMID: 37981461.
117. Pan J, Cai X, Zheng X, Zhu X, Feng J, Wang X. Luteolin inhibits viability, migration, angiogenesis and invasion of non-small cell lung cancer vascular endothelial cells via miR-133a-3p/purine rich element binding protein B-mediated MAPK and PI3K/Akt signaling pathways. Tissue Cell. 2022 April; 75:101740. doi: 10.1016/j.tice.2022.101740. Epub 2022 Jan. 20. PMID: 35101688.
118. Stutvoet T S, Kol A, de Vries E G, de Bruyn M, Fehrmann R S, Terwisscha van Scheltinga A G, de Jong S. MAPK pathway activity plays a key role in PD-L1 expression of lung adenocarcinoma cells. J Pathol. 2019 September; 249 (1): 52-64. doi: 10.1002/path.5280. Epub 2019 May 21. PMID: 30972766; PMCID: PMC6767771.
119. Serganova I, Moroz E, Vider J, Gogiberidze G, Moroz M, Pillarsetty N, Doubrovin M, Minn A, Thaler H T, Massague J, Gelovani J, Blasberg R. Multimodality imaging of TGFbeta signaling in breast cancer metastases. FASEB J. 2009 August; 23 (8): 2662-72. doi: 10.1096/fj.08-126920. Epub 2009 Mar. 26. PMID: 19325038; PMCID: PMC2717767.
120. Jenkins D E, Hornig Y S, Oci Y, Dusich J, Purchio T. Bioluminescent human breast cancer cell lines that permit rapid and sensitive in vivo detection of mammary tumors and multiple metastases in immune deficient mice. Breast Cancer Res. 2005; 7 (4): R444-54. doi: 10.1186/bcr1026. Epub 2005 Apr. 8. PMID: 15987449; PMCID: PMC1175057.
121. Stachowiak Z, Wojsyk-Banaszak I, Jończyk-Potoczna K, Narożna B, Langwiński W, Szczepankiewicz A. Extracellular vesicles-derived miRNAs as mediators of pulmonary exacerbation in pediatric cystic fibrosis. J Breath Res. 2023 Feb. 10; 17 (2). doi: 10.1088/1752-7163/acb792. PMID: 36720158.
122. An J, McDowell A, Kim Y K, Kim T B. Extracellular vesicle-derived microbiome obtained from exhaled breath condensate in patients with asthma. Ann Allergy Asthma Immunol. 2021 June; 126 (6): 729-731. doi: 10.1016/j.anai.2021.02.030. Epub 2021 Mar. 10. PMID: 33713805.
123. Chiappetta M, Salvatore L, Congedo M T, Bensi M, De Luca V, Petracca Ciavarella L, Camarda F, Evangelista J, Valentini V, Tortora G, Margaritora S, Lococo F. Management of single pulmonary metastases from colorectal cancer: State of the art. World J Gastrointest Oncol. 2022 Apr. 15; 14 (4): 820-832. doi: 10.4251/wjgo.v14.i4.820. PMID: 35582100; PMCID: PMC9048528.
124. Jin L, Han B, Siegel E, Cui Y, Giuliano A, Cui X. Breast cancer lung metastasis: Molecular biology and therapeutic implications. Cancer Biol Ther. 2018; 19 (10): 858-868. doi: 10.1080/15384047.2018.1456599. Epub 2018 Apr. 30. PMID: 29580128; PMCID: PMC6300341.
125. Tyagi A, Sharma S, Wu K, Wu S Y, Xing F, Liu Y, Zhao D, Deshpande R P, D'Agostino R B Jr, Watabe K. Nicotine promotes breast cancer metastasis by stimulating N2 neutrophils and generating pre-metastatic niche in lung. Nat Commun. 2021 Jan. 20; 12 (1): 474. doi: 10.1038/s41467-020-20733-9. PMID: 33473115; PMCID: PMC7817836.
126. Patel H D, Singla N, Ghandour R A, Freifeld Y, Cheaib J G, Woldu S L, Pierorazio P M, Bagrodia A. Site of extranodal metastasis impacts survival in patients with testicular germ cell tumors. Cancer. 2019 Nov. 15; 125 (22): 3947-3952. doi: 10.1002/cncr.32427. Epub 2019 Jul. 29. PMID: 31355922; PMCID: PMC6819223.
127. Zhang J, Kulkarni H R, Singh A, Baum R P. Complete Regression of Lung Metastases in a Patient With Metastatic Castration-Resistant Prostate Cancer Using 177Lu-PSMA Radioligand Therapy. Clin Nucl Med. 2020 January; 45 (1):e48-c50. doi: 10.1097/RLU.0000000000002655. PMID: 31162261.
128. Gardner A B, Charo L M, Mann A K, Kapp D S, Eskander R N, Chan J K. Ovarian, uterine, and cervical cancer patients with distant metastases at diagnosis: most common locations and outcomes. Clin Exp Metastasis. 2020 February; 37 (1): 107-113. doi: 10.1007/s10585-019-10007-0. Epub 2019 Nov. 22. PMID: 31758289.
129. Micalizzi D S, Maheswaran S, Haber D A. A conduit to metastasis: circulating tumor cell biology. Genes Dev. 2017 Sep. 15; 31 (18): 1827-1840. doi: 10.1101/gad.305805.117. PMID: 29051388; PMCID: PMC5695084.
130. Lin D, Shen L, Luo M, Zhang K, Li J, Yang Q, Zhu F, Zhou D, Zheng S, Chen Y, Zhou J. Circulating tumor cells: biology and clinical significance. Signal Transduct Target Ther. 2021 Nov. 22; 6 (1): 404. doi: 10.1038/s41392-021-00817-8. PMID: 34803167; PMCID: PMC8606574.
131. Castro-Giner F, Aceto N. Tracking cancer progression: from circulating tumor cells to metastasis. Genome Med. 2020 Mar. 19; 12 (1): 31. doi: 10.1186/s13073-020-00728-3. PMID: 32192534; PMCID: PMC7082968.
132. Gkountela S, Castro-Giner F, Szczerba B M, Vetter M, Landin J, Scherrer R, Krol I, Scheidmann M C, Beisel C, Stirnimann C U, Kurzeder C, Heinzelmann-Schwarz V, Rochlitz C, Weber W P, Accto N. Circulating Tumor Cell Clustering Shapes DNA Methylation to Enable Metastasis Seeding. Cell. 2019 Jan. 10; 176 (1-2): 98-112.c14. doi: 10.1016/j.cell.2018.11.046. PMID: 30633912; PMCID: PMC6363966.
133. Tayoun T, Faugeroux V, Oulhen M, Aberlenc A, Pawlikowska P, Farace F. CTC-Derived Models: A Window into the Seeding Capacity of Circulating Tumor Cells (CTCs). Cells. 2019 Scp 25; 8 (10): 1145. doi: 10.3390/cells8101145. PMID: 31557946; PMCID: PMC6829286.
134. Abdolahi S, Ghazvinian Z, Muhammadnejad S, Salch M, Asadzadeh Aghdaci H, Baghaci K. Patient-derived xenograft (PDX) models, applications and challenges in cancer research. J Transl Med. 2022 May 10; 20 (1): 206. doi: 10.1186/s12967-022-03405-8. PMID: 35538576; PMCID: PMC9088152.
135. Drosten M, Guerra C, Barbacid M. Genetically Engineered Mouse Models of K-Ras-Driven Lung and Pancreatic Tumors: Validation of Therapeutic Targets. Cold Spring Harb Perspect Med. 2018 May 1; 8 (5): a031542. doi: 10.1101/cshperspect.a031542. PMID: 28778964; PMCID: PMC5932580.
136. Fitzgerald B, Connolly K A, Cui C, Fagerberg E, Mariuzza D L, Hornick N I, Foster G G, William I, Cheung J F, Joshi N S. A mouse model for the study of anti-tumor T cell responses in Kras-driven lung adenocarcinoma. Cell Rep Methods. 2021 Sep. 27; 1 (5): 100080. doi: 10.1016/j.crmeth.2021.100080. Epub 2021 Sep. 16. PMID: 34632444; PMCID: PMC8500377.
137. Foggetti G, Li C, Cai H, Hellyer J A, Lin W Y, Ayeni D, Hastings K, Choi J, Wurtz A, Andrejka L, Maghini D G, Rashleigh N, Levy S, Homer R, Gettinger S N, Dichn M, Wakelee H A, Petrov D A, Winslow M M, Politi K. Genetic Determinants of EGFR-Driven Lung Cancer Growth and Therapeutic Response In Vivo. Cancer Discov. 2021 July; 11 (7): 1736-1753. doi: 10.1158/2159-8290.CD-20-1385. Epub 2021 Mar. 11. PMID: 33707235; PMCID: PMC8530463.
138. Politi K, Fan P D, Shen R, Zakowski M, Varmus H. Erlotinib resistance in mouse models of epidermal growth factor receptor-induced lung adenocarcinoma. Dis Model Mech. 2010 January-February; 3 (1-2): 111-9. doi: 10.1242/dmm.003681. Epub 2009 Dec. 9. PMID: 20007486; PMCID: PMC2806903.
139. Ramelow J, Brooks C D, Gao L, Almiman A A, Williams™, Villalona-Calero M A, Duan W. The oncogenic potential of a mutant TP53 gene explored in two spontaneous lung cancer mice models. BMC Cancer. 2020 Aug. 8; 20 (1): 738. doi: 10.1186/s12885-020-07212-6. PMID: 32770960; PMCID: PMC7414707.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A non-invasive method for targeted capture of a purified population of extracellular vesicles (EVs) derived from lung cancer cellsexhaled breath obtained from a subject at risk for lung cancer for evaluating a cargo of the purified population of EVs comprising:
a) obtaining an expressed breath sample from the subject;
b) condensing the exhaled breath sample in a cooling chamber and collecting the exhaled breath condensate (EBC);
c) preparing a purified population of EVs by contacting the exhaled breath condensate comprising EVs from the subject with a binding agent directed to one or more EV surface antigen; wherein the binding agent is linked to a nucleic acid, and wherein the nucleic acid is immobilized on a solid support;
d) isolating the EV bound by the binding agent from the exhaled breath condensate;
e) releasing the EV bound to the binding agent;
f) eluting the bound EV from the binding agent to form a population of free purified EVs; and
g) evaluating cargo and surface molecules comprising protein, nucleic acids or lipids, of the purified population of EVs.

2. The non-invasive method according to claim 1,
a) wherein the subject is a primary lung tumor bearing animal model wherein the lung tumor is a human tumor comprising mutations including EGFR, KRAS, p53, Ret, Her2, ROS1, Met, BRAF, NRAS, ALK, MAP2K1 or PI3KCA mutations and control animal colonies are established with primary human small airway epithelial cells (HSAECs); or
b) wherein the animal model is a transgenic mouse model, and human tumor cells comprising mutations including EGFR, KRAS, p53, Ret, Her2, ROS1, Met, BRAF, NRAS, ALK, MAP2K1 or PI3KCA mutations and a bioluminescent construct are instilled through the trachea of the animals; or
c) wherein the animal model is a TA-CCSP transgenic model, wherein the CCSP promoter is active in Clara cells, in alveolar type II cells or both; or
d) wherein the animal model is an orthotopic human tumor NOD/SCID mouse model bearing a human lung tumor comprising mutations including EGFR, KRAS, p53, Ret, Her2, ROS1, Met, BRAF, NRAS, ALK, MAP2K1 or PI3KCA mutations, and wherein human NSCLC lung cancer cell lines are transduced for ex vivo bioluminescence, expanded in vitro and administered into the lungs of the NOD/SCID nude mice by tracheal instillation for lung uptake, or
d) wherein the animal model is a PDX human tumor NOD/SCID mouse model established using NSCLC patient-derived tumor cells comprising mutations including EGFR, KRAS, p53, Ret, Her2, ROS1, Met, BRAF, NRAS, ALK, MAP2K1 or PI3KCA mutations, and
wherein the transgenic model, the orthotopic model and the PDX model are complementary.

3. The non-invasive method according to claim 2, wherein
a. the transgenic mouse is transduced to express EGFRL858R protein in bronchiolar Clara cells; or
b. the transgenic mouse is transfected to express KRASG12D protein in Clara cells and alveolar type II cells; or
c. the transgenic mouse is transfected to express CC10 protein for the study of multifocal bronchioloalveolar hyperplasias which develop into mixed solid and papillary adenocarcinomas, adenocarcinomas with focal NE differentiation, epithelial cell hyperplasia and adenomatous hyperplasia and bronchogenic adenocarcinomas; or
d. the transgenic mouse is transfected to express SP-C protein for the study of bronchioloalveolar adenomas and adenocarcinomas.e. the transgenic mouse is transfected to express Trp5 for the study of adenocarcinomas, NE hyperplasia and small-cell carcinoma with metastases; or
f. the transgenic mouse is transfected to express Rb for the study of NE hyperplasia and small-cell carcinoma with metastases.

4. The non-invasive method according to claim 1, step (g) evaluating cargo and surface molecules further comprising one or more of:
(i) identifying proteins specific to a surface of the biological particles; or
(ii) identifying protein cargos; or
(iii) identifying DNA molecules; or
(iv) extracting RNA from the purified population of EVs, and identifying and quantifying expression of small non-coding RNAs comprising microRNAs (miRNAs) encapsulated by the purified population of EVs.

5. The non-invasive method according to claim 4, wherein
the proteins are identified by mass spectrometry;
the DNA is identified by sequencing or quantitative PCR; and
the RNA is identified by digital drop PCR.

6. The non-invasive method according to claim 1, comprising an initial ultrafiltration or ultracentrifugation step to provide a starting pooled heterogeneous population of EVs.

7. The non-invasive method according to claim 1, wherein the binding agent that binds to one or more EV surface antigen is an antibody, an antibody binding fragment, or an aptamer.

8. The non-invasive method according to claim 7, wherein
(a) the aptamer comprises two complementary primers including 5′-Azide (5′Az-AAAAACGAUUCGAGAACGUGACUGCCAUGCCAGCUCGUACUAU CGAA (SEQ ID NO: 1)) and 3′-Biotin (5′Bio-CGAUAGUACGAGCUGGCAUGGCAGUCACGUUCUCGAA UCGUUUU (SEQ ID NO: 2)); or
(b) the aptomer comprises two complementary primers containing specific restriction enzyme recognition sites used for EV-CATCHER including:
BamHI: 5'-Azide (5'Az-
AAAAACGATTCGAGAACGTGAATCTCGTTAACCGCTCAACTGGATCCCC
AGCTCGTACTCCGCGATTCGTGCTCCGTACTCCAATC (SEQ ID NO:
120)) and
BamHI: 3'-Biotin (5'Bio-
CGATTGGAGTACGGAGCACGAATCGCCGAGTACGAGCTGGGGATCCAGT
TGAGCGGTTAACGAGATTCACGTTCTCGAATCGTTT (SEQ ID NO:
121));
HindIII: 5'-Azide
(5'Az- AAAAACGATTCGAGAACGTGAATCTCGTTAACCGCTCAACTA
AGCTTCCAGCTCGTACTCCGCGATTCGTGCTCCGTACTCCAATC (SEQ
ID NO: 122)) and
HindIII: 3'-Biotin (5'Bio-
CGATTGGAGTACGGAGCACGAATCGCCGAGTACGAGCTGGAAGCTTAGT
TGAGCGGTTAACGAGATTCACGTTCTCGAATCGTTT (SEQ ID NO:
123)); or
SpeI: 5'-Azide
(5'Az- AAAAACGATTCGAGAACGTGAATCTCGTTAACCGCTCAACTA
CTAGTCCAGCTCGTACTCCGCGATTCGTGCTCCGTACTCCAATC (SEQ
ID NO: 124)) and
SpeI: 3'-Biotin (5'Bio-
CGATTGGAGTACGGAGCACGAATCGCCGAGTACGAGCTGGACTAGTAGT
TGAGCGGTTAACGAGATTCACGTTCTCGAATCGTTT (SEQ ID NO:
125)).

9. The non-invasive method according to claim 1, wherein the EVsurface antigen comprises CD9, CD63, CD81, CD37, CD82, Alix, Tim4, PLAP, Adiponectin, FABP4, Caveolin-1, Cytokeratins, EPCAM, E-Cadherin, P63, a heterologous cell surface polypeptide, a cell surface marker inherited by the EVs, club cell secretory protein (CCSP), a SFTPC-encoded surface protein or a variant thereof.

10. The non-invasive method according to claim 9, wherein the EV surface antigen is specific to Clara cells or AT2 respiratory cells.

11. The non-invasive method according to claim 10, wherein the EV surface antigen is a club cell secretory protein (CCSP) variant or surfactant protein C (SP-C) variant encoded by the SFTPC gene.

12. The non-invasive method according to claim 1, wherein the nucleic acid comprises DNA, RNA, or a combination thereof.

13. The non-invasive method according to claim 12, wherein the nucleic acid comprises non-natural nucleotides.

14. The non-invasive method according to claim 12, wherein the nucleic acid comprises DNA.

15. The non-invasive method according to claim 12, wherein the DNA comprises a restriction enzyme recognition site.

16. The non-invasive method according to claim 12, wherein the DNA comprises one or more ribonucleic acid nucleotide.

17. The non-invasive method according to claim 12, wherein the one or more ribonucleic acid nucleotide is uracil.

18. The non-invasive method according to claim 12, wherein the nucleic acid further comprises a binding moiety on a first end of the nucleic acid and a binding moiety on a second end of the nucleic acid, and wherein the binding moiety on the first end of the nucleic acid and the binding moiety on the second end of the nucleic acid are different.

19. The non-invasive method according to claim 18, wherein the binding moiety on the first end of the nucleic acid is an avidin, streptavidin or carboxyl binding moiety.

20. The non-invasive method according to claim 18, wherein the binding moiety is biotin.

21. The non-invasive method according to claim 18, wherein the binding moiety on the second end of the nucleic acid is an amine moiety.

22. The non-invasive method according to claim 21, wherein the amine moiety is azide.

23. The non-invasive method according to claim 1, wherein the binding agent to one or more EV surface antigens comprises a dibenzocyclooctyne (DBCO) molecule, 2-IT (2-iminothiolane), MBS (3-maleimidobenzoic acid N-hydroxysuccinimide ester), SPDP (N-succinimidyl 3-(2-pyridyldithio) propionate), SATA (N-succinimidyl S-acetylthioacetate), SMCC (succinimidyl 4-(N-maleimidomethyl) cyclohexane-1-carboxylate), Sulfo-SMCC, or derivatives thereof.

24. The non-invasive method according to claim 1, wherein the solid support is a well plate, polymer, or a surface.

25. The non-invasive method according to claim 1, wherein releasing the isolated EV comprises:
(i) enzymatically cleaving the nucleic acid; or
(ii) displacing a first strand of the nucleic acids connected to the antibody from the second strand of the nucleic acids connected to the support by strand displacement with a complementary nucleic acid to the first or second strand of the nucleic acid and an enzyme having strand displacement activity to release the antibody from the support; or
(iii) separating the annealed DNA strands to allow release of the antibody from the platform without damaging the DNA strand attached to the antibody by a polymerase chain reaction using an oligonucleotide complementary to the region of the DNA attached to the antibody.

26. The non-invasive method according to claim 25,
a) wherein the enzymatic cleaving is with uracil glycosylase; or
b) wherein the enzymatic cleaving is with a restriction enzyme; or
c) wherein the enzyme having strand displacement activity is DNA polymerase, topoisomerase, or helicase.

27. The non-invasive method according to claim 1 comprising detecting, identifying and measuring a level of mRNA or the one or more small non-coding RNAs comprising miRNAs encapsulated in the EVs by next generation sequencing.

28. The non-invasive method according to claim 27, wherein the miRNA encapsulated in the EVs is one or more miRNA listed in Table 5 or in Tables 8-11.

29. A non-invasive method for optimizing therapeutic benefit for a subject at risk of lung cancer, comprising
a) obtaining an exhaled breath sample from the subject and from a healthy control;
b) condensing the exhaled breath in a cooling chamber and collecting the exhaled breath condensate (EBC);
c) purifying EVs derived from the exhaled breath sample contained in the EBC obtained from the subject and the healthy control;
d) measuring a level of expression of each of a plurality of mRNAs, miRNAs or protein cargo in the EVs contained in the EBC sample from the subject and in the EVs contained in the EBC sample from the healthy control;
e) determining that expression of the one or more of the mRNAs, miRNAs or protein cargo in the EVs contained in the EBC sample from the subject is dysregulated compared to the healthy control;
f) identifying the patient as one that can benefit therapeutically from being treated for lung cancer, when the presence of one or more dysregulated mRNAs, miRNAs or protein cargo in the EVs contained in the EBC sample obtained from the subject is detected; wherein the detection may correlate with tumor burden; and
g) tailoring an effective medical treatment for the lung cancer based on genetic, environmental and lifestyle factors of the subject and based on the detection in (f).

30. The non-invasive method according to claim 29, further comprising:
(h) monitoring the lung cancer response or resistance to the treatment in (g) by obtaining exhaled breath samples comprising EVs from the subject over time; and
(i) adjusting the medical treatment as needed to improve clinical outcome.

31. The non-invasive method according to claim 29, wherein the miRNA encapsulated by the EVs is one or more miRNA listed in Table 5 or in tables 8-11.

32. The non-invasive method according to claim 29, wherein the one or more miRNAs is downregulated compared to the healthy control.

33. The non-invasive method according to claim 29, wherein the one or more miRNAs is upregulated compared to the healthy control.

34. The non-invasive method according to claim 29,
a) wherein the subject is a mammalian subject; or
b) wherein the subject is a human subject.

35. The non-invasive method according to claim 29, wherein the detecting in (e) is earlier than detecting of lung cancer by imaging thresholds.

36. The non-invasive method according to claim 29, the method further comprising detecting a level of expression of a protein in the EVs from the EBC sample and determining that expression of the protein is dysregulated compared to the healthy control, wherein the protein includes α-enolase, vimentin, brain abundant membrane attached signal protein 1 (BASP1), aldolase (ALDOA), calreticulin (CALR), proteasome activator subunit 1 (PSME1), proteasome activator subunit (2), major histocompatibility complex class 1C (HLA-C) or glucose 6-phosphate dehydrogenase (G6PD), SH3 domain-containing protein 21, Arf-GAP with SH3 domain, ANK repeat and PH domain-containing protein 2, Histone H4, Vimentin, AHNAK nucleoprotein (desmoyokin), Heat shock protein HSP 90-beta, Annexin A5, Protein S100-A4, Heat shock protein HSP 90-alpha, Alpha-enolase, High mobility group protein HMG-I/HMG-Y, 14-3-3 protein zeta/delta, Hepatoma-derived growth factor, Gelsolin, Integrin alpha-3, 14-3-3 protein epsilon, Annexin A3, 14-3-3 protein theta, Proliferation-associated protein 2G4, 60 kDa heat shock protein, mitochondrial, Protein S100-A14, Vinculin, Ras-related protein Rab-7a, Integrin beta-1, Integrin alpha-6, Cytochrome c, somatic, Interleukin enhancer-binding factor 3, Cell division cycle 34B Protein phosphatase 1 regulatory inhibitor subunit 16B (Fragment); Protein phosphatase 1 regulatory inhibitor subunit 16B and Serine/threonine-protein kinase D.

37. The non-invasive method according to claim 29, wherein
the subject at risk is a smoker, a former smoker, or a non-smoker that is chemonaive;
the subject at risk has been treated for lung cancer and is in remission;
the subject at risk is at risk for a recurrence of lung cancer; or
the subject at risk is at risk for progression of lung cancer.

38. A method for detecting lung colonizing cells derived from a primary tumor during early development of a secondary lung cancer comprising:
(a) identifying a unique cancer surface protein derived from the primary tumor in a subject 1 by:
(i) obtaining an exhaled breath sample from the subject with a primary tumor, wherein the primary tumor has not metastasized and from a healthy control;
(ii) condensing the exhaled breath sample in a cooling chamber and collecting the exhaled breath condensate (EBC);
(iii) purifying EVs derived from the primary tumor and contained in the EBC obtained from the subject 1 and the healthy control;
(iv) evaluating a level of expression of miRNAs, mRNAs, surface proteins or a ratio of any two thereof included in the EVs contained in the EBC sample from the subject 1 and in the EVs contained in the EBC sample from the healthy control;
(v) identifying the unique cancer protein derived from the primary tumor in subject 1;
(b) using the unique cancer protein derived from the primary tumor in (a), obtaining an exhaled breath sample from a subject 2, wherein the subject 2 is at risk for a secondary lung tumor derived from the primary tumor in (a);
(c) condensing the exhaled breath from the subject 2 in a cooling chamber and collecting the exhaled breath condensate;
(d) purifying a population of EVs contained in the EBC from the subject 2,
(e) identifying a therapeutic biosignature for the secondary lung cancer in the EVs contained in the EBCs comprising expression of one or more of miRNAs, mRNAs and surface proteins derived from the secondary lung cancer; and
(f) identifying the patient as one that can benefit therapeutically from being treated for the secondary lung cancer at an early stage.

39. The method according to claim 38, wherein
The first subject is an orthotopic animal model and the second subject is an orthotopic model; or
The first subject is a orthotopic animal model and the second subject is a PDX animal model; or
The first subject is a PDX animal model and the second subject is an orthotopic animal model; or
The first subject is a PDX animal model and the second subject is a PDX animal model; and
control animal colonies are established with primary human small airway epithelial cells (HSAECs).

40. The method according to claim 38, wherein
the orthotopic human tumor NOD/SCID mouse model bearing a human lung tumor comprising mutations including EGFR and KRAS mutations, and wherein human NSCLC lung cancer cell lines are transduced for ex vivo bioluminescence, expanded in vitro and administered into the lungs of the NOD/SCID nude mice by tracheal instillation for lung uptake, or
the PDX model is a human tumor NOD/SCID mouse model established using NSCLC patient-derived tumor cells comprising mutations including EGFR and KRAS mutations.

41. The method according to claim 38, wherein the primary tumor is a colorectal cancer, a breast cancer or a bladder cancer.

42. A non-invasive method for targeted capture of a purified population of extracellular vesicles (EVs) derived from cells infected with tuberculosis and contained in exhaled breath obtained from a subject at risk for continued disease with tuberculosis for evaluating a cargo of the purified population of EVs comprising:
a) obtaining an expressed breath sample from the subject;
b) condensing the exhaled breath sample in a cooling chamber and collecting the exhaled breath condensate (EBC);
c) preparing a purified population of EVs by contacting the exhaled breath condensate comprising EVs from the subject with a binding agent directed to one or more EV surface antigen; wherein the binding agent is linked to a nucleic acid, and wherein the nucleic acid is immobilized on a solid support;
d) isolating the EV bound by the binding agent from the exhaled breath condensate;
e) releasing the EV bound to the binding agent;
f) eluting the bound EV from the binding agent to form a population of free purified EVs; and
g) evaluating cargo and surface molecules comprising protein, nucleic acids or lipids, of the purified population of EVs.

43. A non-invasive method for optimizing therapeutic benefit for a subject at risk of continued disease with tuberculosis, comprising
a) obtaining an exhaled breath sample from the subject and from a healthy control;
b) condensing the exhaled breath in a cooling chamber and collecting the exhaled breath condensate (EBC);
c) purifying EVs derived from the exhaled breath sample contained in the EBC obtained from the subject and the healthy control;
d) measuring a level of expression of each of a plurality of mRNAs, miRNAs or protein cargo in the EVs contained in the EBC sample from the subject and in the EVs contained in the EBC sample from the healthy control;
e) determining that expression of the one or more of the mRNAs, miRNAs or protein cargo in the EVs contained in the EBC sample from the subject is dysregulated compared to the healthy control;
f) identifying the patient as one that can benefit therapeutically from being treated for tuberculosis, when the presence of one or more dysregulated mRNAs, miRNAs or protein cargo in the EVs contained in the EBC sample obtained from the subject is detected; wherein the detection may correlate with tuberculosis disease burden; and
g) tailoring an effective medical treatment for the tuberculosis based on genetic, environmental and lifestyle factors of the subject and based on the detection in (f).

44. The non-invasive method according to claim 29, further comprising:
(h) monitoring the tuberculosis response or resistance to the treatment in (g) by obtaining exhaled breath samples comprising EVs from the subject over time; and
(i) adjusting the medical treatment as needed to improve clinical outcome.

45. A single mouse exhaled breath collection device, comprising:
a chamber configured and dimensioned to at least partially receive a mouse therein;
a restrainer ring disposed within the chamber and separating an inner volume of the chamber into a first section and a second section;
wherein the restrainer ring includes (i) a first restriction section having a first diameter dimensioned to receive at least a head of the mouse, and (ii) a second restriction section having an opening with a second diameter dimensioned to only receive a nose of the mouse therethrough, the second diameter dimensioned smaller than the first diameter.

46. The single mouse exhaled breath collection device of claim 45, wherein the first restriction section prevents movement of the mouse within the chamber.

47. The single mouse exhaled breath collection device of claim 45, comprising a first flow pump/meter connected to a first end of the chamber and providing a flow rate of about 20 ml/min into the chamber.

48. The single mouse exhaled breath collection device of claim 47, comprising a second flow pump/meter connected to a second end of the chamber and providing a flow rate of about 2 ml/min to ensure one-way air flow in the chamber.
